SEQUENCE LISTING (190 sequences)

SEQ ID NO: 1 - skipped

SEQ ID NO: 2 - protein, 17 aa, synthetic construct
  VARIANT 8: note = I or V
  VARIANT 13: note = P or Q
  VARIANT 16: note = M or K
LINPYNGXTTYNXKFXG

SEQ ID NO: 3 - protein, 8 aa, synthetic construct
EGNWEGVY

SEQ ID NO: 4 - protein, 16 aa, synthetic construct
  VARIANT 1: note = K or R
XSSQSLLDSNGKTYLH

SEQ ID NO: 5 - protein, 7 aa, synthetic construct
  VARIANT 4: note = I or K
LVSXLDS

SEQ ID NO: 6 - protein, 9 aa, synthetic construct
VQGTHFPFT

SEQ ID NO: 7 - protein, 5 aa, synthetic construct
GFTMN

SEQ ID NO: 8 - protein, 17 aa, synthetic construct
LINPYNGVTTYNQKFKG

SEQ ID NO: 9 - protein, 7 aa, synthetic construct
LVSILDS

SEQ ID NO: 10 - protein, 16 aa, synthetic construct
RSSQSLLDSNGKTYLH

SEQ ID NO: 11 - protein, 16 aa, synthetic construct
KSSQSLLDSNGKTYLH

SEQ ID NO: 12 - protein, 5 aa, synthetic construct
GYTLN

SEQ ID NO: 13 - protein, 17 aa, synthetic construct
LINPYNGITTYNPKFMG

SEQ ID NO: 14 - protein, 7 aa, synthetic construct
LVSKLDS

SEQ ID NO: 15 - protein, 117 aa, synthetic construct
EVQLVQSGAEVVKPGASVRLSCKASGYSFTGFTMNWVRQALGQGLEWMGLINPYNGVTTYNQKFKGRLTMTADMSTRTVYMDLSSLRYEDTAVYYCTREGNWEGVYWGQGTLVTVSS

SEQ ID NO: 16 - protein, 117 aa, synthetic construct
EVQLVQSGAEVKKPGASVRLSCKASGYSFTGFTMNWVRQALGQGLEWMGLINPYNGVTTYNQKFKGRLTMTADMSTRTVYMDLSSLRYEDTAVYYCTREGNWEGVYWGQGTLVTVSS

SEQ ID NO: 17 - protein, 117 aa, synthetic construct
EVQLVQSGAEVKKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSS

SEQ ID NO: 18 - protein, 117 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSS

SEQ ID NO: 19 - protein, 117 aa, synthetic construct
EVQLVQSGAEVVKPGASVRLSCKASGYSFTGFTMNWVRQALGQGLEWMGLINPYNGVTTYNQKFKGRLTMTADMSTRTVYMDLSSLTYEDTAVYYCTREGNWEGVYWGQGTLVTVSS

SEQ ID NO: 20 - protein, 117 aa, synthetic construct
EVQLVQSGAEVKKPGASVRLSCKASGYSFTGFTMNWVRQALGQGLEWMGLINPYNGVTTYNQKFKGRLTMTADMSTRTVYMDLSSLTYEDTAVYYCTREGNWEGVYWGQGTLVTVSS

SEQ ID NO: 21 - protein, 117 aa, synthetic construct
QVQLVQSGPEVKKPGSSVKVSCKASGYSFTGFTMNWVRQTPGKGLEWIGLINPYNGVTTYNQKFKGRVTITADESTRTAYMELLSLTYEDTAVYYCTREGNWEGVYWGQGTPVTVSA

SEQ ID NO: 22 - protein, 117 aa, synthetic construct
EVQLVQSGAEVKKPGASVRLSCKASGYSFTGFTMNWVRQALGQGLEWMGLINPYNGVTTYNQKFKGRLTMTRDMSTRTVYMDLSSLRYEDTAVYYCTREGNWEGVYWGQGTLVTVSS

SEQ ID NO: 23 - protein, 112 aa, synthetic construct
EIVLTQSPLSLSVTLGQSASISCRSSQSLLDSNGKTYLHWFILRPGQSPRRLIYLVSILDSGVPDRFSGSGSGTDFTLKISRVEAEDVGVYYCVQGTHFPFTFGSGTKLEIK

SEQ ID NO: 24 - protein, 112 aa, synthetic construct
EVVLTQSPLSISVTLGQSASISCRSSQSLLDSNGKTYLHWFILRPGQSPRRLIYLVSILDSGIPDRFSGSGSGTDFTLKISRVEAEDVGVYYCVQGTHFPFTFGGGTKLEIK

SEQ ID NO: 25 - protein, 117 aa, synthetic construct
EVQLQQSGPELVKPGTSIKMSCKASGYSFTGFTMNWVKQSHGKNLEWIGLINPYNGVTTYNQKFKGKATITVDKSSRTAYMELLSLTYEDSAVYYCTREGNWEGVYWGQGTPVTVSA

SEQ ID NO: 26 - protein, 112 aa, synthetic construct
EVVLTQTPLTLSVTIGQSASISCKSSQSLLDSNGKTYLHWFLLRPGQSPKRLIYLVSILDSGVPDRFTGSGSGTDFTLKISRVEAEDLGIYYCVQGTHFPFTFGSGTKLEIK

SEQ ID NO: 27 - protein, 117 aa, synthetic construct
KVQLQQSGPELVKPGTSIKMSCKTSGYSFTGYTLNWVKQSPGKNPEWIGLINPYNGITTYNPKFMGKATLTVDKSSSTAYMELLSLTSEDSAVYYCSREGNWEGVYWGQGTLVTVSA

SEQ ID NO: 28 - protein, 112 aa, synthetic construct
DVVMTQTPLTLSVTIGQPASISCKSSQSLLDSNGKTYLHWLLLRPGQSPKRLIYLVSKLDSGVPDRFTGSGSGTDFTLKISRVEAEDLGVYFCVQGTHFPFTFGSGTKLEIK

SEQ ID NO: 29 - protein, 335 aa, synthetic construct
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIESRRPIPPNSCPPCKECSIFPAPDLLGGPSVFIFPPKIKDVLMISLSPIVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGK

SEQ ID NO: 30 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, synthetic construct [sequence omitted]

SEQ ID NO: 33 - protein, 444 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 219 aa, synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 451 aa, synthetic construct
EVQLLESGGGLVQPGGSLRLSCAASGFTFSSYPMSWVRQAPGKGLEWVSAISGSGGSTYYADSVKGRFTISRDNSKNTLYLQMNSLRAEDTAVYYCAREGGSGSYYNGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 214 aa, synthetic construct
DIQMTQSPSTLSASVGDRVTITCRASQSLGNWLAWYQQKPGKAPKLLIYQASTLESGVPSRFSGSGSGTEFTLTISSLQPDDFATYYCQHYKGSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - skipped

SEQ ID NO: 38 - protein, 17 aa, synthetic construct
  VARIANT 1: note = D or A
  VARIANT 5: note = D or N or A
  VARIANT 6: note = Y or A
  VARIANT 7: note = D or A
  VARIANT 11: note = Y or A
  VARIANT 12: note = N or S
  VARIANT 13: note = E or Q
XINPXXXTTSXXXKFKG

SEQ ID NO: 39 - protein, 14 aa, synthetic construct
  VARIANT 3: note = Y or A
  VARIANT 8: note = Y or A
  VARIANT 9: note = Y or A
  VARIANT 13: note = D or A
  VARIANT 14: note = Y or A
GGXSGSSXXHPMXX

SEQ ID NO: 40 - protein, 17 aa, synthetic construct
  VARIANT 8: note = Y or A
  VARIANT 10: note = T or S
  VARIANT 12: note = Q or R
  VARIANT 15: note = Y or A
KSSQSLLXSXNXKNXLA

SEQ ID NO: 41 - protein, 7 aa, synthetic construct
  VARIANT 1: note = W or A
XASTRES

SEQ ID NO: 42 - protein, 9 aa, synthetic construct
  VARIANT 3: note = Y or A
  VARIANT 4: note = F or Y
  VARIANT 5: note = I or N
  VARIANT 6: note = Y or A
  VARIANT 8: note = R or Y
QQXXXXPXT

SEQ ID NO: 43 - protein, 17 aa, synthetic construct
  VARIANT 1: note = D or A
  VARIANT 5: note = D or A
  VARIANT 6: note = Y or A
  VARIANT 7: note = D or A
  VARIANT 11: note = Y or A
XINPXXXTTSXNEKFKG

SEQ ID NO: 44 - protein, 17 aa, synthetic construct
  VARIANT 8: note = Y or A
  VARIANT 15: note = Y or A
KSSQSLLXSTNQKNXLA

SEQ ID NO: 45 - protein, 9 aa, synthetic construct
  VARIANT 3: note = Y or A
  VARIANT 6: note = Y or A
QQXFIXPRT

SEQ ID NO: 46 - protein, 5 aa, synthetic construct
DYNMD

SEQ ID NO: 47 - protein, 17 aa, synthetic construct
DINPDYDTTSYNEKFKG

SEQ ID NO: 48 - protein, 14 aa, synthetic construct
GGYSGSSYYHPMDY

SEQ ID NO: 49 - protein, 17 aa, synthetic construct
KSSQSLLYSTNQKNYLA

SEQ ID NO: 50 - protein, 7 aa, synthetic construct
WASTRES

SEQ ID NO: 51 - protein, 9 aa, synthetic construct
QQYFIYPRT

SEQ ID NO: 52 - protein, 5 aa, synthetic construct
AYNMD

SEQ ID NO: 53 - protein, 5 aa, synthetic construct
DANMD

SEQ ID NO: 54 - protein, 5 aa, synthetic construct
DYNMA

SEQ ID NO: 55 - protein, 17 aa, synthetic construct
AINPDYDTTSYNEKFKG

SEQ ID NO: 56 - protein, 17 aa, synthetic construct
DINPAYDTTSYNEKFKG

SEQ ID NO: 57 - protein, 17 aa, synthetic construct
DINPDADTTSYNEKFKG

SEQ ID NO: 58 - protein, 17 aa, synthetic construct
DINPDYATTSYNEKFKG

SEQ ID NO: 59 - protein, 17 aa, synthetic construct
DINPDYDTTSANEKFKG

SEQ ID NO: 60 - protein, 14 aa, synthetic construct
GGASGSSYYHPMDY

SEQ ID NO: 61 - protein, 14 aa, synthetic construct
GGYSGSSAYHPMDY

SEQ ID NO: 62 - protein, 14 aa, synthetic construct
GGYSGSSYAHPMDY

SEQ ID NO: 63 - protein, 14 aa, synthetic construct
GGYSGSSYYHPMAY

SEQ ID NO: 64 - protein, 14 aa, synthetic construct
GGYSGSSYYHPMDA

SEQ ID NO: 65 - protein, 17 aa, synthetic construct
KSSQSLLASTNQKNYLA

SEQ ID NO: 66 - protein, 17 aa, synthetic construct
KSSQSLLYSTNQKNALA

SEQ ID NO: 67 - protein, 7 aa, synthetic construct
AASTRES

SEQ ID NO: 68 - protein, 9 aa, synthetic construct
QQAFIYPRT

SEQ ID NO: 69 - protein, 9 aa, synthetic construct
QQYFIAPRT

SEQ ID NO: 70 - protein, 17 aa, synthetic construct
DINPNYDTTSYSQKFKG

SEQ ID NO: 71 - protein, 12 aa, synthetic construct
SEAGNYYWYFDV

SEQ ID NO: 72 - protein, 17 aa, synthetic construct
KSSQSLLYSSNRKNYLA

SEQ ID NO: 73 - protein, 9 aa, synthetic construct
QQYYNYPYT

SEQ ID NO: 74 - protein, 5 aa, synthetic construct
NYWLG

SEQ ID NO: 75 - protein, 17 aa, synthetic construct
DIFPGSDNTYYNEKFKG

SEQ ID NO: 76 - protein, 9 aa, synthetic construct
SGNFYAMDY

SEQ ID NO: 77 - protein, 10 aa, synthetic construct
SASSSVNYMN

SEQ ID NO: 78 - protein, 7 aa, synthetic construct
DTSKLAS

SEQ ID NO: 79 - protein, 9 aa, synthetic construct
FQGSGYPFT

SEQ ID NO: 80 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 81 - protein, 123 aa, synthetic construct
EVQLVQSGAEVKKPGSSVKVSCKASGYIFTAYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 82 - protein, 123 aa, synthetic construct
EVQLVQSGAEVKKPGSSVKVSCKASGYIFTDANMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 83 - protein, 123 aa, synthetic construct
EVQLVQSGAEVKKPGSSVKVSCKASGYIFTDYNMAWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 84 - protein, 123 aa, synthetic construct
EVQLVQSGAEVKKPGSSVKVSCKASGYIFTDYNMDWVRQAPGQGLEWIGAINPDYDTTSYNEKFKGRATLTVDKSTSTAYMELSSLRSEDTAVYYCARGGYSGSSYYHPMDYWGQGTLVTVSS

SEQ ID NO: 85 - protein, 123 aa, synthetic construct
EVQLVQSGAEVKKPGSSVKVSCKASGYIFTDYNMDWVRQAPGQGLEWIGDINPAYDTTSYNEKFKGRATLTVDKSTSTAYMELSSLRSEDTAVYYCARGGYSGSSYYHPMDYWGQGTLVTVSS

SEQ ID NO: 86 - protein, 123 aa, synthetic construct
EVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSS

SEQ ID NO: 87 - protein, 123 aa, synthetic construct
EVQLVQSGAEVKKPGSSVKVSCKASGYIFTDYNMDWVRQAPGQGLEWIGDINPDYATTSYNEKFKGRATLTVDKSTSTAYMELSSLRSEDTAVYYCARGGYSGSSYYHPMDYWGQGTLVTVSS

SEQ ID NO: 88 - protein, 123 aa, synthetic construct
EVQLVQSGAEVKKPGSSVKVSCKASGYIFTDYNMDWVRQAPGQGLEWIGDINPDYDTTSANEKFKGRATLTVDKSTSTAYMELSSLRSEDTAVYYCARGGYSGSSYYHPMDYWGQGTLVTVSS

SEQ ID NO: 89 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGASGSSYYHPMDYWGQGTLVTVSS

SEQ ID NO: 90 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSAYHPMDYWGQGTLVTVSS

SEQ ID NO: 91 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYAHPMDYWGQGTLVTVSS

SEQ ID NO: 92 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMAYWGQGTLVTVSS

SEQ ID NO: 93 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDAWGQGTLVTVSS

SEQ ID NO: 94 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 95 - protein, 113 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGQGTKVEIK

SEQ ID NO: 96 - protein, 113 aa, synthetic construct
DIQMTQSPSSVSASVGDRVTITCKSSQSLLYSTNQKNALAWYQQKPGKAPKLLIYWASTRESGVPSRFSGSGSGTDFTLTISSLQPEDFATYYCQQYFIYPRTFGQGTKVEIK

SEQ ID NO: 97 - protein, 113 aa, synthetic construct
DIQMTQSPSSVSASVGDRVTITCKSSQSLLYSTNQKNYLAWYQQKPGKAPKLLIAWASTRESGVPSRFSGSGSGTDFTLTISSLQPEDFATYYCQQYFIYPRTFGQGTKVEIK

SEQ ID NO: 98 - protein, 113 aa, synthetic construct
DIQMTQSPSSVSASVGDRVTITCKSSQSLLYSTNQKNYLAWYQQKPGKAPKLLIYAASTRESGVPSRFSGSGSGTDFTLTISSLQPEDFATYYCQQYFIYPRTFGQGTKVEIK

SEQ ID NO: 99 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQAFIYPRTFGQGTKVEIK

SEQ ID NO: 100 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIAPRTFGQGTKVEIK

SEQ ID NO: 101 - protein, 123 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSS

SEQ ID NO: 102 - protein, 113 aa, synthetic construct
DIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIK

SEQ ID NO: 103 - protein, 121 aa, synthetic construct
EVQLQQFGAELVKPGTSVKISCKASGYTFTDYNMDWVKQGHGKGLEWIGDINPNYDTTSYSQKFKGKATLTVDKSSSTAYMELRSLTSEDTAVYYCARSEAGNYYWYFDVWGAGTTVTVSS

SEQ ID NO: 104 - protein, 113 aa, synthetic construct
DIVMSQSPSSLAVSVGEKVTMSCKSSQSLLYSSNRKNYLAWYRQKPGQSPKLLIYWASTRESGVPDRFTGSGSGTDFTLTISSVKAEDLAVYSCQQYYNYPYTFGGGTKLEIK

SEQ ID NO: 105 - protein, 118 aa, synthetic construct
QVQLQQSGAELVRPGTSVKISCKASGYAFTNYWLGWVKQRPGHGLEWIGDIFPGSDNTYYNEKFKGKATLTADKSSSTAYMQLSSLASEDSAVYFCARSGNFYAMDYWGQGTSVTVSS

SEQ ID NO: 106 - protein, 106 aa, synthetic construct
ENVLTQSPAIMSASPGEKVTMTCSASSSVNYMNWYQQKSSTFPKLWIYDTSKLASGVPGRFSGSGSGKFYSLTISSMEAEDVATYYCFQGSGYPFTFGSGTKLEIK

SEQ ID NO: 107 - protein, 703 aa, synthetic construct
HHHHHHHHHHGGSQYIKANSKFIGITELGGSCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 108 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 109 - protein, 697 aa, synthetic construct
HHHHHHHHHHSGGGGSGGGGSGGGGSKGVEPKTESERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 110 - protein, 689 aa, synthetic construct
HHHHHHHHHHENLYFQPCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 111 - protein, 694 aa, synthetic construct
HHHHHHHHHHALEVLFQGPCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 112 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 113 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 114 - protein, 697 aa, synthetic construct
HHHHHHHHHHSGGGGSGGGGSGGGGSKGVEPKTESERLAGTESPVREEPGEDFPAARRLYWDDLKRKLSEKLDSTDFTGTIKLLNENSYVPREAGSQKDENLALYVENQFREFKLSKVWRDQHFVKIQVKDSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 115 - protein, 251 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSSGGGGSGGGGSGGGGSDIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIK

SEQ ID NO: 116 - protein, 251 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 117 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - protein, 483 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSSGGGGSGGGGSGGGGSDIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIKGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 483 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 760 aa, synthetic construct
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 122 - protein, 664 aa, synthetic construct
HHHHHHHHHHDYKDDDDKENLYFQGLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 123 - protein, 664 aa, synthetic construct
HHHHHHHHHHDYKDDDDKENLYFQGLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 124 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 125 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 126 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 127 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 128 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVTVSS

SEQ ID NO: 129 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVTVSS

SEQ ID NO: 130 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 131 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVTVSS

SEQ ID NO: 132 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVTVSS

SEQ ID NO: 133 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVTVSS

SEQ ID NO: 134 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVTVSS

SEQ ID NO: 135 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVTVSS

SEQ ID NO: 136 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVTVSS

SEQ ID NO: 137 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVTVSS

SEQ ID NO: 138 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVTVSS

SEQ ID NO: 139 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTTVTVSS

SEQ ID NO: 140 - protein, 118 aa, synthetic construct
QVQLQQSGAELVRPGTSVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVTVSS

SEQ ID NO: 141 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIK

SEQ ID NO: 142 - protein, 113 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGCGTKVEIK

SEQ ID NO: 143 - protein, 113 aa, synthetic construct
DIQMTQSPSSVSASVGDRVTITCKSSQSLLYSTNQKNALAWYQQKPGKAPKLLIYWASTRESGVPSRFSGSGSGTDFTLTISSLQPEDFATYYCQQYFIYPRTFGCGTKVEIK

SEQ ID NO: 144 - protein, 113 aa, synthetic construct
DIQMTQSPSSVSASVGDRVTITCKSSQSLLYSTNQKNYLAWYQQKPGKAPKLLIAWASTRESGVPSRFSGSGSGTDFTLTISSLQPEDFATYYCQQYFIYPRTFGCGTKVEIK

SEQ ID NO: 145 - protein, 113 aa, synthetic construct
DIQMTQSPSSVSASVGDRVTITCKSSQSLLYSTNQKNYLAWYQQKPGKAPKLLIYAASTRESGVPSRFSGSGSGTDFTLTISSLQPEDFATYYCQQYFIYPRTFGCGTKVEIK

SEQ ID NO: 146 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQAFIYPRTFGCGTKVEIK

SEQ ID NO: 147 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIAPRTFGCGTKVEIK

SEQ ID NO: 148 - protein, 113 aa, synthetic construct
DIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGCGTKLEIK

SEQ ID NO: 149 - protein, 113 aa, synthetic construct
DIVMSQSPSSLAVSVGEKVTMSCKSSQSLLYSSNRKNYLAWYRQKPGQSPKLLIYWASTRESGVPDRFTGSGSGTDFTLTISSVKAEDLAVYSCQQYYNYPYTFGCGTKLEIK

SEQ ID NO: 150 - protein, 106 aa, synthetic construct
ENVLTQSPAIMSASPGEKVTMTCSASSSVNYMNWYQQKSSTFPKLWIYDTSKLASGVPGRFSGSGSGKFYSLTISSMEAEDVATYYCFQGSGYPFTFGCGTKLEIK

SEQ ID NO: 151 - protein, 251 aa, synthetic construct
EVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 152 - protein, 251 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIK

SEQ ID NO: 153 - protein, 251 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSS

SEQ ID NO: 154 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 155 - protein, 251 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGQGTKVEIK

SEQ ID NO: 156 - protein, 256 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 157 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 158 - protein, 26 aa, synthetic construct
HHHHHHGGGGSGGGGSGGGGSGGGGS

SEQ ID NO: 159 - protein, 41 aa, synthetic construct
HHHHHHGLNDIFEAQKIEWHEGGGGSGGGGSGGGGSGGGGS

SEQ ID NO: 160 - protein, 219 aa, synthetic construct
EIVLTQSPLSLSVTLGQSASISCRSSQSLLDSNGKTYLHWFILRPGQSPRRLIYLVSILDSGVPDRFSGSGSGTDFTLKISRVEAEDVGVYYCVQGTHFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 446 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 162 - protein, 722 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 163 - protein, 722 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 164 - protein, 722 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 165 - protein, 446 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 15 aa, synthetic construct
GGGGSGGGGSGGGGS

SEQ ID NO: 167 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVTVKS

SEQ ID NO: 168 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVKS

SEQ ID NO: 169 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 170 - protein, 123 aa, synthetic construct
EVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 171 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVKVSS

SEQ ID NO: 172 - protein, 123 aa, synthetic construct
EVQLVQSGAERKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 173 - protein, 123 aa, synthetic construct
EVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 174 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 175 - protein, 123 aa, synthetic construct
EVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 176 - protein, 123 aa, synthetic construct
EVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 177 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 178 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVKS

SEQ ID NO: 179 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 180 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVTVKS

SEQ ID NO: 181 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVKVSS

SEQ ID NO: 182 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 183 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAERKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 184 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 185 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 186 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 187 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 188 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 189 - protein, 722 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVKS

SEQ ID NO: 190 - protein, 722 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVTVKS

(19) United States
(12) Patent Application Publication
(10) Pub. No.: US 2025/0388664 A1
(43) Pub. Date: Dec. 25, 2025

(54) BISPECIFIC BINDING MOLECULE

(71) Applicant: BioArctic AB, Stockholm (SE)

(72) Inventors: Paulina APPELKVIST, Stockholm (SE); Emma BOSTRÖM, Stockholm (SE); Ronny FALK, Stockholm (SE); Per-Ola FRESKGÅRD, Stockholm (SE); Ken HONEK, Stockholm (SE); Martin LARHAMMAR, Stockholm (SE); Hanna LAUDON, Stockholm (SE); Sebastian MEISTER, Stockholm (SE); Christer MÖLLER, Stockholm (SE); Adeline RACHALSKI, Stockholm (SE); Lisa SANDERSJÖÖ, Stockholm (SE); Jessica SIGVARDSON, Stockholm (SE); Linda SÖDERBERG, Stockholm (SE); Karin TEGERSTEDT, Stockholm (SE); James MCCLORY, Banbridge (GB)

(21) Appl. No.: 19/243,198

(22) Filed: Jun. 19, 2025

(30) Foreign Application Priority Data
Jun. 20, 2024 (EP) 24183444.9
Nov. 27, 2024 (EP) 24215702.2

(51) Int. Cl. C07K 16/18; A61K 39/00; C07K 16/28

(52) U.S. Cl. CPC C07K 16/18; C07K 16/2881; A61K 2039/505; C07K 2317/24; C07K 2317/31; C07K 2317/34; C07K 2317/52; C07K 2317/55; C07K 2317/622; C07K 2317/73; C07K 2317/732; C07K 2317/734; C07K 2317/76; C07K 2317/92; C07K 2317/94

(57) ABSTRACT

The present disclosure provides a bispecific binding molecule, which binds to AβpE3, i.e. to an N-terminally truncated and pyroglutamate-modified form of amyloid beta (Aβ), and to the protease-like domain of human transferrin receptor 1 (hTfR1), as well as therapeutic and diagnostic uses thereof.

### CROSS-REFERENCE TO RELATED APPLICATIONS

This application claims benefit of priority to European Application No. EP24215702.2, filed Nov. 27, 2024, and European Application No. EP24183444.9, filed Jun. 20, 2024, the contents of each of which is incorporated by reference in its entirety herein.

### SEQUENCE LISTING

This application contains an electronic Sequence Listing which has been submitted in XML file format with this application, the entire content of which is incorporated by reference herein in its entirety. The Sequence Listing XML file submitted with this application is entitled “20250616_21153938_BAP031-PC_ST26_SeqList.xml”, was created on Jun. 17, 2025, and is 207,132 bytes in size.

### FIELD

The present disclosure relates to a bispecific binding molecule, which binds to AβpE3, i.e. to an N-terminally truncated and pyroglutamate-modified form of amyloid beta (Aβ), and to the protease-like domain of human transferrin receptor 1 (hTfR1), as well as therapeutic and diagnostic uses thereof.

### BACKGROUND

Alzheimer's disease (AD) is a progressive neurodegenerative dementia disorder which exists in a more common late-onset form and an early-onset familial form. AD is characterized by progressive loss of memory and cognitive function. At present, AD treatments are limited to symptomatic management and the prognosis is poor for AD patients. It is estimated that about 18 million people worldwide are presently suffering from AD, and the number of people suffering from AD is expected to increase due to the aging population. The prevalence of AD doubles approximately every 5 years from the age of 60, from 10% of individuals at the age of 65 to 50% of individuals at the age of 85 or more (Solomon (2007), Expert Opin Investig Drugs 16(6):819-828).

Accumulation of Aβ peptide in the brain is thought to play an important role in the neuropathology of AD. Aβ is generated from the amyloid precursor protein (APP) by sequential proteolysis and secreted via major regulated as well as minor constitutive secretory pathways. Aβ is a normal product of cell metabolism, which is present in the plasma and cerebrospinal fluid in healthy individuals. However, abnormal and excessive accumulation of Aβ in the brain leads to the formation of toxic Aβ aggregates that induce synaptic dysfunction and neuronal loss.

The main variants of Aβ detected in the human brain are Aβ1-40 and Aβ1-42. However, a significant proportion of AD brain Aβ also consists of N-terminally truncated species (Aβn-40/42 where n=2 to 11). Most such N-truncated Aβ peptides have been considered to be degradation products of full-length Aβ. It has been demonstrated that amyloid aggregates in AD brain and in brain of cognitively normal elderly subjects have different compositions, and that the toxic effect of these aggregates is correlated with the predominance of the N-terminal truncated species over the full-length Aβ. Pyroglutamate-modified Aβ peptides have been demonstrated to be the predominant components among all N-terminally truncated Aβ species in AD brain. In particular AβpE3, an Aβ peptide having an amino-terminal pyroglutamate at position 3, has been shown to be a major N-truncated/modified constituent of intracellular, extracellular and vascular Aβ deposits in AD brain tissue. Furthermore, it has been demonstrated that AβpE3 progressively accumulates in the brain at the earliest stages of AD even before the appearance of clinical symptoms, which suggests that this peptide plays an important role in the formation of pathological amyloid aggregates. Thus, N-terminally truncated/modified Aβ peptides represent highly desirable and abundant therapeutic targets. This is particularly the case for AβpE3. For review and further references, see Perez-Garmendia and Gevorkian (2013), Curr Neuropharmacol 11:491-498.

Therapeutic antibodies against AβpE3 have been proposed, e.g. in WO2011/001366, WO2012/021469, WO2017/123517, WO2018/194951, WO2010/009987, WO2017/009459, WO2019/149689, WO2020/070225, WO2018/083628 and WO2020/193644.

Treatment modalities for brain and neurological diseases are furthermore limited by the impermeability of the blood vessels of the brain to most substances carried in the bloodstream (Freskgard and Urich (2017), Neuropharmacology 120:38-55; Stanimirovic et al (2018), BioDrugs 32:547-559). The small blood vessels (capillaries) of the brain, referred to collectively as the blood-brain barrier (BBB), are unique when compared to the blood vessels found in the periphery of the body. Tight apposition of BBB endothelial cells (EC) to neural cells, such as astrocytes, pericytes and neurons, induces phenotypic features that contribute to the observed impermeability. Tight junctions between ECs in the BBB limit paracellular transport, while the lack of passive pinocytotic vesicles and fenestrae limit non-specific transcellular transport. These factors combine to restrict molecular flux from the blood to the brain in general to molecules that are less than 500 Da in size and lipophilic. Thus, the otherwise promising prospect of using the large mass transfer surface area (over 20 m² from 600 km of capillaries in a human brain) of the blood stream as a delivery vehicle is made largely infeasible, except in those circumstances where a drug with the desired pharmacological properties fortuitously possesses size and lipophilicity attributes which allow it to pass through the BBB. Because of such restrictions, it has been estimated that more than 98% of all small molecule pharmaceuticals and nearly 100% of the emerging class of protein and gene therapeutics do not cross the BBB.

WO91/03259 proposes a principle for transporting a neuropharmaceutical agent across the BBB, which involves conjugating the agent to an antibody which is reactive with the transferrin receptor. According to this disclosure, binding of the conjugate to the transferrin receptor leads to active transport of the conjugate across the BBB. Later work has developed this concept further, for example as described in WO2012/075037, WO2014/033074, WO2018/011353 and WO2022/258841, all describing different formats for achieving transport of a biopharmaceutical agent across the BBB by utilizing the transferrin receptor.

There exist two forms of the human transferrin receptor. Transferrin receptor 1 (TfR1) is one of the targets for the bispecific binding molecule of the present disclosure. TfR1 is an iron transporter protein, which maintains cellular iron levels by recognizing and internalizing through specific binding of the iron carrier proteins transferrin (Tf) and ferritin (Ft) into cells through endocytosis mediated by clathrin-coated vesicles. TfR1 is expressed in numerous cells and organs, but expression levels vary and, importantly, TfR1 is expressed to a higher degree on BBB endothelial cells than on other endothelial cells, making the receptor a target for neuropharmaceutical delivery. Structurally, TfR1 is a dimeric transmembrane glycoprotein comprising the amino acid sequence SEQ ID NO:121, which has a large ectodomain (residues 89-760), an intramembrane region (residues 62-88) and a cytoplasmic domain (residues 1-61). The ectodomain in turn has three distinct domains held separate from the cell surface by a stalk region (residues 89-120). These three parts of the ectodomain are the helical domain (residues 606-760), the protease-like domain (residues 121-183, 384-605) and the apical domain (residues 184-383) (Lawrence et al (1999), Science 286:779-782).

In the context of BBB transport via the TfR1, antibodies and fragments thereof which have affinity for TfR1 have been described. By way of example, a number of TfR1-binding antibodies are disclosed in WO2014/189973, in which antibodies are grouped according to epitope specificity in classes I-IV (see e.g. FIG. 3 and the associated figure description on page 30 lines 11-15). Classes I-III of WO2014/189973 are denoted “apical binders” whereas the antibody of class IV is denoted a “non-apical binder”. Other TfR1-binding antibodies are disclosed in EP3088518, EP3315606 and EP3560958, however without any information about the epitope specificity of these disclosed antibodies.

Thus, most work on using TfR1 as a target for binding and BBB transport has focused on apical binders. This is thought to be because the apical domain is the structure within TfR1 that seems to provoke a strong immune response and thus to trigger antibody generation in animals when used as an immunogen. Thus, most known antibody binders against TfR1 have epitopes that are located within the apical domain. Another indication that the apical domain contains structures prone to engage with various ligands is that viruses have been described to utilize epitopes within the apical domain to enter cells (Cohen-Dvashi et al (2020), Nat Commun 11:67).

Furthermore, the detailed structure of the TfR1 and ferritin complex was recently determined (Montemiglio et al (2019), Nat Commun 10:1121), showing that the interface between TfR1 and ferritin is located within the apical domain. This suggest that TfR1 apical binders could potentially interfere with the binding of ferritin to TfR1 if used for BBB transport and in this way influence the normal function of ferritin in iron transport. Also, the binding and uptake of H-ferritin have been shown to be mediated by TfR1 (Li et al (2010), Proc Natl Acad Sci USA 107(8):3505-10). Thus, there are reasons to conclude that binders directed against the apical domain of TfR1, and especially binding to the binding site used by ferritin, may negatively influence the important function of ferritin in transporting iron via the binding to TfR1.

It has been reported that TfR1 apical binders can induce both acute clinical signs and decreased in circulating reticulocytes (Couch et al (2013), Sci Transl Med 5:183ra57). The TfR1 has also been described in relation to anemia and iron deficiency (Braga et al (2014), Clin Chim Acta 431:143-147). Anemia due to autoantibodies to TfR1 has also been described (Hyman et al (1984), N Engl J Med 311:214-218). Taken together, the data suggest that TfR1-binding and interfering with iron transporters such as transferrin and/or ferritin could lead to safety issues such as reduction in reticulocyte levels and anemia.

To date, the focus within the field has been to avoid interfering with one of the described TfR1 ligands, namely transferrin. This has guided the field to utilize binding sites in the apical domain of TfR1, distant from the binding site of transferrin. However, such apical binders may still interfere with the other important TfR1 ligand, ferritin, leading to interference in iron transport and function.

Despite the existence of candidate antibodies within the field, there remains a need in the art for novel therapeutic, prophylactic, diagnostic and prognostic tools for detecting and treating AD and other neurodegenerative diseases. There also remains a need in the field for antibodies and other binding molecules which have a binding affinity for TfR1 but which do not exhibit the drawbacks and risks associated with hitherto known binding molecules.

### DISCLOSURE OF THE INVENTION

One object of the disclosure is to provide binding molecules having one or more novel and useful binding specificity/specificities.

Another object of the disclosure is to provide novel candidate molecules for the treatment of neurodegenerative diseases via targeting of the AβpE3 peptide with a beneficial and unique binding profile.

Another object of the disclosure is to enable the diagnosis of AD and other neurodegenerative disorders via detection of AβpE3 implicated in disease formation and/or progression.

Another object of the disclosure is to provide molecules that bind to the AβpE3 peptide with high affinity.

Another object of the disclosure is to provide molecules that bind to the AβpE3 peptide with high specificity.

Another object of the disclosure is to provide molecules that bind to the AβpE3 peptide with high selectivity with respect to other Aβ peptide variants.

Another object of the disclosure is to provide molecules that bind to both monomeric forms of AβpE3 and to the putatively neurotoxic protofibrils comprising AβpE3.

Another object of the disclosure is to provide molecules that bind to all forms of AβpE3, including fibrils and plaques comprising AβpE3.

Another object of the disclosure is to provide AβpE3-binding molecules that combine desirable properties for development into a biopharmaceutical product.

Another object of the disclosure is to provide AβpE3-binding molecules that exhibit little or no immunogenicity upon administration in human subjects.

Another object of the disclosure is to provide AβpE3-binding molecules that show a beneficial pharmacokinetic profile upon administration in human subjects, for example evidenced by one or more of a long half-life, a high total exposure and a low clearance.

Another object of the disclosure is to provide a TfR1-binding molecule which utilizes a different binding site on TfR1 than the naturally occurring ligands.

One such object is to provide a TfR1-binding molecule which utilizes a different binding site on TfR1 than transferrin.

Another such object is to provide a TfR1-binding molecule which utilizes a different binding site on TfR1 than ferritin.

Yet another such object is to provide a TfR1-binding molecule which utilizes a different binding site on TfR1 than HFE (homeostatic iron regulator).

A related object of the disclosure is to provide a TfR1-binding molecule which interacts with TfR1 in a way which minimizes the interference with TfR1 itself and/or its normal function.

A related object of the disclosure is to provide a TfR1-binding molecule which exhibits an improved stability, e.g. in the form of storage stability and/or resistance against multimerization, as compared to other TfR1-binding molecules.

Another object of the disclosure is to provide a TfR1-binding molecule suitable for use as a fusion partner in constructs arranged for transport through the BBB.

It is also an object of the disclosure to combine beneficial properties of different moieties into a bispecific binding molecule in which a therapeutic target in the brain is engaged more effectively through the provision of a moiety which enables transport through the blood-brain barrier.

One or more of these objects, and other objects that are evident to the skilled person from the teachings herein, are met by the various aspects of the disclosure.

Thus, in a first aspect, the present disclosure provides a bispecific binding molecule, comprising
a first moiety M1, which is an AβpE3 binding moiety comprising a VH domain and a VL domain, said VH and VL domains forming a VH/VL pair comprising an antigen-binding surface, in which said antigen-binding surface is composed of three complementarity-determining regions (CDRs) from said VH domain and three CDRs from said VL domain, and in which said CDRs consist of the following amino acid sequences:

VHCDR1:
(SEQ ID NO: 1)
GX₁TX₂N

wherein
X₁ is selected from Y and F; and
X₂ is selected from L and M;

VHCDR2:
(SEQ ID NO: 2)
LINPYNGX₃TTYNX₄KFX₅G

wherein
X₃ is selected from I and V
X₄ is selected from P and Q; and
X is selected from M and K;

VHCDR3:
(SEQ ID NO: 3)
EGNWEGVY
VLCDR1:
(SEQ ID NO: 4)
X₆SSQSLLDSNGKTYLH

wherein
X₆ is selected from K and R;

VLCDR2:
(SEQ ID NO: 5)
LVSX₇LDS

wherein
X₇ is selected from I and K; and

VLCDR3:
(SEQ ID NO: 6)
VQGTHFPFT;

a second moiety M2, which is a human transferrin receptor 1 (hTfR1) binding moiety comprising an immunoglobulin heavy chain variable domain (VH) and an immunoglobulin light chain variable domain (VL), said VH and VL domains forming a VH/VL pair comprising an antigen-binding surface, in which said antigen-binding surface provides the binding protein with the capacity to bind selectively to an epitope located in the protease-like domain of hTfR1 defined by amino acid residues 121-183 and 384-605 in SEQ ID NO:121.

In a second aspect, the present disclosure provides a pharmaceutical composition comprising a bispecific binding molecule in accordance with the first aspect of the invention and a pharmaceutically acceptable excipient or carrier.

In further aspects, the present disclosure provides bispecific binding molecules and/or pharmaceutical compositions comprising the same for use in methods of treatment or for use in methods of detection or diagnosis as described herein.

### AβpE3 Binding Moiety M1

As described above, in a first aspect, the disclosure provides a bispecific binding molecule comprising a first moiety M1, which has affinity for AβpE3, and in which the six CDRs of the VH and VL domain are as defined above with reference to SEQ ID NO:1-6.

The identification of the AβpE3-binding moiety M1 is based on detailed insights into the pathophysiology of diseases characterized by amyloid aggregation, and the identification of particular forms of Aβ in brain tissue from patients suffering from such diseases. As a non-limiting example, soluble forms of AβpE3 were found in extracts from AD brains, which further highlights the importance of obtaining molecules that bind such species in a specific and/or selective manner. However, these insights also point to the potential benefits of having molecules that bind AβpE3 in all forms that are present in connection with disease. These insights have enabled the generation of primary antibodies that are specific and/or selective for AβpE3 in its various forms. Also enabled was the further development of these initial antibodies into humanized antibodies and variants thereof with a number of beneficial properties, including an unexpectedly favorable pharmacokinetic profile. Generation and characterization of exemplary such antibodies is detailed in Examples 1-14. Further development of these antibodies into bispecific binding molecules of the present disclosure is detailed in Examples 30-38.

Without wishing to be bound by theory, it is contemplated that the binding molecules of the disclosure are useful in the diagnosis, prognosis and/or treatment of neurodegenerative diseases such as AD, through specific binding to the putatively disease-causing Aβ variant AβpE3 by way of the AβpE3-binding moiety M1.

As defined herein, embodiments of the bispecific binding molecule of the first aspect of the disclosure are characterized by specific amino acid sequences in the regions determining its binding capability, such as the CDRs of the heavy and/or light chain variable domains of M1 and M2, or indeed the entire VL and/or VH domains or regions of M1 and M2. It is contemplated that the specific sequence information provided for the molecules generated as described in the Examples enables the skilled person to define combinations and variations of these sequences within the scope of the disclosure.

Thus, in one embodiment of the first aspect, the AβpE3-binding moiety M1 comprises VHCDR1, VHCDR2 and VLCDR2 regions which consist of the following amino acid sequences:

VHCDR1:
(SEQ ID NO: 7)
GFTMN
VHCDR2:
(SEQ ID NO: 8)
LINPYNGVTTYNQKFKG
VLCDR2:
(SEQ ID NO: 9)
LVSILDS.

In a more specific embodiment, the AβpE3-binding moiety M1 comprises a heavy chain variable domain and a light chain variable domain, wherein said heavy chain variable domain comprises an amino acid sequence selected from the group consisting of SEQ ID NO:15-22 and amino acid sequences having at least 80% identity to any one of SEQ ID NO:15-22, provided that the three VHCDR regions consist of SEQ ID NO:7, SEQ ID NO:8 and SEQ ID NO:3. For example, the amino acid sequence comprised in the VH in moiety M1 is selected from the group consisting of SEQ ID NO:15-21, such as selected from the group consisting of SEQ ID NO:15-16 and 18-21 or selected from the group consisting of SEQ ID NO:15-20, such as selected from the group consisting of SEQ ID NO:15-16 and 18-20, in particular selected from the group consisting of SEQ ID NO:15 and 18, most particularly being SEQ ID NO:18, or a sequence having at least 80% identity to any one of the listed subgroups, always provided that the three VHCDR regions consist of SEQ ID NO:7, SEQ ID NO:8 and SEQ ID NO:3.

In another embodiment, the AβpE3-binding moiety M1 comprises a VLCDR1 consisting of the amino acid sequence

(SEQ ID NO: 10)
RSSQSLLDSNGKTYLH.

In a more specific such embodiment, moiety M1 comprises a heavy chain variable domain and a light chain variable domain, wherein said light chain variable domain comprises an amino acid sequence selected from the group consisting of SEQ ID NO:23-24 and amino acid sequences having at least 80% identity to any one of SEQ ID NO:23-24, provided that the three VLCDR regions consist of SEQ ID NO:10, SEQ ID NO:9 and SEQ ID NO:6. For example, the amino acid sequence comprised in the VL in moiety M1 is SEQ ID NO:23 or a sequence having at least 80% identity to SEQ ID NO:23.

In one embodiment, moiety M1 comprises both
a heavy chain variable domain comprising an amino acid sequence selected from the group consisting of SEQ ID NO:15-22 and amino acid sequences having at least 80% identity to any one of SEQ ID NO:15-22, provided that the three VHCDR regions consist of SEQ ID NO:7, SEQ ID NO:8 and SEQ ID NO:3; and
a light chain variable domain comprising an amino acid sequence selected from the group consisting of SEQ ID NO:23-24 and amino acid sequences having at least 80% identity to any one of SEQ ID NO:23-24, provided that the three VLCDR regions consist of SEQ ID NO:10, SEQ ID NO:9 and SEQ ID NO:6.

For example, moiety M1 in the bispecific binding molecule of the first aspect comprises a heavy chain variable domain and a light chain variable domain selected from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24; and
h) a heavy chain variable domain comprising SEQ ID NO:22 and a light chain variable domain comprising SEQ ID NO:23,
for example from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23; and
g) a heavy chain variable domain comprising SEQ ID NO:21 and a light chain variable domain comprising SEQ ID NO:24,
or from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23; and
g) a heavy chain variable domain comprising SEQ ID NO:21 and a light chain variable domain comprising SEQ ID NO:24,
for example from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23; and
f) a heavy chain variable domain comprising SEQ ID NO:20 and a light chain variable domain comprising SEQ ID NO:23,
for example from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23; and
e) a heavy chain variable domain comprising SEQ ID NO:19 and a light chain variable domain comprising SEQ ID NO:23,
for example from the group consisting of the following VH/VL combinations:
a) a heavy chain variable domain comprising SEQ ID NO:18 and a light chain variable domain comprising SEQ ID NO:23;
b) a heavy chain variable domain comprising SEQ ID NO:15 and a light chain variable domain comprising SEQ ID NO:23,
for example an antibody or antigen-binding fragment thereof comprising the following VH/VL combination:
a) a heavy chain variable domain comprising SEQ ID NO:18 and a light chain variable domain comprising SEQ ID NO:23.

In another embodiment of the first aspect, the disclosure provides a bispecific binding molecule, in which the amino acid sequence of VLCDR1 in moiety M1 is
KSSQSLLDSNGKTYLH (SEQ ID NO:11).

In a more specific embodiment, moiety M1 comprises a heavy chain variable domain and a light chain variable domain, wherein said heavy chain variable domain comprises the amino acid sequence SEQ ID NO:25 or an amino acid sequence having at least 80% identity to SEQ ID NO:25, provided that the three VHCDR regions consist of SEQ ID NO:7, SEQ ID NO:8 and SEQ ID NO:3.

In another embodiment, moiety M1 comprises a heavy chain variable domain and a light chain variable domain, wherein said light chain variable domain comprises the amino acid sequence SEQ ID NO:26 or an amino acid sequence having at least 80% identity to SEQ ID NO:26, provided that the three VLCDR regions consist of SEQ ID NO:11, SEQ ID NO:9 and SEQ ID NO:6.

In yet another embodiment, moiety M1 in the bispecific binding molecule comprises both
a heavy chain variable domain comprising the amino acid sequence SEQ ID NO:25 or an amino acid sequence having at least 80% identity to SEQ ID NO:25, provided that the three VHCDR regions consist of SEQ ID NO:7, SEQ ID NO:8 and SEQ ID NO:3, and
a light chain variable domain comprising the amino acid sequence SEQ ID NO:26 or an amino acid sequence having at least 80% identity to SEQ ID NO:26, provided that the three VLCDR regions consist of SEQ ID NO:11, SEQ ID NO:9 and SEQ ID NO:6.

In another embodiment of the first aspect, the disclosure provides a bispecific binding molecule, wherein the VHCDR1, VHCDR2, VLCDR1 and VLCDR2 regions in moiety M1 consist of the following amino acid sequences:

VHCDR1:
(SEQ ID NO: 12)
GYTLN;
VHCDR2:
(SEQ ID NO: 13)
LINPYNGITTYNPKFMG;
VLCDR1:
(SEQ ID NO: 11)
KSSQSLLDSNGKTYLH;
and
VLCDR2:
(SEQ ID NO: 14)
LVSKLDS.

In a more specific embodiment, the AβpE3-binding moiety M1 of the bispecific binding molecule of the first aspect comprises a heavy chain variable domain and a light chain variable domain, wherein said heavy chain variable domain comprises the amino acid sequence SEQ ID NO:27 or an amino acid sequence having at least 80% identity to SEQ ID NO:27, provided that the three VHCDR regions consist of SEQ ID NO:12, SEQ ID NO:13 and SEQ ID NO:3.

In another embodiment, the AβpE3-binding moiety M1 comprises a heavy chain variable domain and a light chain variable domain, wherein said light chain variable domain comprises the amino acid sequence SEQ ID NO:28 or an amino acid sequence having at least 80% identity to SEQ ID NO:28, provided that the three VLCDR regions consist of SEQ ID NO:11, SEQ ID NO:14 and SEQ ID NO:6.

In yet another embodiment, moiety M1 comprises both
a heavy chain variable domain comprising the amino acid sequence SEQ ID NO:27 or an amino acid sequence having at least 80% identity to SEQ ID NO:27, provided that the three VHCDR regions consist of SEQ ID NO:12, SEQ ID NO:13 and SEQ ID NO:3, and
a light chain variable domain comprising the amino acid sequence SEQ ID NO:28 or an amino acid sequence having at least 80% identity to SEQ ID NO:28, provided that the three VLCDR regions consist of SEQ ID NO:11, SEQ ID NO:14 and SEQ ID NO:6.

In certain embodiments, the definitions of VH and VL sequences of the AβpE3-binding moiety M1 in the bispecific binding molecule are limited to any one of the listed sequences and sequences having at least 85%, such as at least 90%, such as at least 95%, such as at least 98%, such as at least 100% identity thereto.

In specific embodiments, the combinations of VH/VL in moiety M1 are those present in the antibodies exemplified in Examples 1-13 (see Tables 1, 6 and 12 in particular).

As a person of skill in the art is aware, Aβ peptides exist in various lengths and forms. Of particular relevance to the present disclosure is the N-terminus of the Aβ peptide, in that the AβpE3-binding moiety M1 of the bispecific binding molecule herein binds to Aβ peptides having a truncation of two amino acid residues in comparison to full-length Aβ (i.e. Aβ peptides starting at the aspartic acid residue commonly denoted “D1”). On the other hand, the C-terminus of the peptide has little relevance to the present disclosure, and, as is well known, Aβ peptides may be as long as 43 amino acid residues or end already at e.g. position 28. In the terminology used herein, “AβpE3” is intended to encompass any Aβ peptide that begins with a pyroglutamate (“pE3”) residue corresponding to the third amino acid residue in the full-length peptide, regardless of C-terminus, which may or may not be truncated with respect to the full-length sequence. Likewise, “Aβ1-X” is used interchangeably with “full-length Aβ” and refers to any Aβ peptide that begins with the first amino acid residue (i.e. D1), regardless of its C-terminus.

A person of skill in the art is also aware that Aβ peptides may exist in various forms along the progressive aggregation thereof from monomers to insoluble plaques. Of particular relevance to the present disclosure, soluble forms of Aβ peptides may be present in monomer form, or in various oligomeric or further aggregated forms. Soluble forms of polymerized or aggregated Aβ peptides are collectively referred to as “protofibrils” in the present disclosure. For clarity, “protofibrils” is intended to encompass oligomers and higher order aggregates, but excludes insoluble fibrils or amyloid plaques.

In certain embodiments, a bispecific binding molecule of the first aspect has an affinity for AβpE3 in a form selected from the group consisting of monomers, protofibrils, fibrils and plaques. In a more specific embodiment, the bispecific binding molecule has an affinity for AβpE3 in a form selected from monomers and protofibrils. In yet another embodiment, the bispecific binding molecule has an affinity for AβpE3 monomers. In a further embodiment, the bispecific binding molecule has an affinity for AβpE3 protofibrils. It is noted that a bispecific binding molecule according to the first aspect may have an affinity for AβpE3 generally, e.g. having an affinity for AβpE3 monomers as well as to AβpE3 protofibrils and/or other AβpE3 species.

Alternatively, the bispecific binding molecule may exhibit a preference or selectivity for one form of AβpE3 over another. In one such embodiment, the bispecific binding molecule has a higher binding affinity for protofibrils comprising AβpE3 than for AβpE3 monomers. Without wishing to be bound by theory, such higher affinity for protofibrils in embodiments of the bispecific binding molecule may be due to avidity effects, insofar as the protofibril form of Aβ is thought to present a plurality of epitopes to bind, in comparison to the monomeric form. As such, the affinity of a binding molecule for protofibrils may be measured and reported herein as an “apparent affinity” in a manner known to the skilled person. In one embodiment, the bispecific binding molecule has at least 2× higher binding affinity for protofibrils comprising AβpE3 than for AβpE3 monomers, such as at least 10× higher, such as at least 40× higher, such as at least 100× higher, such as at least 200× higher binding affinity.

In certain embodiments, the bispecific binding molecule of the first aspect binds selectively to AβpE3. As used herein, the term “bind selectively” refers to the preferential binding to the AβpE3 target. In certain embodiments, the bispecific binding molecule of the first aspect does not bind to any appreciable extent to non-truncated amyloid beta, Aβ1-X. Put slightly differently, in one such embodiment, the bispecific binding molecule has a higher binding affinity for AβpE3 monomers than for Aβ1-X monomers. In more specific embodiments, the bispecific binding molecule has at least 2× higher binding affinity for AβpE3 monomers than for Aβ1-X monomers, such as at least 10× higher, such as at least 100× higher, such as at least 1000× higher, such as at least 3000× higher binding affinity.

In one embodiment of the bispecific binding molecule, it has a binding affinity (or an apparent binding affinity) for protofibrils comprising AβpE3 that corresponds to a K_{D} value of no more than 1 nM, such as between 1 and 200 pM, such as between 10 and 100 pM, as determined by SPR.

In another embodiment, the bispecific binding molecule has a binding affinity for AβpE3 monomers that corresponds to a K_{D} value of no more than 100 nM, such as between 0.1 and 50 nM, such as between 0.5 and 10 nM, as determined by SPR.

hTfR1-Binding Moiety M2

As described above, in the first aspect, the present disclosure provides a bispecific binding protein in which moiety M2 is a human transferrin receptor 1 (hTfR1) binding moiety, capable of selective binding to an epitope located in the protease-like domain of hTfR1 defined by amino acid residues 121-183 and 384-605 in SEQ ID NO:121. Without wishing to be bound by theory, the binding by moiety M2 to hTfR1 to an epitope, or binding site, within the protease-like domain is contemplated to offer advantages in terms of avoiding the drawbacks associated with known binders to hTfR1, in particular those known binders which have affinity for epitopes or binding sites located in the apical domain of TfR1.

In a specific embodiment, the epitope or binding site for the hTfR1-binding moiety M2 comprises the amino acid residues 150, 151, 154, 158, 159, 161, 163 and 385 in SEQ ID NO:121. In another embodiment, the epitope or binding site for the hTfR1 binding moiety M2 consists of the amino acid residues 150, 151, 154, 158, 159, 161, 163 and 385 in SEQ ID NO:121. In an alternative specific embodiment, the epitope or binding site for the hTfR1 binding moiety M2 comprises or consists of at least one, at least two, at least three, at least four, at least five, at least six, at least seven or all eight of the amino acid residues 150, 151, 154, 158, 159, 161, 163 and 385 in SEQ ID NO:121. As shown in the examples which follow, for example with reference to FIG. 39, this embodiment of the epitope for the hTfR1 binding moiety disclosed herein ensures binding that does not interfere with the natural hTfR1 ligands transferrin and ferritin.

As known to a person skilled in the art, an epitope (or “antigenic determinant”) is a group of amino acids or other chemical groups exposed on the surface of a molecule, frequently a protein, here hTfR1, which can generate an antigenic response and bind antibody. An epitope is a localized region on the surface of an antigen that is recognized by the immune system, specifically by antibodies. A conformational epitope is composed of neighboring amino acid residues located on an antigenic protein surface structure. Conformational epitopes bind their complementary paratopes in B-cell receptors and/or antibodies. In one embodiment of the disclosure, the epitope bound by the hTfR1-binding moiety M2 of the bispecific binding molecule is a conformational epitope.

In one embodiment, the binding to hTfR1 by the M2 binding moiety is monovalent.

As described above, the hTfR1-binding moiety M2 comprises a VH/VL pair with an antigen-binding surface. For clarity with regard to both M1 and M2, the designation of “VH/VL” as used in relation to a VH/VL pair does not limit the construct to any particular order of the VH and VL domains in the polypeptide chain, but is only used to convey that both the VH and VL domains are present, and that they are capable of pairwise association to form an Ig domain with an antigen-binding surface. As non-limiting alternatives, the term “VH/VL pair” encompasses, for example, constructs in which the VL domain precedes the VH domain in a single chain Fv, constructs in which the VH domain precedes the VL domain in a single chain Fv, and constructs in which the VH and VL domains are non-covalently associated with each other. In a specific embodiment of the binding protein, the VH/VL pair in M2 is arranged such that the VL domain precedes the VH domain in a single chain Fv construct.

The VH/VL pair comprised in moiety M2 comprises an antigen-binding surface. In one embodiment, said antigen-binding surface is composed of three complementarity-determining regions (CDRs) from each of the VH and VL domains. In one embodiment, said CDRs comprise the following amino acid sequences:

VHCDR1:
(SEQ ID NO: 37)
X1X2NMX3,

wherein
X1 is selected from D and A;
X2 is selected from Y and A; and
X3 is selected from D and A;

VHCDR2:
(SEQ ID NO: 38)
X4INPX5X6X7TTSX8X9X10KFKG,

wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;

VLCDR1:
(SEQ ID NO: 40)
KSSQSLLX11SX12NX13KNX14LA,

wherein
X11 is selected from Y and A;
X12 is selected from T and S;
X13 is selected from Q and R; and
X14 is selected from Y and A;

VLCDR2:
(SEQ ID NO: 41)
X15ASTRES

wherein
X15 is selected from W and A; and

VLCDR3:
(SEQ ID NO: 42)
QQX16X17X18X19PX20T

wherein
X16 is selected from Y and A;
X17 is selected from F and Y;
X18 is selected from I and N;
X19 is selected from Y and A; and
X20 is selected from R and Y.

In one embodiment, the CDRs of moiety M2 further comprise:

VHCDR3:
(SEQ ID NO: 39)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

In an alternative embodiment, the CDRs of moiety M2 further comprise:

VHCDR3:
(SEQ ID NO: 71)
SEAGNYYWYFDV

As defined herein, embodiments of the hTfR1-binding moiety M2 in the bispecific binding molecule of the first aspect of the disclosure that comprise a VH/VL pair have specific amino acid sequences in the regions determining its binding capability, such as the CDRs of the heavy and/or light chain variable domain, or indeed the entire VL and/or VH domains or regions. Non-limiting examples of such specific amino acid sequences are provided herein for the specific antibodies and fragments thereof generated and characterized as described in Examples 15-29. Further development of these antibodies into bispecific binding molecules of the present disclosure is detailed in Examples 30-38.

It is contemplated that the specific sequence information provided for the generated binding molecules enables the skilled person to define combinations and variations of these sequences within the scope of the invention, such as including the combinations and variations afforded by the variation in the general CDR sequences provided herein.

In one embodiment, said VHCDR2 of moiety M2 is:

VHCDR2:
(SEQ ID NO: 43)
X4INPX5X6X7TTSX8NEKFKG,

wherein
X4 is selected from D and A;
X5 is selected from D and A;
X6 is selected from Y and A;
X7 is selected from D and A; and
X8 is selected from Y and A.

In one embodiment, said VLCDR1 of moiety M2 is:

VLCDR1:
(SEQ ID NO: 44)
KSSQSLLX11STNQKNX14LA,

wherein
X11 is selected from Y and A; and
X14 is selected from Y and A.

In one embodiment, said VLCDR3 of moiety M2 is:

VLCDR3:
(SEQ ID NO: 45)
QQX16FIX19PRT

wherein
X16 is selected from Y and A;
X19 is selected from Y and A.

In one embodiment, the amino acid sequence of said VHCDR1 in moiety M2 is selected from the group consisting of SEQ ID NO:46 and 52-54.

In one embodiment, the amino acid sequence of said VHCDR2 in moiety M2 is selected from the group consisting of SEQ ID NO:47, 55-59 and 70, for example selected from the group consisting of SEQ ID NO: 47 and 55-59.

In one embodiment, the amino acid sequence of said VHCDR3 in moiety M2 is selected from the group consisting of SEQ ID NO:48, 60-64 and 71, for example selected from the group consisting of SEQ ID NO:48 and 60-64.

In one embodiment, the amino acid sequence of said VLCDR1 in moiety M2 is selected from the group consisting of SEQ ID NO:49, 65, 66 and 72, for example selected from the group consisting of SEQ ID NO:49, 65 and 66.

In one embodiment, the amino acid sequence of said VLCDR2 in moiety M2 is selected from the group consisting of SEQ ID NO:50 and 67.

In one embodiment, the amino acid sequence of said VLCDR3 in moiety M2 is selected from the group consisting of SEQ ID NO:51, 68, 69 and 73, for example selected from the group consisting of SEQ ID NO:51, 68 and 69.

In some embodiments, the CDR sequences can be freely combined among the options listed above. Such embodiments for example include, but are not limited to, those combinations exemplified in Example 23 for alanine substituted variants of the representative M2 moiety h26D3.

In a specific embodiment of a bispecific binding molecule of the disclosure, the amino acid sequences of the six CDRs in moiety M2 are the following:

VHCDR1:
(SEQ ID NO: 46)
DYNMD,
VHCDR2:
(SEQ ID NO: 47)
DINPDYDTTSYNEKFKG,
VHCDR3:
(SEQ ID NO: 48)
GGYSGSSYYHPMDY,
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

In another specific embodiment of a bispecific binding molecule of the disclosure, the amino acid sequences of the six CDRs in moiety M2 are the following:

VHCDR1:
(SEQ ID NO: 46)
DYNMD,
VHCDR2:
(SEQ ID NO: 57)
DINPDADTTSYNEKFKG,
VHCDR3:
(SEQ ID NO: 48)
GGYSGSSYYHPMDY,
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

In another specific embodiment of a bispecific binding molecule of the disclosure, the amino acid sequences of the six CDRs in moiety M2 are the following:

VHCDR1:
(SEQ ID NO: 46)
DYNMD,
VHCDR2:
(SEQ ID NO: 70)
DINPNYDTTSYSQKFKG,
VHCDR3:
(SEQ ID NO: 71)
SEAGNYYWYFDV,
VLCDR1:
(SEQ ID NO: 72)
KSSQSLLYSSNRKNYLA,
VLCDR2:
(SEQ ID NO: 50)
WASTRES,
and
VLCDR3:
(SEQ ID NO: 73)
QQYYNYPYT.

In another specific embodiment of a bispecific binding molecule of the disclosure, the amino acid sequences of the six CDRs in moiety M2 are the following:

VHCDR1:
(SEQ ID NO: 74)
NYWLG,
VHCDR2:
(SEQ ID NO: 75)
DIFPGSDNTYYNEKFKG,
VHCDR3:
(SEQ ID NO: 76)
SGNFYAMDY,
VLCDR1:
(SEQ ID NO: 77)
SASSSVNYMN,
VLCDR2:
(SEQ ID NO: 78)
DTSKLAS,
VLCDR3:
(SEQ ID NO: 79)
FQGSGYPFT.

In one embodiment of the bispecific binding molecule of the disclosure, the VH domain in moiety M2 comprises or consists of an amino acid sequence selected from
(i) the group consisting of SEQ ID NO: 80-93, 101 and 103, for example the group consisting of SEQ ID NO:80-93, for example the group consisting of SEQ ID NO:80 and 86; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i).

In one embodiment of the bispecific binding molecule of the disclosure, the VL domain in moiety M2 comprises or consists of an amino acid sequence selected from
(i) the group consisting of SEQ ID NO:94-100, 102 and 104, for example the group consisting of SEQ ID NO:94-100; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions a sequence defined in (i).

In a particular such embodiment, the VH domain and VL domain in moiety M2 are both as defined immediately above, i.e. a VH comprising or consisting of a sequence selected from SEQ ID NO:80-93 and sequences having at least 80% sequence identity thereto, and a VL comprising or consisting of a sequence selected from SEQ ID NO:94-100 and sequences having at least 80% sequence identity thereto.

In another embodiment of the bispecific binding molecule of the disclosure, said VH domain in moiety M2 comprises or consists of an amino acid sequence selected from SEQ ID NO:105 and a sequence having at least 80%, at least 90%, at least 92%, at least 94%, at least 95%, at least 96%, at least 97%, at least 98% or at least 99% identity to SEQ ID NO:105, provided that the sequences of the CDR regions are 100% identical to those of SEQ ID NO:105.

In another embodiment of the bispecific binding molecule of the disclosure, said VL domain of moiety M2 comprises or consists of an amino acid sequence selected from SEQ ID NO:106 and a sequence having at least 80%, at least 90%, at least 92%, at least 94%, at least 95%, at least 96%, at least 97%, at least 98% or at least 99% identity to SEQ ID NO:106, provided that the sequences of the CDR regions are 100% identical to those of SEQ ID NO:106.

In a particular such embodiment, the M2 VH domain and VL domain are both as defined immediately above, i.e. a VH comprising or consisting of the sequence SEQ ID NO:105 and sequences having at least 80% sequence identity thereto, and a VL comprising or consisting of the sequence SEQ ID NO:106 and sequences having at least 80% sequence identity thereto.

In one embodiment of the bispecific binding molecule of the disclosure, said VH domain in M2 comprises SEQ ID NO:80 and said VL domain in M2 comprises a sequence selected from SEQ ID NO:94-100.

In one embodiment of the bispecific binding molecule of the disclosure, said VH domain in M2 comprises a sequence selected from SEQ ID NO:80-93 and said VL domain in M2 comprises SEQ ID NO:94.

In one embodiment of the bispecific binding molecule of the disclosure, said VH domain in M2 comprises SEQ ID NO:80 and said VL domain in M2 comprises SEQ ID NO:94.

In one embodiment of the bispecific binding molecule of the disclosure, said VH domain in M2 comprises SEQ ID NO:86 and said VL domain in M2 comprises SEQ ID NO:94.

In some embodiments of the bispecific binding molecule of the disclosure, the hTfR1-binding moiety M2 comprises one first cysteine residue in the VH domain thereof, and one second cysteine residue in the VL domain thereof, said first and second cysteine residues being arranged such that they form a disulfide bridge connecting the VH and VL domains.

Without wishing to be bound by theory, the provision of the first and second cysteine residues in the VH and VL domains of moiety M2, respectively, and the resultant disulfide bridge between VH and VL, is contemplated to allow for the formation of a more stable VH/VL pairing. Non-limiting examples of advantages with such increased stability include an improved storage stability and an increased resistance towards multimerization. The introduction of cysteine residues and the resulting formation of a disulfide bridge in moiety M2 in the bispecific binding molecule of the disclosure is contemplated to increase the stability of the bispecific binding molecule. As realized by a person of skill in the art from the context herein, such increased stability may for example be measured as an increase in the monomeric content of the bispecific binding molecule in a sample after storage, compared to the monomeric content of a binding molecule having an identical sequence except for the cysteine residues. Evaluating monomeric content may for example be done using size exclusion liquid chromatography (SEC) after simulated stress conditions and/or long-term storage. This will provide measures of the monomer content and presence of aggregates. In one embodiment, the bispecific binding molecule of the disclosure is defined as stable if it exhibits a monomer content of 90% or more after storage for two weeks at 40° C. as determined by SEC. In another embodiment, the bispecific binding molecule of the disclosure is defined as stable if it exhibits a monomer content of 95% or more after storage for two weeks at 40° C. as determined by SEC. In yet another embodiment, the bispecific binding molecule of the disclosure is defined as stable if it exhibits a monomer content of 98% or more after storage for two weeks at 40° C. as determined by SEC.

Importantly, the increased size and avidity of dimers, or further multimers, of binding molecules comprising pairs of VH and VL domains may cause undesirable cross-linking of targets in vivo and altered pharmacodynamic properties. This is especially important when binding to the transferrin receptor to cross the blood brain barrier, because it is crucial to avoid multimerization, as this leads to down-regulation of the transferrin receptor. Such down-regulation, in turn, reduces the transport capacity over the blood brain barrier and can potentially cause safety problems for a biopharmaceutical product, due to a lower abundance of transferrin receptors on the cell surface. In addition, dimerization and further oligomerization is contemplated to pose considerable challenges with respect to the production, analysis, formulation and storage of biologics in connection with commercial or clinical applications.

Thus, in this embodiment of the bispecific binding molecule of the disclosure, the hTfR1-binding moiety M2 is engineered to comprise a disulfide bridge in order to stabilize the VL/VH or VH/VL forms. This is shown to be crucial for producing antibody constructs that are both stable and only bind in a monomeric form to transferrin receptor. Data show that the presence of only small amounts of dimeric forms of binding molecules leads to avidity binding to the transferrin receptor. Also, without the stabilizing disulfide, the disclosed constructs may be produced in a dimeric or oligomeric form and be unstable over time under various conditions. Thus, by introducing a disulfide bond between the VL/VH or VH/VL domains, the bispecific binding molecules of the disclosure are contemplated to be both more stably produced and in addition prevent avidity binding by moiety M2 to the transferrin receptor.

In one embodiment, said first cysteine (in the M2 VH domain) is located at an amino acid position selected from VH position 39-49 as determined by reference to the Kabat numbering scheme. In a more specific embodiment, said first cysteine is located at an amino acid position selected from VH position 41-47, such as selected from VH position 43-45, all as determined by reference to the Kabat numbering scheme. In a yet more specific embodiment, the first cysteine is located at VH position 44 per Kabat numbering.

In one embodiment, said second cysteine (in the M2 VL domain) is located at an amino acid position selected from VL position 95-105 as determined by reference to the Kabat numbering scheme. In a more specific embodiment, said first cysteine is located at an amino acid position selected from VL position 97-103, such as selected from VL position 99-101, all as determined by reference to the Kabat numbering scheme. In a yet more specific embodiment, the first cysteine is located at VL position 100 per Kabat numbering.

In one exemplary embodiment, said first cysteine residue is located at M2 VH position 44 and said second cysteine residue is located at M2 VL position 100, as determined by reference to the Kabat numbering scheme.

In one embodiment of the bispecific binding molecule of the disclosure, the VH domain in moiety M2 comprises or consists of an amino acid sequence selected from
(i) the group consisting of SEQ ID NO:124-139, for example the group consisting of SEQ ID NO:124-137, for example the group consisting of SEQ ID NO:124 and 130; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44 (Kabat position 44).

In one embodiment of the bispecific binding molecule of the disclosure, the VL domain in moiety M2 comprises or consists of an amino acid sequence selected from
(i) the group consisting of SEQ ID NO:141-149, for example the group consisting of SEQ ID NO:141-147; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106 (Kabat position 100).

In a particular such embodiment, the VH domain and VL domain in moiety M2 are both as defined immediately above, i.e. a VH comprising or consisting of a sequence selected from SEQ ID NO:124-139 and sequences having at least 80% sequence identity thereto, and a VL comprising or consisting of a sequence selected from SEQ ID NO:141-149 and sequences having at least 80% sequence identity thereto, subject to the defined provisos.

In one embodiment of a bispecific binding molecule of the disclosure, the M2 VH domain comprises SEQ ID NO:124 and the M2 VL domain comprises a sequence selected from SEQ ID NO:141-147.

In one embodiment of a bispecific binding molecule of the disclosure, the M2 VH domain comprises a sequence selected from SEQ ID NO:124-137 and the M2 VL domain comprises SEQ ID NO:141.

In one embodiment of a bispecific binding molecule of the disclosure, the M2 VH domain comprises SEQ ID NO:124 and the M2 VL domain comprises SEQ ID NO:141.

In one embodiment of a bispecific binding molecule of the disclosure, the M2 VH domain comprises SEQ ID NO:130 and the M2 VL domain comprises SEQ ID NO:141.

In one embodiment of the bispecific binding molecule of the disclosure, the VH domain in moiety M2 comprises or consists of an amino acid sequence selected from
(i) the group consisting of SEQ ID NO:167-177, for example the group consisting of SEQ ID NO:167-172, for example the group consisting of SEQ ID NO:167-168; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44 (Kabat position 44).

In one such embodiment of the bispecific binding molecule of the disclosure, the VL domain in moiety M2 comprises or consists of an amino acid sequence selected from
(i) the group consisting of SEQ ID NO:141-149, for example the group consisting of SEQ ID NO:141-147; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106 (Kabat position 100).

In a particular such embodiment, the VH domain and VL domain in moiety M2 are both as defined immediately above, i.e. a VH comprising or consisting of a sequence selected from SEQ ID NO:167-177 and sequences having at least 80% sequence identity thereto, and a VL comprising or consisting of a sequence selected from SEQ ID NO:141-149 and sequences having at least 80% sequence identity thereto, subject to the defined provisos.

In one embodiment of a bispecific binding molecule of the disclosure, the M2 VH domain comprises a sequence selected from SEQ ID NO:167-168 and the M2 VL domain comprises SEQ ID NO:141.

In one embodiment of a bispecific binding molecule of the disclosure, the M2 VH domain comprises SEQ ID NO:167 and the M2 VL domain comprises SEQ ID NO:141.

In one embodiment of a bispecific binding molecule of the disclosure, the M2 VH domain comprises SEQ ID NO:168 and the M2 VL domain comprises SEQ ID NO:141.

In another embodiment of the bispecific binding molecule of the disclosure, the VH domain in moiety M2 comprises or consists of an amino acid sequence selected from SEQ ID NO:140 and a sequence having at least 80%, at least 90%, at least 92%, at least 94%, at least 95%, at least 96%, at least 97%, at least 98% or at least 99% identity to SEQ ID NO:140, provided that the sequences of the CDR regions are 100% identical to those of the CDR regions in SEQ ID NO:140, and provided that the sequence comprises a cysteine residue at position 44 (Kabat position 44).

In another of the bispecific binding molecule of the disclosure, the VL domain in moiety M2 comprises or consists of an amino acid sequence selected from SEQ ID NO:150 and a sequence having at least 80%, at least 90%, at least 92%, at least 94%, at least 95%, at least 96%, at least 97%, at least 98% or at least 99% identity to SEQ ID NO:150, provided that the sequences of the CDR regions are 100% identical to those of the CDR regions in SEQ ID NO:150, and provided that the sequence comprises a cysteine residue at position 99 (Kabat position 100).

In a particular such embodiment, the M2 VH domain and M2 VL domain are both as defined immediately above, i.e. a VH comprising or consisting of the sequence SEQ ID NO:140 and sequences having at least 80% sequence identity thereto, and a VL comprising or consisting of the sequence SEQ ID NO:150 and sequences having at least 80% sequence identity thereto, subject to the defined provisos.

### Linkage of M1 and M2

In one embodiment of a bispecific binding molecule of the disclosure, the VH/VL pair of M2 forms part of an antibody construct. In one such embodiment, the VH/VL pair of M2 is present in an antibody fragment selected from the group consisting of a Fab fragment, a single chain Fab (scFab) fragment, an Fv fragment and a single chain (scFv) fragment. In a particular embodiment, said antibody fragment is an scFv.

Thus, in one embodiment of the bispecific binding molecule of the disclosure, the M2 moiety comprises an scFv. In other words, the VH/VL pair in M2 forms part of an scFv, in which the VH and VL domains are coupled together by a peptide scFv linker. In one such embodiment, the scFv linker may either be attached to the C-terminal amino acid residue of the VH domain and to the N-terminal amino acid residue of the VL domain, or to the N-terminal amino acid residue of the VH domain and to the C-terminal amino acid residue of the VL domain. In the first configuration, the VH domain precedes the VL domain in the polypeptide chain constituting the scFv, while in the second configuration, the VL domain precedes the VH domain. The two different configurations are sometimes denoted “VH first” and “VL first” in the present disclosure.

The design and selection of suitable peptide linkers for use within and between domains and moieties of fusion proteins, antibody constructs and other such engineered polypeptides is within the capacity of a person of skill in the art. In some embodiments where the hTfR1-binding moiety M2 comprises or consists of an scFv, the scFv linker is a flexible peptide linker, consisting of from 5 to 40 amino acid residues, for example from 10 to 30 amino acid residues, for example from 15 to 25 amino acid residues, for example about 15 amino acid residues, for example 15 amino acid residues, for example comprising or consisting of the sequence (G₄S)₃ (SEQ ID NO:166).

The same or similar design considerations apply to linkers used to attach the AβpE3-binding moiety M1 to the hTfR1-binding moiety M2. In one embodiment, M1 and M2 are connected to each other by at least one peptide linker between M1 and M2. In one embodiment, said at least one peptide linker between M1 and M2 is attached, on the M2 side, to the C-terminal amino acid residue of the VH domain of M2 or to the N-terminal amino acid residue of the VL domain of M2.

As described above, the design and selection of suitable peptide linkers for use within and between domains and moieties of fusion proteins, antibody constructs and other such engineered polypeptides is within the capacity of a person of skill in the art. In one embodiment of the binding protein of the disclosure, M1 and M2 are linked by at least one flexible peptide linker. In one embodiment, the at least one flexible peptide linker comprises glycine, serine, alanine and/or threonine residues. In a more specific embodiment, said linker(s) has a general formula selected from (GₙSₘ)ₚ and (SₙGₘ)ₚ, wherein, independently, n=1−7, m=0−7, n+m s 8 and p=1−10. In some embodiments, at least one linker is between 10 and 50 amino acid residues long, such as between 10 and 30 amino acid residues long, such as between 15 and 25 amino acid residues long or between 10 and 20 amino acids long. In case M1 and M2 are linked via two or more linkers, all of the disclosed, optional linker designs apply individually to each linker present independently of the other linkers. Thus, for example, if there are two linkers, they may be of the same or different length, and have the same amino acid sequence or different amino acid sequences.

In one embodiment of the bispecific binding molecule of the disclosure, moiety M1 is provided as a knob-into-hole antibody comprising two identical antibody light chains; one antibody hole heavy chain; and one antibody knob heavy chain; and M2 is provided as an scFv linked to the C-terminal amino acid residue of the knob heavy chain of M1.

In one such embodiment of the bispecific binding molecule, the amino acid sequence of the antibody light chain of M1 comprises or consists of SEQ ID NO:160; the amino acid sequence of the antibody hole heavy chain of M1 comprises or consists of SEQ ID NO:161, and the combined amino acid sequence of the M1 antibody knob heavy chain with linked M2 scFv comprises or consists of a sequence selected from SEQ ID NO:162-164. In one embodiment, the amino acid sequence of the antibody light chain of M1 comprises or consists of SEQ ID NO:160, the amino acid sequence of the antibody hole heavy chain of M1 comprises or consists of SEQ ID NO:161, and the amino acid sequence of said M1 antibody knob heavy chain with linked M2 scFv comprises or consists of SEQ ID NO:162. In an alternative embodiment, the amino acid sequence of the antibody light chain of M1 comprises or consists of SEQ ID NO:160, the amino acid sequence of the antibody hole heavy chain of M1 comprises or consists of SEQ ID NO:161, and the amino acid sequence of said M1 antibody knob heavy chain with linked M2 scFv comprises or consists of SEQ ID NO:163. In another alternative embodiment, the amino acid sequence of the antibody light chain of M1 comprises or consists of SEQ ID NO:160, the amino acid sequence of the antibody hole heavy chain of M1 comprises or consists of SEQ ID NO:161, and the amino acid sequence of said M1 antibody knob heavy chain with linked M2 scFv comprises or consists of SEQ ID NO:164.

In certain embodiments, the bispecific binding molecule of the disclosure comprises
(a) an AβpE3 binding moiety M1 comprising:

VHCDR1:
(SEQ ID NO: 7)
GFTMN,
VHCDR2:
(SEQ ID NO: 8)
LINPYNGVTTYNQKFKG,
VHCDR3:
(SEQ ID NO: 3)
EGNWEGVY,
VLCDR1:
(SEQ ID NO: 10)
RSSQSLLDSNGKTYLH,
VLCDR2:
(SEQ ID NO: 9)
LVSILDS,
and
VLCDR3:
(SEQ ID NO: 6)
VQGTHFPFT;

and
(b) a human transferrin receptor 1 hTfR1 binding moiety M2 comprising:

VHCDR1:
(SEQ ID NO: 46)
DYNMD,
VHCDR2:
(SEQ ID NO: 57)
DINPDADTTSYNEKFKG,
VHCDR3:
(SEQ ID NO: 48)
GGYSGSSYYHPMDY,
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

In certain embodiments, the bispecific binding molecule of the disclosure comprises (a) an AβpE3 binding moiety M1 comprising a variable heavy chain comprising an amino acid sequence at least 95% identical, or at least 98% identical, or 100% identical to the amino acid sequence of SEQ ID NO:18 and a variable light chain comprising an amino acid sequence at least 95% identical, or at least 98% identical, or 100% identical to the amino acid sequence of SEQ ID NO:23; and (b) a human transferrin receptor 1 (hTfR1) binding moiety M2 comprising: a variable heavy chain comprising an amino acid sequence at least 95% identical, or at least 98% identical, or 100% identical to the amino acid sequence of SEQ ID NO:130, and a variable light chain comprising an amino acid sequence at least 95% identical, or at least 98% identical, or 100% identical to the amino acid sequence of SEQ ID NO:141.

In certain embodiments, the bispecific binding molecule of the disclosure comprises an AβpE3 binding moiety M1 and a human transferrin receptor 1 (hTfR1) binding moiety M2, wherein the AβpE3 binding moiety M1 comprises a first and a second IgG heavy chain and two IgG light chains, and wherein the hTfR1 binding moiety M2 comprises an scFv, wherein the scFv is fused, via an optional linker, to the first IgG heavy chain and wherein the first and the second IgG heavy chains are paired via knob-into-hole. In certain embodiments, the first IgG heavy chain comprises the amino acid sequence at least 95% identical, or at least 98% identical, or 100% identical to the amino acid sequence of SEQ ID NO:161 and the second IgG heavy chain comprises the amino acid sequence at least 95% identical, or at least 98% identical, or 100% identical to the amino acid sequence of positions 1 to 446 of the amino acid sequence of SEQ ID NO:162. In certain embodiments, the first IgG heavy chain comprises the amino acid sequence at least 95% identical, or at least 98% identical, or 100% identical to the amino acid sequence of positions 1 to 446 of the amino acid sequence of SEQ ID NO:162 and the second IgG heavy chain comprises the amino acid sequence at least 95% identical, or at least 98% identical, or 100% identical to the amino acid sequence of SEQ ID NO:161. In certain embodiments, the two IgG light chains each comprises the amino acid sequence at least 95% identical, or at least 98% identical, or 100% identical to the amino acid sequence of SEQ ID NO:160. In certain embodiments, the scFv comprises the amino acid sequence at least 95% identical, or at least 98% identical, or 100% identical to the amino acid sequence of SEQ ID NO:154.

In certain embodiments, the bispecific binding molecule of the disclosure comprises an AβpE3 binding moiety M1 and a human transferrin receptor 1 (hTfR1) binding moiety M2, wherein the AβpE3 binding moiety M1 comprises a first and a second IgG heavy chain and two IgG light chains, and wherein the hTfR1 binding moiety M2 comprises an scFv, wherein the scFv is fused, via an optional linker, to the first IgG heavy chain and wherein the first and the second IgG heavy chains are paired via knob-in-hole. In certain embodiments, the first IgG heavy chain fused to the scFv comprises the amino acid sequence of the amino acid sequence consisting of SEQ ID NO:162 and the second IgG heavy chain comprises the the amino acid sequence of the amino acid sequence consisting of SEQ ID NO:161. In certain embodiments, the two IgG light chains each comprises the amino acid sequence of the amino acid sequence consisting of SEQ ID NO:160.

### Amino Acid Sequences

In various embodiments of the bispecific binding molecule of the disclosure, the VH and VL sequences, when present in either moiety M1 or moiety M2 of the binding molecule, may be individually selected from any one of the listed sequences and sequences having at least 80%, at least 90%, at least 92%, at least 94%, at least 95%, at least 96%, at least 97%, at least 98%, at least 99% or at least 100% identity thereto. For embodiments wherein variable domains of the binding molecules of the disclosure are defined by such a particular percentage sequence identity to a reference sequence, the VH and/or VL domains may retain the identical CDR sequences of those present in the reference sequence such that the allowed percentage variation is present only within the framework regions.

In various embodiments of the bispecific binding molecule of the disclosure, sequences of complementarity determining regions (CDR regions) and general amino acid position numbering within antibody sequences may suitably be defined using the Kabat convention, which is well known to a person of skill in the art of antibody technology (see e.g. Kabat (1991), Sequences of Proteins of Immunological Interest, 5^{th} edition, NIH Publication no 91-3242 from the US Department of Health and Human Services).

### Affinity for a Target

As used herein, the terms “specific binding to X”, “selective binding to X” and “affinity for X”, wherein X is a target (e.g. an antigen or an epitope, such as the AβpE3 bound by moiety M1 or the hTfR1 bound by moiety M2 of the bispecific binding molecule of the disclosure), refer to a property of a binding molecule which may be tested for example by ELISA, by surface plasmon resonance (SPR), by Kinetic Exclusion Assay (KinExA®) or by bio-layer interferometry (BLI). The skilled person is aware of these methods and others.

For example, binding affinity for antigen or epitope X may be tested in an experiment in which a binding molecule to be tested is captured on ELISA plates coated with target or antigen X, or an antigen exhibiting the epitope X, and a biotinylated detector antibody is added, followed by streptavidin-conjugated horse radish peroxidase (HRP). Alternatively, said detector antibody may be directly conjugated with HRP. Tetramethylbenzidine (TMB) substrate is added and the absorbance at 450 nm is measured using an ELISA multi-well plate reader. The skilled person may then interpret the results obtained by such experiments to establish at least a qualitative measure of the binding affinity of the binding molecule for X. If a quantitative measure is desired, for example to determine the EC50 value (the half maximal effective concentration) for the interaction, ELISA may also be used. The response of the binding molecule against a dilution series of X may be measured using ELISA as described above. The skilled person may then interpret the results obtained by such experiments and EC50 values may be calculated from the results, using for example GraphPad Prism v.9 and non-linear regression.

As used herein, the term “EC50” refers to the half maximal effective concentration of a binding molecule which induces a response halfway between the baseline and maximum after a specified exposure time.

Additionally, inhibition ELISA may be used to obtain a quantitative measure of interaction by determination of the “IC50” (the half maximal inhibitory concentration). In an inhibition ELISA, the concentration of a target or an antigen or epitope X in a fluid sample is measured by detecting interference in an expected signal output. In principle, a known antigen or epitope-bearing substance is used to coat a multi-well plate. In parallel, a binding molecule with putative affinity for the target, antigen or epitope is added and incubated with a solution containing antigen at varied concentrations. Following standard blocking and washing steps, samples containing the mixture of the binding molecule and the antigen or epitope are added to the well. Labeled detection antibody with affinity for the antigen- or epitope-binding molecule is then applied for detection using relevant substrates (for example TMB). In principle, if there is a high concentration of antigen or epitope in the fluid sample, a significant reduction in signal output will be observed. In contrast, if there is very little antigen or epitope in the fluid sample, there will be very little reduction in the expected signal output. The skilled person appreciates that the signal output is also dependent on the affinity of the binding molecule for said antigen or epitope.

As used herein, the term “IC50” refers to the half maximal inhibitory concentration of a binding molecule which induces a response halfway between the baseline and maximum inhibition after a specified exposure time. Herein, a lower IC50 value indicates that a lower concentration of antigen or epitope is required to interfere with the binding of the detection antibody to the known antigen or epitope coated on the plate, as compared to a higher IC50 value. Thus, a lower IC50 value typically corresponds to a higher affinity of the binding molecule.

The binding affinity of a binding molecule may also be tested by SPR. For example, said binding affinity may be tested in an experiment in which antigen or epitope X is immobilized on a sensor chip of the instrument, and the sample containing the binding molecule to be tested is passed over the chip. Alternatively, the binding molecule to be tested may be immobilized on a sensor chip of the instrument, and a sample containing X is passed over the chip. The skilled person may then interpret the results obtained by such experiments to establish at least a qualitative measure of the binding affinity of the moiety for X. If a quantitative measure is desired, for example to determine a K_{D} value for the interaction, SPR may also be used. Binding values may for example be defined in a Biacore (Cytiva) or ProteOn XPR 36 (Bio-Rad) instrument. The antigen or epitope is suitably immobilized on a sensor chip of the instrument, and samples of the binding molecule whose affinity is to be determined are prepared by serial dilution and injected. K_{D} values may then be calculated from the results using for example the 1:1 Langmuir binding model of the Biacore Insight Evaluation Software 2.0 or other suitable software, typically provided by the instrument manufacturer.

Another method for determining binding affinity of a binding molecule to antigen or epitope X is the Kinetic Exclusion Assay (KinExA; Sapidyne Instruments Inc; Darling and Brault, Assay and Drug Dev Tech (2004) 2(6):647-657) for measurements of the equilibrium binding affinity and kinetics between unmodified molecules in solution. A KinExA K_{D} analysis requires immobilization of one interaction partner (e.g. the titrated binding partner) to a solid phase, which is then used as a probe to capture the other interaction partner (e.g. the constant binding partner) free in solution once an equilibrium is reached.

The binding affinity may also be measured by bio-layer interferometry (BLI), a label-free technology for measuring biomolecular interactions within the interactome. It is an optical analytical technique that analyzes the interference pattern of white light reflected from two surfaces: a layer of immobilized protein on the biosensor tip, and an internal reference layer. The binding between a ligand (antigen or epitope X) immobilized on the biosensor tip surface and an analyte (such as a binding molecule with affinity for X) in solution produces an increase in optical thickness at the biosensor tip resulting in a wavelength shift, Δλ, which is a direct measure of the change in thickness of the biological layer. Interactions are measured in real time, providing the ability to monitor binding specificity, rates of association and dissociation, or concentration, with precision and accuracy.

The skilled person is aware of the above-mentioned and other methods for measuring the affinity of a binding molecule for target, antigen or epitope X, either qualitatively or quantitatively or both.

### Polynucleotides, Vectors and Cells

In one aspect, the present disclosure also provides a nucleotide sequence encoding a bispecific binding molecule disclosed herein. In certain embodiments, provided herein is a set of nucleotide sequences wherein the set encodes a bispecific binding molecule disclosed herein. In a specific embodiment, each nucleotide sequence of such a set encodes one polypeptide of a bispecific binding molecule disclosed herein. In one specific embodiment, such a set comprises a nucleotide sequence encoding the amino acid sequence of SEQ ID NO:162; a nucleotide sequence encoding the amino acid sequence of SEQ ID NO:161; and a nucleotide sequence encoding the amino acid sequence of SEQ ID NO:160.

Additionally provided are polynucleotides or a set of polynucleotides encoding a bispecific binding molecule disclosed herein or a portion thereof, a polynucleotide or a set of polynucleotides complementary thereto. In certain embodiments, the set of polynucleotides together encode a bispecific binding molecule disclosed herein. The set of polynucleotides encoding the bispecific binding molecule disclosed herein may encompass two or more polynucleotides, each encoding a portion of the bispecific binding molecule. Polynucleotides disclosed herein can be RNA or DNA (e.g., cDNA, genomic DNA, or synthetic DNA), and can be double-stranded or single-stranded, and if single stranded can be the coding strand or non-coding (anti-sense) strand.

The present disclosure further provides a vector or a set of vectors comprising a polynucleotide, or a set of polynucleotides disclosed herein. Such vectors are useful, for example, for amplifying the polynucleotide or set of polynucleotides in host cells to create useful quantities thereof, and for expressing the bispecific binding molecule disclosed herein. Any suitable vectors can be used to introduce one or more polynucleotides disclosed herein into a cell. Exemplary vectors include, but not limited to, lentivirus vectors, adeno-associated viral (AAV) vectors, adenoviral (AV), and liposomal vectors.

The present disclosure further provides a cell (e.g., a host cell) comprising any one or more of: a bispecific binding molecule disclosed herein, a polynucleotide or a set of polynucleotides disclosed herein, or a vector or a set of vectors disclosed herein. In certain embodiments, the cell replicates the polynucleotide or et of polynucleotides disclosed herein or the vector or set of vectors disclosed herein. Cells disclosed herein can be Escherichia coli, mammalian cells (e.g., myeloma cells, Chinese Hamster Ovary (CHO) cells, or hybridoma cells), yeast cells, insect cells, or plant cells. Mammalian cells may provide translational modifications (e.g., glycosylation, truncation, lipidation, or phosphorylation) that may confer optimal biological activity on recombinant expression products.

The present disclosure further provides a method of making a bispecific binding molecule disclosed herein, comprising culturing the cell under conditions that result in the expression of the bispecific binding molecule, and isolating the bispecific binding molecule.

### Pharmaceutical Compositions

In a second aspect, there is provided a pharmaceutical composition comprising a bispecific binding molecule as described herein and at least one pharmaceutically acceptable excipient or carrier.

Techniques for formulating antibodies, fragments thereof and other related binding molecules for human therapeutic use are well known in the art and are reviewed, for example, in Wang et al. (2007), J Pharm Sci, 96:1-26, the contents of which are incorporated herein in their entirety.

Pharmaceutically acceptable excipients that may be used to formulate the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene block polymers, polyethylene glycol and wool fat.

In certain embodiments, the pharmaceutical compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual) and transdermal administration. In preferred embodiments, the composition is formulated for intravenous or subcutaneous administration.

### Methods of Prevention, Treatment, Diagnosis, Prognosis and Detection

The present disclosure provides a method for treating a disease or disorder in a subject in need thereof, comprising administering to the subject a therapeutically effective amount of a bispecific binding molecule disclosed herein, or a pharmaceutical composition comprising thereof. In certain embodiments, provided herein is a bispecific binding molecule disclosed herein or a pharmaceutical composition comprising thereof, for use in the manufacture of a medicament for the treatment of a disease or disorder in a subject in need thereof.

The bispecific binding molecules according to the present disclosure may be useful as therapeutic and/or diagnostic agents.

Hence, in a further aspect of the disclosure, there is provided a bispecific binding molecule according to the first aspect, or a pharmaceutical composition according to the second aspect, for use as a medicament.

In yet another aspect of the disclosure, there is provided a bispecific binding molecule according to the first aspect, or a pharmaceutical composition according to the second aspect, for use as a diagnostic agent.

In yet another aspect of the disclosure, there is provided a bispecific binding molecule according to the first aspect, or a pharmaceutical composition according to the second aspect, for use as a prognostic agent.

Also provided are methods of preventing, treating or diagnosing disease or assessing disease prognosis, wherein a bispecific binding molecule as disclosed herein is administered to a subject, typically a human subject.

Also provided is the use of the disclosed bispecific binding molecule for the manufacture of compositions (such as medicaments) for use in the prevention, treatment, diagnosis and/or prognosis of any one of the listed diseases.

Also provided are methods of detecting or diagnosing a disease in a subject, wherein the methods comprise contacting a sample obtained from the subject with a bispecific binding molecule as described herein. These methods are typically in vitro methods.

Thus, said bispecific binding molecule, or pharmaceutical composition comprising it, is useful in the treatment, prevention and/or diagnosis of a condition selected from neurological disorders or conditions characterized by accumulation and/or aggregation of Aβ, such as formation of amyloid plaques. Such diseases or conditions include but are not limited to Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type); as well as other diseases which are based on or associated with amylogenic proteins, such as progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.

Thus, in one embodiment, there is provided a bispecific binding molecule, or pharmaceutical composition comprising it, for use in the treatment, prevention, diagnosis and/or prognosis of an Aβ peptide-associated condition. In one embodiment, there is provided a bispecific binding molecule, or pharmaceutical composition comprising it, for use in the treatment, prevention, diagnosis and/or prognosis of an Aβ peptide-associated condition, selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.

In one specific embodiment, said bispecific binding molecule, or pharmaceutical composition comprising it, is provided for use in the treatment, prevention, diagnosis and/or prognosis of Alzheimer's disease.

In another aspect, there is provided a method of treatment, prevention, diagnosis and/or prognosis of an Aβ peptide-associated condition in a mammal having, or being at risk of developing, said disorder, comprising administering to said mammal an amount, such as a therapeutically effective amount, of a bispecific binding molecule, or pharmaceutical composition comprising it.

In one embodiment, said Aβ peptide-associated condition is, for example, selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration. In a more specific embodiment, said Aβ peptide-associated condition is Alzheimer's disease.

With regard to therapeutic or preventive use of the disclosed bispecific binding molecule for the treatment of neurodegenerative diseases, there are several putative mechanisms of action. Without wishing to be bound by theory, non-limiting and independently possible mechanisms of action are for example binding to AβpE3 monomers to prevent seeding of Aβ aggregation, binding to and removal of soluble neurotoxic AβpE3-containing aggregates (protofibrils) and/or clearance of AβpE3-containing amyloid plaques to alleviate amyloidosis and improve cognitive function.

With regard to diagnostic or prognostic use of the disclosed bispecific binding molecule in neurodegenerative diseases, the putatively harmful AβpE3 species can be detected and measured in patients at risk of disease or showing signs of incipient disease. One such method is PET scan using a radio-labelled antibody of the disclosure. Another method for diagnosis and prognosis is biochemical analysis analyzing the levels of AβpE3 in blood, plasma, CSF and other fluids, using such methods as ELISA, Mesoscale Discovery (MSD) or Simoa.

### Kits

Also provided herein are kits comprising a bispecific binding molecule disclosed herein, or a composition provided herein, packaged into suitable packaging material. A kit optionally includes a label or packaging insert comprising a description of the components and/or instructions for use in vitro, in vivo, or ex vivo, of the components therein. Kits provided herein can additionally include other components. Each component of the kit can be enclosed within an individual container, and all of the various containers can be within a single package.

### INCORPORATION BY REFERENCE

Various publications are cited in the present application, each of which is incorporated by reference herein in its entirety.

### BRIEF DESCRIPTION OF THE FIGURES

FIG. 1 shows binding of the indicated hybridoma clones to monomeric Aβ1-42, Aβ32-42, Aβ33-42, AβpE3-42 and Aβ4-42 using ELISA, as described in Example 1.

FIG. 2 shows binding of the indicated hybridoma clones to AβpE3-42 protofibrils using ELISA, as described in Example 1.

FIG. 3 shows inhibition-response curves of the indicated recombinant antibodies against monomeric AβpE3-40 using inhibition ELISA, as described in Example 3.

FIG. 4 shows the inhibition-response curves of the indicated recombinant antibodies against monomeric Aβ1-40 using inhibition ELISA, as described in Example 3.

FIG. 5 shows the inhibition-response curves of the indicated recombinant antibodies against AβpE11-40 using inhibition ELISA, as described in Example 3.

FIG. 6 shows the inhibition-response curves of the indicated recombinant antibodies against AβpE3-42 protofibrils (PF) using inhibition ELISA, as described in Example 3.

FIG. 7 shows binding interactions for the indicated recombinant antibodies against AβpE3-40 monomers, measured by SPR as described in Example 3.

FIG. 8 shows binding interactions for the indicated recombinant antibodies against AβpE3-42 protofibrils, measured by SPR as described in Example 3.

FIG. 9 is a diagram showing the depletion of AβpE3-40 levels in soluble AD brain extracts by the indicated recombinant antibodies using immunoprecipitation, as described in Example 4.

FIG. 10 is a diagram showing the depletion of AβpE3-42 levels in soluble AD brain extracts by the indicated recombinant antibodies using immunoprecipitation, as described in Example 4.

FIG. 11 shows the binding of the indicated recombinant antibodies to amyloid plaques in sections of human brain by immunohistochemistry, as described in Example 4.

FIG. 12 shows the binding of the indicated humanized antibodies to monomeric AβpE3-28, Aβ1-28, Aβ2-28, Aβ3-28, Aβ4-28, Aβ5-28 and AβpE11-28 using inhibition ELISA, as described in Example 6.

FIG. 13 shows the binding of the indicated humanized antibodies to AβpE3-40 and Aβ1-40 monomers (M) and AβpE3-42 and Aβ1-42 protofibrils (PF) using inhibition ELISA, as described in Example 6.

FIG. 14 shows binding interactions for the indicated humanized antibodies against AβpE3-40 monomers, measured by SPR as described in Example 6.

FIG. 15 shows binding interactions for the indicated humanized antibodies against AβpE3-42 protofibrils, measured by SPR as described in Example 6.

FIG. 16 is a diagram showing the immunoprecipitation of AβpE3-x levels in soluble AD brain extracts by the indicated humanized antibodies using immunoprecipitation, as described in Example 7.

FIG. 17 shows the binding of the indicated humanized antibodies to amyloid plaques in sections of human brain by immunohistochemistry, as described in Example 7.

FIG. 18 shows the concentration-response effects of the indicated humanized antibodies on AβpE3 aggregation, as described in Example 8.

FIG. 19 shows the effects of the indicated humanized antibodies on Aβ plaque clearance in AD brain sections, as described in Example 8.

FIG. 20 shows the pharmacokinetic plasma concentration-time profile in mouse of the indicated murine and humanized antibodies, as described in Example 9.

FIG. 21 shows the pharmacokinetic plasma concentration-time profile in mouse of the indicated humanized antibodies, as described in Example 10.

FIG. 22 shows binding interactions for the indicated humanized antibodies against AβpE3-40 monomers, measured by SPR as described in Example 11.

FIG. 23 shows binding interactions for the indicated humanized antibodies against AβpE3-42 protofibrils, measured by SPR as described in Example 11.

FIG. 24 is a diagram showing the immunoprecipitation of AβpE3-x levels in soluble AD brain extracts by the indicated humanized antibodies using immunoprecipitation, as described in Example 12.

FIG. 25 shows the binding of the indicated humanized antibodies to amyloid plaques in sections of human brain by immunohistochemistry, as described in Example 12.

FIG. 26 shows the concentration-response effects of the indicated humanized antibodies on AβpE3 aggregation, as described in Example 13.

FIG. 27 shows the effects of the indicated humanized antibodies on Aβ plaque clearance in AD brain sections, as described in Example 13.

FIG. 28 shows the results of a binding screen of the indicated IgG antibodies from the immunization described in Example 15 towards human (hTfR1), cyno (cTfR1) and mouse (mTfR1) TfR1 in crude hybridoma supernatants by biolayer interferometry (BLI).

FIG. 29 shows the result of the BLI binding analysis described in Example 16 for the indicated Fab fragments of mouse antibodies 24B4, 26D3 and 37D10 as well as for a Fab fragment of control antibody 8D3.

FIGS. 30A-30E show mapping of antibody binding epitopes to the protease-like domain of hTfR1 as described in Example 16, by selective antibody binding to ELISA plates coated with either human, mouse or one of three different chimeric human/mouse TfR1 receptors. Antibodies 24B4, 26D3 and 37D10 bind to hTfR1 (FIG. 30A) but not to mTfR1 (FIG. 30B). In addition, 24B4, 26D3 and 37D10 also bind to h/m protease like domain chimera (FIG. 30D), but not to any of the plates coated with the other chimeric receptors (FIG. 30C and FIG. 30E).

FIG. 31 illustrates the epitope binning assay described in Example 16, with the following main four steps: Step 1—immobilization of bio-TfR1 on sensor chip; Step 2—wash of non-binding material; Step 3—binding of competing binder to TfR1; Step 4—association of binders to the TfR1:binder complex formed in Step 3. The data in Step 4 determines whether the two investigated binders compete in binding to hTfR1.

FIGS. 32A-32C show the result of carrying out the epitope binning assay as described in Example 16, showing the degree of competition between antibodies for simultaneous binding to hTfR1. Binding of antibody 26D3 (FIG. 32A), antibody 24B4 (FIG. 32B) and control antibody 15G11-1 (FIG. 32C) to preformed complexes of hTfR1 and either of the indicated antibodies. Binding responses for all antibodies are normalized to the binding response measured to free hTfR1 (no competing antibody).

FIGS. 33A-33B show binding by the indicated binders to hTfR1 on the surface of cells, studied as described in Example 16. The Y axes of both diagrams show the mean fluorescence intensity when staining cells with whole antibodies (FIG. 33A) and Fab fragments (FIG. 33B) of the indicated binders. No background staining is detected with the negative isotype control IgG (FIG. 33A) or the non-related Fab fragment, Ly128 (FIG. 33B).

FIGS. 34A-34C show the result of competition analysis of indicated binders with ferritin and transferrin as described in Example 17. The diagrams show MFI of the indicated binders binding to TfR1 expressed on THP-1 cell surfaces (FIG. 34A), MFI of ferritin on cell surface when exposed to the indicated binders (FIG. 34B), with the positive control antibody MA-712 competing with ferritin, and MFI of transferrin on cell surfaces when exposed to the indicated binders (FIG. 34C).

FIG. 35 is a collection of sensorgrams showing the result of SPR analysis of original 26D3 and 26D3 humanized as described in Example 18 (h26D3) in Fab formats when binding to hTfR1 and cTfR1 as indicated.

FIGS. 36A-36B show the result of BLI and ELISA binding studies carried out on mouse and humanized versions of 26D3 in an scFv format as described in Example 18. FIG. 36A shows sensorgrams obtained by BLI measurement of binding of the indicated constructs to hTfR1. FIG. 36B shows binding responses from ELISA measurement of binding of the indicated constructs to coated TfR1.

FIGS. 37A-37B are depictions of the x-ray structure of the complex of 26D3-Fab and hTfR1, determined as described in Example 19. The chain names as used in the coordinate files are indicated. FIG. 37A shows refined structure showing overall folds of three independent complexes in the asymmetric unit. FIG. 37B shows example of electron density (2m|Fo|−D|Fc|) contoured at the 1 σ level. The protein chains are drawn in cartoon representation while sugar moieties are shown in stick representation.

FIG. 38 is a ribbon representation of the h26D3-Fab human TfR1 complex determined with x-ray crystallography as described in Example 19. h26D3-Fab is depicted in dark gray and hTfR1 in white. The binding interface (epitope/paratope) is encircled.

FIG. 39 is a surface area representation of hTfR1 with the binding sites for the natural ligands ferritin and transferrin indicated, as well as the epitope for the binder 26D3 of the present disclosure. The different binding sites and epitope are depicted with a circle around each specific site.

FIGS. 40A-40C illustrate the work on generating and characterizing an hTfR1-KI mouse model as described in Example 20. FIG. 40A represents the schematic illustration of the transgenic hTfR1-KI mouse construct. The extracellular domain of human TFRC was inserted in the murine Tfrc gene by homologous recombination. FIG. 40B shows quantitative reverse transcription PCR (RT-qPCR) analysis of mouse Tfrc and human TFRC gene expression in brain (N=3/genotype). hTfR1-KI mice (grey circles) express human TFRC and mouse Tfrc in total brain homogenate, WT littermates only express mouse Tfrc (white). FIG. 40C shows western blot analysis for hTfR1, total TfR1, and β-actin in brain. hTfR1-KI animals at 6-8 months (N=5) and 15 months (N=4) express comparable levels of hTfR1 protein. Total TfR1 levels are comparable between hTfR1-KI transgenic and WT littermates (N=3).

FIGS. 41A-41C show the results of in vivo brain and plasma exposure analysis of various indicated hTfR1-binding molecules in hTfR1-KI transgenic mice as described in Example 21. FIG. 41A shows the result of Brain exposure 24 h after i.v. administration of the indicated hTfR1 binders. FIG. 41B shows the results of Plasma exposure 24 h after i.v. administration of the indicated hTfR1 binders. FIG. 41C shows the result of Brain:Plasma ratio 24 h after i.v. administration of the indicated hTfR1 binders. The negative control is denoted “158”, and the positive control “15G11-1”.

FIG. 42 shows the results of in vivo brain exposure analysis of various indicated hTfR1-binding molecules in hTfR1-KI mice by immunohistochemistry as described in Example 22. Cortical brain capillary staining observed for several binding molecules, including h26D3. Reference hTfR1-binder “15G11-1” and non-TfR1 binder “158” were used as positive and negative control, respectively.

FIG. 43 shows BLI sensorgrams for the indicated alanine variants of h26D3 as described in Example 23. Each variant showed a different kinetic profile, illustrating the possibility to generate variants with different affinities against human TfR1 with specific mutations in the CDR regions of the heavy or light chain.

FIG. 44 shows representative SPR sensorgrams of the interaction between the indicated alanine variants of h26D3 with hTfR1 and cTfR1, measured as described in Example 23.

FIG. 45 shows the results of indirect ELISA analysis of the binding of the indicated alanine variants of h26D3 with hTfR1 and cTfR1, measured as described in Example 23.

FIG. 46 shows SPR sensorgrams of the interaction between the indicated alanine variants of h26D3, studied as scFv building blocks within a bispecific protein format as described in Example 23.

FIGS. 47A-47B show chromatograms from preparative SEC of h26D3-HC6_DS (FIG. 47A) and h26D3-HC6 (FIG. 47B), carried out as described in Example 24.

FIGS. 48A-48H show chromatograms from analytical SEC of the indicated scFv proteins after formulation and short-term storage at −80° C., as described in Example 25.

FIGS. 49A-49D show chromatograms from analytical SEC analysis of the indicated scFv proteins kept at −80° C. (T0) and then at 40° C. for 1, 2 and 4 weeks as indicated, carried out as described in Example 26.

FIGS. 50A-50D is a series of bar diagrams showing the percentage of monomeric scFv, as measured by analytical SEC, in samples of the respective indicated scFv molecule subjected to the thermal stability evaluation described in Example 26.

FIGS. 51A-51C show chromatograms from analytical SEC analysis of the indicated scFv proteins kept at −80° C. (T0) and then at 40° C. for 1, 2 and 4 weeks as indicated, carried out as described in Example 26. The asterisk (*) in FIG. 51C highlights a shift in retention time for h26D3-LC1_DS which occurred due to drift in the chromatography equipment. The shift was also seen for a standard size control (not shown) injected on the same column, and is unrelated to the analyzed sample.

FIGS. 52A-52C demonstrate the results of the ELISA experiment described in Example 27, showing hTfR1 binding of a stabilized binding molecule of the disclosure after 48 h incubation in mouse serum at 37° C. and 4° C. in three separate experiments (FIG. 52A), binding curves obtained from the binding molecule incubated in serum, in comparison to incubation in PBS (FIG. 52B), and the ratio of binding activity at 37° C. to the binding activity at 4° C. in serum or PBS as indicated (FIG. 52C).

FIG. 53 shows representative SPR sensorgrams of the interaction between the indicated variants of h26D3 with hTfR1, measured as described in Example 29.

FIG. 54 is a series of bar diagrams showing the percentage of monomeric bispecific binding molecule, as measured by analytical SEC, in samples of the respective indicated molecule subjected to the thermal stability evaluation described in Example 30.

FIGS. 55A-55B show the binding interactions for antibodies BA001 (FIG. 55A) and BA002 (FIG. 55B) against human TfR-1, measured by SPR as described in Example 32. Black line=fit, grey line=experimental data.

FIGS. 56A-56C show the binding interactions for antibodies BA001 (FIG. 56A), BA002 (FIG. 56B) and BA003 (FIG. 56C) against AβpE3-40 monomer, measured by SPR as described in Example 32. Black line=fit, grey line=experimental data.

FIGS. 57A-57B show the inhibition-response curves of the indicated antibodies against AβpE3-40 monomers (FIG. 57A) and AβpE3-42 protofibrils (FIG. 57B) using inhibition ELISA, as described in Example 32.

FIG. 58 is a diagram showing the immunoprecipitation of AβpE3-x levels in a soluble AD brain extract by the indicated humanized antibodies, as described in Example 33.

FIG. 59 shows the concentration-response effects of the indicated antibodies on uptake of Dylight650-labelled AβpE3-40 monomers in THP-1 cells, as described in Example 34.

FIG. 60 shows the concentration-response of the indicated humanized antibodies on Aβ plaque clearance in AD brain sections, as described in Example 34.

FIG. 61 shows a diagram of the antibody-dependent cellular cytotoxicity assay with the indicated humanized antibodies, as described in Example 35.

FIG. 62 shows a diagram of the complement-dependent cytotoxicity assay with the indicated humanized antibodies, as described in Example 35.

FIGS. 63A-63C show diagrams of plasma (filled symbols) and brain (open symbols) concentration versus time profiles after intravenous administration of BA001 (FIG. 63A), BA002 (FIG. 63B) and BA003 (FIG. 63C) in hTfR1-KI mice, as described in Example 36.

FIGS. 64A-64B show frontal cortex 10× and 40× Z-stack images of sagittal brain sections from 5×FAD×hTfR1-KI mice 48 h after intravenous injection with BA001 (FIG. 64A) or BA003 (FIG. 64B) as described in Example 37. Total amyloid β and hIgG representative images.

FIGS. 65A-65C show representative light sheet microscopy images of 5×FAD×hTfR1-KI brains from mice injected with BA003 (FIG. 65A), BA001 (FIG. 65B) and BA002 (FIG. 65C) 72 h post i.v. dosing, as described in Example 37. Scale bar 1.5 mm.

FIG. 66 shows the results of quantification of hIgG immunosignal in 5×FAD×hTfR1-KI brains following intravenous dosing of BA001, BA002 and BA003, analyzed by light sheet microscopy as described in Example 37. Average staining of hIgG signal per voxel is shown for all treatment groups and timepoints in select regions. Values are shown as mean±SD.

FIG. 67 shows the results on the levels of AβpE3-40/Aβ40 from dosing with 10 mg/kg BA001 or vehicle (PBS) i.v. every 4 days for 4 weeks, in 5×FAD×hTfR-KI male mice (age 6.5-7.5 months), as described in Example 38. Values are shown as mean±SD. Student's t-test was used for statistical comparison.

FIG. 68 shows a diagram of competition with transferrin with the indicated humanized antibodies in K562 cells, as described in Example 39.

FIGS. 69A-69B show a diagram of competition with ferritin (H-Ft) with the indicated test compounds in K562 cells, as described in Example 39. BA001, BA003 and positive control anti-CD71 IgG M-A712 were incubated together with H-Ft at either 4° C. (binding; FIG. 69A) or 37° C. (uptake; FIG. 69B) on K562 cells.

FIG. 70 shows a diagram of blood reticulocyte counts (expressed as % of pre-sample average) at 24 h after a single intravenous administration of BA001 or BA003 in hTfR1-KI mice, as described in Example 40. Data is shown as mean±SD.

FIG. 71 shows a diagram of the reticulocyte counts (expressed as % of pre-sample) in serum at 24 h after a single intravenous administration of BA001, BA002 or BA003 in non-human primates, as described in Example 41.

FIGS. 72A-72B show a diagram of the plasma (FIG. 72A) and brain (FIG. 72B) concentrations at 24 h after a single intravenous administration of BA001, BA002 or BA003 in non-human primates, as described in Example 41.

FIGS. 73A-73B show the results of testing BA001 and BA003 in the bridging (FIG. 73A) and competition (FIG. 73B) assays for pre-existing antibodies, as described in Example 42.

FIGS. 74A-74F show stacked chromatograms from analytical SEC of the indicated scFv samples collected as TO or after temperature hold at 40° C. for 1, 2 or 4 weeks respectively, as described in Example 44. The relative absorbance mAU (220 nm) is shown on the X-axis of respective graph.

FIGS. 75A-75L show representative SPR sensorgrams of the interaction between the indicated scFv variants and hTfR1, as described in Example 46.

FIGS. 76A-76L show representative SPR sensorgrams of the interaction between the indicated scFv variants and cTfR1, as described in Example 46.

FIG. 77 shows a diagram of serum reactivity of the indicated tested molecules, analyzed as described in Example 48.

FIGS. 78A-78F show representative SPR sensorgrams of the interaction between bispecific binding molecules and hTfR1 or cTfR1, as described in Example 49. (FIG. 78A) BA001 vs hTfR1; (FIG. 78B) BA001 vs cTfR1; (FIG. 78C) BA007 vs hTfR1; (FIG. 78D) BA007 vs cTfR1; (FIG. 78E) BA006 vs hTfR1; (FIG. 78F) BA006 vs cTfR1.

FIGS. 79A-79C show representative SPR sensorgrams of the interaction between bispecific binding molecules BA006 (FIG. 79A); BA007 (FIG. 79B) or BA001 (FIG. 79C) and AβpE3-40 monomers, as described in Example 50.

FIG. 80 shows the result of flow cytometry analysis of binding on hTfR1 on K562 cell surfaces by the indicated bispecific binding molecules, as described in Example 51. The mean fluorescence intensity (MFI (PE)) is plotted on the y-axis and the concentration of tested molecule (nM) is shown on the x-axis.

### EXAMPLES

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention is not limited to any particular embodiment, but that the invention will include all embodiments falling within the scope of the appended claims.

The invention will be further illustrated by the following non-limiting Examples. They are offered for illustrative purposes only and are not intended to limit the invention in any manner. Those of skill in the art will readily recognize a variety of non-critical parameters which can be changed or modified to yield essentially the same results. Efforts have been made to ensure accuracy with respect to numbers used (e.g. amounts, temperatures, etc.), but some experimental error and deviations may be present. Unless otherwise indicated, the practice of the invention employs conventional methods of protein chemistry, biochemistry, recombinant DNA techniques and pharmacology, within the skill of the art. Such techniques are explained fully in the existing literature. Additionally, it will be apparent to one of skill in the art that the methods for protein engineering applied herein can also be applied to other constructs described herein and contemplated by the present inventors to fall within the scope of the disclosure.

### Example 1

### Generation and Screening of Antibodies to AβpE3

This example describes the immunization of BALB/c mice and subsequent generation and screening of hybridoma cell lines.

### Materials and Methods

Immunogen preparation: Protofibrils produced from the AβpE3-42 peptide were used as immunogen. Briefly, AβpE3-42 peptide (American Peptide) was dissolved in 10 mM NaOH, pH>11 at a concentration of 100 μM. Protofibrils were prepared by neutralizing the AβpE3-42 peptide to pH 7.4 by adding 1:1 of a 2×PBS buffer to a final concentration of 50 μM. The peptide was incubated for 20 min at 37° C. for protofibril formation and was purified from remaining monomers by HPLC on a Superdex 75 10/300 GL size-exclusion column using a mobile-phase of 1×PBS, 0.1% Tween-20, pH 7.4. Prior to injection, the protofibril reaction was centrifuged at 16000×g for 5 min at 4° C. Fibrillar material was pelleted, and the supernatant contained soluble AβpE3-42 protofibrils and monomers. The protofibrils were eluted in the void volume of the column and the void fraction was collected. Repeated injections and collections of the void peaks were carried out to achieve enough material for immunization. A total of 14 injections were made, and 44 ml were collected and concentrated on 50 MWCO Amicon filters (UFC805024, Millipore) to a final concentration of 20 μM. Briefly, the Amicon filter was pre-wetted in PBS, 0.005% Tween-20, pH 7.4 for 2 h prior to sample loading. Protofibrils were added and concentrated 8-fold by centrifugation at 3200×g. Purified protofibrils were stored at −80° C. until use.

Immunization: BALB/c mice at 8 weeks of age (n=3) were immunized with AβpE3-42 protofibrils prepared as described above. AβpE3-42 protofibrils at a concentration of approximately 20 μM (86 μg/ml) were in sterile PBS, pH 7.4, containing 0.1% Tween-20. For each immunization, 400 μl AβpE3-42 protofibrils (˜34 μg) were mixed with 5 μl ISCOM adjuvant (12 μg) and administered subcutaneously, 200 μl on each side. Each mouse received three or four immunizations. Plasma samples were collected between two and three weeks after each immunization. Once antibodies reactive against AβpE3-40 with end-point titers of around 1/100000 were detected in plasma, one final booster injection (immunogen without ISCOM adjuvant) was given intraperitoneally. After three days, mice were sacrificed, and spleens collected.

Tissue collection: Blood was collected from the tail vein at all time-points, except at the time of sacrifice when blood was collected from the heart. For sacrifice, mice were anesthetized with 300 mg/kg ketamine and 4 mg/kg medetomidine and blood was sampled from the right atrium into Microtainer tubes followed by centrifugation of the samples at 2400×g for 10 min and transfer of plasma to pre-labeled low-binding Eppendorf tubes. Plasma samples were immediately frozen on dry ice and stored at −80° C. The spleens were collected by opening the abdominal cavity and dissecting out the intact spleens. The spleens were placed in a 15 ml test tube containing 5 ml DMEM media (2× PenStrep) at room temperature (RT) and transported on ice within an hour for preparation of a spleen cell suspension.

Plasma screening by direct ELISA: Plasma samples were analyzed by direct ELISA for reactivity against AβpE3-40 monomers after each immunization to determine when to stop immunizations and initiate hybridoma generation. The AβpE3-40 peptide (Anaspec) was dissolved to 100 μM in 10 mM NaOH+0.005% Tween-20 and stored in aliquots at −80° C. For coating, a 0.5 μM solution was prepared by diluting newly thawed 100 μM AβpE3-40 peptide in PBS, and an ELISA microtiter plate was coated with 50 μl AβpE3-40 per well overnight at +4° C. The plate was washed four times with 1×ELISA washing buffer (containing 0.28 mM NaH₂PO₄, 2.5 mM Na₂HPO₄, 150 mM NaCl, 0.1% Tween-20 and 0.0075% Kathon CG), followed by blocking of the residual binding capacity of the plate by adding 100 μl/well of Pierce blocking buffer and incubation for 1 h at RT with shaking (900 rpm). After discarding the blocking buffer, samples (mouse plasma) or standard was added to each well. Plasma samples from immunized mice were diluted at least 1:1000 and further diluted 1:2 in the wells in seven steps. Plasma from an un-immunized mouse was used as a negative control. The control antibody mAb6E10 (Covance #SIG-39320, epitope: amino acids 1-16 of Aβ) was used to make a standard curve by 2-fold serial dilution starting at 1 ng/ml (final concentration range of 0.016-1 ng/ml). The samples were incubated for 90 min at RT with shaking (900 rpm). The plate was washed as above, and 50 μl of HRP-conjugated anti-mouse IgG (diluted 1/10 000 in incubation buffer consisting of 1× Dulbecco's PBS with 0.1% BSA and 0.05% Tween-20) was added to each well, followed by incubation for 1 h at RT with shaking (900 rpm). For detection, the plate was once again washed as above, and 50 μl of room tempered TMB was added to each well and the plate was incubated at RT for 15 min without shaking. The reaction was stopped by adding 50 μl of 2 M H₂SO₄ and the plate was read at a wavelength of 450 nm within 15 min. An end point titer of 1/100000 was considered high enough, and after this had been reached, no more immunizations were performed.

Generation of hybridomas: Isolated splenocytes from sacrificed mice were fused with cells from an immortalized cell line (SP2/0) to generate hybridomas. Briefly, a single cell suspension of the spleen from an immunized mouse was prepared and mixed with SP2/0 cells at a 1:2 ratio. The cells were fused using polyethylene glycol and the cells were dispensed in 96 well cell culture plates. Media was changed at 7 days and 10-14 days post fusion. The wells/hybridomas were screened for reactivity against AβpE3-42 protofibrils using ELISA. Positive clones were diluted using limiting dilution assays to certify monoclonality. Clones of interest were cryopreserved, expanded for production of antibody, and sequenced.

Antigens used for hybridoma screening by ELISA: AβpE3-42 and Aβ1-42 monomers (American Peptide) and AβpE3-42 and Aβ1-42 protofibrils were used for screening of hybridoma clones. The protofibrils were generated using the respective monomers. Briefly, to generate AβpE3-42 protofibrils, the AβpE3-42 peptide was dissolved in 10 mM NaOH, 0.005% Tween-20, pH>11 at a concentration of 100 μM. Protofibrils were prepared by neutralization of the AβpE3-42 peptide to pH 7.4 by adding 1:1 of a 2×PBS buffer to a final concentration of 50 μM. The peptide was incubated for ˜30 min at 37° C. for protofibril formation and purified from remaining monomers by HPLC on a Superdex 75 Increase 3.2/300 size-exclusion column using a mobile phase of 1×PBS, 0.1% Tween-20, pH 7.4. Prior to injection, the protofibril reaction was centrifuged at 16000×g for 5 min at 4° C. The void peak containing AβpE3-42 protofibrils was collected and the concentration was determined using SEC and a calibration curve of an Aβ protofibril standard with a known concentration. The same procedure was used to produce Aβ1-42 protofibrils from the corresponding monomers.

Hybridoma screening by ELISA: Supernatants from generated hybridomas were characterized using ELISA with Aβ1-42 or AβpE3-42 monomers or protofibrils as capture antigens. An ELISA microtiter plate was coated with a polyclonal rabbit anti-Aβ42 antibody (diluted to 0.5 μg/ml in PBS and 50 μl added per well) and incubated overnight at 4° C. or for 1 h at 37° C. The plate was washed 4 times with 1×ELISA washing buffer followed by blocking of the residual binding capacity of the plate by adding 100 μl/well of Pierce blocking buffer and incubating for 1 h at RT with shaking (900 rpm). The blocking buffer was discarded, and each antigen was added (diluted in incubation buffer to 5 nM and 50 μl added per well) and the plate was incubated for 1 h at RT with shaking (900 rpm). The plate was washed 4 times as described above, followed by addition of sample. Supernatants from hybridoma cell cultures were added undiluted or diluted 1:2 in incubation buffer to each well at a volume of 50 μl/well. The samples were incubated for 1 h at RT with shaking (900 rpm). Following a washing step, an HRP-conjugated anti-mouse IgG antibody (diluted 1/5000 in incubation buffer) was added to each well (50 μl/well) and the plate was incubated for 1 h at RT with shaking (900 rpm). After an additional washing step, detection was performed by adding 50 μl of room tempered TMB to each well, followed by incubation at RT in the dark for 10 min without shaking. The reaction was stopped by adding 50 μl of 2 M H₂SO₄ and the microtiter plate was read at a wavelength of 450 nm, preferably within 15 min. Positive clones with specific binding to AβpE3-42 were further characterized using Aβ1-42, Aβ2-42, Aβ3-42, AβpE3-42 and Aβ4-42 monomers (all purchased from Anaspec) as capture antigens in ELISA, according to the method described above. The supernatants were diluted 1/243 in ELISA regardless of antibody concentration, so measured OD₄₅₀ values did not necessarily correlate to binding affinity.

Antibody concentration determination: The antibody concentration in the respective hybridoma supernatants was measured using a standard sandwich ELISA. Microtiter plates were coated with an anti-mouse IgG antibody recognizing the F(ab′)₂ part of mouse IgG (0.5 μg/ml, 50 μl per well) overnight at 4° C. without shaking. The residual binding capacity of the plate was blocked by adding 200 μl/well of blocking buffer and incubating for 1 h at RT with shaking (900 rpm). The plate was washed three times with 1×ELISA washing buffer, followed by addition of the samples. Hybridoma supernatants were diluted 1/250 and added in duplicates to the ELISA plate (200 μl/well) and serially diluted 2-fold in seven steps to ensure that the sample dilution was within the range of the standard. The Aβ protofibril-selective mouse antibody mAb158 (Englund et al (2007), J Neurochem 103(1):334-45) was used to make a standard curve by 2-fold serial dilution to generate a final concentration range of 15-500 pg/ml). The plate was incubated for 2 h at RT with shaking (900 rpm). The plate was washed as above, and 50 μl of HRP-conjugated anti-mouse IgG antibody that was also F(ab)₂ specific (diluted 1/2500 in incubation buffer) was added to each well, followed by incubation for 1 h at RT with shaking (900 rpm). For detection, the plate was once again washed as above, and 100 μl of room tempered TMB was added to each well and the plate was incubated at RT for 5-20 min without shaking. The reaction was stopped by adding 50 μl of 2 M H₂SO₄ and the plate was read at a wavelength of 450 nm within 15 min.

### Results

Generation of monoclonal antibodies by hybridoma technology: Antibodies that bind selectively to AβpE3 were generated by immunization using AβpE3-42 protofibrils. The plasma samples were analyzed by ELISA for reactivity against the AβpE3-40 monomer. When titers were at least 1/100000 the mice were sacrificed, and the spleens were collected and used for hybridoma generation.

A total of 22 AβpE3-42 reactive hybridomas were generated. Their specificity for AβpE3-42 monomers and AβpE3-42 protofibrils, compared to for Aβ1-42 monomers and Aβ1-42 protofibrils, was tested by ELISA. Twelve of the clones bound to all four Aβ forms tested, whereas ten of the clones bound specifically to AβpE3-42. None of the clones were selective for protofibrils but bound equally well to AβpE3-42 monomers as to AβpE3-42 protofibrils.

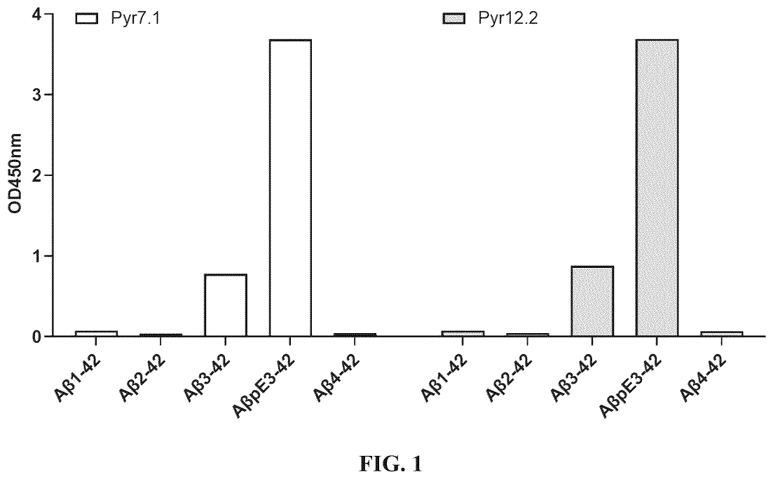

The ten clones that were specific binders to AβpE3-42 were further characterized using Aβ1-42, Aβ2-42, Aβ3-42, AβpE3-42 and Aβ4-42 monomers as capture reagent in ELISA. All ten clones tested were specific for AβpE3-42 monomers, with only some weak cross-reactivity to Aβ3-42 monomers. FIG. 1 shows the result of this experiment for two selected clones, denoted Pyr7.1 and Pyr12.2.

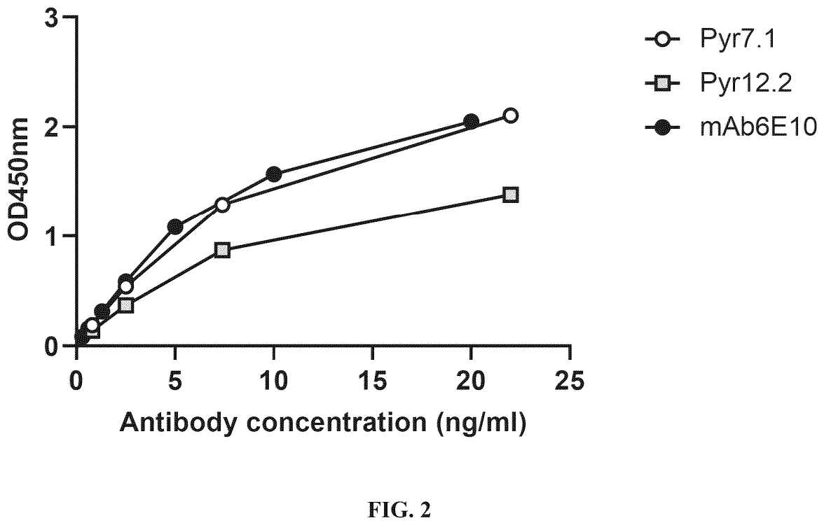

To compare the binding strength of the Pyr7.1 and Pyr12.2 antibody clones, antibody concentration was first determined, and the same amount of each clone was then loaded on an ELISA plate using AβpE3-42 protofibrils as capture. Both clones demonstrated binding to AβpE3-42 protofibrils, with binding comparable to the positive control mAb6E10 (FIG. 2).

### Example 2

### Hybridoma Sequencing and Production of Recombinant Antibodies

### Materials and Methods

Hybridoma sequencing: Hybridoma clones producing monoclonal antibodies, generated and characterized as described in Example 1 and having a demonstrated specificity for AβpE3-42 monomers and protofibrils, were sequenced by whole transcriptome shotgun sequencing. The DNA and protein sequences of mature VH and VL domains were identified.

Expression, production and purification: The variable domains were designed and optimized for expression in mammalian cells (HEK293) prior to being synthesized. The sequences were then subcloned into a cloning and expression vector (Absolute Antibody) for the appropriate isotype and subtype of immunoglobulin heavy and light chains. HEK293 cells were passaged to the optimum stage for transient transfection. Cells were transiently transfected with heavy and light chain expression vectors and cultured for a further 6-14 days. Cultures were harvested and a one-step purification was performed using affinity chromatography, after which the purified antibodies were buffer exchanged into PBS. Antibodies were analyzed for purity by SDS-PAGE and the concentration determined by UV spectroscopy.

### Results

Hybridoma sequencing and recombinant antibody production: Hybridoma clones with a demonstrated specificity for AβpE3-42 monomers and protofibrils were sequenced, and the sequences of selected antibodies Pyr7.1 and Pyr12.2 are reported.

The amino acid sequences of the entire antibodies were obtained. Amino acid sequences for the respective variable heavy (VH) and variable light (VL) chains are given in Table 1 below, as well as the sequences of the constant regions shared by all antibodies. Complementarity determining regions (CDRs) were identified using the Kabat definition.

TABLE 1
Amino acid sequences of selected monoclonal antibodies

| | | | SEQ |
|---|---|---|---|
| Antibody | Region | Amino acid sequence | ID NO: |
| Pyr7.1 | Heavy chain | | |
| | VH | KVQLQQSGPELVKPGTSIKMSCKTSGYSFTGYT | 27 |
| | | LNWVKQSPGKNPEWIGLINPYNGITTYNPKFMG | |
| | | KATLTVDKSSSTAYMELLSLTSEDSAVYYCSRE | |
| | | GNWEGVYWGQGTLVTVSA | |
| | VHCDR1 | GYTLN | 12 |
| | VHCDR2 | LINPYNGITTYNPKFMG | 13 |
| | VHCDR3 | EGNWEGVY | 3 |
| | Light chain | | |
| | VL | DVVMTQTPLTLSVTIGQPASISCKSSQSLLDSNG | 28 |
| | | KTYLHWLLLRPGQSPKRLIYLVSKLDSGVPDRFT | |
| | | GSGSGTDFTLKISRVEAEDLGVYFCVQGTHFPFT | |
| | | FGSGTKLEIK | |
| | VLCDR1 | KSSQSLLDSNGKTYLH | 11 |
| | VLCDR2 | LVSKLDS | 14 |
| | VLCDR3 | VQGTHFPFT | 6 |
| Pyr12.2 | Heavy chain | | |
| | VH | EVQLQQSGPELVKPGTSIKMSCKASGYSFTGFT | 25 |
| | | MNWVKQSHGKNLEWIGLINPYNGVTTYNQKFKG | |
| | | KATITVDKSSRTAYMELLSLTYEDSAVYYCTRE | |
| | | GNWEGVYWGQGTPVTVSA | |
| | VHCDR1 | GFTMN | 7 |
| | VHCDR2 | LINPYNGVTTYNQKFKG | 8 |
| | VHCDR3 | EGNWEGVY | 3 |
| | Light chain | | |
| | VL | EVVLTQTPLTLSVTIGQSASISCKSSQSLLDSNGK | 26 |
| | | TYLHWFLLRPGQSPKRLIYLVSILDSGVPDRFTGS | |
| | | GSGTDFTLKISRVEAEDLGIYYCVQGTHFPFTFGS | |
| | | GTKLEIK | |
| | VLCDR1 | KSSQSLLDSNGKTYLH | 11 |
| | VLCDR2 | LVSILDS | 9 |
| | VLCDR3 | VQGTHFPFT | 6 |
| Murine | Constant | AKTTAPSVYPLAPVCGGTTGSSVTLGCLVKGYFP | 29 |
| IgG2c | heavy chain | EPVTLTWNSGSLSSGVHTFPALLQSGLYTLSSSV | |
| constant | region | TVTSNTWPSQTITCNVAHPASSTKVDKKIESRRP | |
| region | | IPPNSCPPCKECSIFPAPDLLGGPSVFIFPPKIK | |
| | | DVLMISLSPIVTCVVVDVSEDDPDVQISWFVNNV | |
| | | EVHTAQTQTHREDYNSTLRVVSALPIQHQDWMS | |
| | | GKEFKCKVNNRALPSPIEKTISKPRGPVRAPQV | |
| | | YVLPPPAEEMTKKEFSLTCMITDFLPAEIAVDWT | |
| | | SNGHKELNYKNTAPVLDTDGSYFMYSKLRVQKS | |
| | | TWEKGSLFACSVVHEGLHNHHTTKTISRSLGK | |
| Murine | Constant light | RADAAPTVSIFPPSSEQLTSGGASVVCFLNNFYP | 30 |
| kappa | chain region | KDINVKWKIDGSERQNGVLNSWTDQDSKDSTYSM | |
| constant | | SSTLTLTKDEYERHNSYTCEATHKTSTSPIVKSF | |
| region | | NRNEC | |

The monoclonal antibodies Pyr7.1 and Pyr12.2 were selected for production as recombinant IgG2c antibodies. All recombinant antibodies were successfully produced and purified to a final concentration of 1 mg/ml. Antibody purity, as defined by SEC-HPLC, was >98% monomer for all antibodies.

### Example 3

### Characterization of Recombinant Antibodies

This example describes the characterization of the affinity, selectivity and specificity of the recombinant antibodies produced in Example 2 by inhibition ELISA and SPR.

### Materials and Methods

Aβ monomer species and Aβ protofibrils: The following Aβ peptides were used for characterization of the binding of the recombinant antibodies to different Aβ species: AβpE3-40, Aβ1-40, Aβ1-28 and AβpE11-40, all purchased from Bachem, and Aβ2-28, Aβ3-28, Aβ4-28 and AβpE11-28, all custom-made by and purchased from Innovagen. Aβ peptides from Bachem were dissolved in 10 mM NaOH, 0.005% Tween-20, pH>11 at a concentration of 100 μM. Aβ peptides from Innovagen were dissolved in 1×PBS, pH 7.4 at a concentration of 300 μM. Aliquots were made and stored at −80° C. until analysis. All peptides were verified to be monomeric by size-exclusion chromatography.

Protofibrils were prepared using the AβpE3-42 peptide from Bachem. Briefly, the AβpE3-42 peptide was dissolved in 10 mM NaOH, 0.005% Tween-20, pH>11 at a concentration of 100 μM. Protofibrils were prepared by neutralizing the AβpE3-42 peptide to pH 7.4 by adding 1:1 of a 2×PBS buffer to a final concentration of 50 μM. The peptide was incubated for ˜30 min at 37° C. for protofibril formation and purified from remaining monomers by HPLC on a Superdex 75 Increase 3.2/300 size-exclusion column using a mobile-phase of 1×PBS, 0.1% Tween-20, pH 7.4. Prior to injection, the protofibril reaction was centrifuged at 16000×g for 5 min at 4° C. to remove insoluble fibrils. The void peak containing the AβpE3-42 protofibrils was collected and the concentration determined using SEC and a calibration curve of an Aβ protofibril standard with a known concentration.

Selectivity evaluation and IC₅₀ determination by inhibition ELISA: The binding of selected recombinant antibodies Pyr7.1 and Pyr12.2 to different Aβ antigens (AβpE3-40, Aβ1-40 and AβpE11-40 monomers and AβpE3-42 protofibrils) was evaluated by inhibition ELISA. For binding to Aβ1-40 and AβpE11-40, the positive control 4G8 (Covance #SIG-39320, epitope: amino acids 17-24 of Aβ) was included. The recombinant antibodies were incubated at a fixed concentration (0.05 μg/ml) with titrating concentrations of the different Aβ antigens. After incubation for 45 min at 900 rpm to reach equilibrium, the antibody-Aβ samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

Affinity and specificity evaluation and K_{D} determination by surface plasmon resonance: Binding interactions between antigens and antibodies were evaluated by SPR using a Biacore 8K instrument (Cytiva) according to standard procedures. The binding of selected recombinant antibodies Pyr7.1 and Pyr12.2 to AβpE3-40 monomers and protofibrils and their selectivity against Aβ1-28 monomers was evaluated. Additionally, the specificity towards different N-truncated forms of Aβ (Aβ2-28, Aβ3-28, Aβ4-28 and AβpE11-28 monomers) was evaluated.

Single cycle kinetics with the antibodies immobilized on a CM5 chip was used to measure the binding of the antibodies to the different monomers (AβpE3-40, AβpE11-28, Aβ1-28, Aβ2-28, Aβ3-28 and Aβ4-28). For the measurements, 5 μg/ml of analyte antibody was immobilized on the chip. The AβpE3-40 monomer was then injected over the chip using a 2-fold dilution in five steps starting at 250 nM for AβpE3-40 with a dissociation time of 20 min and at 2500 nM for all other monomers with a dissociation time of 10 min. Regeneration of the surface between cycles was done by injecting 30 μl 3 M MgCl₂. The binding data was fitted to a 1:1 interaction model.

Single cycle kinetics was also used to measure binding of the antibodies to AβpE3-42 protofibrils. The AβpE3-42 protofibrils (138 ng/ml) were coupled to a CM5 chip using general Biacore coupling chemistry (immobilization low levels). For binding to AβpE3-42 protofibrils, the antibodies were injected over the chip using a 3-fold dilution series in five steps starting at 700 nM, using 2 min injection of every antibody concentration and a 60 min dissociation time. Regeneration of the surface between cycles was done by injecting 30 μl 10 mM glycine-HCl pH 1.7. The binding data was fitted to a 1:1 interaction model. The use of the 1:1 interaction model gives the K_{D} for the binding of the antibodies to Aβ monomers and an apparent K_{D} for the binding of the antibodies to Aβ protofibrils.

In all SPR experiments, 1×HBS-EP+ (Cytiva, cat. no. BR100669) was used as running buffer and to dilute antibodies and target antigens. Experiments were performed at 25° C.

### Results

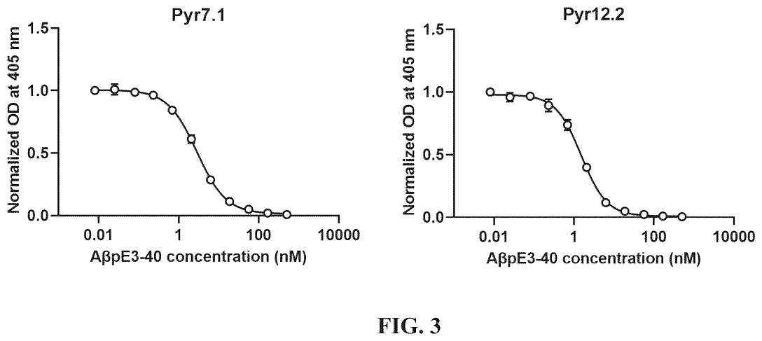

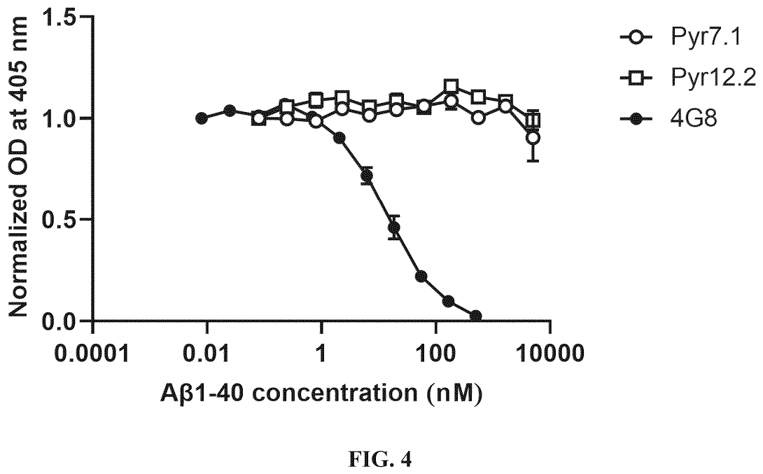

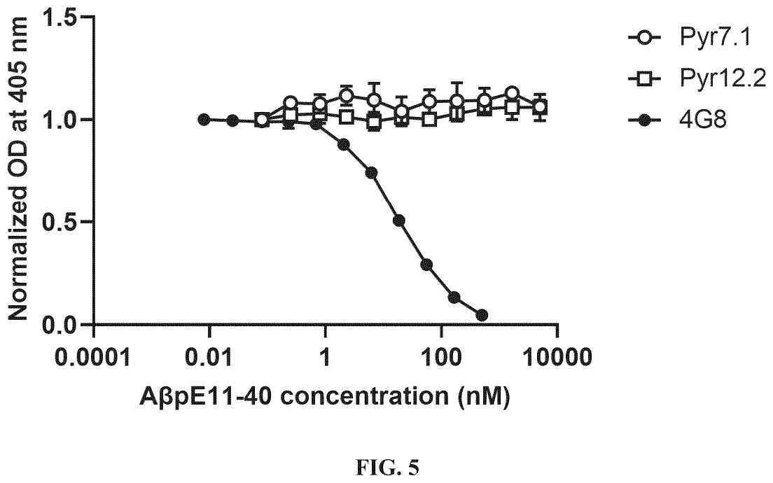

Selectivity evaluation and IC₅₀ determination by inhibition ELISA: The binding of the recombinant antibodies to AβpE3-40 and selectivity versus Aβ1-40 and AβpE11-40 monomers was initially evaluated using inhibition ELISA. Recombinant antibodies Pyr7.1 and Pyr12.2 demonstrated binding to AβpE3-40 monomer in solution (FIG. 3). None of the antibodies bound Aβ1-40 in solution at concentrations up to 5 μM, suggesting the IC₅₀ values were >5 μM (FIG. 4). The positive control antibody 4G8 demonstrated binding to Aβ1-40, as expected. Recombinant antibodies Pyr7.1 and Pyr12.2 were also tested for binding to AβpE11-40 monomers using inhibition ELISA. Here, no binding was observed at antigen concentrations up to 5 μM, suggesting the IC₅₀ values were >5 μM (FIG. 5). The positive control antibody 4G8 demonstrated binding to AβpE11-40, as expected. The calculated IC₅₀ values are listed in Table 2.

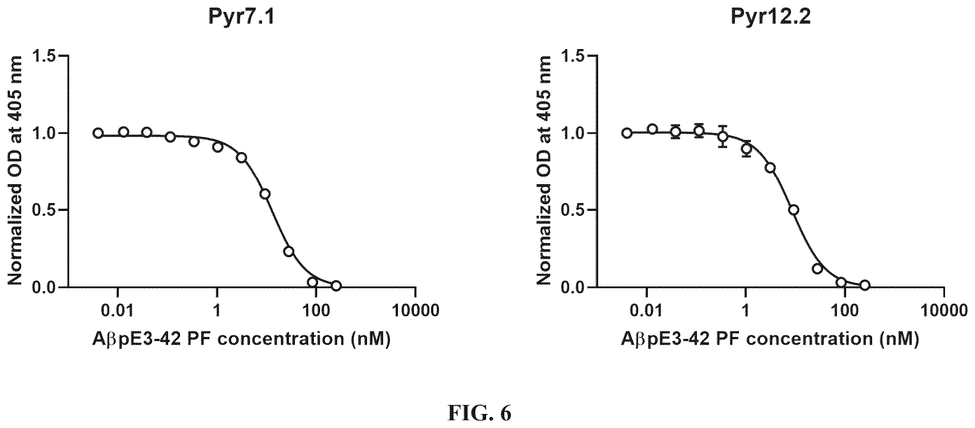

The binding of the recombinant antibodies to AβpE3-42 protofibrils was evaluated using inhibition ELISA. Both antibodies demonstrated binding to AβpE3-42 protofibrils in solution (FIG. 6). The calculated IC₅₀ values are listed in Table 2.

TABLE 2
Summary of results from inhibition ELISA

| | AβpE3-40 | Aβ1-40 | AβpE11-40 | AβpE3-42 |
|---|---|---|---|---|
| | monomer | monomer | monomer | protofibril |
| Antibody | IC₅₀ (nM) | IC₅₀ (nM) | IC₅₀ (nM) | IC₅₀ (nM) |
| Pyr7.1 | 2.85 | >5000 | >5000 | 12.8 |
| Pyr12.2 | 1.52 | >5000 | >5000 | 8.43 |

Affinity and specificity evaluation and K_{D} determination by surface plasmon resonance: The recombinant antibodies Pyr7.1 and Pyr12.2 were evaluated by SPR for affinity and specificity and their K_{D} values were determined.

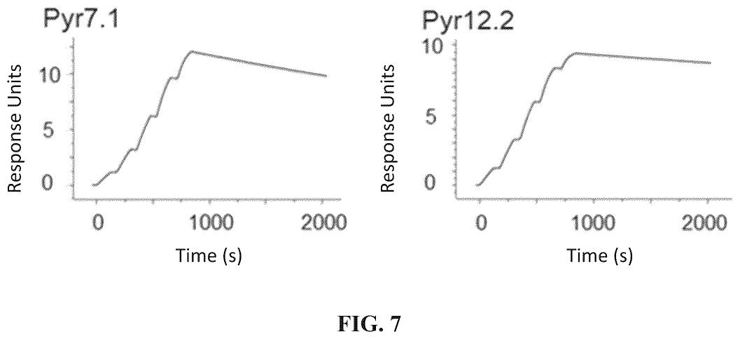

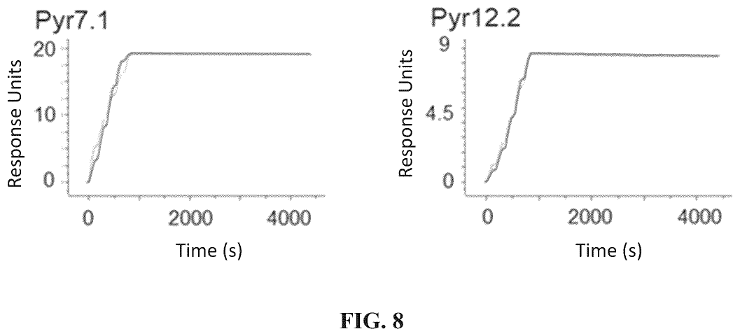

Both antibodies demonstrated binding to AβpE3-40 monomers and AβpE3-42 protofibrils. The apparent affinities of Pyr7.1 and Pyr12.2 for AβpE3-42 protofibrils were probably underestimated, because the k_{d} values for most of the binding experiments were outside the instrument's detection limits. All kinetic data from such binding experiments were excluded when calculating affinities. The calculated kₐ, k_{d} and (apparent) K_{D} values are shown in Table 3 and Table 4 below. Representative sensorgrams are shown in FIG. 7 and FIG. 8.

TABLE 3
Summary of SPR analysis of binding to AβpE3-40 monomers

AβpE3-40 monomer

| | kₐ (M⁻¹s⁻¹) | k_{d} (s⁻¹) | K_{D} (nM) |
|---|---|---|---|
| Antibody | Mean ± SD | Mean ± SD | Mean ± SD |
| Pyr7.1 | 5.32 ± 0.11 e4 | 1.22 ± 0.20 e−4 | 2.30 ± 0.40 |
| Pyr12.2 | 7.18 ± 0.22 e4 | 4.12 ± 1.41 e−5 | 0.57 ± 0.19 |

TABLE 4
Summary of SPR analysis of binding to AβpE3-42 protofibrils

AβpE3-42 protofibril

| | kₐ (M⁻¹s⁻¹) | k_{d} (s⁻¹) | K_{D} (pM) |
|---|---|---|---|
| Antibody | Mean ± SD | Mean ± SD | Mean ± SD |
| Pyr7.1 | 7.82 ± 1.48 e4 | 2.29 ± 0.89 e−6 | 31.0 ± 15.2 |
| Pyr12.2 | 6.31 ± 1.22 e4 | 3.31 ± 1.93 e−6 | 53.6 ± 31.0 |

The recombinant antibodies did not show any binding to Aβ1-28 up to 2500 nM of monomer.

Specificity of recombinant antibodies evaluated by surface plasmon resonance: The specificity of the recombinant antibodies to different N-truncated forms of Aβ monomers was evaluated by SPR. Pyr7.1 and Pyr12.2 had no binding to the Aβ2-28 monomer in the concentration range tested. Pyr12.2 bound the Aβ3-28 monomer with nM affinity, whereas no binding was detected for Pyr7.1. Both antibodies bound the Aβ4-28 monomer with μM affinity. No binding was observed for any of the antibodies to AβpE11-28 up to a concentration of 2500 nM. The calculated kₐ, k_{d} and K_{D} values are shown in Table 5.

TABLE 5
Summary of SPR analysis of binding to different Aβ monomers

| | kₐ (M⁻¹s⁻¹) | k_{d} (s⁻¹) | K_{D} (μM) |
|---|---|---|---|
| Antibody | Mean ± SD | Mean ± SD | Mean ± SD |
| | Aβ2-28 monomer | | |
| Pyr7.1 | — | — | No binding |
| Pyr12.2 | — | — | No binding |
| | Aβ3-28 monomer | | |
| Pyr7.1 | — | — | No binding |
| Pyr12.2 | 1.75 ± 0.94 e3 | 5.77 ± 1.22 e−5 | 42.6 ± 18.9 |
| | Aβ4-28 monomer | | |
| Pyr7.1 | 1.02 ± 0.81 e5 | 4.54 ± 2.55 e−1 | 8.31 ± 2.55 |
| Pyr12.2 | 7.82 ± 1.95 e4 | 4.81 ± 0.60 e−1 | 6.76 ± 2.62 |
| | AβpE11-28 monomer | | |
| Pyr7.1 | — | — | No binding |
| Pyr12.2 | — | — | No binding |

### Example 4

### Target Binding of Recombinant Antibodies in Brain from Human Alzheimer's Disease Patients and Non-Demented Controls

This Example describes target binding of recombinant antibodies Pyr7.1 and Pyr12.2, produced as described in Example 2, as tested by immunoprecipitation on human brain extracts and immunohistochemistry on human brain sections from AD patients and NDE controls.

### MATERIALS and Methods

Brain tissue homogenization and sample preparation: Fresh frozen human brain cortical tissue from Alzheimer's disease (AD) patients and non-demented (NDE) controls were homogenized in a Potter-Elvehjem homogenizer at 1:10 weight:volume in Tris-buffered saline (TBS) buffer followed by centrifugation at 16000×g for 1 h. The resulting supernatant was frozen at −80° C. until analysis.

Target binding in human Alzheimer's disease brain extracts by immunoprecipitation: Antibody binding to target in human AD brain was analyzed by immunodepletion, a method for removal of target protein in a sample using an antibody specific for the target molecule. Briefly, each of the recombinant antibodies Pyr7.1 and Pyr12.2, covalently coupled to magnetic Dynabeads, was incubated with soluble TBS brain extracts from AD and non-demented (NDE) control cases for 1 h at RT with rotation. The bead-bound target was separated by a magnet and depleted from the extracts. The depleted brain extracts (supernatants) were analyzed using an AβpE3-40 kit and an MSD assay for measurements of AβpE3-42 levels. Target binding by the recombinant antibodies was evaluated by the reduction of measured AβpE3-40 or AβpE3-42 levels in depleted samples compared to levels in non-depleted brain extracts. A complete target binding was demonstrated when levels in depleted brain extracts were below the lower limit of quantification (LLOQ) for the assay.

A sandwich ELISA kit from Immunobiological Laboratories (product code: 27418) was used to measure levels of AβpE3-40 in human AD brain. Each assay kit contains all necessary components including antibodies, standard calibrator and plates precoated with a human anti-Aβ mouse IgG monoclonal capture antibody (epitope at amino acid positions 35-40 of Aβ). Briefly, diluted standard calibrator and test samples were allowed to bind to the plate during incubation at 4° C. overnight. After a wash step, an HRP conjugated anti-human AβpE3 antibody (8E1, included in the kit) was added, followed by an incubation for 1 h at 4° C. After an additional wash step, TMB was added as a coloring agent (chromogen) and the plate was incubated for 30 min before the reaction was stopped and absorbance measured at 450 nm. The strength of coloring was proportional to the quantities of human AβpE3-40.

An MSD assay was used to measure AβpE3-42. An MSD standard plate was coated over night at 4° C. with Pyr7.1 as a capture antibody (3 μg/ml per well). Free binding sites were blocked by incubation with 1% blocker A solution prior to incubation for 2 h (900 rpm shaking) with diluted standard (AβpE3-42, 7.1-1000 μg/ml) and test samples. An anti-Aβ42 rabbit polyclonal antibody (produced in-house) was added to the plate (1.5 μg/ml per well) and allowed to incubate for 1 h, followed by a final 1 h incubation with a goat anti-rabbit MDS SULFO-TAG antibody (diluted 1:1000). The plate was washed between blocking and each antibody incubation step. The plate was read in an MSD sector imager where a light signal was generated and measured. The signal strength was correlated to the amount of AβpE3-42 in the sample.

Target binding in human Alzheimer's disease brain by immunohistochemistry: Immunohistochemistry (IHC) analyses were performed on brain tissue from AD and non-demented control. Postmortem human brain tissue of temporal cortex was obtained from the Netherlands Brain Bank (NBB) and had been collected at autopsy with local ethical committee approval.

The mouse anti-human Aβ antibodies 6E10 (Covance #SIG-39320) and 4G8 (Covance #SIG-39200) were used for detection of Aβ pathology in brain sections. As reference antibody, a purified mouse monoclonal anti-AβpE3 IgG1 antibody (Glu3) was used (Biolegend, #822301). Human target binding in AD brain was evaluated for antibodies Pyr7.1 and Pyr12.2, obtained as described in the Examples above.

For IHC staining of Aβ, an automated staining robot and a HRP-3,3′-diaminobenzidine (DAB) based detection system was used (Discovery XT and OmniMap DAB kit, Ventana Medical Systems). IHC analyses were performed on formalin-fixed paraffin embedded tissue sections and on a subset of fresh frozen tissue sections. All tissues were sectioned to 4-8 μm thick sections and mounted onto Superfrost Plus slides (Thermo Fisher). For fresh frozen brain samples, the tissue was sectioned on Superfrost Plus slides and airdried for 30 min, transferred directly to ice cold acetone 50% for 30 s followed by acetone 100% for 5 min and finally 1×PBS for 5 min before being wet-loaded in the Ventana robotic platform. The working concentration used for Pyr7.1 and Pyr12.2 was 1 μg/ml, and the reference antibody Glu3 was used at 0.5 μg/ml. Visualization of the primary/secondary antibody complex was done by addition of hydrogen peroxide and DAB, resulting in an insoluble brown staining precipitate at the site of antibody binding. Counterstaining was done with hematoxylin (HTX). The stained slides were scanned in bright field using a Pannoramic 250 FLASH II slide scanner. The resulting image files were uploaded into a viewer software (Pannoramic Viewer) and adjusted for optimal brightness and contrast for manual assessment of the staining result.

### Results

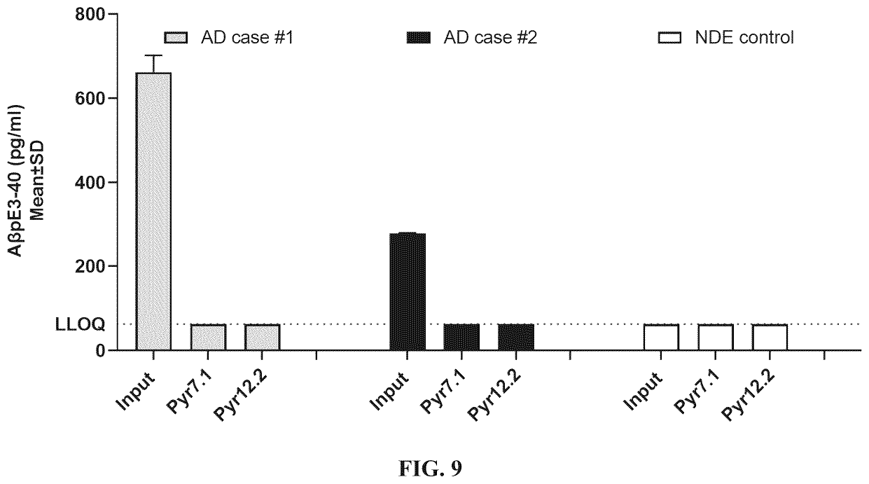

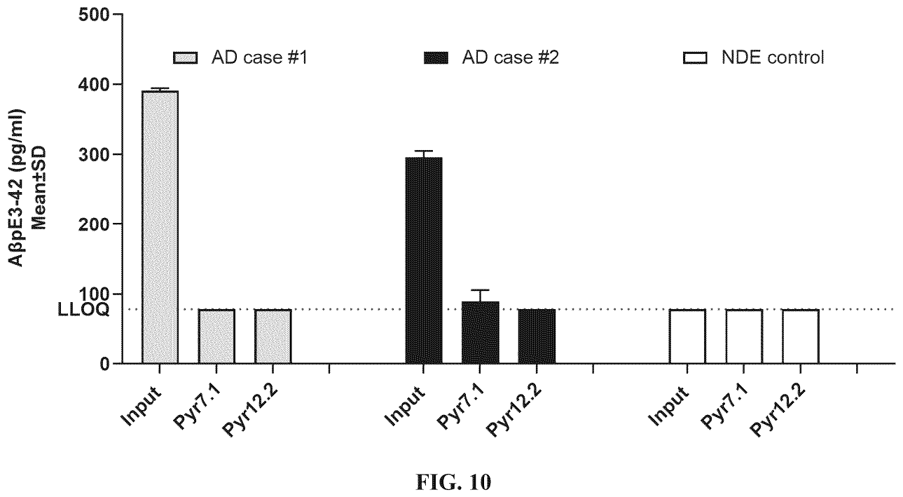

Target binding in human Alzheimer's disease brain extracts by immunoprecipitation: The recombinant antibodies Pyr7.1 and Pyr12.2 were tested for their ability to bind selectively to AβpE3-40 and AβpE3-42 in solution, in human brain extracts from AD patients. Immunoprecipitation (IP) of TBS brain extracts from AD patients using the recombinant antibodies demonstrated depletion of AβpE3-40 (FIG. 9) and AβpE3-42 (FIG. 10) levels by both antibodies. No measurable levels of AβpE3-40 and AβpE3-42 could be detected in brain TBS extract from the NDE control case.

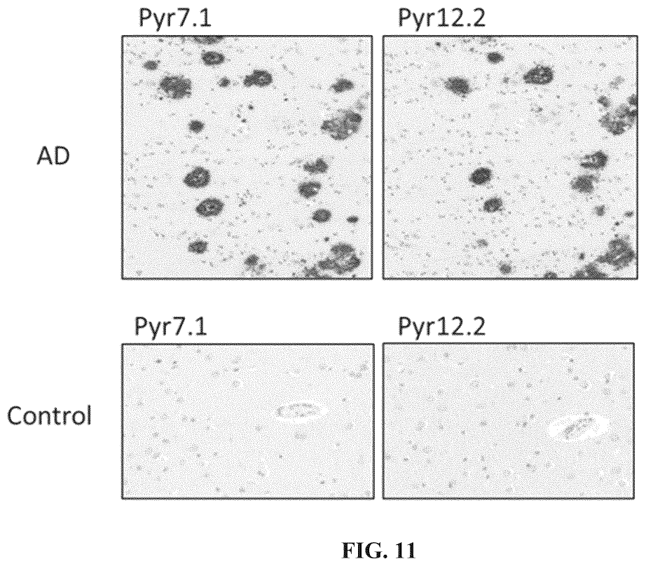

Target binding in human Alzheimer's disease brain by immunohistochemistry: Immunohistochemical staining of brain sections from AD individuals (confirmed to have Aβ pathology by IHC staining with 6E10/4G8, not shown) with recombinant antibodies Pyr7.1 and Pyr12.2 resulted in specific binding by both antibodies to core and diffuse plaques in AD brain, with an identical staining pattern. No binding was observed to NDE control brain. Representative images from immunostaining with Pyr7.1 and Pyr12.2 on adjacent sections from formalin-fixed paraffin embedded AD or NDE control brain are shown in FIG. 11.

### Example 5

### Humanization of Pyr12.2

This example describes the humanization of the murine AβpE3-specific antibody Pyr12.2 described in Examples 2-4, and subsequent production of humanized Pyr12.2 variants.

### Materials and Methods

Humanization: Pyr12.2 was humanized by grafting the CDRs into the IGHV1-46*01 and IKKV2-30*02 human variable domains and making different back-mutations to the mouse residues at various positions. Additional beneficial mutations were performed in the framework regions for one of the variants.

Expression of each variant from transient transfection: The humanized antibodies were expressed in CHO cells and purified by affinity chromatography followed by buffer exchange into phosphate buffered saline (PBS) solution. The purified antibodies were characterized using SDS-PAGE, SEC and UV protein determination.

### Results

Humanization and generation of antibodies: The sequences for humanized VH and VL and the common, human constant regions of the heavy and light chains are given in Table 6 for the two humanized Pyr12.2 variants designated H2L7 and H9L8.

TABLE 6
Amino acid sequences of humanized variants of Pyr12.2

| | | | SEQ |
|---|---|---|---|
| Antibody | Region | Amino acid sequence | ID NO: |
| H2L7 | Heavy chain | | |
| | VH (“VH2”) | EVOLVQSGAEVKKPGASVRLSCKASGYSFTGFT | 22 |
| | | MNWVRQALGQGLEWMGLINPYNGVTTYNQK | |
| | | FKGRLTMTRDMSTRTVYMDLSSLRYEDTAVYYC | |
| | | TREGNWEGVYWGQGTLVTVSS | |
| | VHCDR1 | GFTMN | 7 |
| | VHCDR2 | LINPYNGVTTYNQKFKG | 8 |
| | VHCDR3 | EGNWEGVY | 3 |
| | Light chain | | |
| | VL (“VL7”) | EIVLTQSPLSLSVTLGQSASISCRSSQSLLDSNGKT | 23 |
| | | YLHWFILRPGQSPRRLIYLVSILDSGVPDRFSGSG | |
| | | SGTDFTLKISRVEAEDVGVYYCVQGTHFPFTFGS | |
| | | GTKLEIK | |
| | VLCDR1 | RSSQSLLDSNGKTYLH | 10 |
| | VLCDR2 | LVSILDS | 9 |
| | VLCDR3 | VQGTHFPFT | 6 |
| H9L8 | Heavy chain | | |
| | VH (“VH9”) | QVQLVQSGPEVKKPGSSVKVSCKASGYSFTGFT | 21 |
| | | MNWVRQTPGKGLEWIGLINPYNGVTTYNQKF | |
| | | KGRVTITADESTRTAYMELLSLTYEDTAVYYCTR | |
| | | EGNWEGVYWGQGTPVTVSA | |
| | VHCDR1 | GFTMN | 7 |
| | VHCDR2 | LINPYNGVTTYNQKFKG | 8 |
| | VHCDR3 | EGNWEGVY | 3 |
| | Light chain | | |
| | VL (“VL8”) | EVVLTQSPLSISVTLGQSASISCRSSQSLLDSNGK | 24 |
| | | TYLHWFILRPGQSPRRLIYLVSILDSGIPDRFSGS | |
| | | GSGTDFTLKISRVEAEDVGVYYCVQGTHFPFTFG | |
| | | GGTKLEIK | |
| | VLCDR1 | RSSQSLLDSNGKTYLH | 10 |
| | VLCDR2 | LVSILDS | 9 |
| | VLCDR3 | VQGTHFPFT | 6 |
| Human | Constant | ASTKGPSVFPLAPSSKSTSGGTAALGCLVKDYFP | 31 |
| IgG1 | heavy chain | EPVTVSWNSGALTSGVHTFPAVLQSSGLYSLSS | |
| constant | region | VVTVPSSSLGTQTYICNVNHKPSNTKVDKKVEP | |
| region | | KSCDKTHTCPPCPAPELLGGPSVFLFPPKPKDTL | |
| | | MISRTPEVTCVVVDVSHEDPEVKFNWYVDGVE | |
| | | VHNAKTKPREEQYNSTYRVVSVLTVLHQDWLN | |
| | | GKEYKCAVSNKALPAPIEKTISKAKGQPREPQVY | |
| | | TLPPSRDELTKNQVSLTCLVKGFYPSDIAVEWES | |
| | | NGQPENNYKTTPPVLDSDGSFFLYSKLTVDKSR | |
| | | WQQGNVFSCSVMHEALHNHYTQKSLSLSPGK | |
| Human | Constant light | RTVAAPSVFIFPPSDEQLKSGTASVVCLLNNFYP | 32 |
| kappa | chain region | REAKVQWKVDNALQSGNSQESVTEQDSKDSTYSL | |
| constant | | SSTLTLSKADYEKHKVYACEVTHQGLSSPVTKS | |
| region | | FNRGEC | |

Expression and purification of the humanized antibody variants were performed as described in Example 2 for the recombinant mouse antibodies.

### Example 6

### Characterization of Affinity, Selectivity and Specificity of Humanized Antibodies

This example describes the characterization of the affinity, selectivity and specificity of the humanized Pyr12.2 variant antibodies H2L7 and H9L8 generated and produced in Example 5 by inhibition ELISA and SPR.

### Materials and Methods

Aβ monomer species and Aβ protofibrils: The following Aβ peptides were used for characterization of the binding of the humanized antibodies to different Aβ species: AβpE3-40, Aβ1-40, Aβ1-28 and AβpE11-40, all purchased from Bachem, and Aβ2-28, Aβ3-28, Aβ4-28, Aβ5-28 and AβpE11-28, all custom-made by and purchased from Innovagen. Aβ peptides were dissolved in 10 mM NaOH, 0.005% Tween-20, pH>11 at a concentration of 100 μM. Aliquots were made and stored at −80° C. until analysis. All peptides were verified to be monomeric by size-exclusion chromatography.

Protofibrils were prepared using an AβpE3-42 peptide from Bachem. Briefly, the AβpE3-42 peptide was dissolved in 10 mM NaOH, 0.005% Tween-20, pH>11 at a concentration of 100 μM. Protofibrils were prepared by neutralizing the AβpE3-42 peptide to pH 7.4 by adding 1:1 of a 2×PBS buffer to a final concentration of 50 μM. The peptide was incubated for ˜30 min at 37° C. for protofibril formation and purified from remaining monomers by HPLC on a Superdex 75 Increase 3.2/300 size-exclusion column using a mobile-phase of 1×PBS, 0.1% Tween-20, pH 7.4. Prior to injection, the protofibril reaction was centrifuged at 16000×g for 5 min at 4° C. to remove insoluble fibrils. The void peak containing the AβpE3-42 protofibrils was collected and the concentration determined using SEC and a calibration curve of an Aβ protofibril standard with a known concentration. The same procedure was used to produce Aβ1-42 protofibrils from the corresponding Aβ1-42 monomer (Bachem).

Specificity evaluation and IC₅₀ determination by inhibition ELISA: The specificity of humanized antibodies H2L7 and H9L8 towards AβpE3-28 compared to N-terminally intact Aβ (Aβ1-28) and different N-truncated forms of Aβ (Aβ2-28, Aβ3-28, Aβ4-28, Aβ5-28 and AβpE11-28 monomers) was evaluated by inhibition ELISA. The humanized antibodies were incubated at a fixed concentration (0.5 μg/ml) with titrating concentrations of the different Aβ antigens. After incubation for 45 min at 900 rpm to reach equilibrium, the antibody-Aβ samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

Selectivity evaluation and IC₅₀ determination by inhibition ELISA: The binding of humanized antibodies H2L7 and H9L8 to AβpE3-40 and AβpE3-42 protofibrils and their selectivity towards Aβ1-40 monomers and Aβ1-42 protofibrils were evaluated by inhibition ELISA. The humanized antibodies were incubated at a fixed concentration (0.1 μg/ml) with titrating concentrations of the different Aβ antigens. After incubation for 45 min at 900 rpm to reach equilibrium, the antibody-Aβ samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

Affinity evaluation and K_{D} determination by surface plasmon resonance: Binding interactions between antigens and antibodies were evaluated by SPR using a Biacore 8K instrument (Cytiva) according to standard procedures. The binding of humanized antibodies H2L7 and H9L8 to AβpE3-40 monomers and AβpE3-42 protofibrils was evaluated.

Single cycle kinetics with the antibodies immobilized on a CM5 chip was used to measure the binding of the antibodies to the AβpE3-40 monomer. For the measurements, 5 μg/ml of analyte antibody was immobilized on the chip. The monomer was then injected over the chip using a 2-fold dilution in five steps starting at 250 nM, using 2 min injection of every antibody concentration and a 20 min dissociation time. Regeneration of the surface between cycles was done by injecting 30 μl 3 M MgCl₂. The binding data was fitted to a 1:1 interaction model.

Single cycle kinetics was also used to measure binding of the antibodies to AβpE3-42 protofibrils. The AβpE3-42 protofibrils (138 ng/ml) were coupled to a CM5 chip using general Biacore coupling chemistry (immobilization low levels). For binding to AβpE3-42 protofibrils, the antibodies were injected over the chip using a 4-fold dilution series in five steps starting at 150 nM, using 2 min injection of every antibody concentration and a 20 min dissociation time. Regeneration of the surface between cycles was done by injecting 30 μl 10 mM glycine-HCl pH 1.7. The binding data was fitted to a 1:1 interaction model. The use of the 1:1 interaction model gives the K_{D} for the binding of the antibodies to Aβ monomers and an apparent K_{D} for the binding of the antibodies to Aβ protofibrils.

In all SPR experiments, 1×HBS-EP+ (Cytiva, cat. no. BR100669) was used to dilute antibodies and target antigens. Experiments were performed at 25° C.

### Results

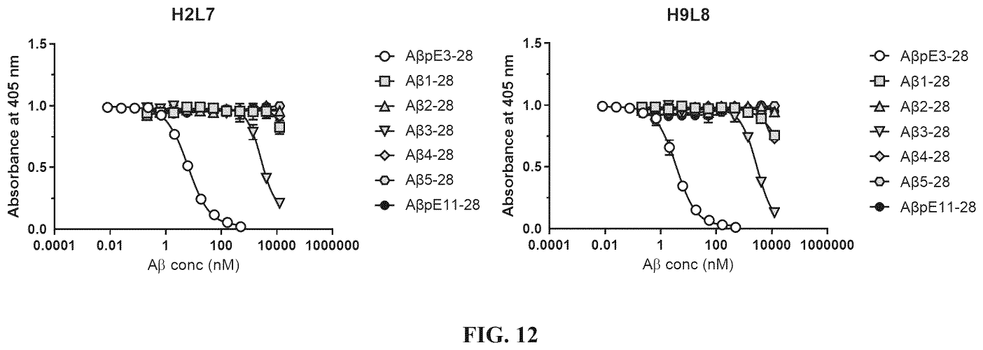

Specificity evaluation and IC₅₀ determination by inhibition ELISA: The specificity of humanized antibodies H2L7 and H9L8 towards AβpE3-28 compared to N-terminally intact Aβ (Aβ1-28) and different N-truncated forms of Aβ (Aβ2-28, Aβ3-28, Aβ4-28, Aβ5-28 and AβpE11-28 monomers) was evaluated by inhibition ELISA. Humanized antibodies H2L7 and H9L8 demonstrated highest binding to AβpE3-28 monomer in solution, with some cross-reactivity to Aβ3-28 (FIG. 12). None of the antibodies bound Aβ1-28, Aβ2-28, Aβ4-28, Aβ5-28 or AβpE11-28 monomers in solution at concentrations up to 12.5 μM, suggesting the IC₅₀ values were >12.5 μM for these antibodies. The calculated IC₅₀ values are listed in Table 7.

TABLE 7
Specificity evaluation using inhibition ELISA (Mean ± SD)

| | Aβ1-28 | Aβ2-28 | Aβ3-28 | AβpE3-28 | Aβ4-28 | Aβ5-28 | AβpE11-28 |
|---|---|---|---|---|---|---|---|
| | IC₅₀ | IC₅₀ | IC₅₀ | IC₅₀ | IC₅₀ | IC₅₀ | IC₅₀ |
| Ab | (nM) | (nM) | (nM) | (nM) | (nM) | (nM) | (nM) |
| H2L7 | >12500 | >12500 | 1872 ± 1387 | 5.5 ± 2.8 | >12500 | >12500 | >12500 |
| H9L8 | >12500 | >12500 | 1788 ± 1333 | 3.5 ± 0.8 | >12500 | >12500 | >12500 |

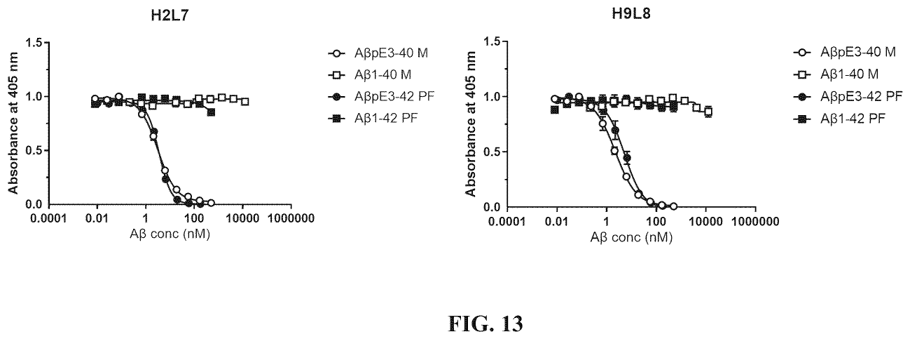

Selectivity evaluation and IC₅₀ determination by inhibition ELISA: The binding of the humanized antibodies H2L7 and H9L8 to AβpE3-40 monomers and AβpE3-42 protofibrils and selectivity versus Aβ1-40 monomers and Aβ1-42 protofibrils was evaluated using inhibition ELISA. Humanized antibodies H2L7 and H9L8 demonstrated binding to AβpE3-40 monomer and AβpE3-42 protofibril in solution (FIG. 13). None of the antibodies bound Aβ1-40 monomer in solution at concentrations up to 12.5 μM, suggesting the IC₅₀ values were >12.5 μM for these antibodies. None of the antibodies bound Aβ1-42 protofibrils in solution at concentrations up to 500 nM, suggesting the IC₅₀ values were >500 nM for these antibodies. The calculated IC₅₀ values are listed in Table 8.

TABLE 8
Selectivity analysis using inhibition ELISA (Mean ± SD)

| | AβpE3-40 | AβpE3-42 | Aβ1-40 | Aβ1-42 |
|---|---|---|---|---|
| | monomer | protofibril | monomer | protofibril |
| Antibody | IC₅₀ (nM) | IC₅₀ (nM) | IC₅₀ (nM) | IC₅₀ (nM) |
| H2L7 | 2.6 ± 0.7 | 2.5 ± 0.9 | >12500 | >500 |
| H9L8 | 2.1 ± 0.3 | 4.9 ± 1.1 | >12500 | >500 |

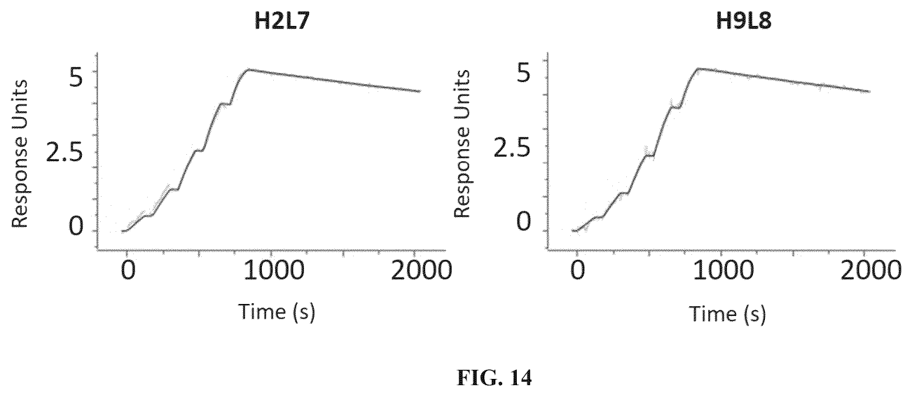

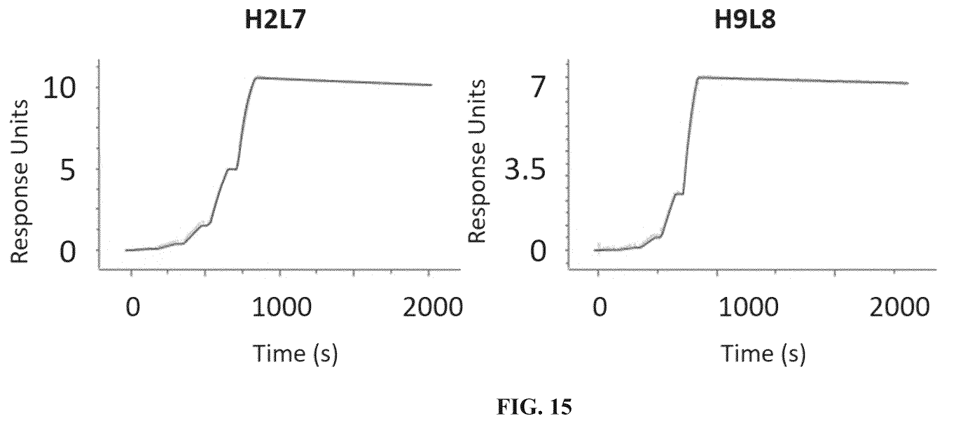

Affinity evaluation and K_{D} determination by surface plasmon resonance: The binding of humanized antibodies H2L7 and H9L8 to AβpE3-40 monomers and AβpE3-42 protofibrils was evaluated in SPR and their K_{D} values were determined. Both antibodies demonstrated binding to AβpE3-40 monomers and AβpE3-42 protofibrils. The calculated kₐ, k_{d} and (apparent) K_{D} values are shown in Table 9 and Table 10 below. Representative sensorgrams are shown in FIG. 14 and FIG. 15.

TABLE 9
SPR analysis of binding to AβpE3-40 monomers

AβpE3-40 monomer

| | kₐ (M⁻¹s⁻¹) | k_{d} (s⁻¹) | K_{D} (nM) |
|---|---|---|---|
| Antibody | Mean ± SD | Mean ± SD | Mean ± SD |
| H2L7 | 5.05 ± 0.81 e4 | 1.25 ± 0.24 e−4 | 2.54 ± 0.60 |
| H9L8 | 5.37 ± 1.69 e4 | 1.39 ± 0.44 e−4 | 2.70 ± 0.67 |

TABLE 10
SPR analysis of binding to AβpE3-42 protofibrils

| | AβpE3-42 protofibril | | |
|---|---|---|---|
| | kₐ (M⁻¹s⁻¹) | k_{d} (s⁻¹) | Apparent K_{D} (pM) |
| Antibody | Mean ± SD | Mean ± SD | Mean ± SD |
| H2L7 | 1.07 ± 0.08 e5 | 1.79 ± 0.84 e−5 | 173 ± 99.4 |
| H9L8 | 4.43 ± 0.34 e4 | 2.05 ± 0.51 e−5 | 464 ± 117 |

### Example 7

### Target Binding of Humanized Antibodies in Brain from Human Alzheimer's Disease Patients and Non-Demented Controls

This example describes target binding of humanized antibodies H2L7 and H9L8, generated as described in Example 5 and tested by immunoprecipitation on human brain extracts and immunohistochemistry on human brain sections from AD patients and NDE controls.

### Materials and Methods

Target binding in human Alzheimer's disease brain extracts by immunoprecipitation: Antibody binding to target in human AD brain was analyzed by immunoprecipitation, which is a method for removal of target protein in a sample using an antibody specific for the target molecule. Briefly, each of the humanized antibodies was incubated with magnetic Protein A Dynabeads and soluble 16000×g TBS brain extracts from an AD case. The TBS brain extracts were prepared as described in Example 4. The bead-bound target was separated by a magnet and eluted from the beads using 70% formic acid. After neutralization, the pellet (IP fraction) was analyzed using in house developed MSD assay for measurements of total AβpE3-x levels. Briefly, MSD GOLD 96-well small spot streptavidin plate was coated with biotinylated Pyr12.2 antibody for 1 h at RT. After a washing step, free binding sites were blocked by incubation with Diluent 35. The plate was washed again and incubated further for 2 h (900 rpm shaking) with a dilution series of a standard (AβpE3-40 monomer, 3.125-400 pg/ml) and with test samples. After another washing step, detection antibody anti-Aβ4G8 SULFO tag conjugated was added to the plate for 1 h at a concentration of 1 μg/ml. Finally, the plate was washed and read using an MSD sector imager (S 600 MM, MSD). Obtained signal was correlated to the amount of AβpE3-x in the sample.

Target binding in human Alzheimer's disease brain by immunohistochemistry: Immunohistochemistry (IHC) analyses were performed on brain tissue from AD patients. Postmortem human brain tissue of temporal cortex was obtained from the Netherlands Brain Bank (NBB) and had been collected at autopsy with local ethical committee approval.

The mouse anti-human Aβ antibodies 6E10 (Covance #SIG-39320) and 4G8 (Covance #SIG-39200) were used for detection of Aβ pathology in brain sections. Human target binding in AD brain was evaluated for humanized antibodies H2L7 and H9L8.

For IHC staining of Aβ, an automated staining robot and a HRP-3,3′-diaminobenzidine (DAB) based detection system was used (Discovery XT and OmniMap DAB kit, Ventana Medical Systems). IHC analyses were performed on fresh frozen tissue sections. All tissues were sectioned in 4-8 μm sections which were mounted onto Superfrost Plus slides (ThermoFisher). The sections were airdried for 30 min, transferred directly to ice cold acetone 50% for 30 s followed by acetone 100% for 5 min and finally 1×PBS for 5 min before being wet-loaded in the Ventana robotic platform. The working concentration used for tested antibodies H2L7 and H9L8 and for reference antibodies 6E10 and 4G8 was 1 μg/ml. Visualization of the primary/secondary antibody complex was done by addition of hydrogen peroxide and DAB, resulting in an insoluble brown staining precipitate at the site of antibody binding. Counterstaining was done with hematoxylin (HTX). The stained slides were scanned in bright field using a Pannoramic 250 FLASH II slide scanner. The resulting image files were uploaded into a viewer software (Pannoramic Viewer) and adjusted for optimal brightness and contrast for manual assessment of the staining result.

### Results

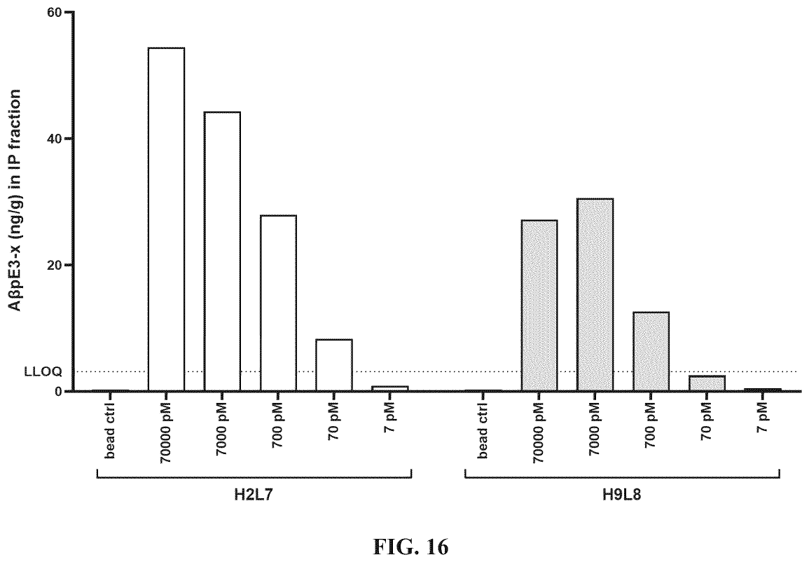

Target binding in human Alzheimer's disease brain extracts by immunoprecipitation: The humanized antibodies H2L7 and H9L8 were tested for their ability to bind to AβpE3 in solution, in brain extracts from human AD patients. Immunoprecipitation (IP) of TBS brain extracts from AD patients using the humanized antibodies demonstrated a concentration-dependent IP of AβpE3-x by both antibodies (FIG. 16).

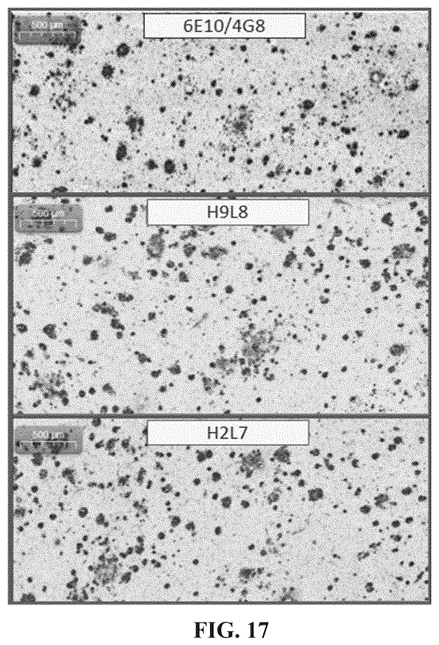

Target binding in human Alzheimer's disease brain by immunohistochemistry: Immunohistochemical staining of brain sections from AD individuals (confirmed to have Aβ pathology by IHC staining with 6E10/4G8) with humanized antibodies H2L7 and H9L8 resulted in specific binding by both antibodies to core and diffuse plaques in AD brain, with an identical staining pattern. No binding was observed to NDE control brain (data not shown). Representative images from immunostaining with H2L7 and H9L8 on adjacent sections from fresh-frozen AD brain are shown in FIG. 17.

### Example 8

### Characterization of Functional Effects of Humanized Antibodies

This example describes the functional effects of humanized AβpE3 antibodies H2L7 and H9L8 generated and produced in Example 5. The ability of the humanized antibodies to inhibit aggregation of AβpE3 and to clear amyloid plaques ex vivo in AD brain sections was evaluated.

### Materials and Methods

Inhibition of aggregation of AβpE3-42: The effect of the humanized antibodies on aggregation of AβpE3-42 monomers was evaluated in an aggregation assay using thioflavin T (ThT, Sigma T3516). AβpE3-42-NH4+ monomers (Bachem, H4916, 2 μM) were mixed with ThT (5 μM) and with either H2L7 or H9L8 humanized antibody (25-800 nM) or an IgG1 isotype control antibody (CrownVivo, C-00012, 800 nM) in phosphate buffer saline (PBS) pH 8.2, 200 mM NaCl, 10 μM EDTA in 384-well plates (Thermo scientific #242764) on ice. The plates were then transferred to a Tecan SPARK enhanced plate reader equipped with a 448±7 nm excitation filter and a 485±20 nm emission filter at 37° C. for recordings of ThT fluorescence over 36-72 hrs. The data was background corrected and the maximum fluorescence plotted against antibody concentration to calculate the IC₅₀. The experiment was repeated 3-5 times. Statistical significance was tested with two-way ANOVA.

Ex vivo phagocytosis in AD brain: An ex vivo phagocytosis assay was used to investigate whether the humanized antibodies could induce plaque clearance by macrophages. Fresh frozen AD brain tissue was cryosectioned (20 μm) and sections were collected onto poly-D-lysine (Gibco A38904-01, 50 μg/ml) coated 12 mm glass coverslips. Sections were then incubated with humanized antibody (1 μg/ml) or an IgG1 isotype control antibody (CrownVivo, C-00012, 1 μg/ml) for 1 h at 37° C. 5% CO₂. Sections were then washed once and incubated for 24 h with 5×10⁵ to 1×10⁶ primary human macrophages isolated from buffy coats. Plaque clearance was evaluated after immunohistochemistry with the mouse anti-human Aβ antibodies 6E10 (Covance #SIG-39320) and 4G8 (Covance #SIG-39200) by measuring the immunopositive area on each section. Experiments were repeated 2 to 5 times with macrophages isolated from different buffy coats. Statistical significance was tested with one-way ANOVA.

### Results

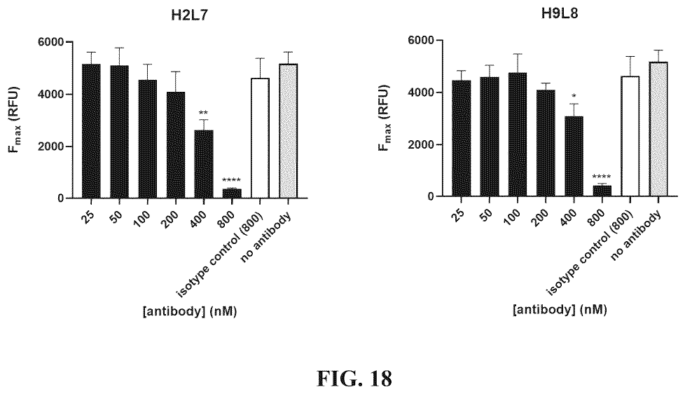

Inhibition of aggregation of AβpE3-42: The ability of the humanized antibodies H2L7 and H9L8 to inhibit aggregation of AβpE3-42 was evaluated. Both H2L7 and H9L8 concentration-dependently inhibited fibril formation of AβpE3-42, as demonstrated by the decrease in the maximum ThT fluorescence signal (Fmax) in the presence of the antibodies (FIG. 18).

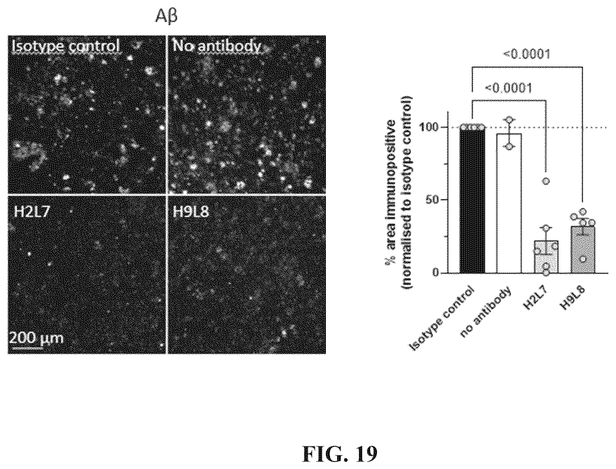

Ex vivo phagocytosis in AD brain: The ability of the humanized antibodies H2L7 and H9L8 to induce clearance of Aβ plaques by macrophages in AD brain was evaluated. Compared to negative control samples pre-incubated without antibodies or with an isotype control IgG1 antibody, Aβ plaques were significantly reduced after pre-incubation with H2L7 or H9L8. The results indicate that both antibodies can induce plaque clearance by macrophages (FIG. 19).

### Example 9

### Pharmacokinetic Profile of Murine and Humanized AβpE3 Antibodies

This example describes the pharmacokinetic (PK) profile in mouse after dosing with murine antibody Pyr12.2, generated and produced as described in Example 2, or with humanized antibodies H2L7 and H9L8, generated and produced as described in Example 5.

### Materials and Methods

Administration of antibody and sample collection: Each respective antibody was administered intravenously (i.v.) at a dose of 10 mg/kg via the tail vein to 8 weeks old female C57BL/6J mice (five mice per antibody). Blood samples were collected at 5 min, 4 h, 24 h, 72 h, 168 h (7 days), 336 h (14 days), 672 h (28 days) and 840 h (35 days) after i.v. injection. Blood was collected into Microvette EDTA tubes and put on wet ice immediately after collection. The samples were centrifuged at 2400×g for 10 min at +4° C. shortly after collection (within 30 min). Plasma was collected and stored at −80° C. until bioanalysis.

Determination of antibody concentrations in plasma: Concentrations of Pyr12.2 were determined in EDTA plasma samples using an MSD based method. Briefly, MSD standard 96-well plates were coated with 0.5 μM monomeric AβpE3-40 (Bachem) overnight at 4° C. Free binding sites were blocked by incubation with 1% Blocker A (MSD Blocker A in 1×PBS-Tween 20) for 1 h at RT with shaking. Washing was performed before blocking and before each subsequent incubation step. Standard and plasma samples were added and incubated for 2 h at RT with shaking. Bound antibodies were detected by incubation with an MSD SULFO-TAG labelled goat anti-mouse IgG antibody (R32AC-1, 0.5 μg/ml) for 1 h at RT with shaking. Read buffer T (MSD 2×) was added and the plates were read using an MSD sector imager. The signal strength was correlated to the amount of Pyr12.2 in the samples.

Concentration levels of H2L7 and H9L8 were determined in EDTA plasma samples using a commercial kit from MSD for measuring Human/NHP IgG (cat no: K150JLD). Standard and plasma samples were added to a pre-coated anti-human-IgG MSD plate and incubated for 2 h in RT with shaking. An anti-human/NHP IgG antibody conjugated with a SULFO-TAG label was added after a washing step and incubated at 2 h, after which the MSD read buffer was added after a washing step. When read in an MSD sector imager a light signal was generated and measured. The signal strength was correlated to the amount of the H2L7 or H9L8 in the samples. Pharmacokinetic analysis: The software Phoenix WinNonlin was used for non-compartment analysis (NCA) of the plasma concentration data. Individual observed plasma concentration versus time profiles were subjected to PK evaluation. Nominal dose and time points were used in the analysis. The maximum concentration, Cmax, was directly derived from the observed concentrations versus time curves. The calculation method in the NCA was set to Linear up log down, where linear trapezoidal rule is used when concentration versus time data is increasing, and the logarithmic trapezoidal rule is used when the concentration data is decreasing. Calculated parameters included the area under the concentration versus time curve (AUC) to the last observed time point (AUClast) or to infinity (AUCinf), calculated as AUClast+Ct/λz, where Ct is the observation at the last time point and λz is the elimination rate constant estimated using log-linear regression during the terminal elimination phase. Terminal half-life (t½) was calculated as In(2)/Δz, clearance (CL) was calculated as Dose/AUCinf.

### Results

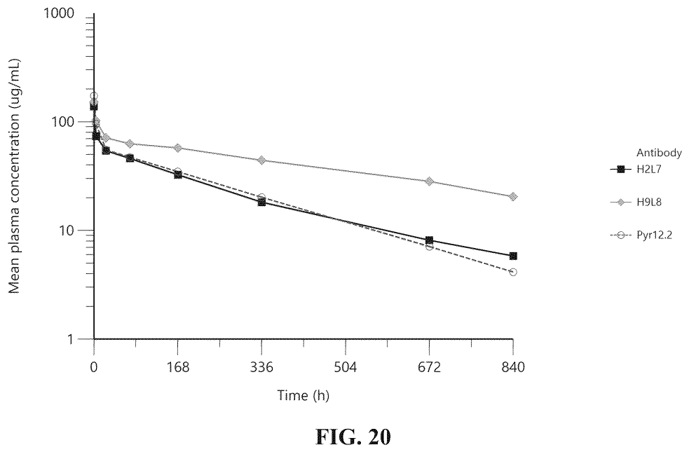

The plasma PK profiles for the murine Pyr12.2 antibody and humanized antibodies H2L7 and H9L8 were evaluated after a single i.v. bolus injection into C57BL/6 mice. The profiles of plasma concentration as a function of time are shown in FIG. 20 and the calculated PK parameters are shown in Table 11. While H2L7 showed a similar plasma PK profile in mouse as the murine antibody Pyr12.2, the H9L8 variant surprisingly demonstrated a better plasma PK profile with a longer half-life, a higher total exposure (AUC) and lower clearance (CL).

TABLE 11
Summary of PK parameters (Mean)

| | Half-life | Cmax | AUCinf | CL |
|---|---|---|---|---|
| Antibody | (days) | (μg/ml) | (mg/ml*h) | (ml/h/kg) |
| Pyr12.2 | 9.3 | 173 | 19.3 | 0.52 |
| H2L7 | 11.5 | 140 | 19.6 | 0.54 |
| H9L8 | 19.6 | 152 | 49.7 | 0.20 |

### Example 10

### Generation of Humanized Antibodies with an Improved Pharmacokinetic Profile

This example describes the generation of variants of humanized antibody H2L7 having an improved pharmacokinetic (PK) profile.

### Material and Methods

Design and expression of H2L7 variants: The HC and LC sequences of the humanized antibodies H2L7 and H9L8 of the preceding Examples were compared in order to identify positively charged amino acids that could potentially cause the difference in PK profile between the two antibodies (Table 11). Sequences were also compared to the murine parental antibody Pyr12.2. The structures for H2L7 and H9L8 were modeled in silico, and surface analysis was performed. Amino acids in H2L7 that were identified to contribute to positively charged patches were mutated to neutral amino acids. Different mutations were combined to generate several new antibodies. New surface analyses were performed for mutated antibodies. A total of six antibodies with mutations that reduced positively charged patches in the surface analysis were expressed and purified as described in Example 5 for H2L7 and H9L8, with an additional preparative grade SEC step.

Administration of antibody and sample collection: The antibody variants were administered at a 10 mg/kg dose i.v. via the tail vein to 8 weeks old female C57BL/6J mice (five mice per antibody). Blood samples were collected at 5 min, 4 h, 24 h, 72 h, 168 h (7 days), 336 h (14 days), 672 h (28 days) and 840 h (35 days) after the i.v. injection. Blood was collected into Microvette EDTA tubes and put on wet ice immediately after collection. The samples were centrifuged at 2400×g for 10 minutes at 4° C. shortly after collection (within 30 min). Plasma was collected and stored at −80° C. until bioanalysis.

Determination of antibody concentration in plasma: Concentrations of the antibody variants were determined in EDTA plasma samples using a commercial kit from MSD for measuring Human/NHP IgG (catalog number: K150JLD), as described in Example 9 for H2L7 and H9L8.

Pharmacokinetic analysis: Individual, observed profiles of plasma concentration versus time were subjected to PK evaluation as described in Example 9 for H2L7 and H9L8.

### Results

Design of H2L7 variants with an improved pharmacokinetic profile: From the sequence alignment of VH2 (SEQ ID NO:22) and VH9 (SEQ ID NO:21), residues R71 and R83 found in VH2 were considered to be highly likely to contribute to the higher clearance for H2L7 in comparison to VH9 in H9L8 (comprising A71 and T83). K12 in VH2 was also considered as a candidate for mutation, because the corresponding residue at this position in the parental murine Pyr12.2 antibody is a neutral valine residue. It is worth noting that VH9 also has a K12 residue, and thus the discrepancy in clearance between H2L7 and H9L8 may not stem from this position. With respect to the light chain, there were no obvious residue differences between VL7 (SEQ ID NO:23) and VL8 (SEQ ID NO:24) that were deemed to contribute to the increased net positive charge in H2L7 compared to H9L8. Based on a sequence alignment, it was concluded that the VH domain was likely responsible for the notable difference in clearance between H2L7 and H9L8. It was therefore hypothesized that making mutations in VH2 to remove the positively charged residues could reduce clearance of H2L7 to be more in line with the clearance observed for H9L8.

Protein surface analysis of H2L7 and H9L8 revealed distinct, positively charged regions in H2L7 which are either missing or significantly reduced in H9L8. R83 in the VH domain of H2L7 contributes to a large positively charged patch which is absent in H9L8, because the corresponding residue at that position (threonine, T83) is a neutral amino acid. Similarly, R71 in VH of H2L7 contributes to a positively charged patch, while the corresponding residue in H9L8 is alanine (A71) which does not contribute to a positive protein surface patch. Also, position 12 in the VH domain of H2L7 was mutated from the K12 present in both VH2 and VH9 back to the neutral V12 present in the murine ancestor Pyr12.2. This could also contribute to a reduced overall positive protein surface.

Taking the above analysis into account, variants of H2L7 with the following mutations in the heavy chain variable domain (VH) were postulated to have an improved PK: H2L7-R71A, H2L7-R83T, H2L7-K12V/R71A, H2L7-K12V/R83T, H2L7-R71A/R83T and H2L7-K12V/R71A/R83T. The VH sequences are shown in Table 12. The amino acid sequences of the VL and constant domains of the variant antibodies were identical to the sequences of the corresponding domains in H2L7 given in Table 6. Of these, H2L7-R71A, H2L7-R83T, H2L7-K12V/R71A, H2L7-K12V/R83T, H2L7-R71A/R83T and H2L7-K12V/R71A/R83T were produced.

TABLE 12
Amino acid sequences of VH domain variants of H2L7

| | | SEQ |
|---|---|---|
| | | ID |
| Antibody | VH domain amino acid sequence | NO: |
| H2L7-R71A | EVQLVQSGAEVKKPGASVRLSCKASGYSFTGFTMNWVR | 16 |
| | QALGQGLEWMGLINPYNGVTTYNQKFKGRLTMTADMS | |
| | TRTVYMDLSSLRYEDTAVYYCTREGNWEGVYWGQGTLV | |
| | TVSS | |
| H2L7-R83T | EVQLVQSGAEVKKPGASVRLSCKASGYSFTGFTMNWVR | 17 |
| | QALGQGLEWMGLINPYNGVTTYNQKFKGRLTMTRDMS | |
| | TRTVYMDLSSLTYEDTAVYYCTREGNWEGVYWGQGTLV | |
| | TVSS | |
| H2L7-K12V/ | EVQLVQSGAEVVKPGASVRLSCKASGYSFTGFTMNWVR | 15 |
| R71A | QALGQGLEWMGLINPYNGVTTYNQKFKGRLTMTADMS | |
| | TRTVYMDLSSLRYEDTAVYYCTREGNWEGVYWGQGTLV | |
| | TVSS | |
| H2L7-K12V/ | EVQLVQSGAEVVKPGASVRLSCKASGYSFTGFTMNWVR | 18 |
| R83T | QALGQGLEWMGLINPYNGVTTYNQKFKGRLTMTRDMS | |
| | TRTVYMDLSSLTYEDTAVYYCTREGNWEGVYWGQGTLV | |
| | TVSS | |
| H2L7-R71A/ | EVQLVQSGAEVKKPGASVRLSCKASGYSFTGFTMNWVR | 20 |
| R83T | QALGQGLEWMGLINPYNGVTTYNQKFKGRLTMTADMS | |
| | TRTVYMDLSSLTYEDTAVYYCTREGNWEGVYWGQGTLV | |
| | TVSS | |
| H2L7-K12V/ | EVQLVQSGAEVVKPGASVRLSCKASGYSFTGFTMNWVR | 19 |
| R71A/R83T | QALGQGLEWMGLINPYNGVTTYNQKFKGRLTMTADMS | |
| | TRTVYMDLSSLTYEDTAVYYCTREGNWEGVYWGQGTLV | |
| | TVSS | |

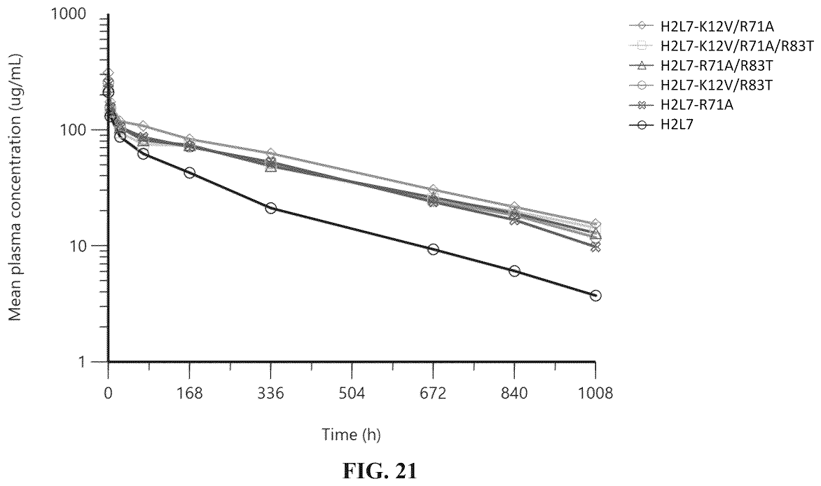

Evaluation of Pharmacokinetic profile of H2L7 and variants in mouse: The plasma PK profile for H2L7 and five H2L7 variants after a single i.v. bolus injection into C57BL/6 mice was evaluated. The profiles of plasma concentration versus time are shown in FIG. 21 and the calculated PK parameters are shown in Table 13. All variants of H2L7 showed an improved plasma PK profile in mouse, exhibiting longer half-life, higher total exposure (AUC) and lower clearance (CL) than the original H2L7 antibody.

TABLE 13
Summary of PK parameters (Mean)

| | Half-life | AUCinf | Cmax | CL |
|---|---|---|---|---|
| Antibody | (days) | (mg/ml*h) | (μg/ml) | (ml/h/kg) |
| H2L7 | 10.8 | 24.7 | 211.0 | 0.41 |
| H2L7-K12V/R71A | 14.0 | 60.4 | 310.1 | 0.17 |
| H2L7-R71A | 12.5 | 47.9 | 251.1 | 0.21 |
| H2L7-K12V/R83T | 13.4 | 49.2 | 256.8 | 0.21 |
| H2L7-R71A/R83T | 14.3 | 50.2 | 234.2 | 0.20 |
| H2L7-K12V/R71A/R83T | 15.2 | 50.4 | 216.9 | 0.20 |

### Example 11

### Affinity, Selectivity and Specificity of H2L7 Variant Antibodies

This example describes characterization, by inhibition ELISA and SPR, of the affinity, selectivity and specificity of the H2L7 variant antibodies generated and produced as described in Example 10.

### Materials and Methods

Aβ monomer species and Aβ protofibrils: The Aβ monomer species as described in Example 6 were used for characterization of binding to monomeric Aβ species. AβpE3-42 and Aβ1-42 protofibrils were prepared as described in Example 6 and used for characterization of binding to aggregated Aβ species.

Specificity evaluation and IC₅₀ determination by inhibition ELISA: The specificity towards AβpE3-28 compared to N-terminally intact Aβ (Aβ1-28) and different N-truncated forms of Aβ (Aβ2-28, Aβ3-28, Aβ4-28, Aβ5-28 and AβpE11-28 monomers) was evaluated by inhibition ELISA as described for H2L7 and H9L8 in Example 6.

Selectivity evaluation and IC₅₀ determination by inhibition ELISA: The binding of the antibodies to AβpE3-40 and AβpE3-42 protofibrils and selectivity against Aβ1-40 monomers and Aβ1-42 protofibrils was evaluated by inhibition ELISA as described for H2L7 and H9L8 in Example 6.

Affinity evaluation and K_{D} determination by surface plasmon resonance: Binding interactions between antigens and antibodies were evaluated by SPR using a Biacore 8K instrument (Cytiva) according to standard procedures. The binding of the H2L7 variant antibodies to AβpE3-40 monomers and AβpE3-42 protofibrils was evaluated as described for H2L7 and H9L8 in Example 6, with the exception of using immobilization of 10 μg/ml of analyte antibody on the chip for measurement of binding to AβpE3-40 monomer.

### Results

Specificity evaluation and IC₅₀ determination by inhibition ELISA: The specificity of H2L7, H2L7-K12V/R71A and H2L7-K12V/R83T towards AβpE3-28 compared to N-terminally intact Aβ (Aβ1-28) and different N-truncated forms of Aβ(Aβ2-28, Aβ3-28, Aβ4-28, Aβ5-28, and AβpE11-28 monomers) was evaluated by inhibition ELISA. H2L7 and both H2L7 variants demonstrated the highest binding to AβpE3-28 monomer in solution, with some cross-reactivity to Aβ3-28. The calculated IC₅₀ values are listed in Table 14.

TABLE 14
Specificity evaluation using inhibition ELISA

| | Aβ1-28 | Aβ2-28 | Aβ3-28 | AβpE3-28 | Aβ4-28 | Aβ5-28 | AβpE11-28 |
|---|---|---|---|---|---|---|---|
| | IC₅₀ | IC₅₀ | IC₅₀ | IC₅₀ | IC₅₀ | IC₅₀ | IC₅₀ |
| Antibody | (nM) | (nM) | (nM) | (nM) | (nM) | (nM) | (nM) |
| H2L7 | >12500 | >12500 | 2347 | 3.6 | >12500 | >12500 | >12500 |
| H2L7- | 10179 | >12500 | 2820 | 3.0 | >12500 | >12500 | >12500 |
| K12V/R71A | | | | | | | |
| H2L7- | >12500 | >12500 | 2260 | 5.0 | >12500 | >12500 | >12500 |
| K12V/R83T | | | | | | | |

Selectivity evaluation and IC₅₀ determination by inhibition ELISA: The binding of H2L7 and five different H2L7 variants to AβpE3-40 monomers AβpE3-42 protofibrils and selectivity versus Aβ1-40 monomers and Aβ1-42 protofibrils was evaluated using inhibition ELISA. H2L7 and its variants demonstrated binding to AβpE3-40 monomer and AβpE3-42 protofibril in solution. None of the antibodies bound Aβ1-40 monomer in solution at concentrations up to 500 nM, suggesting that the corresponding IC₅₀ values were >500 nM. None of the antibodies bound Aβ1-42 protofibrils in solution at concentrations up to 132 nM, suggesting that the corresponding IC₅₀ values were >132 nM. The calculated IC₅₀ values are listed in Table 15.

TABLE 15
Selectivity evaluation using inhibition ELISA (Mean ± SD, n = 2)

| | AβpE3-40 | AβpE3-42 | Aβ1-40 | Aβ1-42 |
|---|---|---|---|---|
| | monomer | protofibril | monomer | protofibril |
| Antibody | IC₅₀ (nM) | IC₅₀ (nM) | IC₅₀ (nM) | IC₅₀ (nM) |
| H2L7 | 2.35 ± 0.02 | 3.00 ± 0.40 | >500 | >132 |
| H2L7-K12V/R71A | 1.69 ± 0.07 | 3.58 ± 0.58 | >500 | >132 |
| H2L7-R71A | 2.54 ± 0.32 | 4.59 ± 0.19 | >500 | >132 |
| H2L7-K12V/R83T | 2.27 ± 0.47 | 3.71 ± 0.08 | >500 | >132 |
| H2L7-R71A/R83T | 2.77 ± 0.22 | 5.44 ± 0.66 | >500 | >132 |
| H2L7-K12V/R71A/ | 2.29 ± 0.21 | 4.90 ± 0.04 | >500 | >132 |
| R83T | | | | |

Affinity evaluation and K_{D} determination by surface plasmon resonance: The binding of H2L7 and the H2L7 variants to AβpE3-40 monomers and AβE3-42 protofibrils was evaluated by SPR and their K_{D} values were determined.

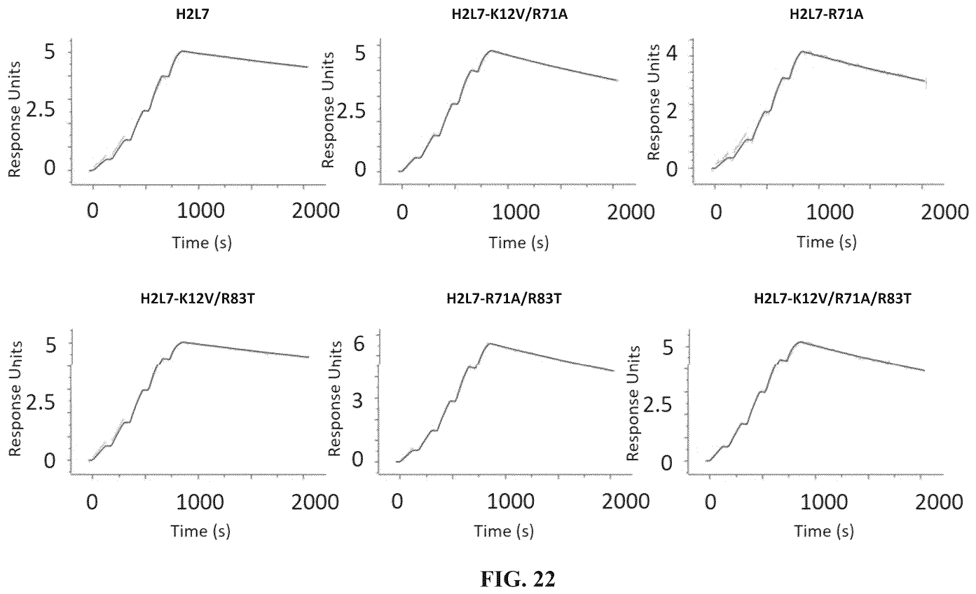

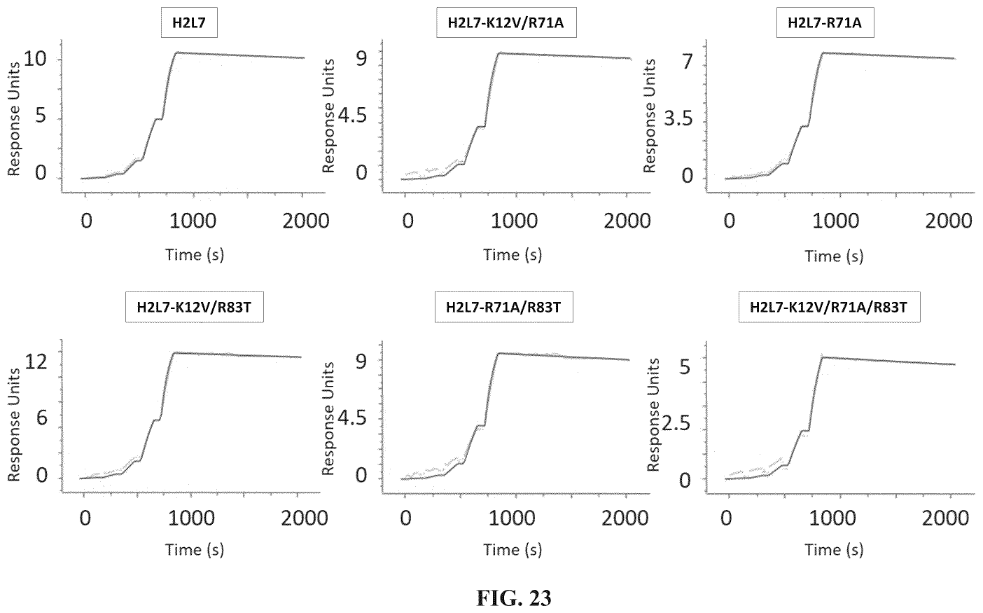

All H2L7 antibody variants demonstrated binding to AβpE3-40 monomers and AβpE3-42 protofibrils. The calculated kₐ, k_{d} and (apparent) K_{D} values are shown in Table 16 and Table 17 below. Representative sensorgrams are shown in FIG. 22 and FIG. 23.

TABLE 16
Summary of SPR analysis of binding to AβpE3-40 monomers

| | AβpE3-40 monomer | | |
|---|---|---|---|
| | kₐ (M⁻¹s⁻¹) | k_{d} (s⁻¹) | K_{D} (nM) |
| Antibody | Mean ± SD | Mean ± SD | Mean ± SD |
| H2L7 | 5.05 ± 0.81 e4 | 1.25 ± 0.24 e−4 | 2.54 ± 0.60 |
| H2L7-K12V/R71A | 5.90 ± 1.09 e4 | 2.33 ± 0.31 e−4 | 4.14 ± 1.23 |
| H2L7-R71A | 5.01 ± 1.04 e4 | 2.25 ± 0.27 e−4 | 4.62 ± 0.88 |
| H2L7-K12V/R83T | 5.62 ± 0.93 e4 | 1.20 ± 0.15 e−4 | 2.20 ± 0.47 |
| H2L7-R71A/R83T | 5.09 ± 0.75 e4 | 2.35 ± 0.23 e−4 | 4.72 ± 0.89 |
| H2L7-K12V/R71A/ | 5.87 ± 0.67 e4 | 2.34 ± 0.38 e−4 | 4.04 ± 0.73 |
| R83T | | | |

TABLE 17
Summary of SPR analysis of binding to AβpE3-42 protofibrils

| | AβpE3-42 protofibril | | |
|---|---|---|---|
| | kₐ (M⁻¹s⁻¹) | k_{d} (s⁻¹) | K_{D} (pM) |
| Antibody | Mean ± SD | Mean ± SD | Mean ± SD |
| H2L7 | 1.07 ± 0.08 e5 | 1.79 ± 0.84 e−5 | 173 ± 99.4 |
| H2L7-K12V/R71A | 6.12 ± 0.85 e4 | 2.92 ± 0.68 e−5 | 477 ± 90.6 |
| H2L7-R71A | 6.77 ± 0.70 e4 | 2.54 ± 0.62 e−5 | 376 ± 85.5 |
| H2L7-K12V/R83T | 8.11 ± 0.61 e4 | 1.62 ± 0.48 e−5 | 200 ± 55.7 |
| H2L7-R71A/R83T | 5.74 ± 0.94 e4 | 2.73 ± 0.87 e−5 | 471 ± 95.0 |
| H2L7-K12V/R71A/ | 5.76 ± 0.89 e4 | 2.45 ± 1.13 e−5 | 427 ± 190 |
| R83T | | | |

### Example 12

### Binding of H2L7 Variant Antibodies to Target in Brain from Human Alzheimer's Disease Patients and Non-Demented Controls

This example describes target binding of H2L7 and of the H2L7 variants generated in Example 10, as tested by immunoprecipitation on human brain extracts and immunohistochemistry on human brain sections from AD patients and NDE controls.

### Materials and Methods

Target binding in human Alzheimer's disease brain extracts by immunoprecipitation: Antibody binding to target in human AD brain was analyzed by immunoprecipitation as described for H2L7 and H9L8 in Example 7.

Target binding in human Alzheimer's disease brain by immunohistochemistry: Immunohistochemistry (IHC) analyses were performed on brain tissue from AD as described for H2L7 and H9L8 in Example 7.

### Results

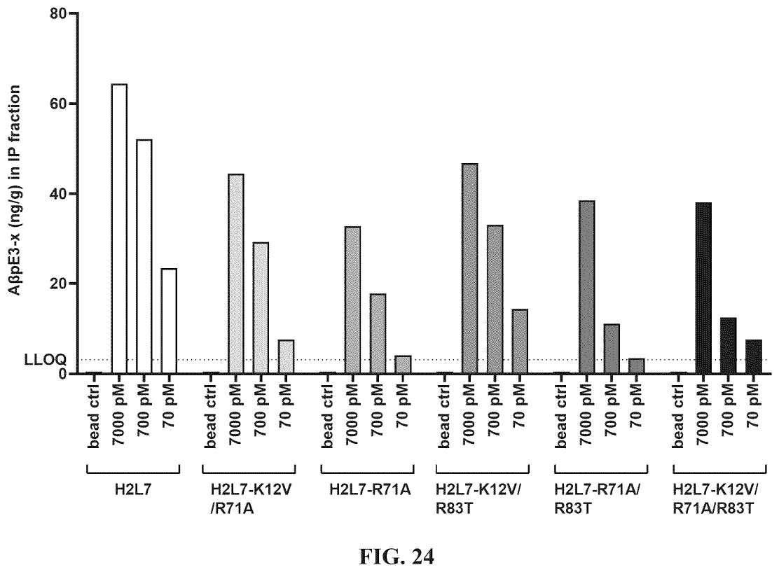

Target binding in human Alzheimer's disease brain extracts by immunoprecipitation: Antibody H2L7 and variants H2L7-K12V/R71A, H2L7-R71A, H2L7-K12V/R83T, H2L7-R71A/R83T and H2L7-K12V/R71A/R83T were tested for their ability to bind to AβpE3 in solution, in human brain extracts from AD patients. Immunoprecipitation (IP) of TBS brain extracts from AD patients demonstrated a concentration dependent IP of AβpE3-x by all tested antibodies (FIG. 24).

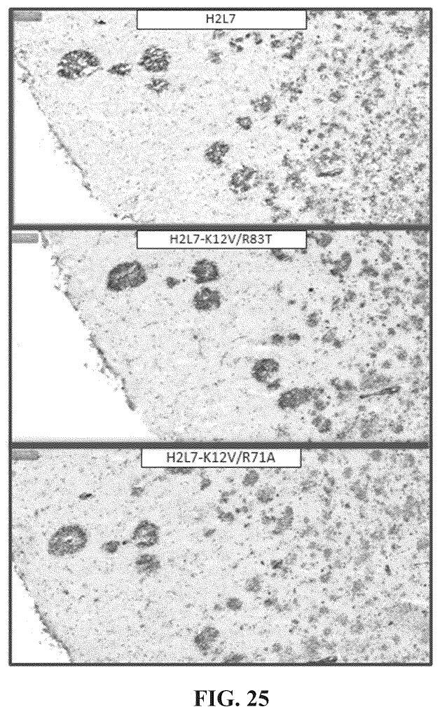

Target binding in human Alzheimer's disease brain by immunohistochemistry: Immunohistochemical staining of brain sections from AD individuals (confirmed to have Aβ pathology by IHC staining with 6E10/4G8, not shown) with H2L7 and variants H2L7-K12V/R71A and H2L7-K12V/R83T resulted in specific binding to core and diffuse plaques in AD brain, with an identical staining pattern. No binding was observed to NDE control brain (data not shown). Representative images from immunostaining with H2L7, H2L7-K12V/R71A, and H2L7-K12V/R83T on adjacent sections from fresh-frozen AD brain are shown in FIG. 25.

### Example 13

### Characterization of Functional Effects of H2L7 Variant Antibodies

This example describes the functional effects of H2L7 variant antibodies generated as described in Example 10. The ability of the variant antibodies to inhibit aggregation of AβpE3 and to clear amyloid plaques in AD brain sections ex vivo was evaluated.

### Materials and Methods

Inhibition of aggregation of AβpE3-42: The effect of the variant antibodies on aggregation of AβpE3-42 monomers was evaluated in an aggregation assay as described for H2L7 and H9L8 in Example 8.

Ex vivo phagocytosis in AD brain: An ex vivo phagocytosis assay was used to investigate whether the variant antibodies can induce plaque clearance by macrophages, as described for H2L7 and H9L8 in Example 8.

### Results

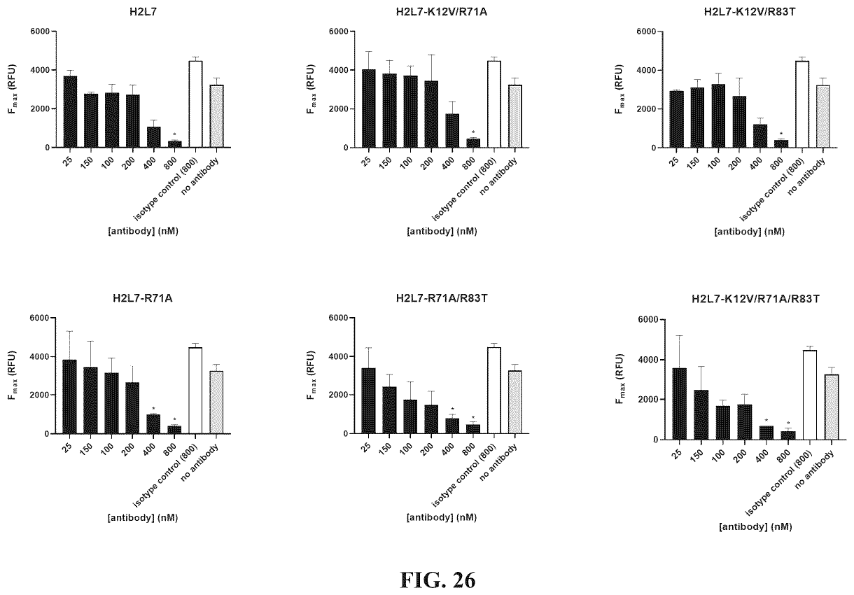

Inhibition of aggregation of AβpE3-42: The ability of the H2L7 variant antibodies to inhibit aggregation of AβpE3-42 was evaluated. All tested antibodies concentration-dependently inhibited fibril formation of AβpE3-42, as demonstrated by the decrease in the maximum ThT fluorescence signal (Fmax) in the presence of the antibodies (FIG. 26).

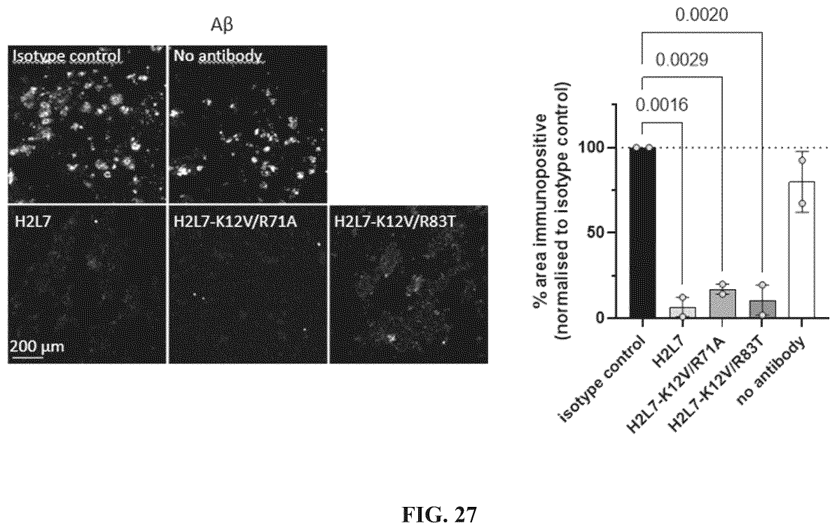

Ex vivo phagocytosis in AD brain: The ability of the H2L7 variant antibodies H2L7-K12V/R71A and H2L7-K12V/R83T to induce clearance of Aβ plaques by macrophages in AD brain was evaluated. Compared to negative control samples pre-incubated without antibodies or with an isotype control IgG1 antibody, Aβ plaques were significantly reduced after pre-incubation with H2L7, H2L7-K12V/R71A and H2L7-K12V/R83T, indicating that the tested antibodies induced plaque clearance by macrophages (FIG. 27).

### Example 14

### Immunogenicity of H2L7 Variant Antibodies

This example describes the assessment of the potential immunogenicity of H2L7 variant antibodies generated as described in Example 10. The ability of the variant antibodies to induce CD4+ T cell responses was evaluated using the Episcreen™ time course assay.

### Materials and Methods

Isolation of peripheral blood mononuclear cells (PBMCs): PBMCs were isolated from healthy community donor buffy coats (from blood drawn within 24 h) obtained under consent from commercial vendors. Cells were separated by density centrifugation using Lymphocyte separation medium (StemCell Technologies Inc, London, UK) and CD8+ T cells were depleted using CD8+ RosetteSep™ (StemCell Technologies Inc). Donors were characterized by identifying HLA-DR and HLA-DQ haplotypes to 4-digit resolution by SSO HLA typing (VHBio, Gateshead, UK). T cell responses to the neo-antigen KLH (Invitrogen, Paisley, UK) were also determined. PBMCs were then frozen and stored in the vapour phase of nitrogen until required.

Preparation of samples: Endotoxin levels in the antibody samples were measured using a LAL chromogenic kinetic assay kit (Charles River, Margate, UK) according to the manufacturer's instructions and found to be within the limit acceptable for the assay (<3 EU/mg).

The antibody samples were diluted to 0.6 μM in AIM-V® culture medium (Invitrogen) prior to use (final assay concentration 0.3 μM). KLH was used as a reproducibility control and stored at −20° C. as a 10 mg/ml stock solution in water. For the studies, an aliquot of KLH was thawed immediately before use and diluted to 200 μg/ml in AIM-V® (final concentration 100 μg/ml). A further high immunogenicity control, CEFT (pool of 13 peptides from Pepscan Ltd, Lelystad, The Netherlands) was used as a high responding control and this was stored at −20° C. as a 1.538 mg/ml stock solution and diluted in AIM-V® to 2 μg/ml before use (final concentration 1 μg/ml). Herceptin® (Bionical Ltd, Willington, UK) was used as a negative clinical control and this was stored at −80° C. as a 20 mg/ml stock solution (final assay concentration 50 μg/ml).

Time course proliferation assay: A cohort of 50 donors was selected for the assay. PBMCs from each donor were thawed, counted and viability assessed using an acridine orange (AO) and 4′,6-diamidino-2-phenylindole (DAPI) (Chemometec Ltd, Allerod, Denmark) dye exclusion. Cells were revived in room temperature AIM-V® culture medium, washed and resuspended in AIM-V® to 4-6×10⁶ PBMC/ml for use as the proliferation cell stock. For each donor, bulk cultures were established in which 1 ml of the proliferation cell stock was added to the appropriate wells of a 24 well plate. 1 ml of each sample was added to the PBMCs to give a final sample concentration of 0.3 μM. For each donor, a reproducibility control well (cells incubated with 100 μg/ml KLH), a further high immunogenicity control (cells incubated with 1 μg/ml CEFT peptide pool), a low immunogenicity control (cells incubated with 50 μg/ml Herceptin®) and a culture medium only well were also included. Cultures were incubated for a total of 8 d at 37° C. with 5% CO₂. On days 5, 6, 7 and 8, the cells in each well were gently resuspended by mixing 5× using an electronic pipette and 3×100 μl aliquots transferred to each well of a round bottomed 96 well plate. The cultures were pulsed with 0.75 μCi [3H]-Thymidine (Perkin Elmer, Beaconsfield, UK) in 100 μl AIM-V® culture medium and incubated for a further 18 h before harvesting onto filter mats (Perkin Elmer) using a TomTec Mach III cell harvester. CPM for each well were determined by Meltilex™ (Perkin Elmer) scintillation counting on a 1450 Microbeta Wallac Trilux Liquid Scintillation Counter (Perkin Elmer) in paralux, low background counting.

Assessment of cell viability: On day 7, bulk cultures (previously established for the proliferation assay) were gently resuspended by mixing 5× using an electronic pipette and 50 μl was removed from each well and mixed with 2.5 μl acridine orange (AO) and 4′,6-diamidino-2-phenylindole (DAPI) (Chemometec Ltd) dye exclusion. Cells were then assessed for viability using a NucleoCounter® NC-250™ automated cell analyser (Chemometec Ltd).

Data analysis: For proliferation assays, an empirical threshold of a stimulation index (SI) equal to or greater than 1.9 (SI≥1.90) had been previously established, whereby samples inducing responses above this threshold are deemed positive. For proliferation analysis, donors that were positive on at least one time point during the time course assay were deemed positive donors and counted towards the “% Response” parameter.

### Results

The ability of the H2L7 variant antibodies H2L7-K12V/R71A and H2L7-K12V/R83T to induce CD4+ T cell responses was evaluated. Reference antibodies A and B were used for comparison. Reference antibody A comprises the heavy chain amino acid sequence SEQ ID NO:33 and the light chain amino acid sequence SEQ ID NO:34. Reference antibody B comprises the heavy chain amino acid sequence SEQ ID NO:35 and the light chain amino acid sequence SEQ ID NO:36.

EpiScreen™ analysis of the frequency and magnitude of the responses showed that H2L7-K12V/R71A and H2L7-K12V/R83T induced responses slightly above the response for the low immunogenicity control Herceptin® and were therefore considered to have a relatively low risk for immunogenicity. Both reference antibodies A and B induced clearly higher proliferation responses compared to Herceptin® and are therefore considered to have a higher risk of immunogenicity in the clinic.

TABLE 18
Summary of healthy donor proliferation

| Antibody | Mean SI | SD | % Response |
|---|---|---|---|
| H2L7-K12V/R71A | 3.21 | 1.86 | 14 |
| H2L7-K12V/R83T | 3.04 | 1.36 | 18* |
| Reference A | 3.77 | 1.96 | 50 |
| Reference B | 3.97 | 2.55 | 32 |
| Herceptin ® | 3.05 | 0.92 | 10 |
| CEFT | 5.92 | 6.62 | 90 |
| KLH | 11.94 | 12.88 | 100 |

*calculated based on 28 donors

### Example 15

### Identification of Binders of Human TfR1 by Immunization and Screening

### Immunization and Hybridoma Screening

To identify monoclonal antibodies that bind human transferrin receptor 1 (hTfR1), four 6-10 weeks old Balb/c or C57BL/6 mice were immunized subcutaneously with immunogen together with adjuvant. The hTfR1 immunogen was designed to contain the ectodomain of the human TfR1 protein, N-terminally fused to a T-cell epitope from tetanus toxin, P2 (Kovacs-Nolan and Mine (2006), Biochim Biophys Acta 1760:1884-1893) via a GSS linker, and an N-terminal 10×histidine tag (His₁₀-P2-hTfR1; SEQ ID NO:107). Following gene construction, recombinant His₁₀-P2-hTfR1 protein was generated by transient transfection in Hek293 cells using the Expi293™ Expression system (Gibco), purified on a nickel column (HisTrap FF, cat. no. 17-5255-01, GE Healthcare), buffer exchanged to PBS and concentrated to 1 mg/ml. Expressed TfR1 immunogen was aliquoted and stored at −80° C. until use. Quil-A adjuvant (vac-quil, InvivoGen) was used for all immunizations except for the final booster injection in which no adjuvant was included. For use, Quil-A was resuspended in ddH₂O at a concentration of 1 mg/ml, sterile filtered and aliquoted in 0.1 ml aliquots stored at −80° C. Quil-A was administered at a dose of 10 μg/mouse.

Animals were immunized every month with the recombinantly produced immunogen, His₁₀-P2-hTfR1, mixed and co-administered with Quil-A. Three weeks after each immunization, blood samples were collected, and the plasma was analyzed for presence of antibodies reactive towards recombinantly produced human TfR1 and mouse TfR1. Titers were considered high enough when the ELISA response at 1/100,000 dilution exceeded the average of the blanks (i.e. background) plus 3 standard deviations of the blanks. The four mice used in this study received between 4 and 6 immunizations each.

Three days before fusion, the final intraperitoneal booster injection was given to the mice in absence of adjuvant. At sacrifice, mice were anesthetized with isoflurane. Intact spleens were collected by opening the abdominal cavity and dissected. Briefly, a single cell suspension of the spleen from an immunized mouse was prepared and mixed with Sp2/0 cells at a 3:1 ratio. The cells were fused using PEG and the cells were added to a bottle of ClonaCell™-HY Medium D (STEMCELL Technologies). 60-70 μl per well was then dispensed into 96-well plates. After 6-7 days, 150 μl HAT-medium was added to each well in the semi solid 96-well plates. The day after, 120 μl of supernatant was discarded from each well and 100 μl fresh HAT-medium was added. The next day, 100 μl of the supernatant of each well was taken and transferred to a storage plate and tested for presence of antibodies against mouse TfR1 using indirect ELISA on nickel-coated plates according to the protocol below. A repeated screen of the hybridoma plates was performed by adding 120 μl HAT-medium on day 12 and by 3 days later transferring 25 μl supernatant to ELISA plates to screen for reactivity against mouse TfR1 (both screens referred to as “primary screen”). Clones that were positive towards mouse TfR1 with OD>0.2 were transferred to 24-well plates, cultured for at least 3 days, and subjected to a secondary screen for reactivity towards murine, human and cynomolgus TfR1 in solution using biolayer interferometry (BLI) (referred to as “secondary screen”). Whereas binding of both hTfR1 and cynomolgus TfR1 was indicated, only very weak or no binding was detected for mTfR1 in the secondary screen. Supernatants from 24-well plates were also screened for binding towards His-tagged hTfR1 as well as lack of binding towards His-tagged amyloid-β precursor protein (APP; negative control) using both direct coated TfR1 plates and nickel-coated plates as described below. Binding towards cynomolgus TfR1 (cTfR1) was also analyzed using direct TfR1 coat. Notably, ELISA responses (OD450 values) were very low for mTfR1 compared to hTfR1 and cTfR1, indicating weaker binding to mTfR1 compared to the binding to hTfR1 and cTfR1 for all positive clones.

Selected clones were diluted using limiting dilution assays (LDA) to reach monoclonality. Reactivity against mouse TfR1 and human TfR1 were re-tested by ELISA on monoclonal cultures following LDA and expansion.

### Indirect ELISA Screening

ELISA assays were performed according to standard ELISA protocols in order to screen plasma samples for reactivity towards the target antigens after immunizations, or to identify hybridoma clones producing antibodies with reactivity against the TfR1 target protein. Briefly, 96-well half area plates (Corning) were coated with 1 μg/ml His₁₀-mTfR1 (SEQ ID NO:108) or His₁₀-hTfR1 (SEQ ID NO:109). His₁₀-mTfR1 and His₁₀-hTfR1 were recombinantly produced and purified using the procedure described above for the His₁₀-P2-hTfR1 immunogen. The plates were blocked with 150 μl/well of protein free blocking solution (Pierce) for 1 h at room temperature with shaking (600-900 rpm). The plates were washed four times with PBS containing 0.1% TWEEN®-20 and Kathon™. Plasma samples serially diluted from a starting dilution of 1/450 or hybridoma supernatants diluted 1/2 were added to the plates (50 μl/well; dilution buffer: PBS with 0.1% BSA and 0.05% TWEEN®-20) and incubated for 2 h at room temperature and then the plates were washed four times. Detection antibody (HRP-conjugated anti-mouse IgG, Southern Biotech, cat. no. 1030-05, diluted 1/5000 in dilution buffer) was added at 50 μl/well, and the plates were incubated for 1 h at room temperature. After another wash (as above), 50 μl/well TMB substrate (K-Blue® Aqueous, Neogen) was added, and the reaction was stopped after 10-15 min with 50 μl/well of 0.5 M H₂SO₄. The optical density at 450 nm was read using a plate reader (Tecan). The endpoint titers were defined as the dilution above the average of the blank wells (background) plus 3 standard deviations of the blank wells.

The primary screen of hybridoma clones producing antibodies with reactivity against the target protein was performed using nickel-coated ELISA plates. Briefly, 96-well Ni-coated plates (PIERCE) supplied pre-blocked with BSA were incubated with 3 μg/ml (100 μl) His₁₀-mTfR1 without shake overnight at 4° C. The plates were washed four times with PBS containing 0.1% TWEEN®-20 and Kathon™. Hybridoma supernatants diluted 1/4 were added to the plates (dilution buffer: PBS with 0.1% BSA and 0.05% TWEEN®-20) and incubated for 2 h at room temperature and then the plates were washed four times. Detection antibody (HRP-conjugated anti-mouse IgG, Southern Biotech, cat. no. 1030-05, diluted 1/5000 in dilution buffer) was added at 100 μl/well, and the plates were incubated for 1 h at room temperature. After another wash (as above), 100 μl/well of K-Blue® Aqueous substrate (Neogen) was added, and the reaction was stopped after 10-15 min with 100 μl/well of 0.5 M H₂SO₄. The optical density at 450 nm was read using an ELISA plate reader (Tecan).

Examples of clones considered to be positive in binding mouse TfR1 and human TfR1 are shown in Table 19. These clones were also confirmed to bind both His-tagged hTfR and cTfR by ELISA, and to lack binding to His-tagged APP (negative control). Selected clones were further characterized in various assays.

TABLE 19
Examples of identified clones from hybridoma screening

| | mTfR1 | | | | |
|---|---|---|---|---|---|
| | specificity | | | | |
| | by indirect | hTfR | cynoTfR | mTfR1- | Isotype |
| | ELISA | binding | binding by | binding | (from |
| Clone | (Ni capture) | by BLI | BLI | by BLI | sequencing) |
| 24B4 | OD > 0.2 | Yes | Yes | No/weak | IgG2a/κ |
| 26D3 | OD > 0.2 | Yes | Yes | No/weak | IgG2a/κ |
| 37D10 | OD > 0.2 | Yes | Yes | No/weak | IgG2a/κ |

### Biolayer Interferometry Measurements

Selected clones were investigated using biolayer interferometry (BLI) on an Octet instrument (Octet Red384, ForteBio). In the setup used, the adopted method involves capture of IgG from the respective clone on the individual sensor tips to allow for detection of antibodies that bind to target in solution. In addition to providing a measure of binding, BLI measurements provide more details about the overall binding properties, because they include estimates of the on-rate and off-rate.

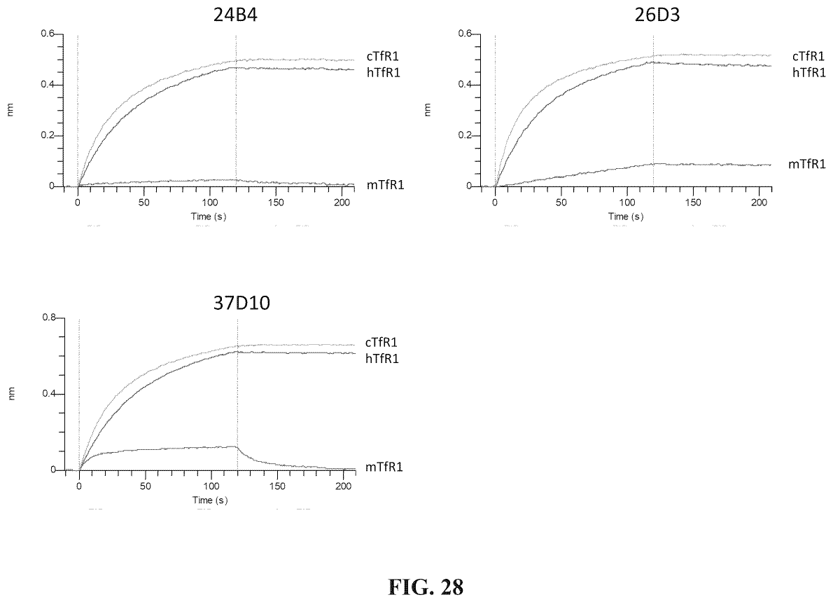

FIG. 28 shows the results of BLI measurements for three selected clones provided as examples, with binding measured directly in the crude hybridoma supernatant. Briefly, mouse IgG antibody clones in hybridoma supernatants, diluted 1:1 in running buffer (PBS, 0.02% TWEEN®-20 and 0.01% BSA), were captured on anti-mouse capture biosensors (anti-mouse capture, AMC, Molecular devices, Cat. 18-5580). Next, sensors with immobilized IgGs were briefly washed for 10 s before incubation in running buffer to establish a baseline signal. Association to target antigens were measured by incubating sensors for 120 s in wells of the assay plate containing the following concentrations of respective target antigen: 500 nM mTfR1, 250 nM hTfR1 and 250 nM cTfR1. All proteins were diluted in running buffer. Target dissociation was measured by incubating the biosensors in running buffer for 90 s. All tested clones, i.e. 24B4, 26D3 and 37D10, bind to both human and cynomolgus TfR1 but very weakly to mouse TfR1. Overall, most clones showed more cross-reactivity towards human and cynomolgus TfR1 than against mouse TfR1.

### Sequencing of Selected Clones

Clones of interest were cryopreserved and sequenced by whole transcriptome shotgun sequencing. Among the sequenced hybridoma clones were clones denoted 26D3, 24B34 and 37D10. The amino acid sequences obtained for the respective heavy chain variable (VH) and light chain variable (VL) domains of these antibodies are given in Table 20 below:

TABLE 20
Variable domain amino acid sequences for selected primary antibodies

| | | |
|---|---|---|
| | | SEQ |
| | Antibody | ID |
| Region | Amino acid sequence | NO: |

26D3

| | | |
|---|---|---|
| VH | EVQLQQFGVELVKPGASVKISCKASGYIFTDYNMDWVKQSHGKSLEWIGDINP | 101 |
| | DYDTTSYNEKFKGKATLTVDKSSSTAYMELRSLTSEDTAVYYCARGGYSGSSY | |
| | YHPMDYWGQGTSVTVSS | |
| VL | DIVMSQSPSSLAVSVGEKITMSCKSSQSLLYSTNQKNYLAWYQQKPGQSPELLI | 102 |
| | YWASTRESGVPDRFTGSGSGTDFTLTISNVRAEDLAVYYCQQYFIYPRTFGGGT | |
| | KLEIK | |

24B4

| | | |
|---|---|---|
| VH | EVQLQQFGAELVKPGTSVKISCKASGYTFTDYNMDWVKQGHGKGLEWIGDINP | 103 |
| | NYDTTSYSQKFKGKATLTVDKSSSTAYMELRSLTSEDTAVYYCARSEAGNYYW | |
| | YFDVWGAGTTVTVSS | |
| VL | DIVMSQSPSSLAVSVGEKVTMSCKSSQSLLYSSNRKNYLAWYROKPGQSPKLLI | 104 |
| | YWASTRESGVPDRFTGSGSGTDFTLTISSVKAEDLAVYSCQQYYNYPYTFGGGT | |
| | KLEIK | |

37D10

| | | |
|---|---|---|
| VH | QVQLQQSGAELVRPGTSVKISCKASGYAFTNYWLGWVKQRPGHGLEWIGDIFP | 105 |
| | GSDNTYYNEKFKGKATLTADKSSSTAYMQLSSLASEDSAVYFCARSGNFYAMD | |
| | YWGQGTSVTVSS | |
| VL | ENVLTQSPAIMSASPGEKVTMTCSASSSVNYMNWYQQKSSTFPKLWIYDTSKL | 106 |
| | ASGVPGRFSGSGSGKFYSLTISSMEAEDVATYYCFQGSGYPFTFGSGTKLEIK | |

The complementarity determining regions (CDRs) of these antibodies were identified using the Kabat definition, and are given in Table 21 below.

TABLE 21
CDR sequences of primary antibodies

| Antibody | | | |
|---|---|---|---|
| | VHCDR1 | VHCDR2 | VHCDR3 |
| 26D3 | DYNMD | DINPDYDTTSYNEKFKG | GGYSGSSYYHPMDY |
| | (SEQ ID NO: 46) | (SEQ ID NO: 47) | (SEQ ID NO: 48) |
| 24B4 | DYNMD | DINPNYDTTSYSQKFKG | SEAGNYYWYFDV |
| | (SEQ ID NO: 46) | (SEQ ID NO: 70) | (SEQ ID NO: 71) |
| 37D10 | NYWLG | DIFPGSDNTYYNEKFKG | SGNFYAMDY |
| | (SEQ ID NO: 74) | (SEQ ID NO: 75) | (SEQ ID NO: 76) |
| | VLCDR1 | VLCDR2 | VLCDR3 |
| 26D3 | KSSQSLLYSTNQKNYLA | WASTRES | QQYFIYPRT |
| | (SEQ ID NO: 49) | (SEQ ID NO: 50) | (SEQ ID NO: 51) |
| 24B4 | KSSQSLLYSSNRKNYLA | WASTRES | QQYYNYPYT |
| | (SEQ ID NO: 72) | (SEQ ID NO: 50) | (SEQ ID NO: 73) |
| 37D10 | SASSSVNYMN | DTSKLAS | FQGSGYPFT |
| | (SEQ ID NO: 77) | (SEQ ID NO: 78) | (SEQ ID NO: 79) |

The VH and VL domains of the identified antibodies are mutated to introduce cysteine residues for the provision of a disulfide bridge between the VH and VL domains. The resulting sequences, variously denoted “disulfide stabilized variants”, “DS stabilized variants”, “DS versions” or similar herein, are given in Table 22.

TABLE 22
Disulfide stabilized variants of primary antibodies

| | | |
|---|---|---|
| | | SEQ |
| | Antibody | ID |
| Region | Amino acid sequence | NO: |

26D3_DS

| | | |
|---|---|---|
| VH | EVQLQQFGVELVKPGASVKISCKASGYIFTDYNMDWVKQSHGKCLEWIGDIN | 138 |
| | PDYDTTSYNEKFKGKATLTVDKSSSTAYMELRSLTSEDTAVYYCARGGYSGSSY | |
| | YHPMDYWGQGTSVTVSS | |
| VL | DIVMSQSPSSLAVSVGEKITMSCKSSQSLLYSTNQKNYLAWYQQKPGQSPELLI | 148 |
| | YWASTRESGVPDRFTGSGSGTDFTLTISNVRAEDLAVYYCQQYFIYPRTFGCGT | |
| | KLEIK | |

24B4_DS

| | | |
|---|---|---|
| VH | EVQLQQFGAELVKPGTSVKISCKASGYTFTDYNMDWVKQGHGKCLEWIGDIN | 139 |
| | PNYDTTSYSQKFKGKATLTVDKSSSTAYMELRSLTSEDTAVYYCARSEAGNYYWY | |
| | FDVWGAGTTVTVSS | |
| VL | DIVMSQSPSSLAVSVGEKVTMSCKSSQSLLYSSNRKNYLAWYROKPGQSPKLLI | 149 |
| | YWASTRESGVPDRFTGSGSGTDFTLTISSVKAEDLAVYSCQQYYNYPYTFGCGT | |
| | KLEIK | |

37D10_DS

| | | |
|---|---|---|
| VH | QVQLQQSGAELVRPGTSVKISCKASGYAFTNYWLGWVKQRPGHCLEWIGDIF | 140 |
| | PGSDNTYYNEKFKGKATLTADKSSSTAYMQLSSLASEDSAVYFCARSGNFYAMD | |
| | YWGQGTSVTVSS | |
| VL | ENVLTQSPAIMSASPGEKVTMTCSASSSVNYMNWYQQKSSTFPKLWIYDTSKL | 150 |
| | ASGVPGRFSGSGSGKFYSLTISSMEAEDVATYYCFQGSGYPFTFGCGTKLEIK | |

### Example 16

### In Vitro Binding to Human and Cynomolgus TfR1 and Epitope Screen

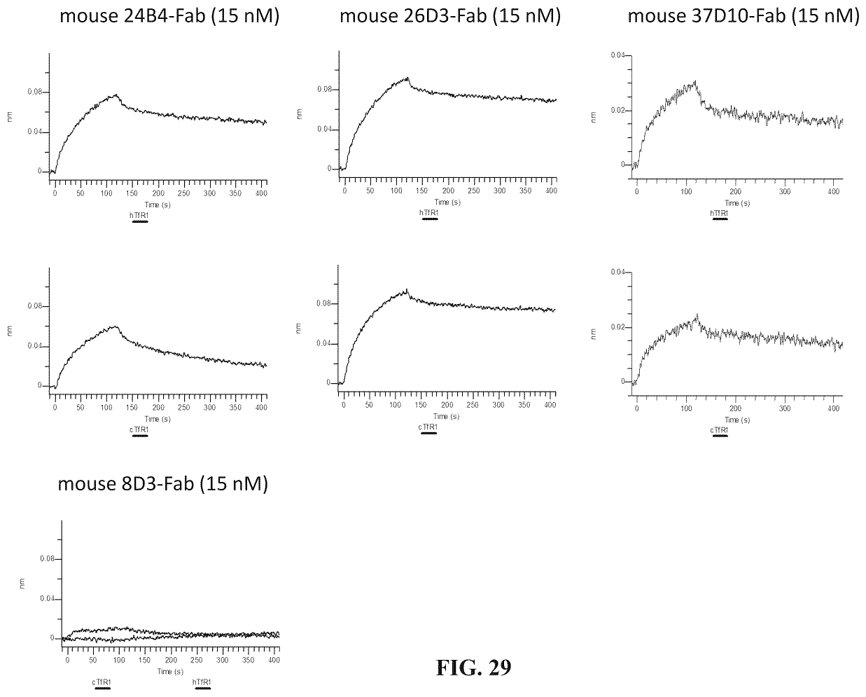

A more detailed binding analysis by BLI was performed on purified, selected antibodies. Binding of Fab fragments from the murine antibodies 26D3, 24B4 and 37D10 to human TfR1 and cynomolgus TfR1 was investigated. For example, the BLI instrument Octet Red384 was used to measure binding between immobilized TfR1 and the tested Fab fragments in solution. Antibody binding to TfR1 was measured with TfR1 complexed to the human transferrin ligand (Tf). Tf/TfR1-complexes were formed on streptavidin biosensors by first loading sensors with biotinylated human holo-transferrin followed by a complex-formation step by capturing either hTfR1 or cTfR1 on the sensors. Final complex density on the sensors was similar for both hTfR1 and cTfR1. Antibody binding to TfR1 was measured during an association phase of 120 s and a dissociation phase of 300 s. FIG. 29 shows sensorgrams for 15 nM of each of 24B4-Fab, 26D3-Fab and 37D10-Fab, as well as for a Fab derived from the known TfR1-binding antibody 8D3 (Boado et al (2009), Biotechnol Bioeng 102:1251-1258). The data indicate a similar binding profile against human and cTfR1 for both 24B4-Fab and 26D3-Fab, and cross-reactive binding to both species is also detected for 37D10-Fab, while no significant binding of 8D3-Fab against human or cynomolgus TfR1 was detected. Importantly, the experiment shows that 24B4-Fab, 26D3-Fab and 37D10-Fab all bind to TfR1 when the natural ligand transferrin is in complex with TfR1.

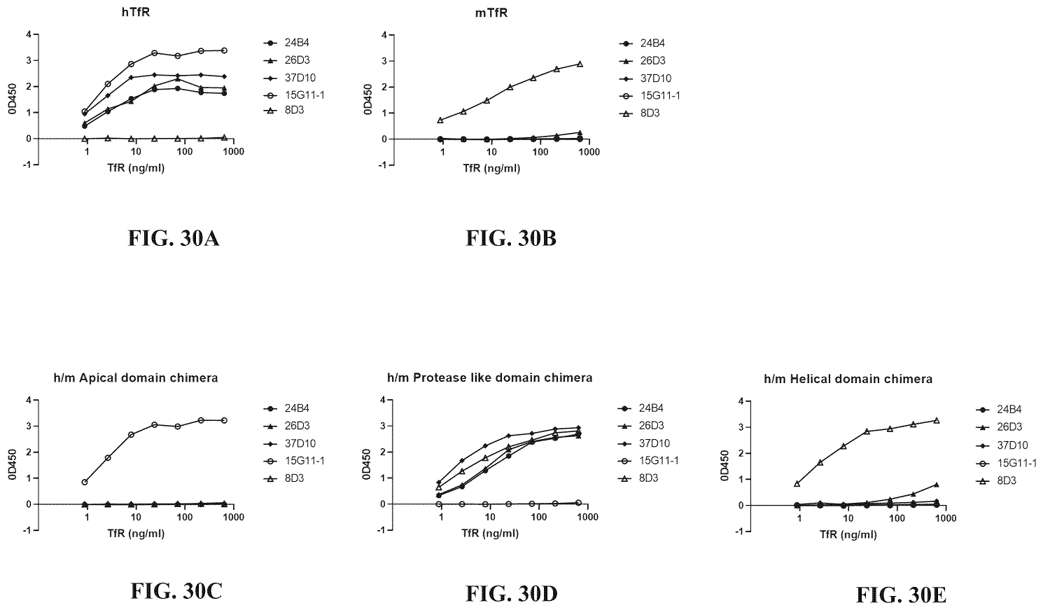

Next, an ELISA experiment showed that antibodies 26D3, 24B4 and 37D10 bind to the protease-like domain of TfR1. In the ELISA experiment, human, mouse or three different chimeric TfR1 receptors were used to coat ELISA plates (FIGS. 30A-30E). The ELISA protocol was slightly modified as follows from the indirect ELISA described in Example 15. Briefly, ELISA plates were coated with the following His-tagged antigens at 1 μg/ml: ectodomain of human TfR1 (His10-hTfR1; SEQ ID NO:110), ectodomain of mouse TfR1 (His10-mTfR1; SEQ ID NO:111), chimeric TfR1 consisting of human apical domain grafted on mouse TfR1 ectodomain (h/m apical domain chimera, mhHD_TFR1; SEQ ID NO:112), chimeric TfR1 consisting of human helical domain grafted on mouse TfR1 ectodomain (h/m helical domain chimera, mhHD_TfR1; SEQ ID NO:113) or chimeric TfR1 consisting of human protease like domain grafted on mouse TfR1 ectodomain (h/m protease-like domain chimera, mhPLD_TfR1; SEQ ID NO:114). The coated plates were then blocked. Dilution series of mouse IgG of the analyzed antibodies were prepared in PBS and incubated on the ELISA plates. Unbound antibodies were then washed off before incubating wells with a HRP-conjugated secondary, anti-mouse-IgG for 1 h. Plates were then washed again before addition of HRP substrate TMB for development and detection of antibody binding to the wells. TMB development was stopped by adding 0.5 M H₂SO₄ to the wells and ELISA responses measured as the OD at 450 nm in an ELISA plate reader. As illustrated, 26D3, 24B4 and 37D10 only bind hTfR1 (FIG. 30A) and not mTfR1 (FIG. 30B). There is no binding of 26D3, 24B4 or 37D10 to the construct with the human apical domain grafted onto the rest of the mTfR1 ectodomain (FIG. 30C). The control antibody 15G11-1 (Yu et al (2014), Sci Transl Med 6:261ra154) known to bind to the human apical domain shows binding to the h/m apical domain chimera as expected (FIG. 30C). In addition, 26D3, 24B4 and 37D10 bind to the h/m protease-like domain chimera (FIG. 30D), but not to any of the plates coated with the other chimeric receptors (FIG. 30C and FIG. 30E). Further, the control antibody 8D3, with an epitope in the apical domain of mTfR1, binds to all plates coated with TfR1 antigens including this domain, namely mTfR1 (FIG. 30B), h/m protease-like domain chimera (FIG. 30D) and h/m helical domain chimera (FIG. 30E). In summary, the experiment demonstrates that the epitope or epitopes for 26D3, 24B4 and 37D10 lie(s) predominantly within the protease-like domain of hTfR1, and that this is in contrast to the control antibodies 15G11-1 and 8D3.

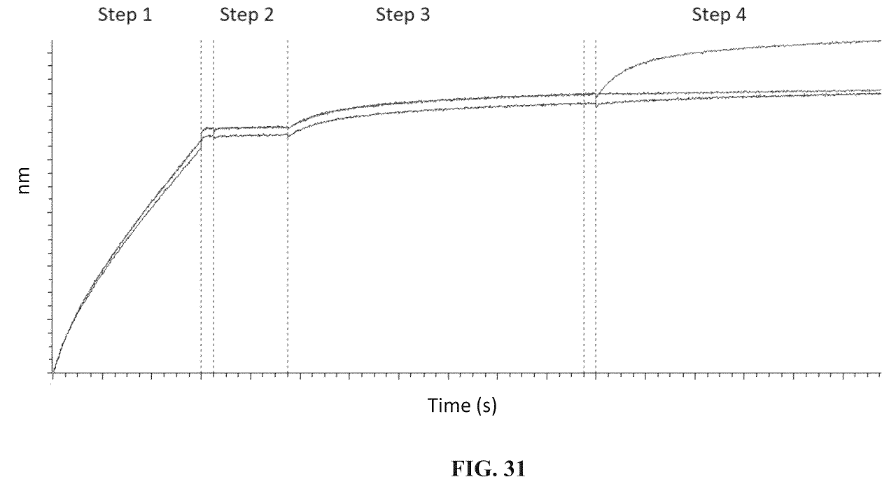

In a further BLI experiment carried out for the purpose of epitope binning (binding competition), it was then shown that binding by both 26D3 and 24B4 is targeted to the same or overlapping regions of hTfR1, with an epitope located outside the apical domain (FIG. 31). The epitope binning experiment by BLI was conducted on an Octet Red384 instrument (ForteBio) by first (Step 1) immobilizing biotinylated hTfR1 to streptavidin biosensors (High precision biosensors, ForteBio). Next (Step 2), a washing step was carried out. Then (Step 3), hTfR1 loaded sensors were incubated in either buffer (non-competitive reference) or 200 nM of the respective antibody (Ab) to form hTfR1:Ab complexes on the sensors. Finally (Step 4), sensors with free hTfR1 (reference) or respective preformed hTfR1:Ab complex was incubated in 200 nM of respective antibody to measure binding to hTfR1 in complex with the competing antibody. FIG. 31 shows representative BLI sensorgrams obtained during the indicated main assay steps. The signal in Step 4 is indicative of the degree of competition between the two analyzed antibodies. If the antibodies compete for binding to the same or overlapping epitope, there is no increase in the signal of the sensorgram in Step 4. Conversely, if the two tested antibodies bind to distinct and different epitopes, there will be an increased signal from Step 4.

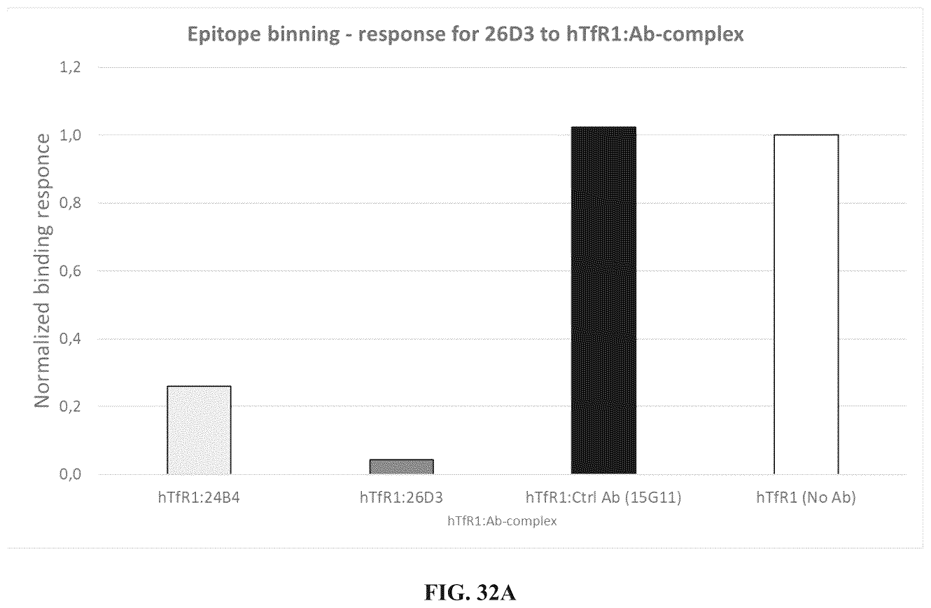

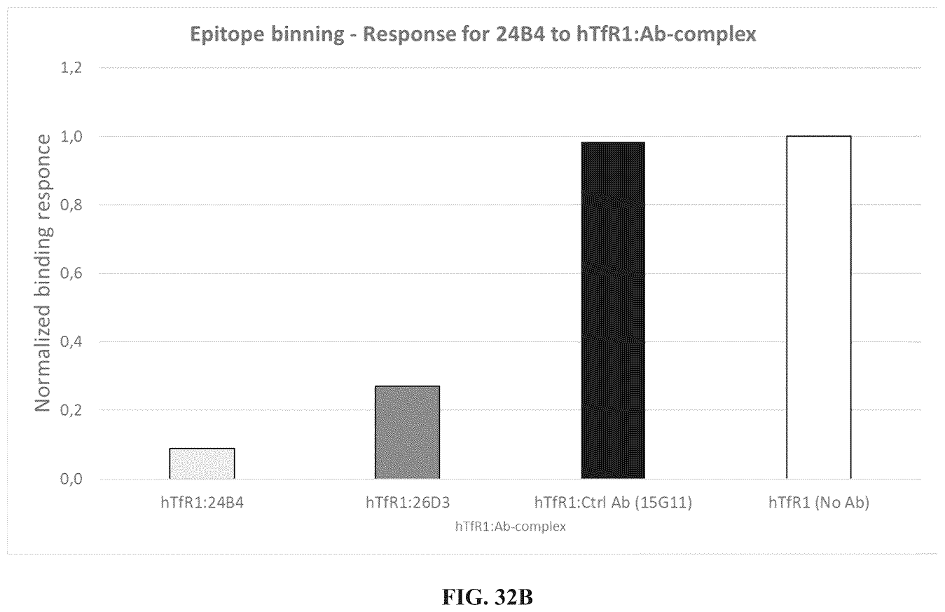

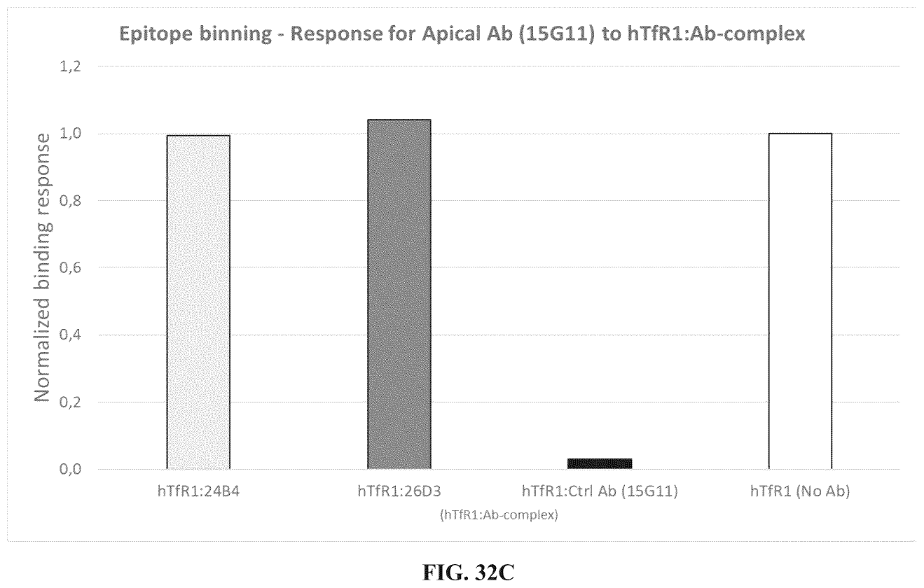

The results of competitive screening of antibody binding to epitopes on hTfR1 by epitope binning as described above is illustrated in FIGS. 32A-32C. Antibodies 26D3 (dark grey bars) and 24B4 (light grey bars) were shown to bind to an overlapping epitope, which is distinct from the hTfR1 apical domain epitope of control antibody 15G11-1 (black bars). FIG. 32A shows that the binding response for 26D3 is reduced by over 70% when hTfR1 is in complex with 24B4. As expected, binding of 26D3 to pre-formed hTfR1:26D3-complex is nearly fully inhibited, illustrating that it blocks itself. Similarly, FIG. 32B shows that the binding response for 24B4 is 70% lower when hTfR1 is in complex with 26D3 and nearly fully inhibited by itself. Both 24B4 and 26D3 retain the full binding response to hTfR1 when hTfR1 is in complex with the control antibody 15G11-1, which has its binding epitope within the apical domain of hTfR1 (FIGS. 32A-32B, black bars). As shown in FIG. 32C, the control antibody 15G11-1 has similar binding responses to the apical domain of hTfR1, regardless of whether it is tested against hTfR1 without competition antibody or when the receptor is in complex with 24B4 or 26D3. In FIGS. 32A-32C, all responses were normalized to the respective antibody's maximal binding response to free hTfR1.

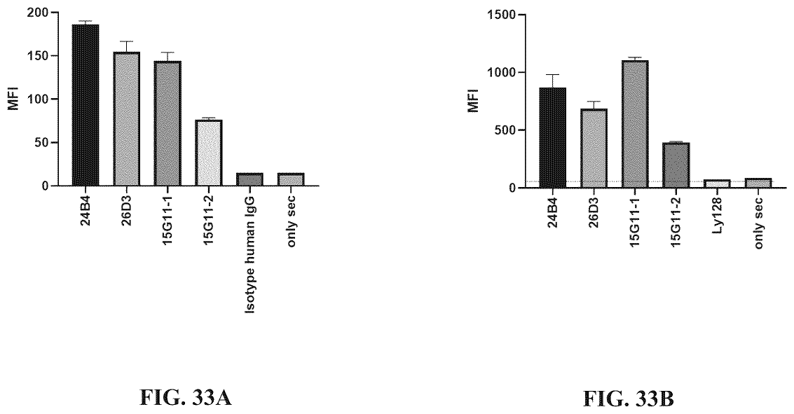

Furthermore, antibody binding to endogenous hTfR1 on brain endothelial cells was studied. Binding to endogenous hTfR1 on cell surfaces was monitored using flow cytometry and human hCMEC/D3 cells (Weksler et al (2013), Fluids Barriers CNS 10:16), which are known to express significant levels of hTfR1 on their surface. Cells that stained positively were plotted and the mean fluorescence intensity (MFI) is shown in FIG. 33A-33B. Both FIG. 33A (IgG1 antibodies) and FIG. 33B (Fab fragments) show that cells were positively stained for hTfR1 with 24B4 and 26D3 to a similar degree (MFI) compared to the positive control antibody 15G11-1 having a high hTfR1 affinity and to a higher degree than the low affinity control antibody 15G11-2 (Yu et al (2014), supra). No background staining was detected with the negative isotype control (FIG. 33A) or the non-related Fab fragment Ly128 (FIG. 33B). These data illustrate that both 24B4 and 26D3 bind to hTfR1 expressed on a cell surface.

### Example 17

### Competition for hTfR1-Binding with Ferritin and Transferrin

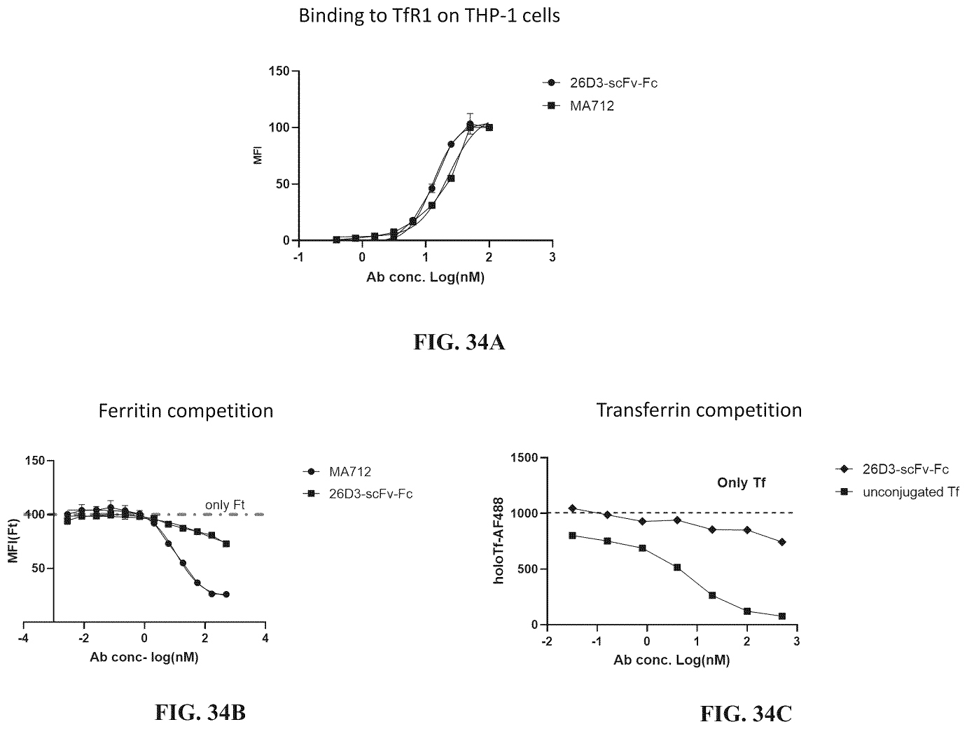

The unique binding to hTfR1 of the binders according to the disclosure, binding to the protease-like domain of hTfR1 and identified as described in Example 15, was evaluated for competition with natural TfR1 ligands ferritin (Ft) and transferrin (Tf). In order to test ferritin competition with antibody, the human monocytic cell line THP-1 (Sigma/ECACC) was used. Binding of the scFv-Fc format (see Example 18 below) and control antibody (M-A712) to hTfR1 on the THP-1 cell surface was confirmed, as shown in FIG. 34A. For evaluating the competition between ferritin and the disclosed binders, cells were incubated with serially diluted test binders along with ferritin from human liver (BioRad, 4420-4804) for 1 h at 4° C. After incubation, ferritin that had bound to hTfR1 on the cell surface was captured using a primary sheep antibody against human liver ferritin (BioRad, AHP2179G) and analyzed using flow cytometry. The results are displayed in FIG. 34B, and show that the 26D3 scFv-Fc does not compete with ferritin on the cell surface, whereas the control antibody anti-CD71, clone M-A712, known to bind to the same epitope on hTfR1 as Ft (Maier et al (2016), Mol Ther Nucleic Acids 5:e321) clearly competes with Ft binding. Also for the identified 26D3 hTfR1 binder, the impact on Ft binding is much less, illustrating that 26D3 has a different epitope on hTfR1 than the binding site for Ft (FIG. 34B).

For transferrin competition, K562 lymphoblast cells (Sigma/ECACC) were used. Cells were incubated with serially diluted test constructs along with Alexa Fluor 488 conjugated, human holo-transferrin (Thermo Fisher; T13342) and incubated for 1 h at 4° C. Transferrin bound to hTfR1 on cell surfaces was captured using flow cytometry, and the mean fluorescence intensity was plotted. FIG. 34C shows that there is no competition between the 26D3 binder and transferrin. When non-labeled (unconjugated) Tf was used as positive control for competition, the binding of labeled (AF488) Tf signal was reduced in a concentration dependent way. The experiment illustrates that a binder directed against the protease-like domain of TfR1 does not compete directly for the same epitope as transferrin.

Overall, this example shows that binding of 26D3 to hTfR1 does not negatively affect the ability of the two endogenous ligands ferritin and transferrin to bind to the receptor.

### Example 18

### Humanization of hTfR1 Binder 26D3

The Fab sequence of mouse antibody 26D3, identified and characterized as described in Examples 15-17, was analyzed and an in silico model of the 26D3 Fab 3D structure was generated using Bioluminate Software (Schrödinger). This murine Fab model was used as input for humanization. In this process, the CDRs of the VH and VL domains of 26D3 (see Table 21; SEQ ID NO:46-51) were grafted in silico into various human variable domains and some residues were back mutated to murine framework at some positions. Three variants having the fewest back mutations and otherwise desirable characteristics were generated and extracted from the software. One such humanized variant was selected for expression and denoted h26D3. h26D3 has the VH domain sequence defined in SEQ ID NO:80 and the VL domain sequence defined in SEQ ID NO:94. A DS version of h26D3 has the VH and VL amino acid sequences SEQ ID NO:124 and 141, respectively.

The humanized version h26D3 and the murine original sequence 26D3 were both expressed as His-tagged Fabs by transient transfection of Chinese Hamster Ovary cells (ExpiCHO; Thermo Fisher Scientific) according to the manufacturer's instructions. The harvested supernatant was purified using HiTrap IMAC Sepharose FF (Cytiva) followed by a size exclusion chromatography on HiLoad Superdex 200pg 26/600 (Cytiva). The following buffers were used: Ni-NTA wash buffer: 20 mM Tris pH 8.0, 10 mM imidazole and 200 mM NaCl; Ni-NTA elution buffer: 20 mM Tris pH 8.0, 200 mM NaCl and 500 mM imidazole; size-exclusion buffer (SEC): 1×dPBS (Thermo Fisher).

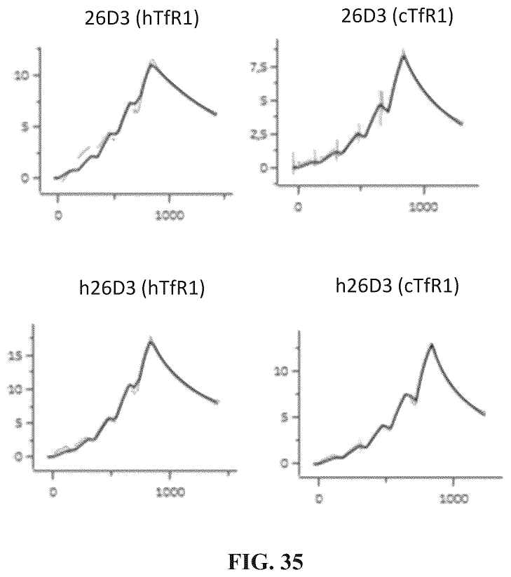

Binding of the purified Fabs to human and cynomolgus TfR1 was evaluated using surface plasmon resonance (SPR) on a Biacore 8K instrument (Cytiva) and the results are shown in FIG. 35. 1 μg/ml of human TfR1 (truncated hTfR1 of SEQ ID NO:122) or cynomolgus TfR1 (truncated cTfR1 of SEQ ID NO:123) was immobilized on a Cm5 sensor chip (Cytiva, #BR100399) using the amine coupling kit type 2 (Cytiva, #BR100633) according to the manufacturer's instruction. The h26D3 and 26D3 Fabs were injected over the chip using a 2-fold dilution series in five steps starting at 25 nM. The interaction was measured using the single cycle kinetics method with a contact time of 120 s at a flow rate of 30 μl/ml followed by a dissociation time of 600 s. Regeneration of the surface between cycles was done by injecting 3M MgCl₂. The binding data were fitted to a 1:1 interaction model. The Fabs were diluted in HBS-EP+ (Cytiva, #BR100669). Experiments were performed at 25° C. The data confirm that the humanized variant of 26D3, i.e. h26D3, retained binding capacity for human and cynomolgus TfR1 (FIG. 35). The kinetic parameters obtained in the experiment are given in Table 23 below.

TABLE 23
SPR analysis of murine and humanized
26D3 Fabs vs. hTfR1 and cTfR1

| Tested binder | Target | kₐ (1/Ms) | k_{d} (1/s) | K_{D} (M) |
|---|---|---|---|---|
| 26D3 | hTfr1 | 2.8 × 10⁶ | 1.0 × 10⁻³ | 3.6 × 10⁻⁹ |
| 26D3 | cTfR1 | 5.7 × 10⁶ | 6.2 × 10⁻³ | 1.0 × 10⁻⁸ |
| h26D3 | hTfr1 | 1.2 × 10⁶ | 4.5 × 10⁻³ | 3.2 × 10⁻⁹ |
| h26D3 | cTfR1 | 2.7 × 10⁶ | 1.5 × 10⁻³ | 5.8 × 10⁻⁹ |

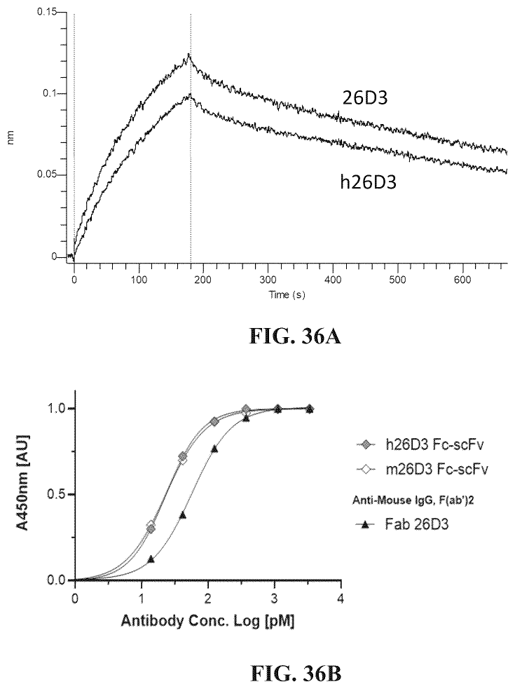

Both murine 26D3 and the humanized variant h26D3 were converted to the scFv format and confirmed to have maintained target binding as scFv (FIGS. 36A-36B). Murine and humanized 26D3 were reformatted to scFv (SEQ ID NO:115 and SEQ ID NO:116 respectively) and produced as monovalent Fc-fused scFv antibody fragments by employing the knob-into-hole (KiH) technology. In this format, one scFv fragment is fused only to the knob half of the Fc (SEQ ID NO:117), while the hole half of Fc (SEQ ID NO:118) is left unfused. The resulting antibody format is a one-armed scFv-Fc. The 26D3 scFv fused to the knob half of the Fc has the complete amino acid sequence SEQ ID NO:119, whereas the h26D3 scFv fused to the knob half of the Fc has the complete amino acid sequence SEQ ID NO:120. The binding profiles for murine and humanized 26D3 in this scFv format are similar and confirm binding activity in the scFv format. Binding responses agree with those of the antibody in Fab format. This was confirmed by several methods, including a kinetic experiment using BLI (results shown in FIG. 36A) and an ELISA (results shown in FIG. 36B). Binding kinetics for murine and humanized 26D3-scFv-Fc were measured by BLI by first immobilizing biotinylated hTfR1 to streptavidin biosensors (Fortebio). Sensors were then washed in buffer (Kinetics buffer, Fortebio) before measuring association of 26D3-scFv-Fc (murine) and h26D3-scFv-Fc (humanized) at 25 nM concentrations followed by a 500 s dissociation phase. In the ELISA experiment, hTfR1 was used to coat the plates for standard binding ELISA experiments using the protocol for indirect ELISA described in Example 15.

### Example 19

### Crystallization and Structure Determination of h26D3-Fab in Complex with hTfR1

This example describes crystallization of a complex between h26D3-Fab and hTfR1 and determination of the binding interface. Ectodomain of human TfR1 (SEQ ID NO:110) was expressed by transient transfection of human embryonic kidney cells (Expi297; Thermo Fisher Scientific) according to the manufacturer's instructions. The harvested supernatant was purified using HiTrap IMAC Sepharose FF (Cytiva) followed by size exclusion chromatography on HiLoad Superdex 200pg 26/600 (Cytiva). The buffers used and purification of the humanized Fab were as described in Example 18.

The formation of a complex between humanized h26D3-Fab and hTfR1 was done by mixing of the two components at a molar ratio of 1:1 in 1×dPBS and incubation at room temperature for 1 h. Subsequently, the complex was purified using size exclusion chromatography on HiLoad Superdex 200pg 26/600 (Cytiva) as described in Example 18.

Crystallization was performed using a stock solution of hTfR1-h26D3 at 15 mg/ml in PBS which was diluted to 4 mg/ml in PBS supplemented with 4 mM β-mercaptoethanol. A 100+100 nl drop was set up using the additive screen in reservoir: 0.1 M sodium potassium phosphate pH 6.5, 10% PEG 3000, 0.05% dichloromethane and 2 mM β-mercaptoethanol. The crystal was flash-frozen in reservoir solution supplemented by 8% glycerol and 16% PEG 400.

X-ray data collection and refinement were performed as follows. Data was collected to 3.87 Å at Diamond Light Source beamline 104. The beamline was equipped with a DECTRIS Eiger2 XE 16M detector. The data set was integrated using XDS (Kabsch (2010), Acta Crystallogr D Biol Crystallogr 66:125-132) with STARANISO anisotropic scaling (Tickle et al (2018), Global Phasing Ltd) and diffracted to 3.87 Å along the c* direction of the reciprocal lattice, and to 4.82 Å in the a*/b* plane. Three complexes were found in the asymmetric unit. The structure was refined using the Buster refinement software and model building was carried out in Coot. Data collection and refinement parameters and statistics are given in Table 24 below.

TABLE 24
X-ray diffraction data coIlection and refinement statistics

| Resolution (Å) | 110.81-3.87 | (4.27-3.87) |
|---|---|---|
| Wavelength (Å) | 0.97950 | |
| Space group | P31 2 1 | |
| Unit cell (Å) | a = b = 127.95, c = 472.91 | |
| Spherical completeness (%) | 63.9 | (12.7) |
| Ellipsoidal completeness (%) | 93.8 | (74.0) |
| No. of observations / unique reflections | 546922 / 27520 | (27874 / 1378) |
| Redundancy | 19.9 | (20.2) |
| <I/σ(I) > | 9.8 | (1.7) |
| CC(1/2) (%) | 99.9 | (81.5) |
| Rmerge (I) (%) | 20.6 | (200.9) |
| Rpim (I) (%) | 6.6 | (62.8) |
| Rmodel (F) (%) | 26.0 | (31.6) |
| Rfree (F) (%) | 31.4 | (34.3) |

| No. of non-hydrogen atoms | 24858 |
|---|---|
| No. of water molecules | 0 |
| rms deviations from ideal geometry | |
| Bond lengths (Å) | 0.009 |
| Bond angles (°) | 1.1 |
| Mean B-factor protein chain | 76.9 / 110.0 / 103.0 / 132.2 / |
| A/B/C/D/E/F/G/H/L (Å²) | 134.4 / 87.2 / 101.0 / 79.9 / 83.4 |
| Mean B-factor glycosylations (Å²) | 70.7 |
| Ramachandran plot quality | |
| Favored regions (%) | 93.4 |
| Allowed regions (%) | 4.3 |
| Outliers (%) | 2.3 |

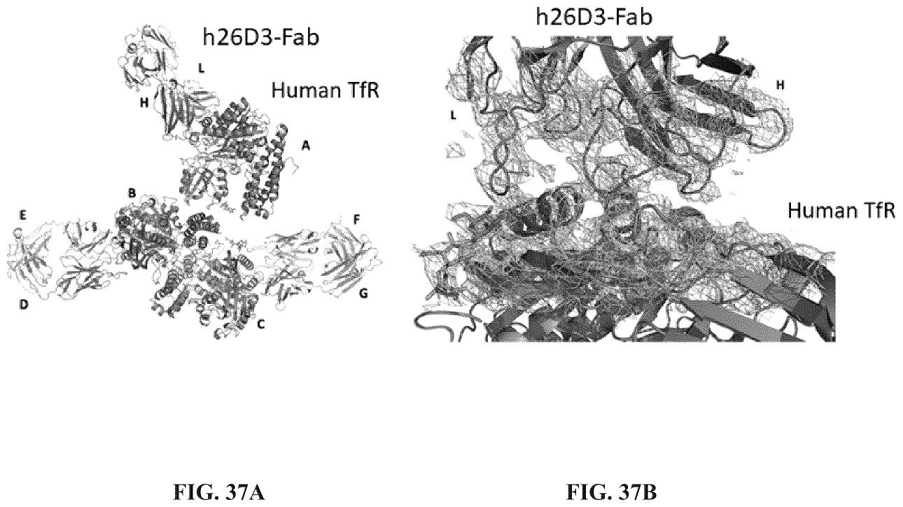

The final, refined structure of the complexes showing the overall folds is depicted in FIGS. 37A-37B. As shown in FIG. 37A, there were three independent complexes in the asymmetric unit. The chain names as used in the coordinate files are indicated. FIG. 37B shows an example of the electron density contoured at the interface between hTfR1 and heavy/light chain of h26D3-Fab. The protein chains are drawn in cartoon representation while sugar moieties are shown in stick representation. The binding interface interaction between h26D3 and human TfR1 was extracted from the X-ray structure and described in the following to provide information about the precise binding of h26D3 to human TfR1.

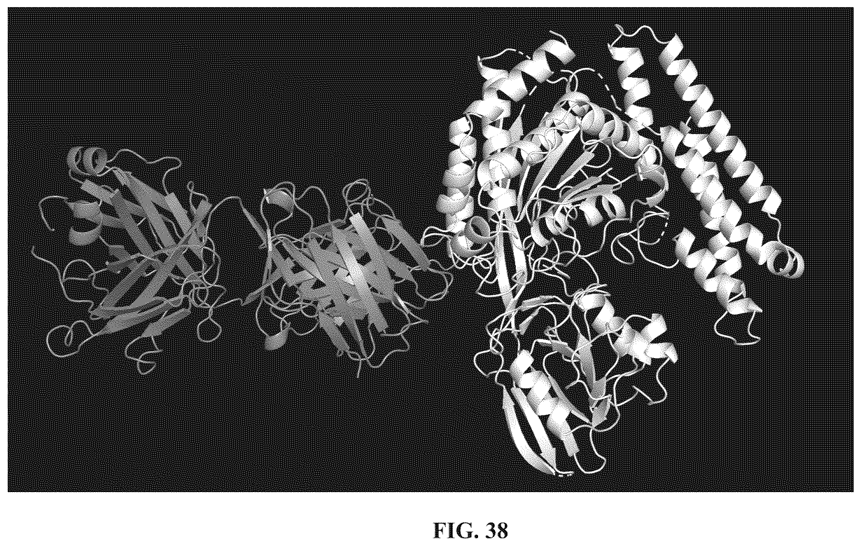

The binding interface between hTfR1 and h26D3-Fab is depicted in FIGS. 37A-37B and FIG. 38, and interaction was observed between the amino acid residues indicated in Table 25.

TABLE 25
Amino acid residues involved in interaction
between h26D3 and hTfR1

| | | Human TfR1 |
|---|---|---|
| h26D3 heavy chain | | |
| 50ASP | interacts with | 150Asn |
| 57Thr | interacts with | 150Asn |
| 59Ser | interacts with | 150Asn |
| 105Ser | interacts with | 154Pro |
| 105Ser | interacts with | 159Ser |
| 105Ser | interacts with | 161Lys |
| 106Tyr | interacts with | 161Lys |
| 106Tyr | interacts with | 158Gly |
| 107Tyr | interacts with | 151Ser |
| h26D3 light chain | | |
| 32Ser | interacts with | 163Glu |
| 32Ser | interacts with | 385Lys |
| 33Thr | interacts with | 160Gln |

Table 25 describes the key residues from both sides involved in the epitope/paratope interface as determined from the crystal structure. Additional residues in the vicinity are also likely to be important for the binding between h26D3 and hTfR1. In addition, as described in Example 23 below, several positions outside the observed binding interaction show important participation in binding of h26D3 to hTfR1.

In Table 26 below, the amino acids of human TfR1 that are involved in the respective interactions with h26D3, Ft and Tf are listed. Notably, no amino acids involved in the binding of h26D3 form part of any of the binding interfaces for the endogenous ligands. This illustrates that the binders of the present disclosure, as exemplified by h26D3, bind to hTfR1 outside the binding sites used by Ft and Tf.

TABLE 26
Amino acid residues in hTfR1 which
interact with the respective ligand

| h26D3 | Ferritin* | Transferrin# |
|---|---|---|
| 150Asn | 195Ser | 121Arg |
| 151Ser | 197Gln | 123Tyr |
| 154Pro | 199Ser | 125Asp |
| 158Gly | 201Ile | 126Asp |
| 159Ser | 208Arg | 622Val |
| 161Lys | 209Leu | 623Arg |
| 163Glu | 210Val | 626Asn |
| 385Lys | 212Leu | 629Arg |
| | 215Asn | 640Gln |
| | 343Glu | 643Tyr |
| | 343Lys | 651Arg |
| | 344Lys | 661Gly |
| | 348Asn | 662Asn |
| | 374Lys | 663Ala |
| | | 664Glu |
| | | 667Asp |
| | | 757Asp |
| | | 758Asn |

*Montemiglio et al (2019), Nat Commun 10: 1121
#Eckenroth et al (2011), Proc Natl Acad Sci USA 108: 13089

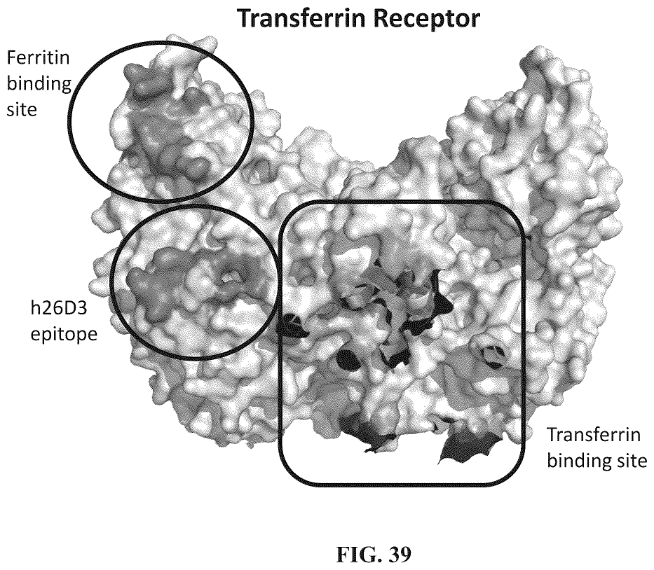

The different epitopes on the hTfR1 structure (pdb: 1SUV) are illustrated further in FIG. 39. As shown in FIG. 39, the Ft binding site is located on the apical domain of hTfR1, the Tf binding site is mainly located on the helical domain of hTfR1 and the h26D3 epitope is located on the protease-like domain of hTfR1. The structure illustrates that the different ligands and binder use distinct, specific surface areas on the hTfR1 structure. hTfR1 is a homodimer having two identical chains, and the epitopes are only indicated on one of these chains.

### Example 20

### Generation and Characterization of hTFR1 Knock-In Mice

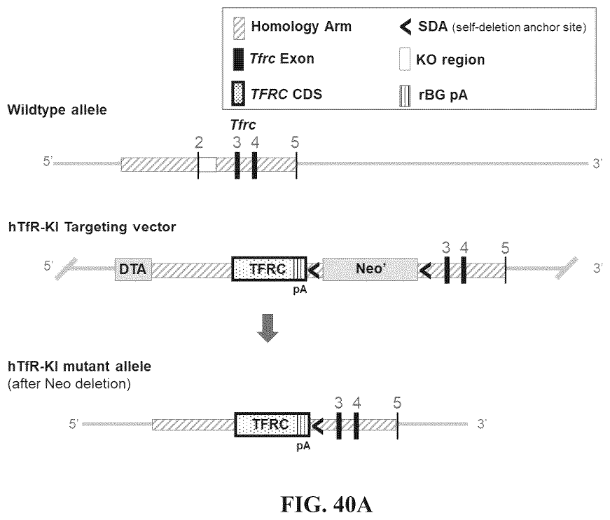

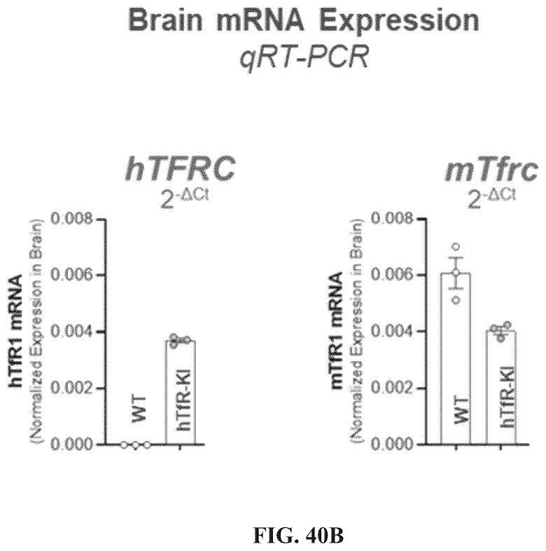

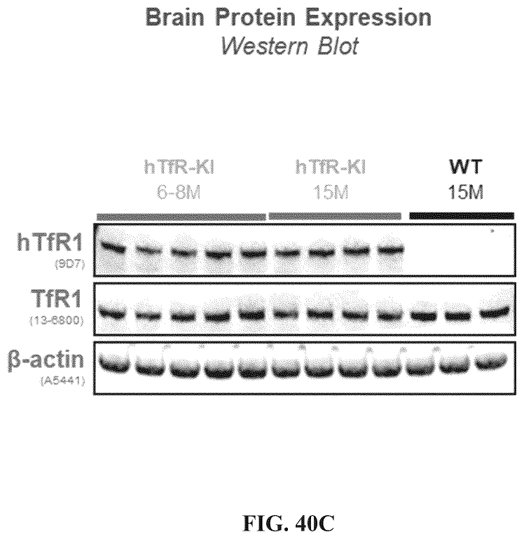

Human TfR1 knock-in (hTfR1-KI; TFR1C-KI) mice were generated by homologous recombination (experimental work performed at Cyagen US). A cDNA vector carrying the TFR1C (NCBI Reference Sequence: NM_001128148.3) ectodomain and murine Tfrc transmembrane and intracellular domain were introduced by pronuclear microinjection in C57BL/6N ES cells Tfrc. The coding region of Tfrc exon 2 plus partial intron 2 were replaced with the TFR1C chimeric cassette (FIG. 40A). Correct insertion of hTfR1 cDNA was verified by Southern blot and PCR. Transgene expression in hTfR1-KI mice was confirmed in brain tissue by qRT-PCR (FIG. 40B) and western blot (FIG. 40C), indicating endogenous expression levels. hTfR1-KI mice were maintained on a C57BL/6N background and only heterozygous hTfR1-KI mice were used for experiments.

### Example 21

### Brain Uptake of hTfR1-Binding Constructs In Vivo

To evaluate hTfR1-mediated brain uptake in vivo, monovalent Fc-scFv constructs (see Example 18) were produced for four different binding proteins. A known binder to hTfR1, 15G11-1, was used as a control (Yu et al (2014), supra). This hTfR1 binder has been described to be active in vivo and is used as a positive reference control for brain uptake. In addition, a construct containing a non-hTfR1 scFv binder based on the anti-amyloid R antibody mAb158 was designed and included as a negative control in the form of an Fc fusion construct (Fc-scFv158, also referred to as simply “158” here and in the figures). The different Fc-scFv constructs were injected intravenously (i.v.) into hTfR1 knock-in (hTfR1-KI) mice produced as described in Example 20 (n=4 per construct) at equimolar doses of 30 nmol/kg (corresponding to approximately 2.3 mg/kg). Plasma and brain exposure was assessed 24 h after dose.

The animals were anaesthetized using isoflurane and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with a minimum of 50 ml of cold PBS. Following perfusion, brains were extracted and the olfactory bulbs removed. The brains were separated into left and right hemispheres and cerebellum was removed from the left hemisphere, after which the left hemisphere was weighed and snap frozen on dry ice and stored at −80° C. until further preparation and analysis of the concentrations of injected constructs using a Meso Scale Discovery (MSD) based assay. The right hemispheres were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing (see Example 22 below).

For brain concentration measurements, frozen left hemispheres were thawed on ice and homogenized in TBS by automated bead homogenization. Triton was added to the homogenate to a final Triton concentration of 0.5% before centrifugation at 16 000×g, after which supernatants were collected.

Brain and plasma concentrations of anti-hTfR1 Fc-scFv were determined using a custom build MSD assay detecting the human Fc. A standard 96-well MSD plate (MSD, #L15XA-3) was coated with 0.5 μg/ml goat anti-human IgG, Fcγ fragment specific antibody (Jackson Immuno Research Europe Ltd, #109-005-098) diluted in 1×PBS (Medicago AB, #09-9400-100). After incubation at 4° C. overnight, the plate was washed 4× in 1×PBS-TWEEN (Fisher Scientific, #09-9410-100) and blocked with 150 μl 1% BlockerA in PBS-TWEEN (MSD, #R93BA-4) per well. Samples and corresponding standards, ranging from 400 μM to 0.1 μM in 1:4 dilution steps, were added and incubated for 2 h and 900 rpm at room temperature. A 1 h incubation step with mouse anti-human IgG (Mabtech, 3850-1-1000, MT145) diluted to 0.5 μg/ml was included, followed by 1 h incubation of SULFO-TAG conjugated anti-mouse antibody (MSD, R32AC-1) diluted to 0.5 μg/ml when the plate was incubated for another hour at room temperature and 900 rpm. 150 μl MSD read buffer (MSD, #R92TC) per well was added before reading the plates in an MSD SECTOR Imager. Between each incubation step, a 4× wash in 1×PBS-TWEEN was performed. All antibodies and samples, except the coating antibody, were diluted in 1% Blocker A in PBS-TWEEN and added in a volume of 50 μl/well. The concentration of the analytes in the samples were evaluated with the MSD workbench software, using a 4 PL curve fitting algorithm and curve weighting 1/Y2 for the standard curve. Statistical analysis was performed in GraphPad Prism (v. 9.0.0) using one way ANOVA with Tukey's post hoc test.

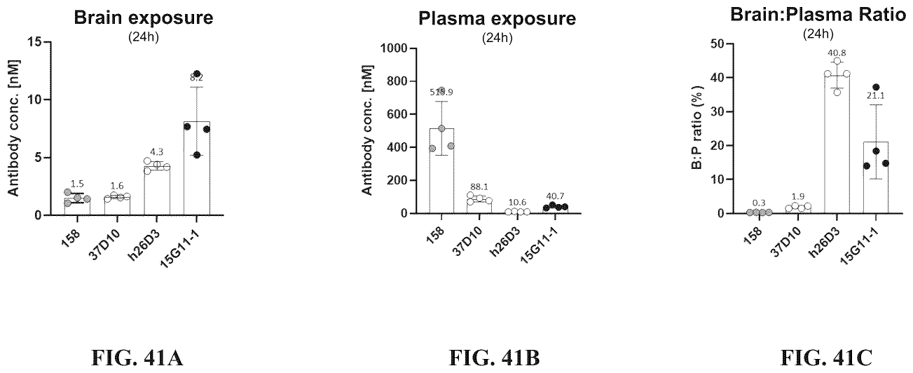

The results are shown in FIGS. 41A-41C. As shown in FIG. 41A, substantially higher brain concentrations were observed for the two test constructs and the positive control 15G11-1, compared to the negative control (158) at 24 h after dose. As shown in FIG. 41B, the plasma concentrations of the two test constructs and the positive control 15G11-1 were lower at 24 h compared to that of 158, indicating that hTfR1 engagement leads to a faster plasma clearance. The brain-to-plasma concentration ratios are shown in FIG. 41C. The two test constructs and the positive control 15G11-1 showed a significantly enhanced brain exposure relative to plasma in comparison to the negative control. Taken together, the data supports hTfR1-mediated BBB transport in this experiment for the tested, novel hTfR1 binders.

### Example 22

### Immunohistochemistry Data on Brain Exposure

In vivo engagement of hTfR1 by the Fc-scFv construct was studied further using a qualitative immunohistochemistry (IHC) analysis. In brief, coronal brain sections at a thickness of 20 μm were obtained from PBS-perfused brain hemispheres of the mice described in Example 21 using a cryostat (Microm NX50 CryoStar, Epredia). The sections were collected on Superfrost plus slides (Menzel-Gläser, #J1800AMNZ) and air-dried prior to IHC. The brain sections were washed with PBS (pH 7.4) for 15 min and incubated in blocking buffer (5% BSA, 0.25% Triton-X in PBS) for 2 h at room temperature. To visualize i.v. dosed constructs, brain sections were incubated with a secondary goat anti-human IgG (heavy and light chain specific) conjugated to Alexa Fluor 488 (Invitrogen, #A11013) for 120 min at room temperature followed by 3×15 min wash in PBS. Slides were mounted with Fluoromount-G (Invitrogen, #00-4958-02) for imaging analysis. Confocal images from cerebral cortex were captured using a Leica Stellaris 5 confocal system equipped with a HC PL APO 40×/1.25 GLYC motCORR CS2 objective (Leica, #11506423).

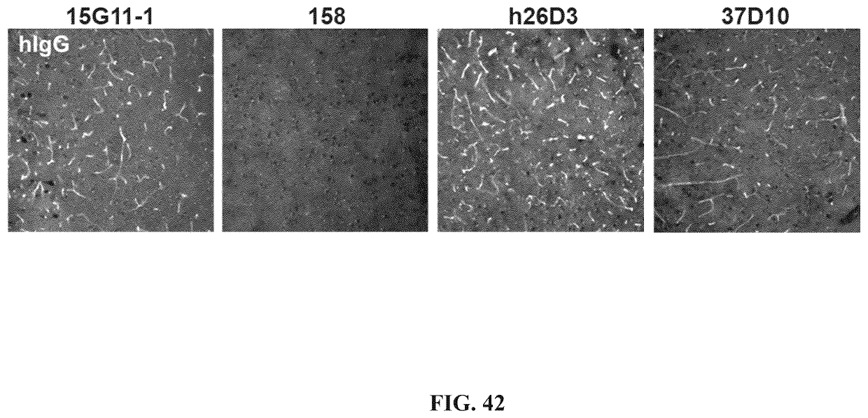

Distinct IHC immunofluorescence signals were observed in brain capillaries with positive reference module 15G11-1, while a minimal IHC signal was detected in brain sections from mice injected with negative control 158 (FIG. 42). Brain capillary IHC signal was observed for the two test constructs h26D3 and 37D10, of which h26D3 showed the strongest immunofluorescence signal, comparable to the positive control 15G11-1. Taken together, the MSD (Example 21) and IHC (this Example) analyses demonstrate that the hTfR1 binders of the disclosure in a scFv format exhibit an increased brain exposure in hTfR1-KI mice.

### Example 23

### Generation of Affinity Variants and Affinity Determinations

Several variants of the parental antibody h26D3 were generated by substituting tyrosine, tryptophan and aspartic acid residues in the CDRs one by one for alanine residues. The resulting variant VH domains were denoted HCl-HC13 and their amino acid sequences are provided in the sequence listing as SEQ ID NO:81-93, respectively. Disulfide stabilized versions of these variant VH domains have the amino acid sequences SEQ ID NO:125-137, respectively. Variant CDR sequences comprised in these variant VH domains are listed as SEQ ID NO:52-64, respectively. The resulting variant VL domains were denoted LC1-LC6 and their amino acid sequences are provided in the sequence listing as SEQ ID NO:95-100, respectively. Disulfide stabilized versions of these variant VL domains have the amino acid sequences SEQ ID NO:142-147, respectively. Variant CDR sequences comprised in these variant VL domains are listed as SEQ ID NO:65-69, respectively. Table 27 below provides a summary of the specific mutations in each of the alanine variants.

TABLE 27
Alanine substitution variants of VH and VL of h26D3

| Variant | Mutation |
|---|---|
| LC1 | Y31A |
| LC2 | Y38A |
| LC3 | Y55A |
| LC4 | W56A |
| LC5 | Y97A |
| LC6 | Y100A |
| HC1 | D31A |
| HC2 | Y32A |
| HC3 | D35A |
| HC4 | D50A |
| HC5 | D54A |
| HC6 | Y55A |
| HC7 | D56A |
| HC8 | Y60A |
| HC9 | Y101A |
| HC10 | Y106A |
| HC11 | Y107A |
| HC12 | D111A |
| HC13 | Y112A |

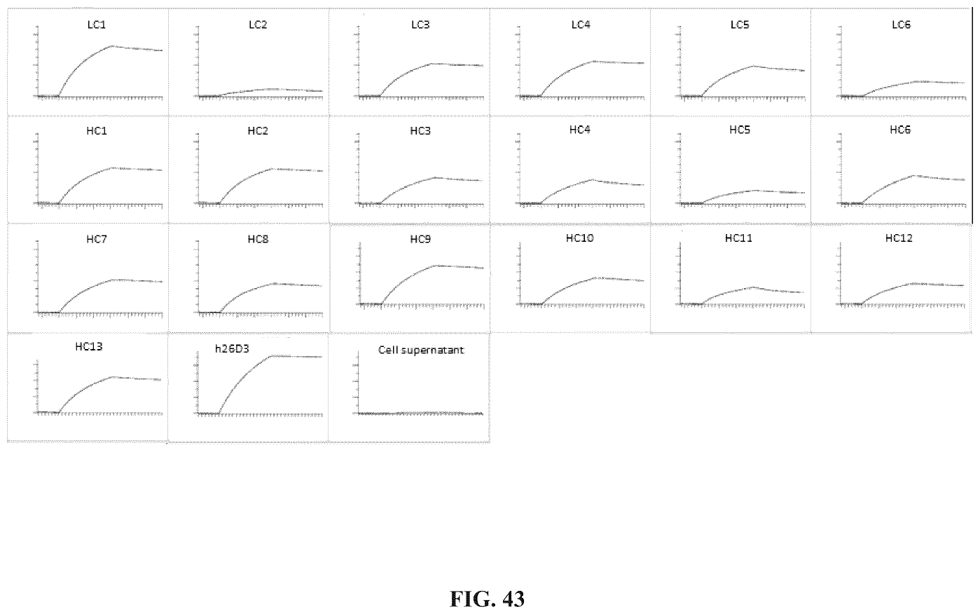

The generated alanine variants were expressed as single mutant, His-tagged Fabs by transient transfection of Chinese hamster ovary cells (ExpiCHO; Thermo Fisher Scientific) according to the manufacturer's instructions. Clarified media, into which the Fabs had been secreted, was used to assess binding to hTfR1 by BLI (Octet RED384, ForteBio). The expressed Fabs were loaded from the cell supernatants onto anti-Fab biosensors during 240 s. Thereafter, association of ectodomain of hTfR1 (SEQ ID NO:110), diluted to 3.75 μg/ml in 1× Kinetics buffer (ForteBio), to the loaded sensors was measured for 300 s, followed by dissociation for 300 s. All variants were confirmed to bind hTfR1 but were affected to different extents (FIG. 43).

Variants showing affected binding to hTfR1 in the screen were selected for further characterization. In addition, double mutants were generated by combining heavy and light chains with alanine substitutions. Table 28 below provides a summary of the specific mutations in each of the alanine variants that were selected.

TABLE 28
Variants of VH and VL of h26D3 selected
for further characterization

| Variant | Mutation VL | Mutation VH |
|---|---|---|
| LC1 | Y31A | None |
| LC5 | Y97A | None |
| LC6 | Y100A | None |
| HC2 | None | Y32A |
| HC3 | None | D35A |
| HC4 | None | D50A |
| HC5 | None | D54A |
| HC6 | None | Y55A |
| HC8 | None | Y60A |
| HC10 | None | Y106A |
| HC11 | None | Y107A |
| HC3 / LC1 | Y31A | D35A |
| HC3 / LC5 | Y97A | D35A |
| HC6 / LC1 | Y31A | Y55A |
| HC6 / LC5 | Y97A | Y55A |
| HC8 / LC1 | Y31A | Y60A |
| HC8 / LC5 | Y97A | Y60A |
| HC10 / LC1 | Y31A | Y106A |
| HC10 / LC5 | Y97A | Y106A |

The selected variants were expressed as His-tagged Fabs by transient transfection of Chinese hamster ovary cells (ExpiCHO; Thermo Fisher Scientific) according to the manufacturer's instructions. The Fabs were purified at small scale with HisPur™ Ni-NTA Magnetic Beads (Thermo Scientific) according to the manufacturer's instructions followed by buffer exchange into DPBS pH 7.4. Selected variants were also purified at a larger scale by application on a HisTrap Excel column (Cytiva), which was washed with 20 mM Tris, 200 mM NaCl and 5 mM imidazole. The proteins were eluted with 20 mM Tris, 200 mM NaCl and 500 mM imidazole, followed by buffer exchange to DPBS pH 7.4 using a HiPrep 26/10 Desalting column (Cytiva). The proteins were concentrated using an Amicon Ultra centrifugal concentrator (30 MWCO; Millipore). Selected variants were further polished by size exclusion chromatography (SEC; HiLoad 26/600 Superdex 200; Cytiva) in DPBS pH 7.4. Analytical characterization of the protein was done by UV protein determination, SDS-PAGE and HPLC-SEC.

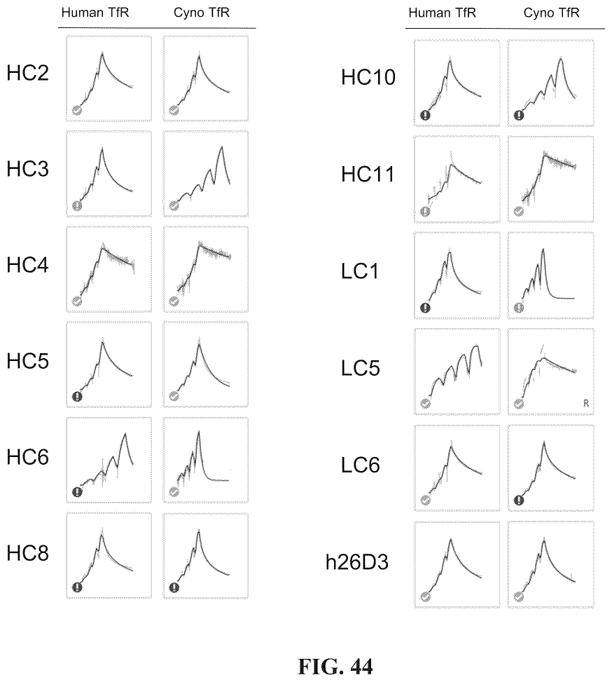

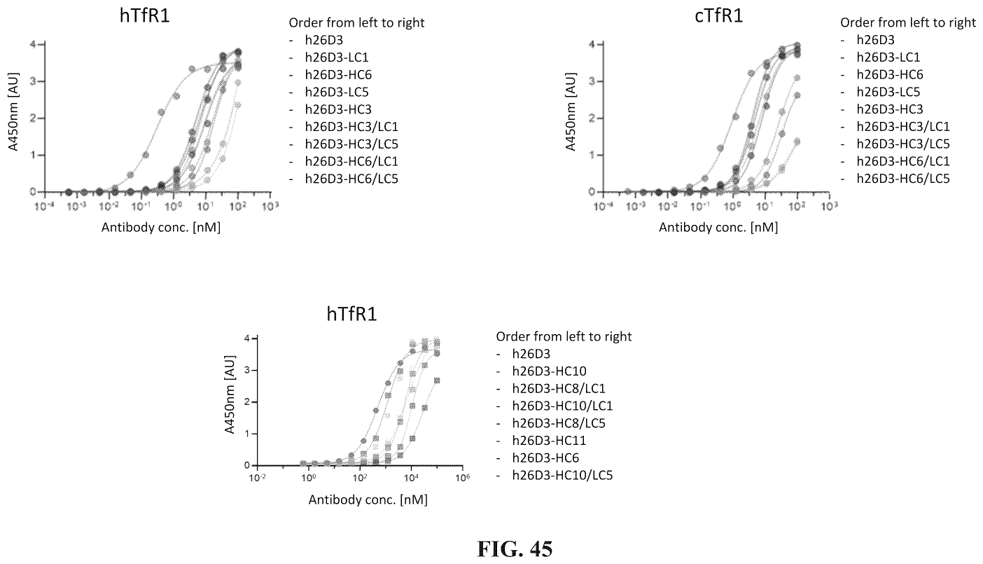

Binding of the purified Fabs to human and cynomolgus TfR1 was evaluated using either SPR (FIG. 44) or indirect ELISA (FIG. 45). For SPR, a Biacore 8K instrument (Cytiva) was used. 1 μg/ml of hTfR1 (SEQ ID NO:122) or cTfR1 (SEQ ID NO:123) was immobilized on a Cm5 sensor chip (Cytiva, #BR100399) using the amine coupling kit type 2 (Cytiva, #BR100633) according to the manufacturer's instruction. The Fabs were injected over the chip using a 2-fold dilution series in four steps starting at 100 nM. The interaction was measured using the single cycle kinetics method with a contact time of 120 s at a flow rate of 30 μl/min followed by a dissociation time of 1000 s. Regeneration of the surface between cycles was done by injecting 3M MgCl₂. The binding data was fitted to a 1:1 interaction model. The Fabs were diluted in HBS-EP+ (Cytiva, #BR100669). Experiments were performed at 25° C. The results are shown in FIG. 44, and the calculated K_{D} values are given in Table 29 below.

TABLE 29
SPR analysis of variant h26D3 Fabs vs. hTfR1 and cTfR1

| Variant | hTfR1 | cTfR1 |
|---|---|---|
| h26D3 Fab | K_{D} (nM) | K_{D} (nM) |
| HC2 | 8.7 | 12 |
| HC3 | 16 | 168 |
| HC4 | 5.1 | 8 |
| HC5 | 20 | 39 |
| HC6 | 156 | 164 |
| HC8 | 10 | 19 |
| HC10 | 9.2 | 4.6 |
| HC11 | 388 | 2.7 |
| LC1 | 14 | 70 |
| LC5 | 240 | ND |
| LC6 | 8.5 | 9.4 |
| h26D3 | 10 | 12 |

For the indirect ELISA, half area 96-well plates (Corning, #3690) were coated with 1 μg/ml recombinant ectodomain of hTfR1 (SEQ ID NO:110) in PBS overnight at 4° C. The coated plates were blocked using Pierce protein-free blocking solution (Thermo Fisher Scientific, #37572) for 1 h at room temperature with shaking and washed four times in PBS containing 0.1% TWEEN-20. Serial dilutions (1:3) of various expressed constructs in incubation buffer (1% BSA, 0.1% TWEEN-20 in PBS) were incubated for 1 h at room temperature. Following the four wash steps, bound test constructs were detected by addition of anti-human-IgG F(ab′)₂-HRP antibody (Jackson Immuno Research, #109-036-003) at 1:5000 dilution in incubation buffer (1 h, room temperature). Following four wash steps, K-Blue® Aqueous TMB substrate (Neogen, #331177) was added to the wells for 15 min at room temperature before the reaction was stopped with 1:1 dilution of 0.5 M H₂SO₄. The optical density at 450 nm was recorded (Spark, Tecan) and background signal was subtracted before analysis. The obtained results are shown in FIG. 45.

Based on the Biacore and ELISA measurements, several variants were identified within a wide range of affinities for human TfR1. Many variants exhibited a retained cross-reactivity to cynomolgus TfR1.

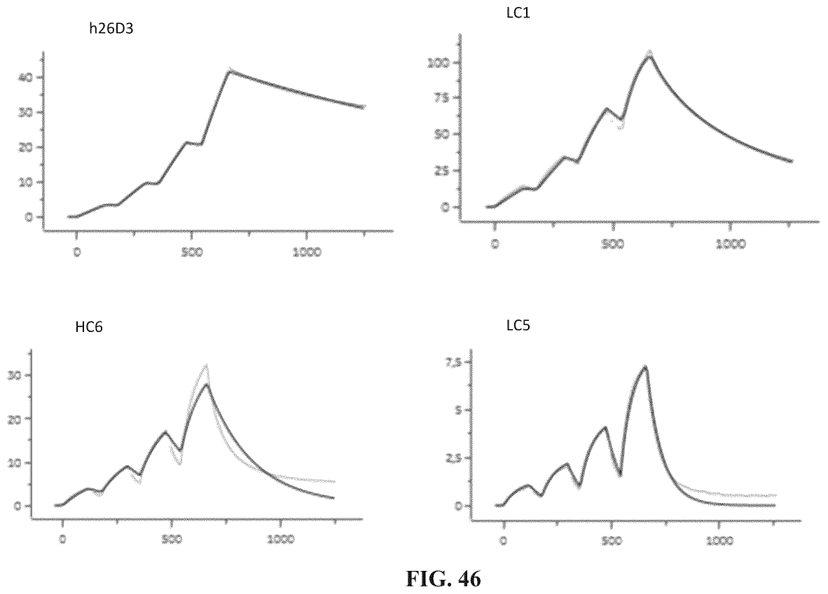

Finally, selected variants were reformatted to scFv and used in the context of the bispecific binding molecule format disclosed in WO2022/258841. Bispecific binding molecules comprising scFv modules constructed from h26D3 and selected alanine mutants were expressed in ExpiCHO cells as described above. Filtered supernatants were applied to a MabSelect SuRe column (Cytiva) which was subsequently washed with DPBS pH 7.4. Expressed binding molecules were eluted by application of 0.7% HAc pH 2.5, followed by immediate neutralization of the sample to pH 7.5. Purified samples were polished further by subjecting them to size exclusion chromatography (SEC; HiLoad 26/600 Superdex 200; Cytiva) in DPBS pH 7.4. The purified constructs were concentrated using centrifugal concentrators Amicon Ultra (30 MWCO, Millipore). Each purified expressed construct was characterized using SDS-PAGE, size-exclusion chromatography (Superdex 200 Increase 3.2/300; Cytiva) and UV protein determination. Binding to hTfR1 was evaluated using SPR as described above with adjustments of the concentration interval depending on the variant. As shown in FIG. 46 and in Table 30 below, the different tested variants exhibited a range of affinities for the hTfR1 target.

TABLE 30
SPR analysis of variant h26D3 scFv in bispecific format vs. hTfR1

| Variant | K_{D} vs hTfR1 (nM) |
|---|---|
| h26D3 | ~10 |
| LC1 | ~40 |
| HC6 | ~100-200 |
| LC5 | ~400-500 |

### Example 24

### Design, Production and Preparative SEC of Disulfide-Stabilized hTfR1-Binding Molecules

A panel of hTfR1-binding molecules in the scFv format were designed, produced and purified. The designed hTfR1-binding scFv molecules are listed in Table 31.

TABLE 31
hTfR1-binding scFv molecules and their amino acid sequences

| Designation | Amino acid sequence |
|---|---|
| h26D3-HC6 | SEQ ID NO: 151 |
| h26D3-HC6_DS | SEQ ID NO: 152 |
| h26D3-HC6, VL-first | SEQ ID NO: 153 |
| h26D3-HC6_DS, VL-first | SEQ ID NO: 154 |
| h26D3-LC1 | SEQ ID NO: 155 |
| h26D3-LC1_DS, VL-first | SEQ ID NO: 156 |
| h26D3wt | SEQ ID NO: 116 |
| h26D3wt_DS, VL-first | SEQ ID NO: 157 |

The scFv variants whose respective designation includes the “_DS” suffix all comprise two mutations which introduce cysteine residues at position 44 of the VH domain and at position 106 of the VL domain of the respective starting sequences. It is contemplated that these introduced cysteine residues cause the formation of a stabilizing disulfide bond between the VH and VL domains.

The test items were produced as His₆ tagged scFv constructs with the His₆ tag spaced from the remainder of the scFv by a flexible (G₄S)₄ linker (combined tag sequence given by SEQ ID NO:158), by transient transfection of CHO cells in 400 ml culture volume per scFv. One of the test items, “h26D3-wt_DS, VL-first”, was also produced with both an His₆ tag and an Avi tag (combined tag sequence given by SEQ ID NO:159) for site directed in vivo biotinylation, and was expressed in 11 culture volume.

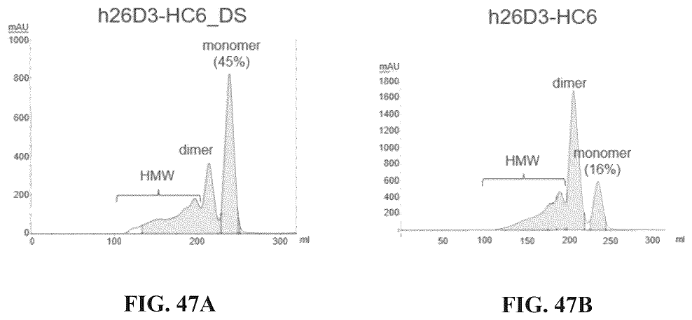

For purification, all scFv proteins were recovered by immobilized metal ion affinity chromatography (IMAC) purification from clarified cell supernatants. For IMAC, supernatants were loaded on a HisTrap excel 5 ml column (Cytiva) and unbound material washed out with wash buffer (PBS, 350 mM NaCl and 10 mM imidazole). Bound scFv was then eluted in elution buffer (PBS, 350 mM NaCl, 0.5 M imidazole, pH 7.5). Next, the eluted proteins were passed over a preparative SEC column (HiLoad 26/600 Superdex 200 pg; Cytiva) with PBS, pH 7.4 as running buffer. SEC fractions containing monomeric scFv were collected and brought to 1 mg/ml final concentration in PBS, pH 7.4. Representative chromatograms from this preparative SEC are shown in FIG. 47A for h26D3-HC6_DS and in FIG. 47B for h26D3-HC6, and show that the scFv molecules are recovered with different degrees of aggregated forms during the initial IMAC purification. For h26D3-HC6_DS (FIG. 47A), 45% of the material elutes in the main peak and contain the monomeric, desired scFv. This is in contrast to h26D3-HC6 (FIG. 47B), for which the distribution between dimer and monomer is the opposite, showing scFv dimer in the main peak, and only 16% of the material in the monomer peak. The size distribution of higher molecular weight (HMW) species is similar for both constructs.

### Example 25

### Analytical SEC of Disulfide-Stabilized hTfR1-Binding Molecules

Following three freeze/thaw cycles between room temperature and −80° C., 1 μg of each scFv variant produced in Example 24 was injected to a SEC column (Waters BioSuite 250 UHR SEC 4 μm, 4.6×300 mm). Analyses were done with a running buffer of 0.2 M potassium phosphate, 0.25 M KCl, pH 6.2 at a flow rate of 0.35 ml/min.

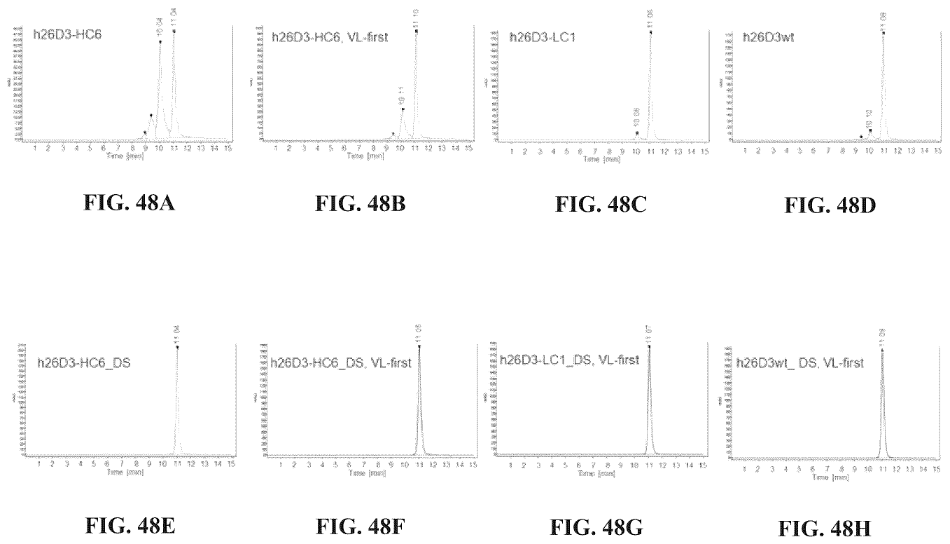

The results of the analytical SEC experiment are shown in FIGS. 48A-48H and Table 32. The monomeric form of all scFv samples have a retention time of 11 min (FIGS. 48A-48H). In scFv molecules lacking the DS mutations, additional peaks, corresponding to multimerized forms of scFv are detected (FIGS. 48A-48D). In all samples with DS mutations, 100% of the respective molecule migrate at 11 min as monomeric scFv (FIGS. 48E-48H). The percentage distribution of integrated peak areas from the analytical SEC samples are listed in Table 32. Again, for the four samples with stabilizing DS mutations, 100% of injected proteins are detected in the monomer peak, whereas additional peaks of multimeric forms are detected for corresponding samples without DS mutations.

TABLE 32
Distribution of peak areas from SEC chromatograms

| | Chromatogram | Monomer | Dimer | Tetramer | Hexamer |
|---|---|---|---|---|---|
| Molecule | in FIG. 48 | (%) | (%) | (%) | (%) |
| h26D3-HC6 | A | 38 | 47 | 13 | 2 |
| h26D3-HC6, VL-first | B | 73 | 23 | 4 | |
| h26D3-LC1 | C | 94 | 6 | | |
| h26D3wt | D | 90 | 9 | 1 | |
| h26D3-HC6_DS | E | 100 | | | |
| h26D3-HC6_DS, VL-first | F | 100 | | | |
| h26D3-LC1_DS, VL-first | G | 100 | | | |
| h26D3wt_DS, VL-first | H | 100 | | | |

### Example 26

### Thermal Stability of Disulfide-Stabilized hTfR1-Binding Molecules

Monomer stability of scFv samples was evaluated by HPLC SEC analysis. The panel of scFv molecules produced and studied in Examples 24-25, purified and stored in PBS, were subjected to temperature hold for one, two or four weeks at temperatures 4° C., 40° C. and frozen at −80° C., except for h26D3-HC6_DS, VL-first and h26D3-LC1, VL-first, which were held frozen at −70° C. and −75° C. respectively. At each timepoint, samples of each variant from each temperature were analyzed by HPLC-SEC as described in Example 25. At the initiation of the study, frozen samples were thawed and analyzed, and are denoted TO.

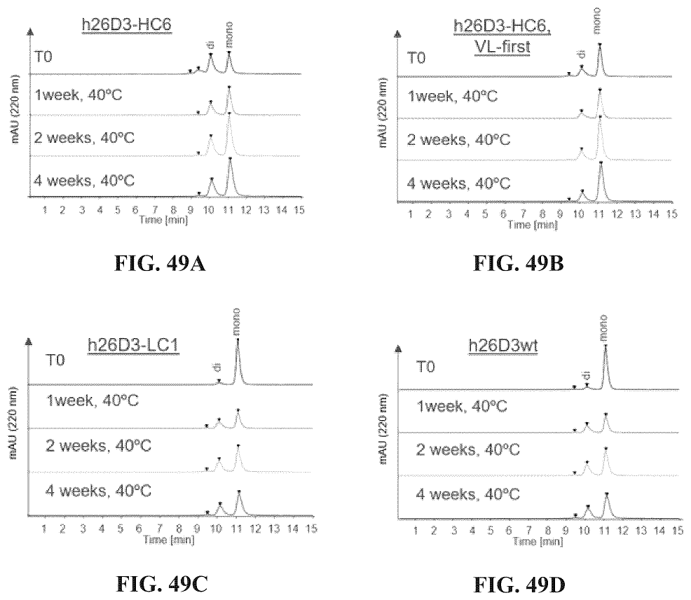

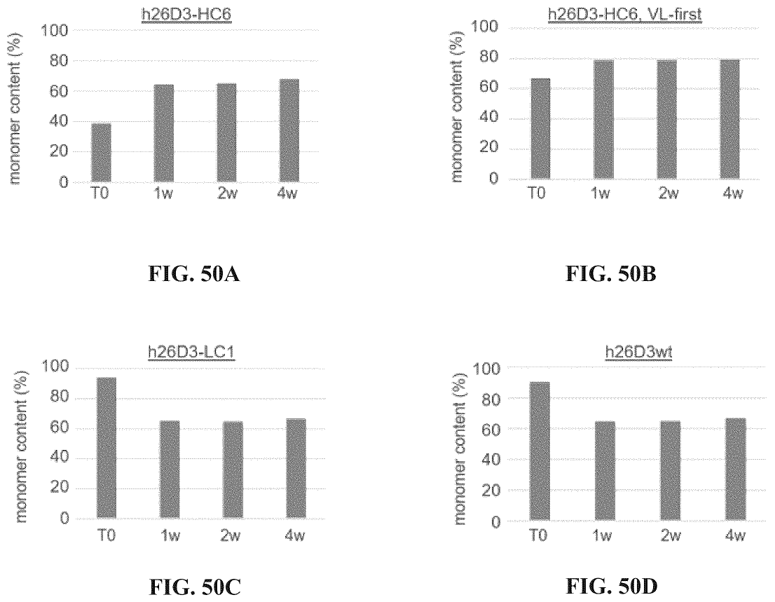

The results for scFv molecules without DS mutations after storage at 40° C. for 1-4 weeks are shown in FIGS. 49A-49D and FIGS. 50A-50D. All samples were isolated as pure monomers in the preceding preparative SEC purification described in Example 24. However, analytical SEC revealed that all samples contain both monomer (retention at approximately 11 min) and dimer (retention at approximately 10 min) forms already at the initial time point TO (FIGS. 49A-49D). The share of dimers is the most significant for h26D3-HC6 (FIG. 49A) and h26D3-HC6, VL-first (FIG. 49B), while the majority of scFv molecules are monomeric at TO for h26D3-LC1 (FIG. 49C) and h26D3 wt (FIG. 49D). The chromatograms show that the distribution between monomeric and dimeric forms shifts gradually during the study. In samples from 4 weeks, the monomer/dimer distributions are more similar between the different molecules as compared to the corresponding distributions at T0 (FIGS. 49A-49D). Multimers are observed for all scFv molecules as a minor peak with a retention time between 9-10 min (FIGS. 49A-49D). The results indicate that, despite having been isolated in the pure monomeric form, scFv molecules without the stabilizing DS mutations form multimers during storage.

The same pattern is shown by the percentage proportions of monomeric scFv molecules exhibited in FIGS. 50A-50D. The molecules h26D3-HC6 (FIG. 50A) and h26D3-HC6, VL-first (FIG. 50B) have lower proportions of monomeric scFv at TO (reference sample kept at −80° C.). The degree of monomer increases for these molecules in samples kept at 40° C. for 1-4 weeks (FIGS. 50A-50B). The opposite is seen for h26D3-LC1 (FIG. 50C) and h26D3 wt (FIG. 50D), where monomer content is high at TO and then decreases during storage at 40° C. for 1-4 weeks. The observation indicates that scFv molecules without DS mutations reach an equilibrium between monomeric and dimeric states during storage. For h26D3-HC6 and h26D3-HC6 VL-first, the monomer content increased over the course of the study, while for h26D3-LC1 and h26D3 wt, the monomer content decreased as compared to T0.

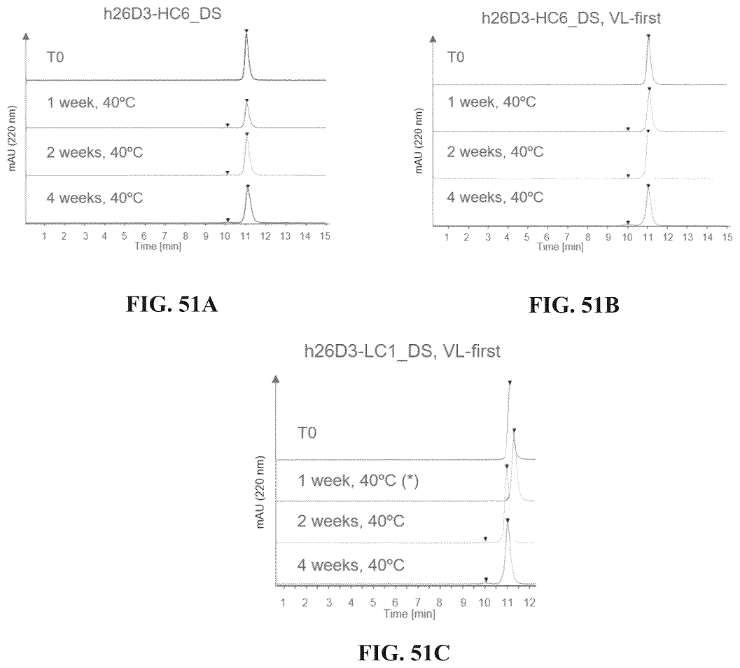

Corresponding chromatograms for scFv molecules with DS mutations after storage at 40° C. for 1-4 weeks are shown in FIGS. 51A-51C. As seen here, the molecules with introduced DS mutations are highly stable as monomers over the studied period, with uniform peaks of monomeric scFv detected at a retention time just above 11 min for all the variants (FIGS. 51A-51C). Only in chromatograms from samples incubated for 4 weeks at 40° C. (FIG. 51C), a very small peak is observed at 10 min retention time. The results demonstrate that the monomeric state of DS-stabilized scFv molecules is highly stable also at an extended storage time at 40° C.

As can be seen in Table 33 below, the high stability of the scFv variants with DS mutations as compared to corresponding variants without DS mutations was also seen for samples kept at 4° C. or frozen.

TABLE 33
Percentage of monomer forms of scFv samples

| | h26D3-HC6 | | | h26D3-HC6, VL-first | | |
|---|---|---|---|---|---|---|
| | | Retention | | | Retention | |
| Timepoint | Temp | time | Monomer % | Temp | time | Monomer % |
| T0 | | 11.04 | 38.58 | | 11.099 | 66.86 |
| 1 w | +4° C. | 11.048 | 47.16 | +4° C. | 11.104 | 74.81 |
| 2 w | +4° C. | 11.047 | 50.75 | +4° C. | 11.107 | 72.22 |
| 4 w | +4° C. | 11.087 | 54.25 | +4° C. | 11.148 | 70.69 |
| 1 w | +40° C. | 11.048 | 64.23 | +40° C. | 11.106 | 78.68 |
| 2 w | +40° C. | 11.055 | 64.95 | +40° C. | 11.114 | 78.72 |
| 4 w | +40° C. | 11.099 | 67.56 | +40° C. | 11.156 | 79.02 |
| 1 w | −80° C. | 11.055 | 41.54 | −80° C. | 11.107 | 52.6 |
| 2 w | −80° C. | 11.053 | 45.24 | −80° C. | 11.114 | 49.96 |
| 4 w | −80° C. | 11.089 | 45.19 | −80° C. | 11.155 | 56.69 |
| | h26D3-LC1 | | | h26D3 wt | | |
| | | Retention | | | Retention | |
| Timepoint | Temp | time | Monomer % | Temp | time | Monomer % |
| T0 | | 11.065 | 93.96 | | 11.079 | 90.26 |
| 1 w | +4° C. | 11.079 | 88.36 | +4° C. | 11.092 | 82.78 |
| 2 w | +4° C. | 11.081 | 83.04 | +4° C. | 11.093 | 76.23 |
| 4 w | +4° C. | 11.126 | 75.98 | +4° C. | 11.139 | 68.6 |
| 1 w | +40° C. | 11.08 | 65.18 | +40° C. | 11.094 | 64.55 |
| 2 w | +40° C. | 11.087 | 64.61 | +40° C. | 11.101 | 64.72 |
| 4 w | +40° C. | 11.133 | 66.67 | +40° C. | 11.145 | 66.8 |
| 1 w | −80° C. | 11.078 | 87.63 | −80° C. | 11.091 | 89.57 |
| 2 w | −80° C. | 11.084 | 88.89 | −80° C. | 11.095 | 86.32 |
| 4 w | −80° C. | 11.128 | 91.28 | −80° C. | 11.141 | 78.9 |

| | h26D3-HC6_DS | | | h26D3-HC6_DS, VL-first | | | h26D3-LC1_DS, VL-first | | |
|---|---|---|---|---|---|---|---|---|---|
| | | Retention | | | Retention | | | Retention | |
| Timepoint | Temp | time | Monomer % | Temp | time | Monomer % | Temp | time | Monomer % |
| T0 | | 11.034 | 100 | | | | | 11.151 | 99.47 |
| 1 w | +4° C. | 11.046 | 100 | +4° C. | 11.095 | 100 | +4° C. | 11.342 | 100 |
| 2 w | +4° C. | 11.05 | 100 | +4° C. | 11.021 | 100 | +4° C. | 10.997 | 98.63 |
| 4 w | +4° C. | 11.092 | 99.36 | +4° C. | 11.029 | 100 | +4° C. | 11.03 | 99.19 |
| 1 w | +40° C. | 11.048 | 99.42 | +40° C. | 11.095 | 100 | +40° C. | 11.329 | 98.91 |
| 2 w | +40° C. | 11.056 | 98.83 | +40° C. | 11.027 | 99.2 | +40° C. | 11.008 | 97.69 |
| 4 w | +40° C. | 11.098 | 98.24 | +40° C. | 11.040 | 98.36 | +40° C. | 11.052 | 97.34 |
| 1 w | −80° C. | 11.045 | 100 | −70° C. | 11.1 | 100 | −75° C. | 11.364 | 100 |
| 2 w | −80° C. | 11.051 | 100 | −70° C. | 11.024 | 100 | −75° C. | 10.998 | 98.82 |
| 4 w | −80° C. | 11.091 | 99.54 | −70° C. | 11.032 | 100 | −75° C. | 11.029 | 99.43 |

### Example 27

### Serum Stability of Disulfide-Stabilized hTfR1 Binding Molecules

Serum stability is a critical attribute for antibodies and different fragments such as scFv-containing biotherapeutics (Worn and Pluckthun (2001), J Mol Biol 305(5):989-1010; Austerberry et al (2017), Eur J Pharm Biopharm 115:18-30; Willuda et al (1999), Cancer Res 59:5758-67). In order to assess the stability in serum of scFv molecules with DS mutations, the variant h26D3 wt_DS, VL-first expressed with His₆ and Avi tags (see Example 23) was incubated in mouse serum (Capricon, MOU-1B) and 1×PBS (#09-9400-100, Medicago AB) respectively at both 4° C. and 37° C. using a thermal mixer (Eppendorf ThermoMixer C, Eppendorf). After 48 h incubation, binding of the scFv to hTfR1 was evaluated by ELISA. In brief, a half area 96-well plate (#3690, Corning) was coated overnight at 4° C. with hTfR1 diluted in 1×PBS, followed by blocking with Pierce Protein-Free Blocking Buffer (#37572, ThermoScientific) for 1 h at room temperature (RT) with shaking. h26D3 wt_DS, VL-first was diluted or serially diluted in mouse serum or ELISA incubation buffer (EIB): 1×PBS-0.05% Tween20, 0.1% BSA (PBS-T, #09-9410-100, Medicago AB, A7030-100G, Sigma-Aldrich), added to plate and incubated for 2 h at 4-8° C. with shaking. Bound biotinylated scFv was detected using streptavidin-horseradish-peroxidase (#3310-9-100, Mabtech) in EIB for 1 h at RT with shaking, followed by TMB (#331177, Neogen). The reaction was stopped by 1:1 addition of 0.5 M sulfuric acid (#35354-1L, Honeywell). Optical density at 450 nm was obtained using a microplate reader (Spark, Tecan) and the collected data was plotted using GraphPad Prism software (GraphPad Software Inc). Serum stability of the scFv is displayed as % binding to hTfR1 and determined using the following equation:

Serum stability=(ELISA OD450 at 37° C.)/(ELISA OD450 at 4° C.)×100%

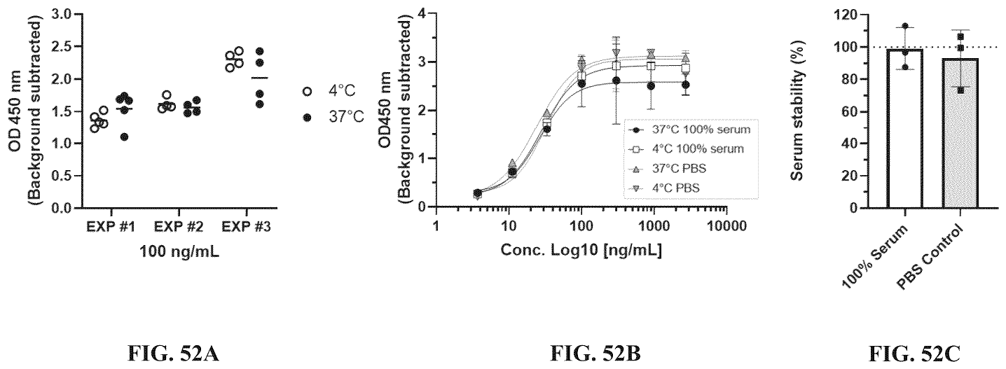

The results are shown in FIGS. 52A-52C, and demonstrate stability and a highly retained hTfR1 binding ability of the tested scFv variant after incubation in mouse serum for 48 h at the tested temperatures. The results can be compared to other published scFv stability data in mouse serum (Liu et al (2022), mAbs 14:1, 2073632).

### Example 28

### Dynamic Light Scattering Analysis of Disulfide-Stabilized hTfR1 Binding Molecules

Dynamic light scattering (DLS) analysis of scFv variants h26D3-HC6_DS, VL-first; h26D3-HC6 and h26D3-HC6_DS (see Example 23) was performed at 25° C. or 20° C. using an Uncle instrument (Unchained Labs). Average hydrodynamic diameter and polydispersity index (PDI) were calculated from analyses run in triplicates h26D3-HC6_DS, VL-first) or duplicates (h26D3-HC6 and h26D3-HC6_DS). Samples were diluted to 1 mg/ml in PBS prior to analysis.

The results are shown in Table 34. The scFv variant without DS mutations (h26D3-HC6) exhibits a larger average hydrodynamic diameter than either of the two variants with DS mutations (h26D3-HC6_DS, VL-first and h26D3-HC6_DS). The results are expected from the high monomeric content for DS stabilized scFv as shown by analytical SEC (FIGS. 48A-48H and Table 32), and agree with other reported DLS analyses of scFv (Morioka et al (2019), Molecules 24(14):2620). The observed PDI values are around 0.1 in all variants, indicating monodisperse (PDI<0.1) or a low degree of polydispersity (PDI 0.1-0.2).

TABLE 34
Dynamic light scattering analysis of scFv variants

| | Average hydrodynamic | |
|---|---|---|
| Molecule | diameter (nm) | PDI |
| h26D3-HC6_DS, VL-first | 4.8 | 0.13 |
| h26D3-HC6 | 6.9 | 0.08 |
| h26D3-HC6_DS | 4.6 | 0.09 |

### Example 29

### Surface Plasmon Resonance Analysis of Disulfide-Stabilized hTfR1-Binding Molecules

Binding to hTfR1 of six different purified scFv variants from Example 24 was evaluated by surface plasmon resonance (SPR) using Biacore 8K (Cytiva). 30 μg/ml of DYKDDDDK Tag mAb FG4R (Thermo Fisher Scientific, MA1-91878) in 10 mM acetate buffer, pH 4.5 was immobilized on a CM5 sensor chip (Cytiva, #BR100399) using the amine coupling kit type 2 (Cytiva, #BR100633) according to the manufacturer's instruction, resulting in immobilization levels of approximately 10000 RU. hTfR1 was captured via an N-terminal DYKDDDDK tag. To this end, the receptor was injected over the chip with a contact time of 60 s at a flow rate of 10 μl/min. Each of the six scFv variants h26D3-HC6; h26D3-HC6, VL-first; h26D3-LC1; h26D3-HC6_DS; h26D3-HC6_DS, VL-first and h26D3-LC1_DS, VL-first were injected over the chip using a 3-fold dilution series in five steps starting at 700 nM or 400 nM. Interaction was measured using the single cycle kinetics method with a contact time of 120 s at a flow rate of 30 μl/min followed by a dissociation time of 600 s. Regeneration of the surface between cycles was done by injecting 10 mM glycine-HCl pH 1.7 with a contact time of 30 s and a flow rate of 30 μl/min. The binding modules were diluted in HBS-EP+ (Cytiva, #BR100669). Experiments were performed at 25° C.

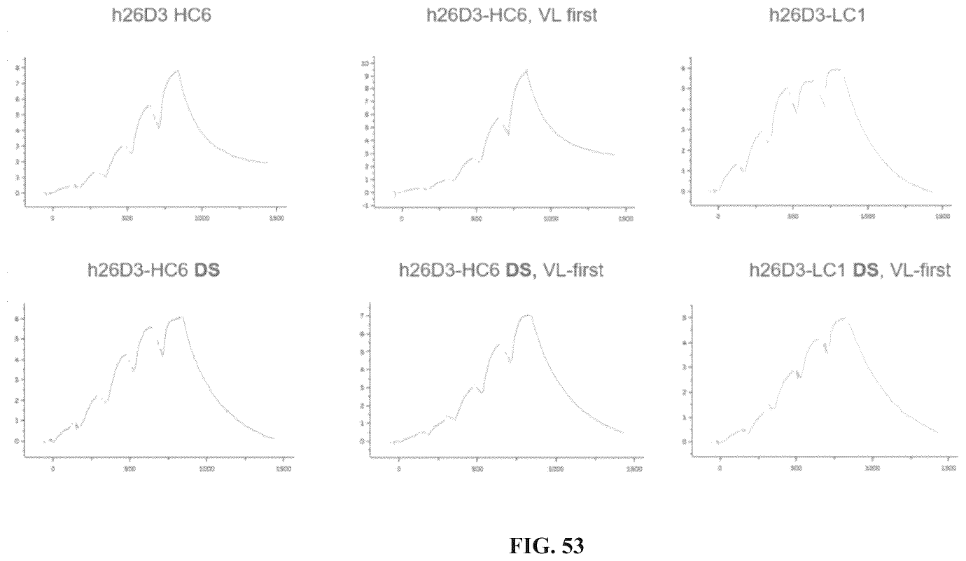

The resulting binding curves are shown in FIG. 53. As can be seen from the SPR diagrams, introduction of stabilizing DS mutations does not disrupt the binding of hTfR1 by the scFv variants. It can also be seen that the scFv variants without DS mutations dissociate from the hTfR1 antigen in a biphasic manner, which may be due to an element of avidity interaction observed for dimeric forms present in the samples.

### Example 30

### Design and Characterization of Bispecific Binding Molecules with Stabilized hTfR1-Binding scFv Domain

Three different bispecific binding molecules were designed as knob-into-hole antibody constructs containing a hTfR1-binding scFv with stabilizing DS mutations linked to the C-terminal amino acid residue of the knob heavy chain of the antibody. The same antibody light chain and hole heavy chain sequences were used in all three constructs, and the knob heavy chain sequences only differed with respect to which scFv variant was used. The tested molecules are listed in Table 35.

TABLE 35
Amino acid sequences of tested bispecific binding molecules

| | LC | Hole HC | Knob HC | |
|---|---|---|---|---|
| Designation | (SEQ ID NO:) | (SEQ ID NO:) | (SEQ ID NO:) | scFv variant |
| BA001 | 160 | 161 | 162 | h26D3-HC6_DS, VL first |
| BA002 | 160 | 161 | 163 | h26D3-LC1_DS, VL first |
| BA004 | 160 | 161 | 164 | h26D3wt_DS, VL first |

The tested bispecific binding molecules were formulated at 3.5 mg/ml (20 μM) concentration in Gibco 1×DPBS (Fisher Scientific, 14190-136) and subjected to incubation at 4° C., 25° C. or 40° C. for one, two or four weeks using a Memmert cooled incubator.

Samples from each temperature were taken at one, two and four weeks and immediately analyzed by analytical size exclusion chromatography using a 1260 Infinity II LC system (Agilent) equipped with a TSKG3000SW column (7.8×300 mm, 5 μm particle size; Tosoh Bioscience) coupled to an in-line filter. Samples were analyzed by injecting 10 μg of sample at a flow rate of 0.5 ml/min of 0.2M sodium phosphate at pH 7.0. The percentage monomer elution peak area was quantified using Agilent OpenLab Data Analysis version 2.5.

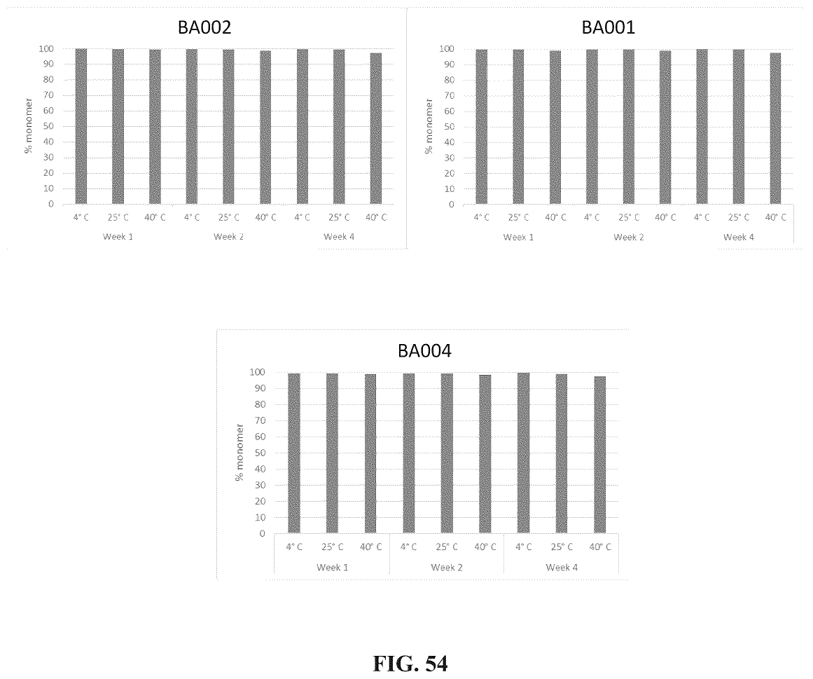

The results are shown in FIG. 54 and demonstrate that bispecific binding molecules comprising any one of the three different, disulfide-stabilized hTfR1-binding scFv domains maintain a high stability at all tested temperatures and time periods, as evidenced by a very high percentage of monomeric content.

### Example 31

### Generation of Bispecific Binding Molecules

This example describes the design and production of bispecific binding molecules which incorporate the humanized AβpE3-specific antibodies described in Example 10 and the humanized, stabilized hTfR1-binding scFv variants described in Example 24.

### Materials and Methods

Design of constructs: Bispecific binding molecules BA001 and BA002 were designed as knob-into-hole variants of the AβpE3-specific antibodies, with an hTfR1-binding scFv linked to the C-terminal amino acid residue of the knob heavy chain of the antibody. As control, the AβpE3-specific standard antibody BA003 was used. The amino acid sequences of the tested molecules are listed in Table 36.

TABLE 36
Amino acid sequences of tested bispecific binding and control molecules

| | LC | Hole HC | Knob HC | |
|---|---|---|---|---|
| Designation | (SEQ ID NO:) | (SEQ ID NO:) | (SEQ ID NO:) | scFv variant |
| BA001 | 160 | 161 | 162 | h26D3-HC6_DS, VL first |
| BA002 | 160 | 161 | 163 | h26D3-LC1_DS, VL first |
| | | HC | | |
| | | (SEQ ID NO:) | | |
| BA003 | 160 | 165 | | |

Expression from transient transfection: The tested molecules were expressed in CHO cells and purified by affinity chromatography, followed by preparative size-exclusion chromatography (SEC) and buffer exchange into phosphate buffered saline (PBS) solution. The purified molecules were characterized using SDS-PAGE, SEC, and UV protein determination.

### Results

Both bispecific binding molecules were successfully produced and purified to a final concentration of 10 mg/ml. Protein purity, as defined by SEC-HPLC, was >98% monomer for both constructs.

### Example 32

### Characterization of Target Binding

This example describes the binding of the bispecific binding molecules generated and produced in Example 31 to human TfR1 and to AβpE3 monomers and protofibrils by SPR and inhibition ELISA.

### Materials and Methods

Human TfR1: The human transferrin receptor 1 (hTfR1) was expressed in human embryonic kidney (HEK293) cells (Expi293; ThermoFisher) according to the manufacturer's instructions. The harvested supernatant was purified using HiTrap IMAC Sepharose FF (Cytiva) followed by SEC on HiLoad Superdex 200pg 26/600 (Cytiva). The following buffers were used: Ni-NTA wash buffer: 20 mM Tris pH 8.0, 10 mM imidazole and 200 mM NaCl; Ni-NTA elution buffer: 20 mM Tris pH 8.0, 200 mM NaCl and 500 mM imidazole; size-exclusion buffer (SEC): 1×dPBS (Thermo Fisher). Purified hTfR1 (SEQ ID NO:122) was concentrated to 2 mg/ml and stored at −80° C. until analysis.

AβpE3 monomers and AβpE3 protofibrils: AβpE3-40 monomers were purchased from Bachem and dissolved in 10 mM NaOH, 0.005% Tween-20, pH>11 at a concentration of 100 μM. Aliquots were made and stored at −80° C. until analysis. The AβpE3-40 peptides were verified to be monomeric by size-exclusion chromatography. Protofibrils were prepared using an AβpE3-42 peptide from Bachem, as described in Example 6.

Affinity evaluation and K_{D} determination by surface plasmon resonance: Binding interactions between antigens and binding molecules were evaluated by SPR using a Biacore 8K or 8K+ instrument (Cytiva) according to standard procedures.

Single cycle kinetics using capture was used to measure binding to hTfR1. For measurement, 30 μg/ml FLAG-Tag mAb FG4R (Invitrogen) was immobilized on a CM5 chip. 5 μg/ml hTfR1 was captured for each cycle followed by injection of the binding molecules using a 3-fold dilution in five steps starting at 2000 nM for BA001 and 1000 nM for BA002, using a 2 min injection of every concentration and a 10 min dissociation time. Regeneration of the surface between each cycle was done by injecting 30 μl 10 mM Glycine-HCl, pH 1.7 (Cytiva, cat. No. 29215281). The binding data was fitted using a 1:1 interaction model.

Single cycle kinetics with the binding molecules immobilized on a CM5 chip was used to measure binding to the AβpE3-40 monomer. For the measurements, 5 μg/ml of analyte binding molecule was immobilized on the chip. The monomer was then injected over the chip using a 2-fold dilution in five steps starting at 250 nM, using 2 min injection of every monomer concentration and a 20 min dissociation time. Regeneration of the surface between cycles was done by injecting 30 μl 3 M MgCl₂ (Cytiva, cat. No. 29234600). The binding data was fitted to a 1:1 interaction model.

In all SPR experiments, 1×HBS-EP+ (Cytiva, cat. No. BR100669) was used to dilute binding and target molecules. Experiments were performed at 25° C.

IC₅₀ determination by inhibition ELISA: The binding of BA001, BA002 and BA003 to AβpE3-40 monomers and AβpE3-42 protofibrils was evaluated by inhibition ELISA. The tested molecules were incubated at a fixed concentration (0.1 μg/ml) with titrating concentrations of the different AβpE3 antigens. After incubation for 45 min at 900 rpm to reach equilibrium, the samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

### Results

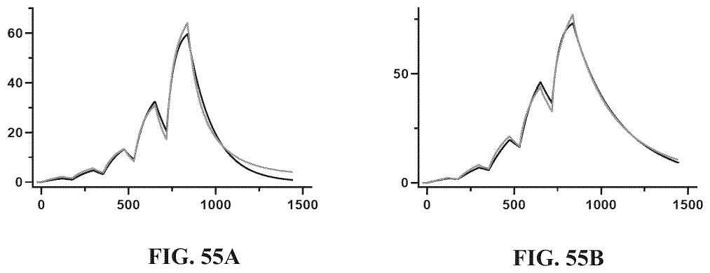

Affinity evaluation and K_{D} determination by surface plasmon resonance: The binding of BA001 and BA002 to hTfR1 was evaluated in SPR and K_{D} values were determined. Both BA001 and BA002 demonstrated binding to hTfR1 with different affinities. The calculated kₐ, k_{d} and K_{D} values are shown in Table 37. Representative sensorgrams are shown in FIGS. 55A-55B.

TABLE 37
SPR analysis of binding to human TfR1

Human TfR1

| Binding | kₐ (M⁻¹s⁻¹) | k_{d} (s⁻¹) | K_{D} (nM) |
|---|---|---|---|
| molecule | Mean ± SD | Mean ± SD | Mean ± SD |
| BA001 | 2.2 ± 0.1 e4 | 3.8 ± 0.2 e−3 | 174 ± 12.3 |
| BA002 | 8.7 ± 0.6 e4 | 3.1 ± 0.5 e−3 | 35.6 ± 2.2 |

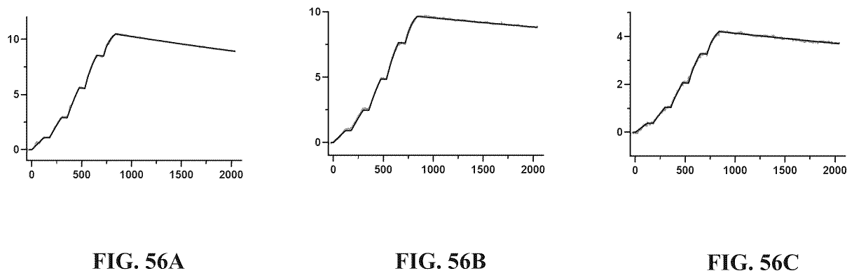

The binding of BA001 and BA002 to AβpE3-40 monomers was evaluated in SPR and their K_{D} values were determined. Both binding molecules demonstrated binding to AβpE3-40 monomers at an affinity similar to that of the comparator antibody BA003. The calculated kₐ, k_{d} and K_{D} values are shown in Table 38. Representative sensorgrams are shown in FIGS. 56A-56C.

TABLE 38
SPR analysis of binding to AβpE3-40 monomers

AβpE3-40 monomers

| Binding | kₐ (M⁻¹s⁻¹) | k_{d} (s⁻¹) | K_{D} (nM) |
|---|---|---|---|
| molecule | Mean ± SD | Mean ± SD | Mean ± SD |
| BA001 | 5.58 ± 0.07 e4 | 1.20 ± 0.05 e−4 | 2.17 ± 0.09 |
| BA002 | 5.35 ± 0.09 e4 | 1.27 ± 0.05 e−4 | 2.38 ± 0.10 |
| BA003 | 5.46 ± 0.25 e4 | 1.27 ± 0.02 e−4 | 2.33 ± 0.11 |

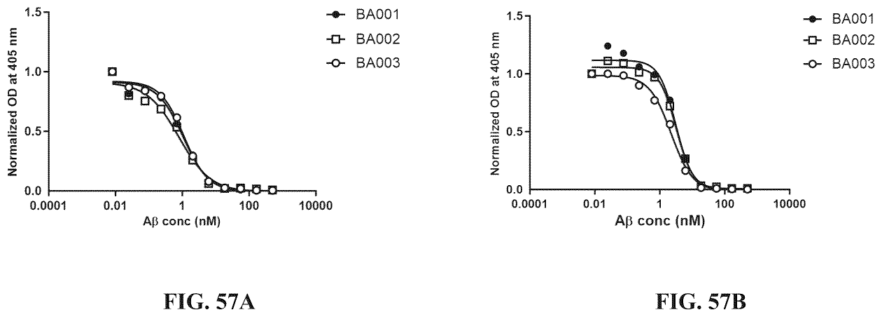

IC₅₀ determination by inhibition ELISA: The binding to AβpE3-40 monomers and AβpE3-42 protofibrils was evaluated using inhibition ELISA. Both BA001 and BA002 demonstrated binding to AβpE3-40 monomer and AβpE3-42 protofibrils in solution (FIGS. 57A-57B), at similar IC₅₀ values as the comparator antibody BA003 (Table 39).

TABLE 39
Binding to AβpE3 monomers and
protofibrils using inhibition ELISA

| | AβpE3-40 | AβpE3-42 |
|---|---|---|
| Binding | monomer | protofibril |
| molecule | IC₅₀ (nM) | IC₅₀ (nM) |
| BA001 | 1.1 | 3.1 |
| BA002 | 0.8 | 3.2 |
| BA003 | 1.1 | 2.2 |

### Example 33

### Target Binding in Brain from Human Alzheimer's Disease Patient

This example describes target binding of bispecific binding molecules, generated as described in Example 31 and tested by immunoprecipitation in a human brain extract from an AD patient.

### Materials and Methods

Brain tissue homogenization and sample preparation: Fresh frozen human brain cortical tissue from an AD patient was homogenized in a Potter-Elvehjem homogenizer at 1:10 weight:volume in Tris-buffered saline (TBS) buffer followed by centrifugation at 16000×g for 1 h. The resulting supernatant was frozen at −80° C. until analysis.

Target binding in human Alzheimer's disease brain extracts by immunoprecipitation: Binding of bispecific binding molecules to target in human AD brain was analyzed by immunoprecipitation, which is a method for removal of target protein in a sample using an antibody specific for the target molecule. Briefly, three concentrations (70, 700 and 7000 μM) of each of the tested molecules were pre-incubated with soluble 16000×g TBS brain extracts from an AD case for 2 h. Magnetic Protein A Dynabeads were added to the antigen-binder complexes and incubation continued for an additional 30 min. The bead-bound target was separated by a magnet and eluted from the beads using 70% formic acid. After neutralization, the pellet (immunoprecipitation fraction) was analyzed using an in house developed MSD assay for measurements of total AβpE3-x levels. Briefly, MSD GOLD 96-well small spot streptavidin plate was coated with biotinylated Pyr12.2 antibody (see Example 1-3) for 1 h at RT. After a washing step, free binding sites were blocked by incubation with Diluent 35. The plate was washed again and further incubated for 2 h (900 rpm shaking) with a dilution series of a standard (AβpE3-40 monomer, 1.5625-400 μg/ml) and with test samples. After another washing step, anti-Aβ4G8 SULFO tag conjugated detection antibody was added to the plate for 1 h at a concentration of 1 μg/ml. Finally, the plate was washed and read using an MSD sector imager (S 600 MM, MSD). Obtained signal was correlated to the amount of AβpE3-x in the sample.

### Results

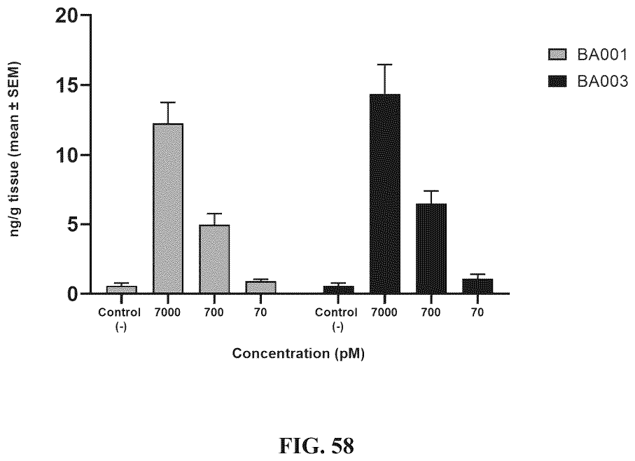

Target binding in human Alzheimer's disease brain extracts by immunoprecipitation: The bispecific binding molecule BA001 and comparator antibody BA003 were tested for their ability to bind to AβpE3 in solution, in a brain extract from a human AD patient. Immunoprecipitation of the TBS brain extract using the humanized antibodies demonstrated a concentration-dependent immunoprecipitation of AβpE3-x by both antibodies (FIG. 58).

### Example 34

### Characterization of Functional Effects

This example describes the functional effects of the bispecific binding molecules generated and produced in Example 31. The potency of the molecules with regard to mediation of uptake of AβpE3-40 monomers into THP-1 cells and clearance of amyloid plaques ex vivo in AD brain sections was evaluated.

### Materials and Methods

Uptake of AβpE3-40 monomers in THP-1 cells: An in vitro uptake assay was used to investigate whether the bispecific binding molecules could induce uptake of AβpE3-40 by human monocytic THP-1 cells. THP-1 cells were purchased from Sigma/ECACC and cultured in RPM11640 (Gibco) supplemented with 10% FBS (Hyclone), 1× GlutaMax (Gibco), 1× Penicillin Streptomycin (Hyclone). The bispecific binding molecules and AβpE3-40 monomers were incubated together for 30 min at RT (final concentrations 25-0.1 nM for binding molecule and 50 nM for AβpE3-40). 200,000 THP-1 cells were added to the wells of a 96-well plate (Corning). Cells were pelleted by centrifugation at 300×g 5 min at RT, resuspended with the binder/AβpE3-40 monomer complexes and incubated at 37° C., 5% CO₂ for 60-120 min. Cells were washed in PBS before data acquisition using a BD FACS Lyric flow cytometer. Data was evaluated using FCS Express 4 Flow Research Edition software (De Novo Software). IC50 values were calculated using non-linear regression with the sigmoidal 4 PL equation on GraphPad Prism.

Ex vivo phagocytosis in AD brain: An ex vivo phagocytosis assay was used to investigate whether the bispecific binding molecules could induce plaque clearance by macrophages. Fresh frozen AD brain tissue was cryosectioned (20 μm) and tissue slices were collected onto poly-D-lysine (Gibco A38904-01, 50 μg/ml) coated 12 mm glass coverslips. Sections were then incubated with different concentrations of humanized antibody (0.42-6.8 nM) or an IgG1 isotype control antibody (CrownVivo, C-00012, 1 μg/ml, 6.8 nM) for 1 h at 37° C. 5% CO₂. Sections were then incubated for 24 h with 5×10⁵ to 8.5×10⁵ primary human macrophages isolated from buffy coats. Plaque clearance was evaluated using immunohistochemistry with the mouse anti-human Aβ antibodies 6E10 (Covance #SIG-39320) and 4G8 (Covance #SIG-39200) by measuring the immunopositive area on selected ROIs analyzed in consecutive sections. Experiments were repeated 7 times with macrophages isolated from different buffy coats and in 3 different patients' temporal cortex with distinct ApoE genotypes (ApoE 3/3, ApoE 3/4 and ApoE 4/4). IC₅₀ values were calculated assessing non-linear regression on GraphPad Prism and “Inhibitor vs. normalized response-Variable slope” analysis was performed.

### Results

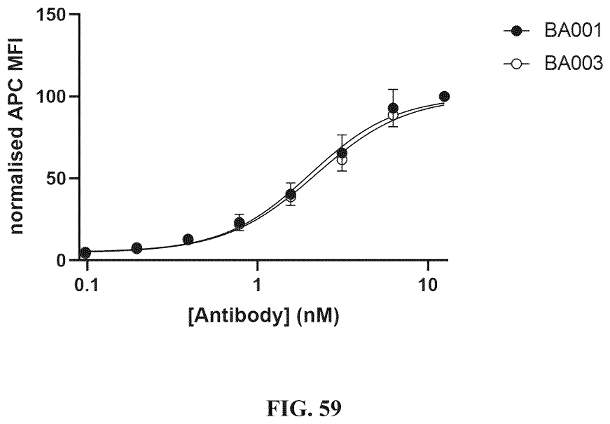

Uptake of AβpE3-40 monomers in THP-1 cells: The ability of the bispecific binding molecule BA001 and comparator antibody BA003 to induce uptake of AβpE3-40 monomers by THP-1 human monocytic cells was evaluated. The results indicate that both binding molecules can induce uptake of AβpE3-40 monomers by THP-1 cells in a concentration-dependent manner (FIG. 59). The calculated EC₅₀ values are listed in Table 40.

TABLE 40
Uptake of AβpE3-40 monomers in THP-1 cells
(Mean ± SEM, n = 3)

| Binding | |
|---|---|
| molecule | EC₅₀ (nM) |
| BA001 | 2.0 ± 0.2 |
| BA003 | 2.2 ± 0.1 |

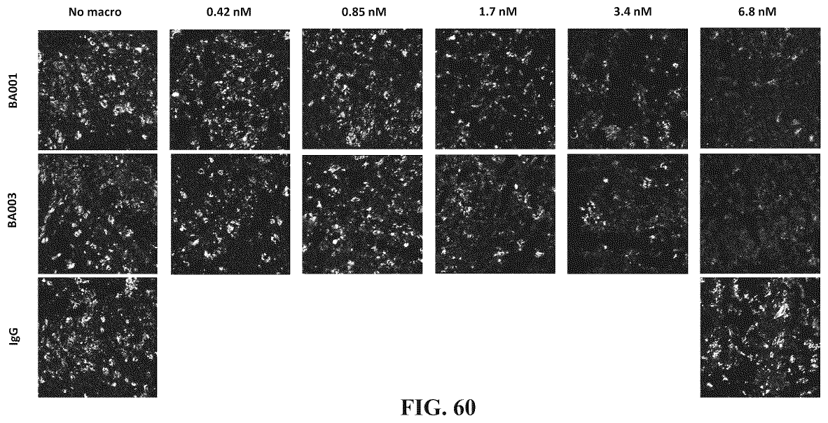

Ex vivo phagocytosis in AD brain: The ability of the bispecific binding molecule BA001 and comparator antibody BA003 to induce clearance of Aβ plaques by macrophages in AD brain was evaluated. Compared to negative control samples pre-incubated with an isotype control IgG1 antibody, Aβ plaques were reduced in a concentration-dependent manner after pre-incubation with BA001 and BA003. The results indicate that both binding molecules can induce plaque clearance by macrophages (FIG. 60). The calculated IC₅₀ values for % Aβ-immunopositive area are listed in Table 41.

TABLE 41
% Aβ-immunopositive area (Mean ± SEM, n = 7)

| Binding | % Aβ-immunopositive |
|---|---|
| molecule | area IC₅₀ (nM) |
| BA001 | 1.2 ± 0.11 |
| BA003 | 1.4 ± 0.13 |

### Example 35

### Evaluation of Immunotoxicity

This example describes the evaluation of immunotoxicity of the bispecific binding molecules generated and produced in Example 31 in cellular assays for antibody-dependent cellular cytotoxicity (ADCC), complement-dependent cytotoxicity (CDC) and in a human blood loop system.

### Materials and Methods

ADCC measurements: To investigate the effector function of bispecific binding molecules, an ADCC reporter assay with Jurkat effector cells (Promega; #G7018) was used. The cells stably express the FcγRIIIa receptor, V158 (high affinity) variant, and an NFAT response element driving expression of firefly luciferase as a measurement of ADCC activity. Antibody coated target cells bind with their antibody Fc part to FcγR on the effector cells, which triggers luciferase activity in the engineered effector cells. Ramos cells (Sigma, cat: 85030802), which express high levels of hTfR1 on the cell surface, were used as target cells. Effector and target cells were used in a ratio of 6:1, with and without serially diluted test constructs. Controls used were the BA003 antibody alone, i.e. lacking an hTfR1-binding scFv, as negative control and the monoclonal antibody rituximab (MabThera; Roche) as positive control. Target cells with test constructs were plated in a 96 well assay plate (Corning, #3917), mixed with effector cells and incubated for 18 h at 37° C. with 5% CO₂. After 18 h incubation, Bio-Glo luciferase reagent was added, and the luciferase signal was quantitated in a SPARK plate reader (Tecan). ADCC fold induction was calculated by dividing the signal obtained in the presence of indicated amount of test construct by the signal obtained in the absence of test construct.

CDC analysis: Complement activity is initiated by C1q binding to an Fc part of e.g. an antibody, which further leads to binding of other complement factors forming membrane attack complex (mac) that leads to cell death. To evaluate whether the bispecific binding molecules of the present disclosure trigger any CDC activity by enabling C1q binding to Fc part of the bound antibody, Ramos cells (Sigma, cat: 85030802) were used as target cells for CDC analysis. To measure the cell death, Ramos cells were labeled with cell viability dye, Calcein-AM (Sigma, 17783). These labeled cells were then treated with serially diluted bispecific test constructs in the presence of pooled human complement serum (Innovative Research Inc, #39337) for 4 h at 37° C., 5% C02. As positive control, rituximab was also tested. Treated cells were acquired using a BD Lyric flow cytometer (BD Biosciences). Samples were analyzed using flowJo software (BD Biosciences). The frequency of cell death was determined on calcein AM quenched gated cells and plotted against concentration of the tested antibodies or control.

Human blood loop: Blood from six healthy human volunteers (above 50 years of age) was used to investigate if and to what extent the test molecules BA001, BA002 and BA003 induced cytokine release, complement activation and cell activation in freshly collected, circulating blood. The assessment was performed using an ex vivo blood loop test system (ID.Flow; Immuneed). The binding molecules were evaluated at the five concentrations: 2 μM, 667 nM, 222 nM, 74.1 nM and 24.7 nM. Appropriate assay controls with known effects on the test parameters were included, i.e., lipopolysaccharide (LPS), alemtuzumab (anti-CD52), cetuximab (anti-EGFR) and ANC28.1 (anti-CD28). Alemtuzumab was included as a reference antibody with manageable cytokine release in the clinic by corticosteroid treatment prior to each administration. Blood parameters, including platelet (PLT), white blood cell (WBC) and red blood cell (RBC) counts, were also evaluated to assess any effects on blood cell viability.

### Results

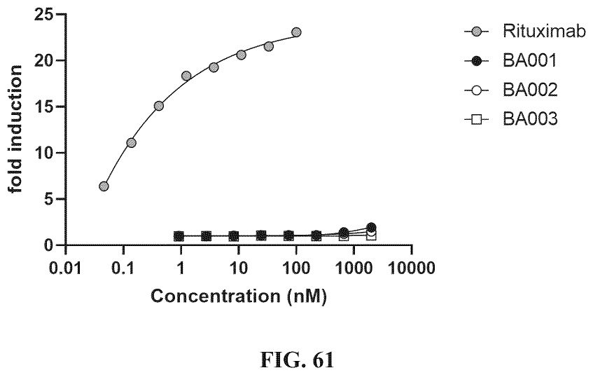

ADCC: The results are shown in FIG. 61. Rituximab is known to be a strong inducer of ADCC, and this was verified in the assay setup. No ADCC activity was observed for the bispecific binding molecules BA001 and BA002, which was similar to the negative control antibody BA003 (comparator antibody without human TfR1-binding module).

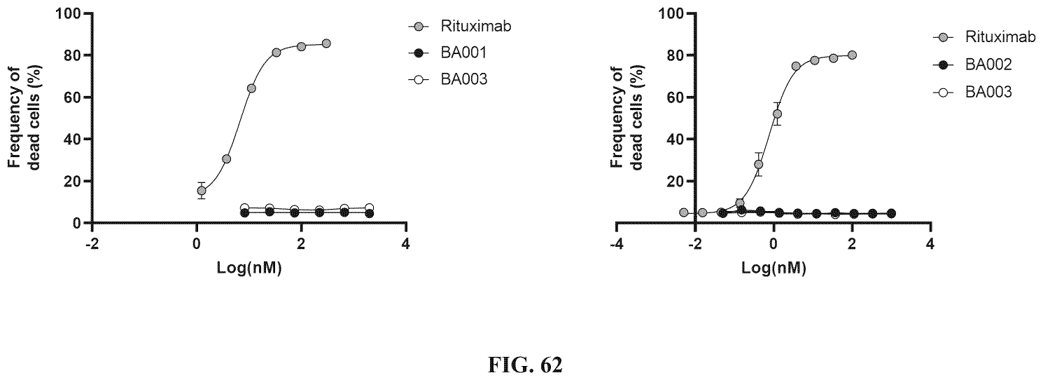

CDC: The results are shown in FIG. 62. The antibody rituximab was used as a positive control to verify CDC activity. The bispecific binding molecules BA001 and BA002 did not mediate any CDC activity, which was similar to the negative control antibody BA003 (comparator antibody without human TfR1-binding module).

Human blood loop: At all concentrations of BA001, BA002 and BA003 tested in the blood loop system, cell counts were similar to those in the vehicle group. In addition, no hemolysis or macroscopic clots were observed in any of the test item samples. Addition of BA001, BA002 or BA003 did not result in any significant cytokine release (IFN-γ, IL-2, IL-6, IL-8 and TNF) or complement activation (C3a and C5a) at any of the five concentrations tested. In addition, neither antibody had any effect on cell activation (i.e., frequency of CD69 positive T, B and NK cells, CD107a positive NK cells, CD11b positive granulocytes and CD83 positive monocytes), compared to the vehicle control. In conclusion, no effect was observed of BA001, BA002 or BA003 on cytokine release, complement activation or cell activation in circulating human blood at any of the concentrations tested.

### Example 36

### Brain and Plasma Exposure in hTfR1 Knock-In Mice

This example describes the exposure profiles of the bispecific binding molecules generated and produced in Example 31 in plasma and brain, following intravenous dosing in hTfR1-KI mice.

### Materials and Methods

To evaluate the brain and plasma exposure of bispecific binding molecules BA001 and BA002 in comparison to the control antibody BA003, brain and plasma concentrations were investigated over time in hTfR1 knock-in (hTfR1-KI) mice.

hTfR1-KI mice (see Example 20) received single intravenous (i.v.) injections of the respective binding molecules at equimolar doses of 57.4 nmol/kg (corresponding to 10 mg/kg for BA001 and BA002, and 8.4 mg/kg for BA003). Exposure in plasma and brain was assessed at 6-7 termination timepoints (n=3-4 mice/timepoint) for each binding molecule (BA001: 4, 24, 48, 72, 168 and 336 h after dose; BA002: 4, 24, 48, 72, 168 and 240 h after dose; BA003: 4, 24, 48, 72, 168, 336 and 504 h after dose). Terminal blood samples were collected from the orbital plexus into BD microtainer K2EDTA tubes from anaesthetized mice. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with ice cold PBS. Following perfusion, brains were extracted, and the olfactory bulbs removed. The brains were separated into left and right hemispheres and cerebellum was removed from the left hemisphere, after which the left hemispheres were weighed and snap frozen on dry ice and stored at −80° C. until further preparation and analysis of the concentrations of injected test constructs, using a Meso Scale Discovery (MSD) based assay.

For brain concentration measurements, frozen left hemispheres were thawed on ice and homogenized in Tris-buffered saline (TBS) containing cOmplete protease inhibitor and PhosSTOP phosphatase inhibitor (#11836145001 and #04906837001, Roche) by automated bead homogenization using MP Biomedical's FastPrep-24 5G system with Lysing Matrix D for 5 s at 6 m/s. Triton X-100 (#X₁₀₀, Merck) was added to the homogenate resulting in a final Triton X-100 concentration of 0.5% and a weight to volume ratio of 1:10. Homogenates were vortexed for 10 s and centrifuged at 16 000×g for 1 h at 4° C., after which supernatants were collected and used for brain exposure measurements.

Brain and plasma concentrations of BA001, BA002 and BA003 were determined using a custom MSD assay detecting the human Fc. A 96-well MSD plate (#L15XA-3) was coated overnight at 4° C. with 25 ng/well goat anti-human IgG, Fcγ fragment specific antibody (#109-005-098, Jackson Immuno Research Europe Ltd) diluted in 1×PBS (#09-9400-100, Medicago AB). The coat was removed, and wells were blocked with 1% Blocker A (#R93BA-4, MSD) in PBS-0.05% Tween20 (PBS-T) (#09-9410-100, Medicago AB). Following 4× wash with 1×PBS-T, the samples and test construct calibrators diluted in 1% Blocker A in PBS-T were added to plate and incubated in room temperature (RT) for 2 h at 900 rpm. Detection of bound molecules was done by sequential incubations for 1 h at 900 rpm RT with the secondary antibody (mouse anti-human IgG #3850-1-1000, MT145, Mabtech), followed by 1 h at 900 rpm RT incubation with the SULFO-TAG conjugated anti-mouse detection antibody (R32AC-1, MSD). Secondary and detection antibodies were both diluted to 25 ng/well in 1% Blocker A in PBS-T and 4× wash with 1×PBS-T was performed between all incubation steps. Following the last wash, 2× Read Buffer T (#R92TC, MSD) was added prior reading the plates in an MSD SECTOR Imager. The test construct concentration in the samples was evaluated with the MSD Discovery Workbench software, using a 4 PL curve fitting algorithm and curve weighting 1/Y2 for the corresponding test construct calibrator curve.

### Results

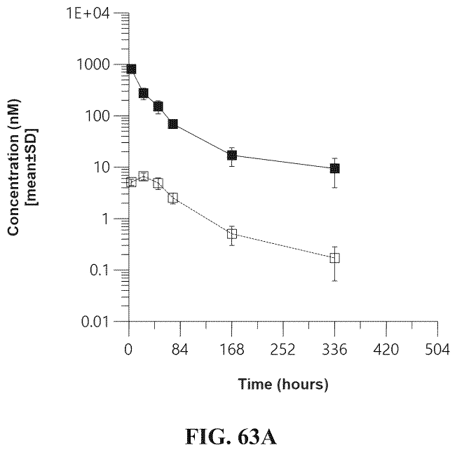

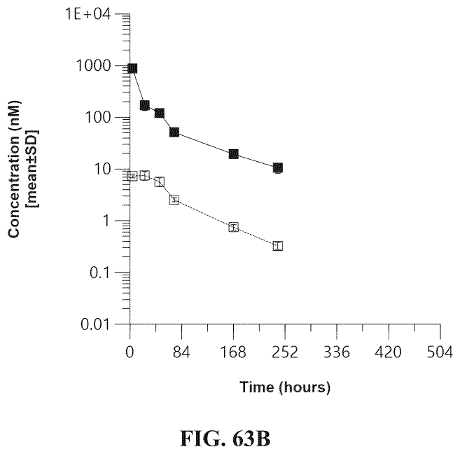

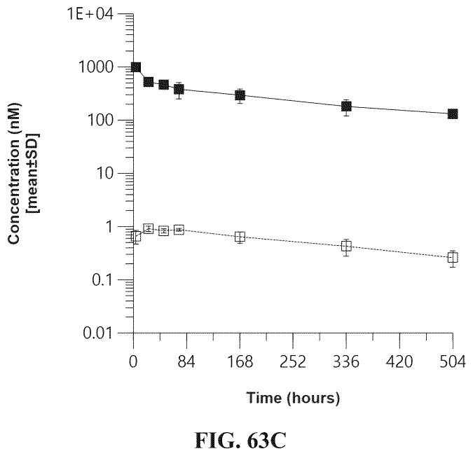

The results are shown in FIGS. 63A-63C. A higher maximum concentration in brain as well as higher brain exposure, in the form of the area under the curve, were observed over the time period studied for BA001 (FIG. 63A) and BA002 (FIG. 63B), as compared to BA003 (FIG. 63C). As shown in FIGS. 63A-63C, the plasma exposure of BA001 and BA002 was lower compared to BA003, indicating hTfR1 engagement and clearance of test constructs from the plasma to hTfR1 expressing tissues. Taken together, the data supports the conclusion that the bispecific binding molecules BA001 and BA002 undergo hTfR1-mediated BBB transport.

### Example 37

### In Vivo Target Engagement

This example describes the target engagement of the bispecific binding molecules generated and produced in Example 31 in brain, following intravenous dosing in 5×FAD×hTfR1-KI mice.

### Materials and Methods

In vivo target engagement with brain amyloid 3 pathology was investigated in two studies in crossed 5×FAD×hTfR1-KI mice. Study 1 was analyzed at 48 h post-dose, using IHC on brain sections followed by confocal microscopy. Study 2 was analyzed at 24 h and 72 h post-dose using whole-brain IHC light-sheet microscopy. The 5×FAD×hTfR1-KI mice were generated by crossing 5×FAD male mice on a C57BL/6J background (Northwestern University) with hTfR1-KI females (see Example 20). The 5×FAD mouse model is an Alzheimer's disease (AD) model with mice expressing human APP and PSEN1 transgenes with a total of five AD-linked mutations, including the Swedish (K670N/M671L), Florida (1716V), and London (V7171) mutations in APP, and the M146L and L286V mutations in PSEN1.

In study 1 (confocal microscopy of brain sections), 5×FAD×hTfR1-KI mice at 9-10 months of age (n=5 per test item) received i.v. injections of equimolar doses of 57.4 nmol/kg of BA001 (corresponding to 10 mg/kg) or BA003 (corresponding to 8.4 mg/kg). The animals were then terminated at 48 h post dose by perfusion. The abdomen of the animals was cut open and a cannula (21 G) was inserted into the left ventricle of the heart. A small cut was made in the right atrium and transcardial perfusion was performed with ice cold PBS. Following perfusion, brains were extracted, and the olfactory bulbs removed. The brains were separated into left and right hemispheres and cerebellum was removed from the left hemisphere, after which the left hemispheres were weighed and snap frozen on dry ice and stored at −80° C. The right hemispheres were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing.

In vivo engagement of amyloid β by the bispecific binding molecule BA001 and control antibody BA003 was studied using a qualitative immunohistochemistry (IHC) analysis. In brief, the right hemispheres in sucrose were embedded in O.C.T. compound (LAMB/OCT, ThermoFisher) and fast-frozen in dry ice. Embedded right hemispheres were sectioned and sagittal 20 μm slides were collected onto Superfrost cryoslides (J1800AMNZ, ThermoFisher) and air-dried prior to IHC. The brain sections were pre-treated for 20 min with 4% PFA (HL96753.1000, HistoLab, Sweden) followed by 5 min wash with dH₂O and 5 min incubation with 70% FA for antigen retrieval. After washing 2×10 min with 1×PBS, the slides were blocked with M.O.M. Mouse IgG Blocking Reagent (MKB-2213-1, Vector Laboratories) for 1 h at room temperature. Primary antibodies were diluted in 1×PBS 0.1% Triton X-100 and incubated over night at 4° C., and secondary antibodies were diluted in 1×PBS and incubated for 1.5 h at room temperature.

Amyloid β was visualized with murine antibodies 6E10 (1 μg/ml) (803002, Biolegend) and 4G8 (1 μg/ml) (800702, Biolegend) and Alexa555 anti-mouse IgG H+L (1:500) (A21424, Invitrogen). The tested compounds were visualized with Alexa647 anti-human IgG H+L (1:500) (A21206, Invitrogen). All incubations were conducted in a PBS humidified chamber. Slides were washed in 1×PBS in a cuvette (usually 5×5 min) after incubation. Sections were mounted with Fluoromount-G (00-4958-02, Invitrogen) and images were captured using a Leica Stellaris 5 confocal system equipped with a HC PL APO 10×/0.4 CS2 and a HC PL APO 40×/1.25 GLYC motCORR CS2 objectives (Leica, #15506407 and #11506423).

In study 2 (whole-brain light sheet microscopy), 5×FAD×hTfR1-KI mice at 11-12 months of age (n=2 per test item and time-point) received i.v. injections of equimolar doses of 57.4 nmol/kg of BA001 or BA002 (corresponding to 10 mg/kg) or BA003 (corresponding to 8.4 mg/kg). The animals were terminated at 24 or 72 h post dose. The animals were anesthetized with 300 mg/kg ketamine and 4 mg/kg medetomidine hydrochloride administered intraperitoneally. Plasma was prepared by centrifugation and stored at −80° C. until analysis. After blood sampling, the animals were kept on mask under deep isoflurane anesthesia until confirmed euthanized through perfusion. The perfusion protocol followed the guidelines for intracardiac perfusion of mouse with paraformaldehyde (PFA) for light sheet microscopy. Following 2 min perfusion with heparinized (15,000 IU/I) PBS for 2 min at a rate of 20-25 ml/min, the input was switched to 4% PFA for approximately 5 min or until PFA induced curving of the tail and stiffening of leg muscles. After fixation, the brain was dissected out. The presence of blood in brain tissue after perfusion was estimated by visual inspection of the intact brain after extraction. The brain was immersion-fixed overnight in PFA at 4° C., and then washed 3×30 min in PBS while shaking and stored until shipping at 4° C. in PBS containing 0.02% sodium azide.

Following brain isolation and fixation, the samples were washed for 1 h in PBS and dehydrated in methanol/H₂O gradient: 20%, 40%, 60%, 80% and 100% methanol, each step 1 h at RT. They were further washed in 100% methanol for 1 h and incubated overnight in 66% Dichloromethane (DCM)/33% methanol at RT. The next day the samples were washed twice in 100% methanol for 30 min, cooled down to 4° C. in 1 h and bleached in chilled fresh 5% H₂O₂ in methanol (1 volume 35% H₂O₂ to 6 times volume methanol) overnight at 4° C. The samples were subsequently rehydrated in a methanol/PBS series: 80%, 60%, 40%, 20%, with 0.2% Triton X-100, 1 h each at RT and washed in PBS with 0.2% Triton X-100 for 2×1 h at RT. The brains were then incubated in permeabilization solution at 37° C. for 3 days. Blocking was carried out in blocking solution at 37° C. for 2 days.

The samples were incubated with the primary antibody Rabbit anti-human Amyloid Beta (IBL, 18584) in PBS/Tween/Heparin buffer (PTwH), 5% DMSO, and 3% donkey serum at 37° C. for 11 days. Next, they were washed in PTwH for 1×10 min, 1×20 min, 1×30 min, 1×1 h, 1×2 h and 1×2 days. The following secondary antibodies were used: donkey anti-rabbit IgG-Cy5 (Jackson ImmunoResearch, 711-175-152), goat-anti-CD31-564 (R&D Systems, AF3628; direct conjugated) and donkey anti-human IgG-790 (Jackson ImmunoResearch, 709-655-149). Samples were incubated with secondary antibodies in PTwH, 3% donkey serum at 37° C. for 20 days, followed by washes in PTwH: 1×10 min, 1×20 min, 1×30 min, 1×1 h, 1×2 h and 1×3 days. All steps were performed in tightly closed tubes to minimize evaporation and oxidation.

The immunostained brains were then cleared in a methanol/H₂O series: 20%, 40%, 60%, 80% and 100% for 1 h each at RT, and then incubated in 100% methanol overnight and next day for 3 h (with shaking) in 66% DCM and 33% methanol at RT and in 100% DCM 15 min twice (with shaking) to remove traces of methanol. The samples were finally transferred to ethyl cinnamate and stored in closed glass vials in the dark.

Brain samples were imaged using Luxendo LCS SPIM microscope (Bruker Corporation) with a 4× C objective at the following wavelengths: CD31 at 546 nm, Aβ plaques at 647 nm, and hIgG (test items) at 790 nm. Ethyl cinnamate was used as clearing agent during acquisition of data. Imaris/Aivia software (Oxford Instruments) was used for 3D visualization of data. Customized software (Gubra) was used for image analysis. The Aβ, hIgG and CD31 specific channels were used for segmentation and mapped to the CF-LSFM_v6 atlas. The total signal and number of plaques were quantified in all anatomical regions using the Aβ and hIgG specific channels. In addition, the hIgG signal was segmented into a CD31 positive and CD31 negative compartment and the signal of the two compartments was quantified in all brain regions. To compare the respective average intensities between the various regions, the total hIgG signal from each brain region was divided by the volume of the respective region to yield the average signal intensity per voxel for each respective region.

### Results

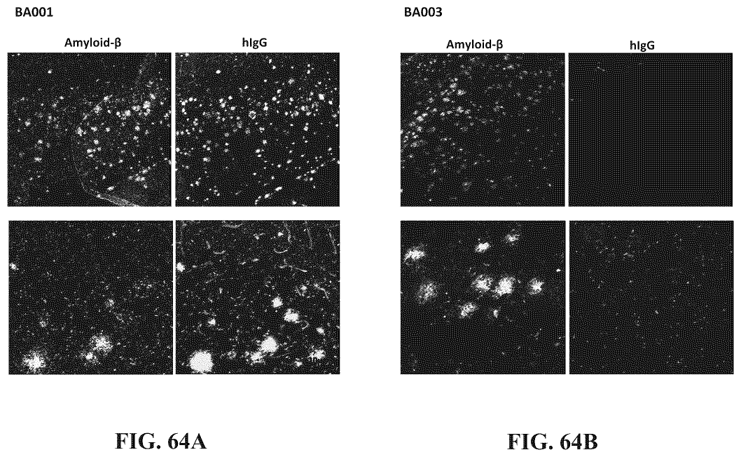

Representative confocal IHC images from study 1 are shown in FIGS. 64A-64B. Co-localization of hIgG and amyloid β antibodies was observed, illustrating extensive amyloid β target engagement for BA001 in 5×FAD/hTfR1-KI brain at 48 h post-dose (FIG. 64A). Core plaques were predominantly positive for BA001 while a low amyloid β plaque co-localization was seen for the BA003 comparator antibody lacking the hTfR1-binding module (FIG. 64B).

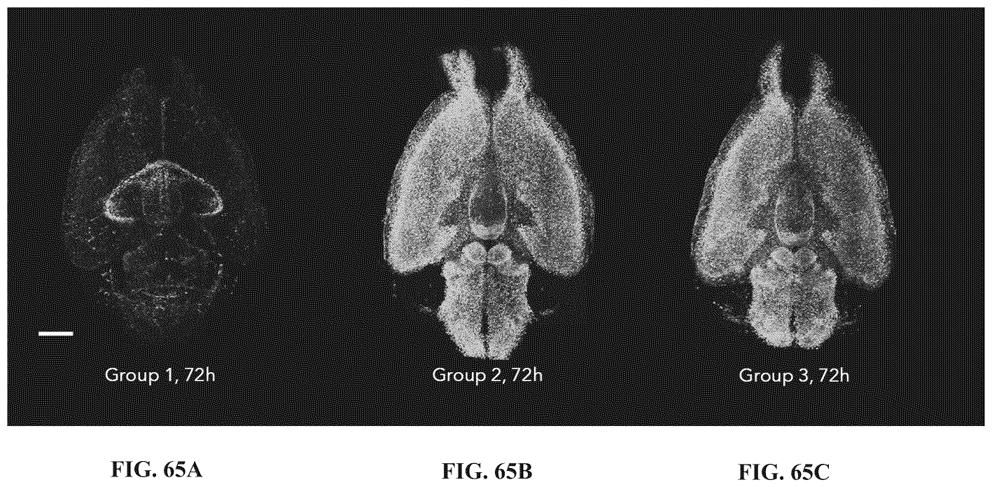

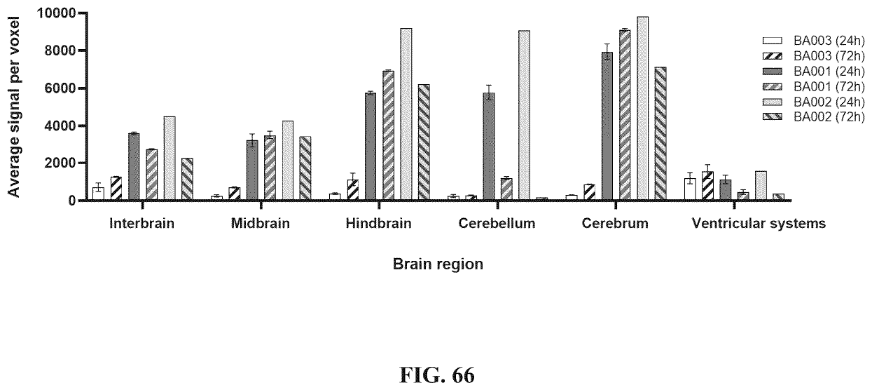

Representative whole-brain light sheet microscopy images from study 2 are shown in FIGS. 65A-65C. Extensive amyloid β plaque target engagement for BA001 and BA002 was demonstrated in whole-brain in 5×FAD×hTfR1-KI brain at 72 h post-dose (FIGS. 65B-65C). Comparator antibody BA003 was most prominent in the ventricular system and blood vessels, with significantly less target engagement (FIG. 65A). Quantification of hIgG signal in select brain regions following light sheet 5 microscopy analysis in 5×FAD×hTfR1-KI is shown in FIG. 66. In BA003 treated mice, the hIgG average staining signal per voxel increased over time with a roughly 2- to 3-fold increase at 72 h versus 24 h. In BA001 and BA002 dosed mice, the average staining signal per voxel increased in general 15- to 20-fold in all regions, except for the ventricular systems, compared to BA003 dosed mice. The average staining signal per voxel diminished in the ventricular systems at 72 h versus 24 h in mice dosed with BA001 or BA002.

### Example 38

### In Vivo Efficacy

This example describes the in vivo efficacy of the bispecific binding molecule BA001 generated and produced in Example 31, following one-month i.v. dosing in 5×FAD×hTfR1-KI mice.

### Materials and Methods

In vivo efficacy was evaluated in 5×FAD×hTfR1-KI transgenic heterozygous male mice treated for one month with BA001. BA001 at 10 mg/kg or vehicle (PBS) was administered i.v. via the tail vein to 6.5-month-old 5×FAD×hTfR1-KI male mice (n=10-12), every 4 days for 4 weeks (7 injections in total). At 72 h after the final injection (7), at an age of 7.5 months, the animals were anaesthetized using isoflurane and perfused with PBS, as described in Example 37. The brains were separated into left and right hemispheres. Cerebellum and olfactory bulb were removed from the left hemisphere, which was weighed and stored at −80° C. until biomarker analysis. Left brain hemispheres were homogenized in TBS buffer using the FastPrep-24 high-speed benchtop homogenizer, resulting in a weight to volume (w/v) ratio of 1:10. Homogenates were centrifuged 1 h at 4° C. and 16 000×g. The resulting pellets were dissolved in 70% formic acid (FA) at a w/v ratio of 1:5 and centrifuged at 100 000×g for 1 h at 4° C. and the supernatants were defined as the insoluble brain extracts. Insoluble brain extracts were neutralized to pH 7.0±0.5 in Trizma base/Na₂HPO₄ buffer prior to downstream analysis of AβpE3 and Aβ levels.

Aβ40 and Aβ42 levels in the insoluble fraction were determined using the V-PLEX Aβ Peptide Panel 1 (6E10) Kit (MSD, K15200E). Neutralized insoluble brain extracts were diluted in assay diluent to final dilutions of 1:16 000 (Aβ38, Aβ40 and Aβ42). Samples and SULFO-TAG anti-human Aβ antibody (6E10) were co-incubated on MSD multi-spot plates pre-coated with anti-Aβ38, Aβ40 and Aβ42 antibodies in independent spots. Following the addition of read buffer, plates were read in an MSD SECTOR imager. The signal strength in the defined spots were correlated to the amount of the corresponding Aβ peptide in the samples.

AβpE3-40 levels in the insoluble fraction were determined using a Human Amyloid β (N3pE-40) ELISA Assay Kit (IBL, 27418). In brief, human AβpE3-40 standard, and neutralized insoluble brain extracts were diluted in assay buffer to a final dilution of 1:180 and incubated over night at 4° C. to facilitate binding to microtiter plate pre-coated with a mouse anti-Aβ35-40 capture antibody. Following a washing step to remove unbound proteins, bound Aβ peptides were incubated with a horseradish peroxidase (HRP) conjugated mouse anti-AβpE3-x mAb (8E1) followed by the addition of 3,3′,5,5′-tetramethylbenzidine (TMB) as a substrate to detect the bound antibody. The reaction was stopped by the addition of 0.5 M H₂SO₄, resulting in a yellow color for which optical density (OD) was measured using a microplate reader at 450 nm. The blank-subtracted average OD450 was correlated to the amount of AβpE3-40 in each sample. Student's t-test was used for statistical comparison of AβpE3-40 normalized to Aβ40.

### Results

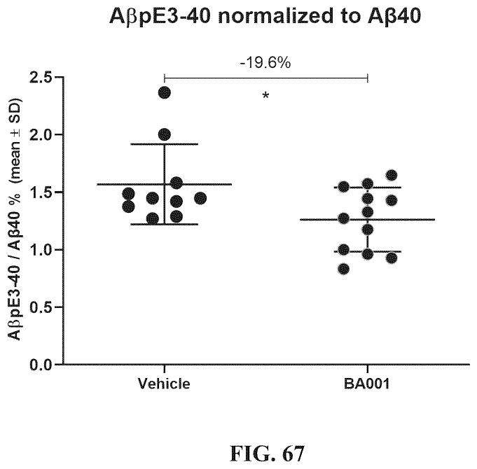

In vivo efficacy of BA001 in 5×FAD×hTfR-KI mice was analyzed following one-month dosing (10 mg/kg BA001, i.v. every 4 days). Treatment with BA001 resulted in a statistically significant reduction of AβpE3-40 levels normalized to total Aβ40 (−20%, p=0.03, Student's t-test), compared to vehicle (FIG. 67).

### Example 39

### Evaluation of Competition with Transferrin and Ferritin

This example describes the competition of the bispecific binding molecules generated and produced in Example 31 with transferrin and ferritin binding to hTfR1.

### Materials and Methods

In order to evaluate the binding competition between transferrin and the disclosed bispecific binding molecules, K562 lymphoblast cells (Sigma/ECACC) were first incubated with human Fc block (BD Pharmingen, 564220) for 15 min at 4° C., to block non-specific Fc Receptor-mediated antibody binding. The cells were subsequently incubated with serially diluted test constructs along with a fixed concentration of Alexa Fluor 647 conjugated, human holo-transferrin (Invitrogen/ThermoFisher, T23366) for 30 min at 4° C. After the incubation, the cells were washed 3 times in staining buffer (1% BSA, 0.1% Sodium Azide in 1×DPBS). Transferrin bound to hTfR1 on cell surfaces was captured using a BD Lyric flow cytometer (BD Biosciences). The samples were analyzed using FlowJo software (BD Bioscience) and the mean fluorescence intensity of bound transferrin was plotted using GraphPad Prism software (GraphPad Software Inc).

In order to evaluate potential binding/uptake competition between H-Ft (heavy-chain ferritin) and the disclosed bispecific binding molecules, K562 cells were used. Fc-blocked cells (2e5 cells/ml) were added to a 96-well V-bottom plate and subsequently incubated with serially diluted test constructs along with a fixed concentration of Alexa Fluor 647 labelled H-Ft for 1 h at 4° C. for assessment of binding interference. For assessment of uptake interference, cells were incubated with 500 nM test compound and a fixed concentration of Alexa Fluor 647 labelled H-Ft in pre-warmed incomplete RPMI for 1 h at 37° C. In both conditions, after the incubation period, cells were washed twice with cold staining buffer and fixed with 4% PFA in PBS for 15 min at 4° C. Following fixation, the cells were washed and resuspended in staining buffer and acquired on a BD Symphony A1 flow cytometer. Anti-CD71 clone M-A712 (BD Biosciences) was used as a positive control for interference with H-Ft binding to hTfR1, and “Ferritin only” samples were used as reference control. The samples were analyzed using FlowJo software (BD Bioscience) and the mean fluorescence intensity of bound H-Ft was plotted using GraphPad Prism software (GraphPad Software Inc).

### Results

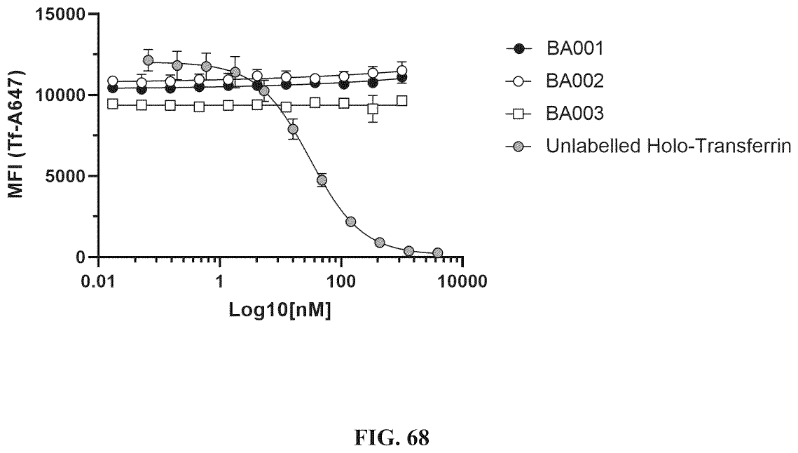

FIG. 68 shows that there is no binding competition to TfR1 between the bispecific binding molecules BA001 or BA002 and transferrin, similar to the negative control antibody BA003 (comparator antibody lacking human TfR1-binding module). When non-labeled holo-transferrin was used as positive control for competition, the signal from binding of labeled (A647) transferrin was reduced in a concentration dependent way. The experiment illustrates that bispecific binding molecules BA001 and BA002, partly directed against hTfR1, do not compete directly for the same epitope as transferrin and does not negatively affect the ability of the endogenous ligand transferrin to bind to the receptor.

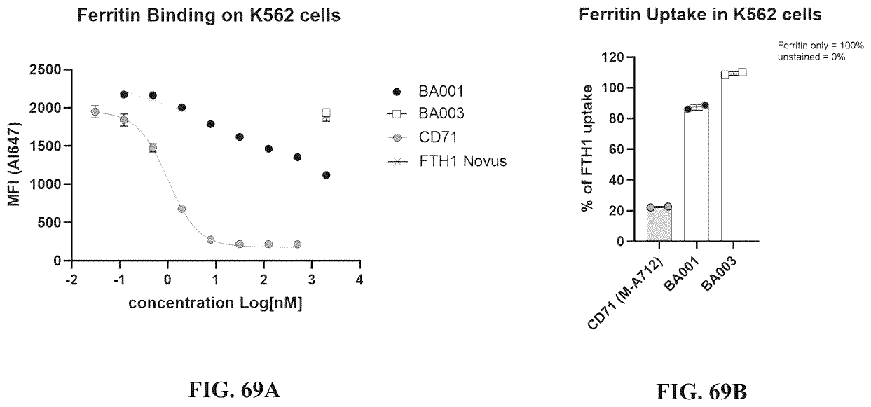

BA001 shows a partial dose-dependent binding interference between H-Ft on K562 cells in vitro (FIG. 69A). However, the uptake of H-Ft was only marginally affected, and close to 90% of H-Ft was taken up by the cells (FIG. 69B). Control anti-CD71 antibody M-A712 showed a strong H-Ft binding interference, whereas BA003 had no impact on H-Ft (FIG. 69A). Incubation with control anti-CD71 antibody M-A712 resulted in only 20% of H-Ft uptake, whereas BA003 had no impact on H-Ft uptake in K562 cells (FIG. 69B).

### Example 40

### In Vivo Hematology Analysis

This example describes the analysis of blood reticulocytes in hTfR1-KI mice, following i.v. dosing of the bispecific binding molecule BA001 and antibody BA003.

### Materials and Methods

Hematology analysis was conducted in hTfR1-KI mice following a single i.v. injection of BA001 10 mg/kg or equimolar dose of BA003 8.4 mg/kg (57 nmol/kg, n=5/group). Blood was collected in K3 EDTA tubes at 7 days prior to i.v. injection from the saphenous vein, and 24 h post i.v. dosing from the orbital plexus of all animals. Within 4 h after blood sampling, a 20 μl sample was diluted with 120 μl of cellpack DCL (1:7 dilution) and analyzed with the Sysmex XN-1000 instrument.

### Results

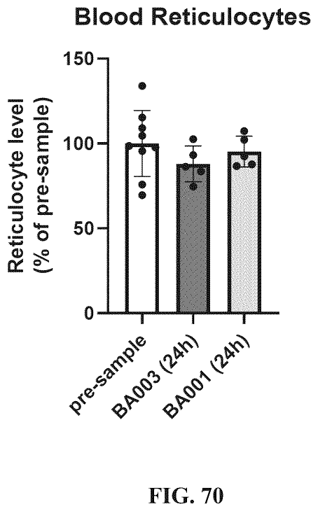

Hematological analysis revealed no overall changes in either the white or red blood cell compartment, platelets, hemoglobin or reticulocytes following i.v. dosing with bispecific antibody BA003 or control antibody BA001. No impact (e.g. depletion) on reticulocytes at 24 h was observed (FIG. 70).

### Example 41

### Evaluation of Brain Uptake in Non-Human Primates

This example describes exposure in plasma and brain of the bispecific binding molecules generated and produced in Example 31 in non-human primates after single intravenous (i.v.) dosing.

### Materials and Methods

Cynomolgus monkeys (n=4 females per group) received a single i.v. bolus injection of BA001 or BA002 at 10 mg/kg, or BA003 at 19 mg/kg. General in-life observations included mortality, cage-side observations, post-dose observations, detailed clinical observations, individual body weights, food consumption, body temperature, electrocardiography and blood pressure. Blood was collected at different time points for hematology, coagulation, clinical chemistry, cytokine/chemokine and complement factor analysis and for concentration measurements of test constructs. Hematology, coagulation and clinical chemistry were analyzed in a pre-test period (at least 7 days before dosing day), pre-dose and at 23 h after dose. Cytokines and complement factors were analyzed at pre-dose and 2 h after dose. Test construct concentrations in plasma were analyzed at 24 h after dose. At termination (24 h after dose), the animals were deeply sedated and subjected to a full body perfusion with PBS by the intracardiac route. The brains were collected, and the left hemisphere was further dissected into parietal cortex, hippocampus, striatum and cerebellum.

Homogenization and extraction of brain tissues were performed using the following process: samples from individual brain regions were added to TBS extraction buffer (w/v 1:5) in lysing matrix D tubes and allowed to thaw for about 20 min. Homogenization was performed by using a FastPrep homogenizer at a speed of 6 m/s for 5 s. Homogenates were transferred to 1.5 ml protein polypropylene LoBind tubes. An equal volume of 1% TBS-T extraction buffer (TBS w. 1% Triton X-100) was added to the homogenate and mixed thoroughly, resulting in a final triton concentration of 0.5%. Samples were then centrifuged for 60 min 4° C. at 16 000×g. Supernatants (w/v 1:10), were aliquoted in protein polypropylene LoBind tubes and stored at −80° C. prior to analysis.

Analysis of the test construct concentrations in plasma and brain tissue was performed according to qualified methods. An electrochemiluminescence (ECL) ligand binding assay was used in which an anti-human IgG antibody (Bethyl Laboratories) was coated at 1.0 μg/ml in a multi-array 96 well standard MSD plate. After a blocking step, samples were added and test construct in the sample bound to the coated antibody. A biotin-labeled Goat Anti-Human IgG antibody (Southern Biotech) was used at 0.05 μg/ml to detect the captured test construct. After washing, addition of SULFO-TAG-labeled streptavidin (0.5 μg/ml) completed the binding complex to allow for detection. After incubation and washing, read buffer 2× was added to the plate. Relative light units (RLU) values were measured using the MSD MESO Quickplex SQ 120. The minimal required dilution (MRD) of the method was 1:100 for plasma and 1:20 for brain tissue. Concentrations were then determined by interpolation from a calibration curve in which RLU values were plotted against test construct concentrations of calibration standards using a 5-parameter logistic (PL) non-linear regression model (weighting factor 1/Y2, no blank subtracted).

### Results

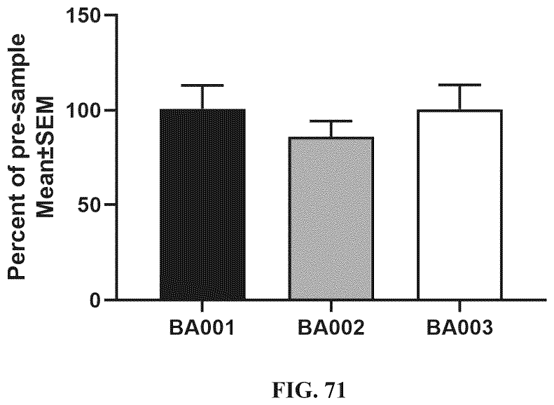

There were no effects related to the test constructs on mortality, clinical signs, body weight, food consumption, body temperature, electrocardiography or blood pressure after i.v. dosing of each respective construct up to the 24 h termination timepoint. Similarly, no test construct-related effects were observed on hematology, coagulation, clinical chemistry, cytokines or complement factors. Reticulocyte counts after dosing were normalized to the pre-dose samples and the mean values are shown in FIG. 71. No changes in reticulocyte counts were observed in cynomolgus monkey at 23 h post dose for any of the test constructs. Additionally, there was no major impact on red blood cell mass parameters (red blood cell counts, hematocrit and hemoglobin concentration) in any of the groups, at 23 h post dosing.

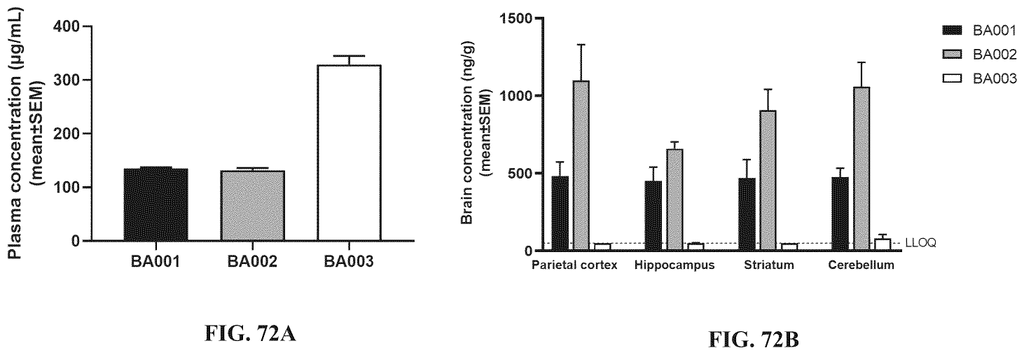

The mean concentrations of BA001, BA002 and BA003 in plasma at 24 h after dose are shown in FIG. 72A. A higher plasma exposure was observed for the control compound BA003 compared to the bispecific binding molecules BA001 and BA002, and this was also evident after normalization to injected dose. The mean concentrations of BA001, BA002 and BA003 in different brain regions (parietal cortex, hippocampus, striatum and cerebellum) were measured at 24 h after dose and are shown in FIG. 72B. A higher exposure was observed in all brain regions for BA001 and BA002 compared to BA003, despite an almost 2-fold higher dose given for BA003. The data provides proof of increased brain uptake of the bispecific binding molecules BA001 and BA002 in cynomolgus monkey.

### Example 42

### Analysis of PE-ADA in Human Serum

### Materials and Methods

Serum samples: Serum was obtained from healthy donors giving blood during 2023-2025 (ethical permission D-nr 2018/804-31 from the regional ethics board in Stockholm, Sweden).

Bridging assay: A bridging assay was set up as follows to detect the potential reactivity to PE-ADA for the test compound BA001 and comparator antibody BA003. One part of the respective protein was biotinylated using a standard biotinylation kit (A39257, Thermo Scientific), and another part was labelled with SULFO™ tag according to the manufacturers' instructions (R91AO-1, Mesoscale). Serum from 107 donors to a final concentration of 2% was incubated with sulfo-tagged and biotinylated protein (3 nM each, final concentration) in 1% Blocker A (R93BA-4, Mesoscale) for 2 h at room temperature with 900 rpm shaking to allow for complexes to form. 25 μl of the mix was added to the wells of 1% Blocker A pre-blocked and washed MSD Gold 96-well small spot streptavidin plates (L45SA-1, Mesoscale). The plates were incubated for 1 h at room temperature with 900 rpm shaking to allow for the biotinylated antibody to bind to streptavidin on the plate. The plates were washed and 150 μl MSD Read Buffer T (2×) (R92TC-1, Mesoscale) was added. The plates were immediately read using a Meso Sector S 600 (Mesocale). Electrochemiluminescence (ECL) counts were plotted for all individuals and assays.

Confirmatory competition assay: Serum (final concentration 2%) from 107 donors was incubated with sulfo-tagged and biotinylated antibody preparations as described for the bridging assay above (3 nM each, final concentration) in 1% Blocker A (R93BA-4, Mesoscale) with or without 500× molar excess of the hTfR1-binding scFv molecule “h26D3-HC6_DS, VL-first” (SEQ ID NO:154), (final concentration 1500 nM) for 2 h at room temperature with 900 rpm shaking to allow for complexes to form. 25 μl per well of the mix was added to the wells of 1% Blocker A pre-blocked and washed MSD Gold 96-well small spot streptavidin plates (L45SA-1, Mesoscale). The plates were incubated for 1 h at room temperature with 900 rpm shaking to allow for the biotinylated antibody to bind to streptavidin on the plate. The plates were washed and 150 μl MSD Read Buffer T (2×) (R92TC-1, Mesoscale) was added. The plates were immediately read using a Meso Sector S 600 (Mesocale). ECL counts were plotted for all individuals and assays.

### Results

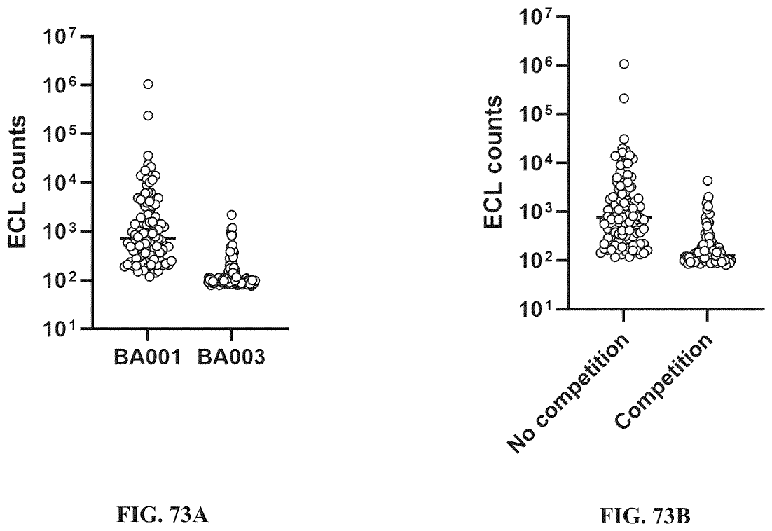

The results from the bridging assay are plotted in FIG. 73A and show that the reactivity from PE-ADA in human serum samples was observed at a higher frequency and at higher ECL-counts for the bispecific binding molecule BA001, as compared to the comparator antibody BA003.

Further, in the confirmatory competition assay, it was demonstrated that the majority of the observed PE-ADA reactivity is directed to the hTfR1 binding module of the bispecific binding molecule BA001 (FIG. 73B), as the majority of the PE-ADA response from serum samples was reduced when subjected to competition by the hTfR1-binding scFv (“Competition”) compared to the non-competed setting (“No competition”).

### Example 43

### Design, Production and SEC Analysis of Additional Stabilized hTfR1 Binding Molecules

In the single-chain variable fragment (scFv) format, hydrophobic patches within the former V/C interface of the full-length antibody become exposed (Nieba et al (1997), Prot Eng 10(4):435-444). These newly accessible hydrophobic areas, particularly within the VH domain, can serve as binding sites for preexisting anti-drug antibodies (PE-ADA) when the scFv, or construct comprising it, is administered to a subject (Holland et al (2013), J Clin Immunol 33:1192-1203). Previous work has shown that mutation of certain hydrophobic residues within the former V/C interface of the VH domain to more hydrophilic residues leads to a reduction of such potential PE-ADA reactivity (Johansson et al (2023), mAbs 15(1):2215887).

Eleven variants of the disulfide-stabilized VH domain of h26D3-HC6 were designed, containing either single or double substitutions of hydrophobic amino acid residues to more hydrophilic and/or less bulky amino acids residues. The variants are listed in Table 42. All VH domain variants were expressed in the form of scFv domains having the VL first orientation. A flexible (G₄S)₄ linker connects the VL domain to the VH domain within each scFv. For purification purposes, a His₆ tag and (G₄S)₄ linker was added to the N-terminus of the VL domain (combined tag sequence given by SEQ ID NO:158).

TABLE 42
VH variants of the hTfR1 binding scFv
molecule h26D3-HC6_DS, VL-first

| | VH AA | scFv AA |
|---|---|---|
| | sequence | sequence |
| Designation | (SEQ ID NO:) | (SEQ ID NO:) |
| h26D3 HC6 DS S122K | 168 | 178 |
| h26D3 HC6 DS T120K | 169 | 179 |
| h26D3 HC6 DS L118S | 177 | 187 |
| h26D3 HC6 DS T91S S122K | 167 | 180 |
| h26D3 HC6 DS T91S T120K | 171 | 181 |
| h26D3 HC6 DS T91A | 174 | 184 |
| h26D3 HC6 DS S16E L118S | 175 | 185 |
| h26D3 HC6 DS S16E | 176 | 186 |
| h26D3 HC6 DS V11R T120K | 170 | 182 |
| h26D3 HC6 DS V11R T91A | 172 | 183 |
| h26D3 HC6 DS V11R | 173 | 188 |

These eleven scFv variants were produced in CHO cells and purified with IMAC plus preparative SEC as described in Example 24. The SEC fractions containing monomeric scFv were collected, sterile filtered and brought to 1 mg/ml final concentration in PBS, pH 7.4. The recovered molecules were analysed by CE-SDS, reducing and non-reducing, by loading 2.5 μg of respective molecule on a LabChip (GXII Touch™ HT Chip, PerkinElmer). Samples were prepared with the ProteinEXact Assay Reagent Kit (PerkinElmer). Instrument Assay used: LabChip GXII Touch HT Protein Characterization System (Perkin Elmer). Sample treatment: Before loading. samples were treated with DTT for reduced conditions, and both non-reduced and reduced samples were heated at 70° C. for 10 min. The results showed bands of expected size and high purity for all molecules.

Each scFv variant produced was then injected onto an SEC column and analyzed as described in Example 25. The results of the analytical SEC experiment are shown in Table 43 and demonstrate that all variants of the molecule were isolated with a high monomer content, similar to the DS-stabilized scFv molecules produced in Example 25.

TABLE 43
Results from aSEC of hTfR1 binding scFv variants

| | Main peak | Main |
|---|---|---|
| | retention | peak |
| scFv variant | time (min) | area (%) |
| h26D3 HC6 DS S122K | 10.905 | 99.85 |
| h26D3 HC6 DS T120K | 10.909 | 99.85 |
| h26D3 HC6 DS L118S | 10.910 | 99.85 |
| h26D3 HC6 DS T91S S122K | 10.910 | 99.86 |
| h26D3 HC6 DS T91S T120K | 10.914 | 99.87 |
| h26D3 HC6 DS T91A | 10.905 | 99.88 |
| h26D3 HC6 DS S16E L118S | 10.903 | 100.00 |
| h26D3 HC6 DS S16E | 10.902 | 100.00 |
| h26D3 HC6 DS V11R T120K | 10.922 | 99.84 |
| h26D3 HC6 DS V11R T91A | 10.919 | 99.88 |
| h26D3 HC6 DS V11R | 10.923 | 99.89 |

### Example 44

### Thermal Stability of Additional Variants of hTfR1 Binding Molecules

From the production and analysis of hTfR binding molecules in Example 43, six variant scFv:s (SEQ ID NO:178-183) were selected and subjected to a thermal stability study. Monomer stability of scFv samples was evaluated by HPLC SEC analysis. The scFv molecules, purified and stored in PBS, were subjected to temperature hold for one, two or four weeks at temperatures 4° C., 25° C., 40° C. and frozen at −75° C. At each timepoint, samples of each variant from each temperature were analyzed by HPLC-SEC as described in Example 43. At the initiation of the study, frozen samples were thawed and analyzed, and denoted “TO”. In addition, for each of the six molecules a sample was subjected to freeze-thaw stress. Molecules were subjected to 3× freeze-thaw cycles. Freezing was done at −75° C. for at least 16 h and thawing was done at ambient temperature until complete thawing of the sample (up to 60 min). Samples are denoted FT_3× and were analyzed immediately after the third freeze-thaw cycle completion.

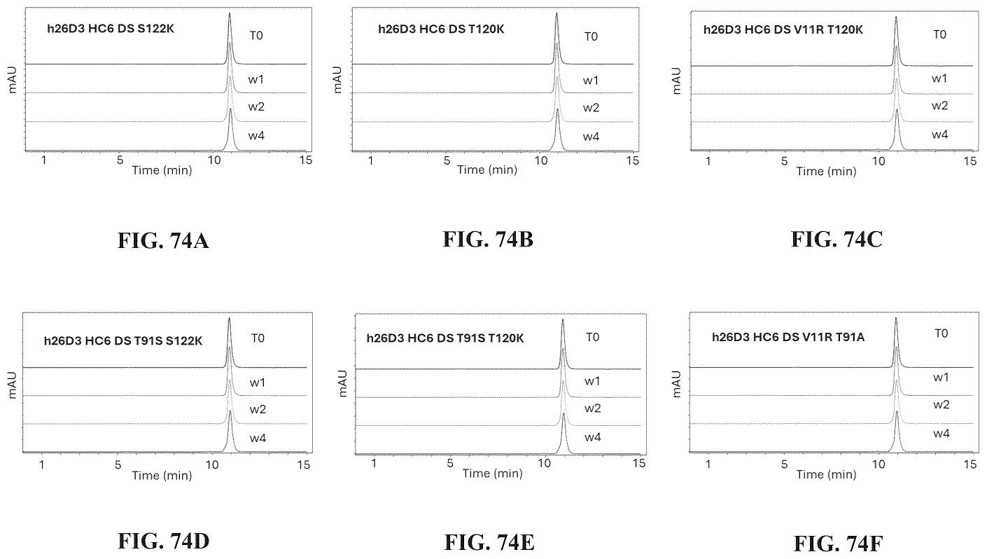

All six scFv molecules were stable monomers at the tested conditions. The results for the six selected scFv molecules from samples collected as TO or after temperature hold at 40° C. for 1-4 weeks respectively are shown as stacked chromatograms in FIGS. 74A-74F. The main peak for all samples has a retention time at ˜11 min, as expected for a monomeric scFv. All samples have a high monomer content at all tested conditions, demonstrating that the stabilized DS design is maintained in all six variants.

### Example 45

### Immunogenicity in Silico of Additional Variants of hTfR1 Binding Molecules

The six scFv sequences analyzed in Example 44 (SEQ ID NO:178-183) and the variant “h26D3 HC6 DS, VL-first” (SEQ ID NO:154; Example 24) were subjected to an in silico analysis of immunogenicity risk, based on identification of potential T cell epitopes in the respective amino acid sequence. The assessment was done using iTope-AI (Abzena Ltd., Cambridge, UK). In addition, the sequences were analysed for homology to known T cell epitopes, previously identified by ex vivo EpiScreen™ analysis of other protein sequences. Homology scores against known T cell epitopes (Bryson et al (2010), BioDrugs 24(1):1-8) are shown as TCED in Table 44.

The immunogenicity assessment of the respective protein sequence was performed using overlapping 9mer peptides, tested against 46 MHC class II allotypes used in the iTope-AI platform. Individual peptides, together spanning the whole sequence, were given a binding score from 0 to 3 for each allotype, and those scores were added together to provide a “Position Risk Score”. The “Total Score” for the respective test protein in Table 44 was calculated by adding the “Position Risk Scores” obtained for all individual peptides. The highest “Position Risk Score” for each sequence was denoted “Hotspot Max” as also shown in Table 44.

Taken together, all seven tested protein sequences show very similar results, indicating that no novel risk sites of concern were identified in any of the assessed variants of the TfR binding molecule.

TABLE 44
In silico immunogenicity assessment
of hTfR1-binding scFv molecules

| | SEQ | Total | Hotspot | TCED |
|---|---|---|---|---|
| scFv variant | ID NO: | Score | Max | Homology |
| h26D3 HC6 DS, VL-first | 154 | 102 | 19 | 6 |
| h26D3 HC6 DS V11R T91A | 183 | 134 | 24 | 7 |
| h26D3 HC6 DS V11R T120K | 182 | 105 | 19 | 6 |
| h26D3 HC6 DS T91S T120K | 181 | 117 | 19 | 7 |
| h26D3 HC6 DS T91S S122K | 180 | 116 | 19 | 7 |
| h26D3 HC6 DS T120K | 179 | 103 | 19 | 6 |
| h26D3 HC6 DS S122K | 178 | 102 | 19 | 6 |

### Example 46

### SPR Analysis of Binding of Additional scFv Variants to hTfR1 and cTfR1

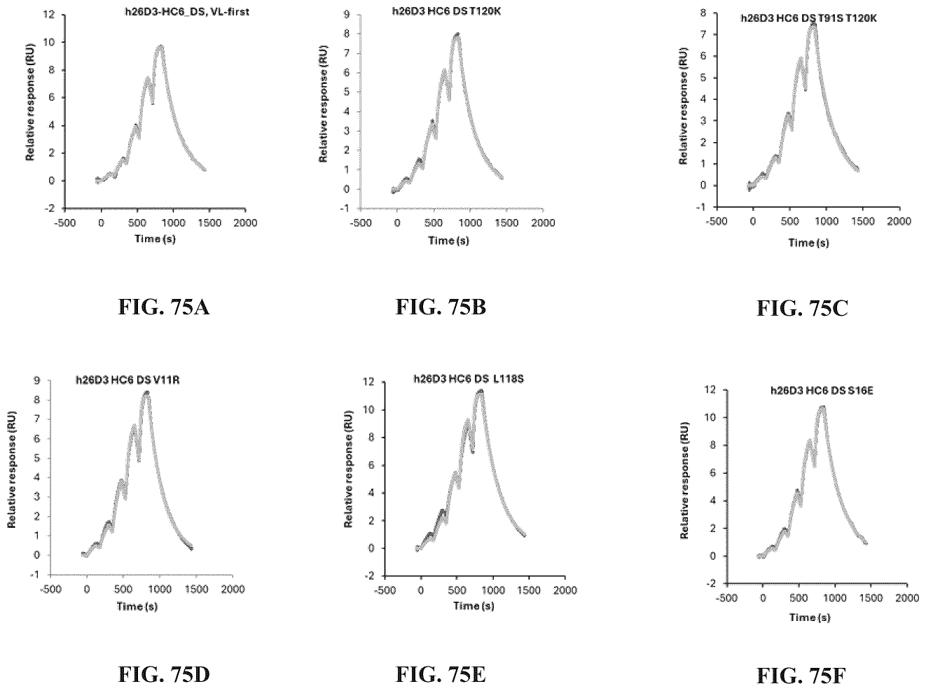

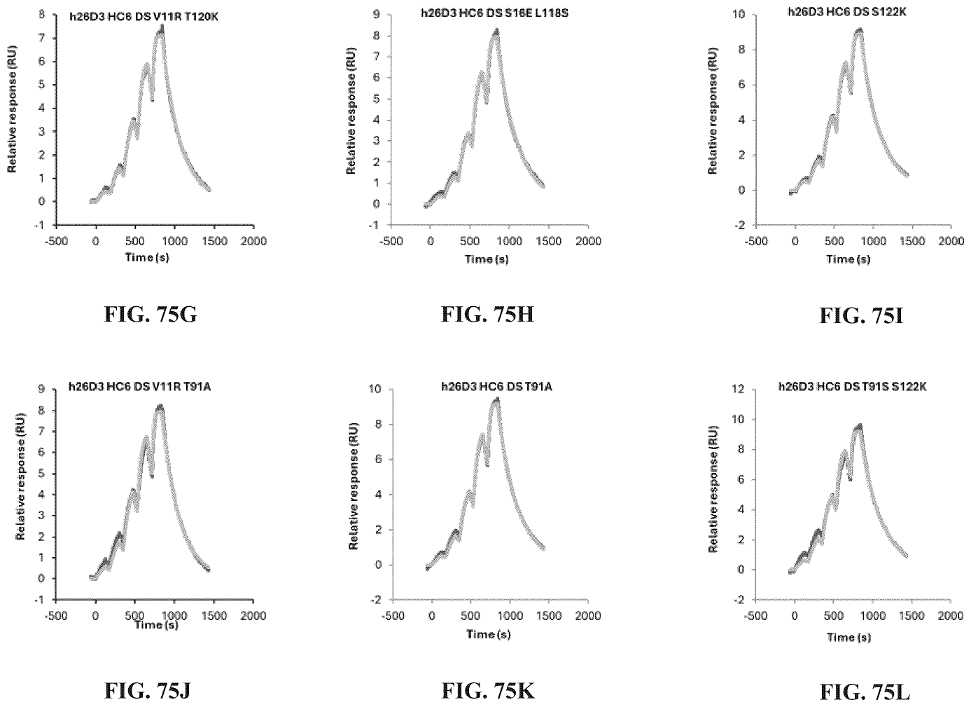

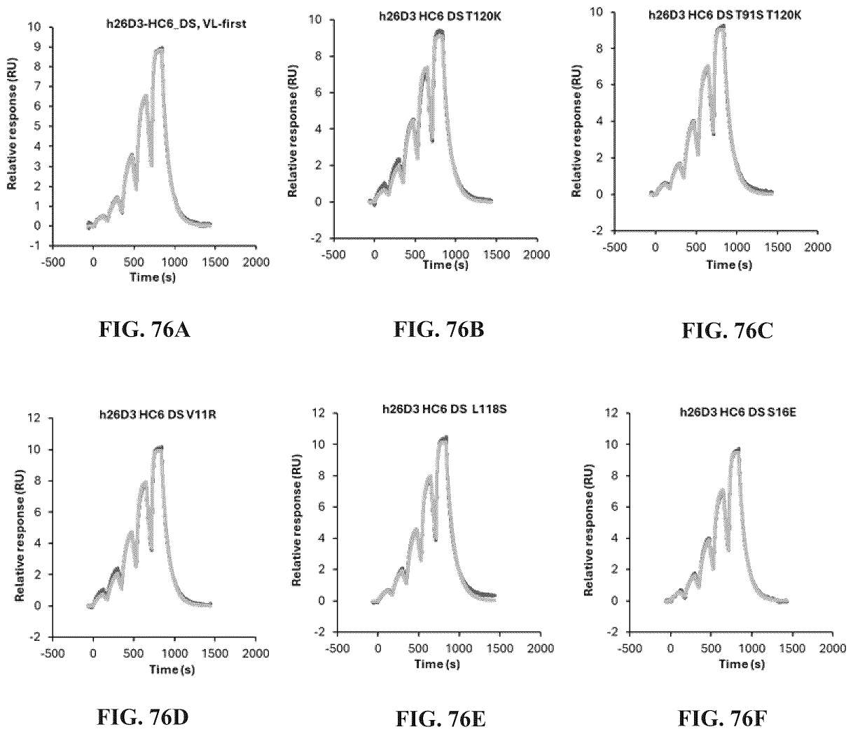

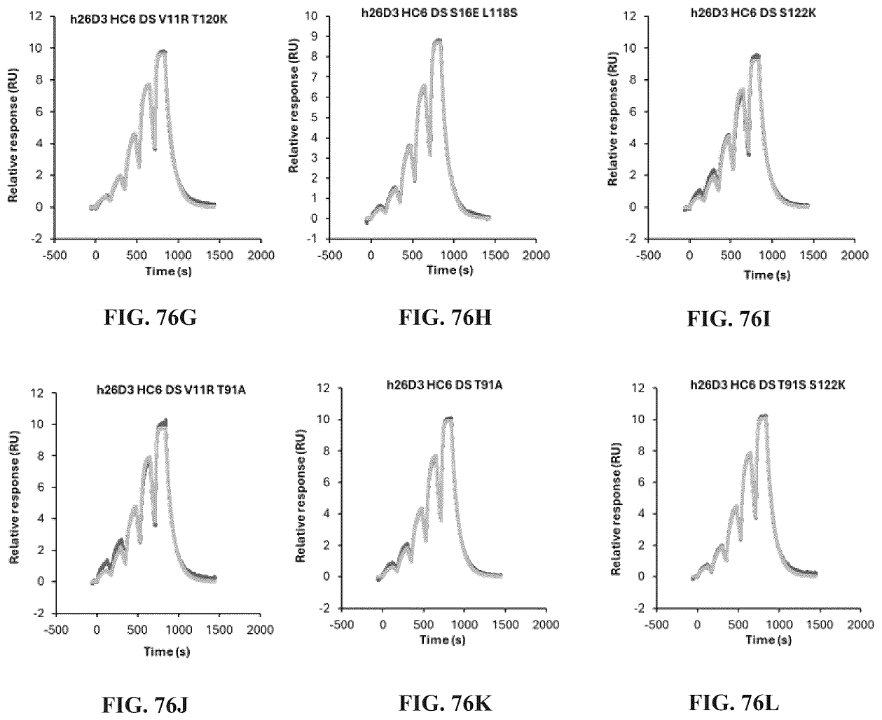

The 11 scFv molecules produced and purified as described in Example 43 and the variant “h26D3 HC6 DS, VL-first” (SEQ ID NO:154; Example 24) were evaluated for binding to human and cynomolgus TfR1 using SPR in a Biacore 8K instrument (Cytiva). For this, 30 μg/ml of DYKDDDDK Tag Monoclonal Antibody (FG4R) (ThermoFisher SCIENTIFIC. #14-6681-80) was immobilized on a CM5 sensor chip (Cytiva. #BR100399) using the amine coupling kit type 2 (Cytiva. #BR100633) according to the manufacturer's instruction. hTfR1 (SEQ ID NO:122) or cTfR1 (SEQ ID NO:123), each containing a FLAG tag, was captured on the chip via injection of a 5 μg/ml solution for 60 s at 10 μl/min flow rate. Each scFv variant was injected over the chip using a 3-fold dilution series in five steps starting at 700 nM (hTfR1) or 1500 nM (cTfR1). The interaction was measured using the single cycle kinetics method with a contact time of 120 s at a flow rate of 30 μl/min followed by a dissociation time of 600 s. Regeneration of the surface between cycles was done by injecting 10 mM glycine-HCl, pH 1.7. The binding data was fitted to a 1:1 interaction model. The scFv variants were diluted in HBS-EP+ (Cytiva. #BR100669). Experiments were performed at 25° C. As expected, the binding to TfR was similar for all analyzed molecules. The results for binding to hTfR are shown in FIGS. 75A-75L, and to cTfR in FIGS. 76A-76L. The calculated K_{D} values for binding to hTfR and cTfR are given below in Tables 45 and 46, respectively.

TABLE 45
Affinity constants for binding to hTfR1

| scFv variant | ka (1/Ms) | kd (1/s) | KD (M) | N |
|---|---|---|---|---|
| h26D3-HC6_DS. VL-first | 6.35 ± 0.75 E+04 | 4.34 ± 0.14 E−03 | 6.89 ± 0.64 E−08 | 4 |
| h26D3 DS L118S | 6.62 ± 1.02 E+04 | 3.93 ± 0.04 E−03 | 6.00 ± 0.86 E−08 | 2 |
| h26D3 DS S16E | 5.77 ± 0.08 E+04 | 3.97 ± 0.01 E−03 | 6.89 ± 0.12 E−08 | 2 |
| h26D3 DS S16E L118S | 5.78 ± 0.05 E+04 | 3.87 ± 0.05 E−03 | 6.71 ± 0.14 E−08 | 2 |
| h26D3 DS S122K | 7.64 ± 1.03 E+04 | 4.07 ± 0.03 E−03 | 5.37 ± 0.69 E−08 | 2 |
| h26D3 DS T91A | 6.50 ± 0.04 E+04 | 3.88 ± 0.05 E−03 | 5.97 ± 0.11 E−08 | 2 |
| h26D3 DS T91S S122K | 7.62 ± 1.14 E+04 | 3.80 ± 0.05 E−03 | 5.05 ± 0.82 E−08 | 2 |
| h26D3 DS T91S T120K | 6.19 ± 0.12 E+04 | 4.09 ± 0.10 E−03 | 6.62 ± 0.30 E−08 | 2 |
| h26D3 DS T120K | 7.51 ± 2.02 E+04 | 4.29 ± 0.04 E−03 | 5.93 ± 1.65 E−08 | 2 |
| h26D3 DS V11R | 9.04 ± 2.74 E+04 | 4.65 ± 0.05 E−03 | 5.40 ± 1.69 E−08 | 2 |
| h26D3 DS V11R T91A | 7.61 ± 1.38 E+04 | 4.58 ± 0.13 E−03 | 6.13 ± 1.28 E−08 | 2 |
| h26D3 DS V11R T120K | 7.33 ± 0.29 E+04 | 4.41 ± 0.02 E−03 | 6.02 ± 0.26 E−08 | 2 |

TABLE 46
Affinity constants for binding to cTfR1

| scFv variant | ka (1/Ms) | kd (1/s) | KD (M) | N |
|---|---|---|---|---|
| h26D3-HC6_DS. VL-first | 4.76 ± 0.89 E+04 | 1.14 ± 0.02 E−02 | 2.46 ± 0.48 E−07 | 4 |
| h26D3 DS L118S | 4.40 ± 0.31 E+04 | 1.07 ± 0.07 E−02 | 2.43 ± 0.33 E−07 | 2 |
| h26D3 DS S16E | 4.38 ± 0.14 E+04 | 1.21 ± 0.07 E−02 | 2.77 ± 0.07 E−07 | 2 |
| h26D3 DS S16E L118S | 4.17 ± 0.09 E+04 | 1.09 ± 0.02 E−02 | 2.61 ± 0.01 E−07 | 2 |
| h26D3 DS S122K | 6.15 ± 0.78 E+04 | 1.03 ± 0.10 E−02 | 1.70 ± 0.38 E−07 | 2 |
| h26D3 DS T91A | 4.62 ± 0.29 E+04 | 1.11 ± 0.05 E−02 | 2.40 ± 0.04 E−07 | 2 |
| h26D3 DS T91S S122K | 4.86 ± 0.11 E+04 | 1.08 ± 0.02 E−02 | 2.21 ± 0.09 E−07 | 2 |
| h26D3 DS T91S T120K | 4.67 ± 0.02 E+04 | 1.15 ± 0.09 E−02 | 2.45 ± 0.18 E−07 | 2 |
| h26D3 DS T120K | 5.74 ± 0.06 E+04 | 1.13 ± 0.04 E−02 | 1.97 ± 0.09 E−07 | 2 |
| h26D3 DS V11R | 5.15 ± 0.69 E+04 | 1.13 ± 0.01 E−02 | 2.22 ± 0.27 E−07 | 2 |
| h26D3 DS V11R T91A | 5.81 ± 0.01 E+04 | 1.20 ± 0.14 E−02 | 2.06 ± 0.24 E−07 | 2 |
| h26D3 DS V11R T120K | 6.13 ± 0.89 E+04 | 1.24 ± 0.21 E−02 | 2.01 ± 0.05 E−07 | 2 |

Next, in order to compare binding parameters with more certainty, a follow-up SPR analysis with additional replicates (N=4), was conducted for six selected molecules (SEQ ID NO:178-183) and the variant “h26D3 HC6 DS, VL-first”. The selected molecules were produced as described above. As shown in Table 47 below, the measured affinity KD values are very similar between all selected variants.

TABLE 47
Affinity constants for binding to hTfR1 measured in SPR with 4 replicates

| scFv variant | ka (1/Ms) | kd (1/s) | KD (M) | N |
|---|---|---|---|---|
| h26D3-HC6_DS, VL-first | 6.71 ± 0.46 E+04 | 4.39 ± 1.73 E−03 | 6.62 ± 2.77 E−08 | 4 |
| h26D3 DS S122K | 8.58 ± 3.29 E+04 | 4.82 ± 0.71 E−03 | 6.26 ± 2.41 E−08 | 4 |
| h26D3 DS T91S S122K | 6.70 ± 0.37 E+04 | 4.63 ± 0.37 E−03 | 6.95 ± 0.88 E−08 | 4 |
| h26D3 DS T91S T120K | 8.72 ± 1.88 E+04 | 4.65 ± 0.36 E−03 | 5.55 ± 1.41 E−08 | 4 |
| h26D3 DS T120K | 6.73 ± 0.47 E+04 | 4.78 ± 0.27 E−03 | 7.13 ± 0.75 E−08 | 4 |
| h26D3 DS V11R T91A | 7.49 ± 1.60 E+04 | 4.19 ± 0.64 E−03 | 5.87 ± 1.78 E−08 | 4 |
| h26D3 DS V11R T120K | 9.57 ± 2.00 E+04 | 4.49 ± 0.79 E−03 | 4.86 ± 1.34 E−08 | 4 |

### Example 47

### Design and Production of Further Bispecific Binding Molecules

Design of constructs: Analogously to Example 30, further bispecific binding molecules BA006 and BA007 were designed as knob-into-hole variants of the AβpE3-specific antibodies, with an hTfR1-binding scFv linked to the C-terminal amino acid residue of the knob heavy chain of the antibody. The amino acid sequences of the tested molecules are listed in Table 48.

TABLE 48
Amino acid sequences of tested bispecific
binding and control molecules

| | | Hole | Knob | |
|---|---|---|---|---|
| | LC (SEQ | HC (SEQ | HC (SEQ | |
| Designation | ID NO:) | ID NO:) | ID NO:) | scFv variant |
| BA006 | 160 | 161 | 189 | h26D3 DS S122K |
| BA007 | 160 | 161 | 190 | h26D3 DS T91S |
| | | | | S122K |

Expression from transient transfection: The tested molecules were transiently produced in CHO cells and purified with protein A affinity purification, cation exchange chromatography and preparative SEC. The fractions containing monomeric protein were collected, and brought to 10 mg/ml final concentration in PBS, pH 7.4. Protein purity, as defined by SEC-HPLC, was >99% monomer content for both constructs.

### Example 48

### Analysis of Serum Antibody Reactivity Against Bispecific Binding Molecules

The additional bispecific binding molecules designed and produced according to Example 47 were analyzed for response by pre-existing antibodies in serum using the bridging assay described in Example 45, with the following procedure to calculate the cut-off point for the assay:

First, technical outliers with a % CV larger than 20 were removed. For this, the mean of the sample duplicates was calculated, followed by calculation of the standard deviation. % CV was calculated as % CV=STDEV/MEAN*100.

The sample population minus technical outliers was then analyzed for statistical outliers by ROUT analysis in GraphPad Prism (Q=1%; allowing for 1% of false positive identified outliers). The sample population minus technical and statistical outliers made up the pseudo-negative population and was used to calculate the assay cut-off point.

In case of high PE-ADA prevalence, the cut-off point was based on the competition assay: cut-off point=mean (of pseudo negative population)+2.33×STDEV (of pseudo negative population), allowing for a 1% false positive rate. This type of cut-off point calculation was applied to BA001.

In case of low PE-ADA prevalence, the cut-off point can be directly calculated from the screening assay: cut-off point=mean (screening population with outliers removed)+1.645×STDEV (of screening population with outliers removed), allowing for a 5% false positive rate. This type of cut-off point calculation was applied to BA003, BA006 and BA007.

### Results

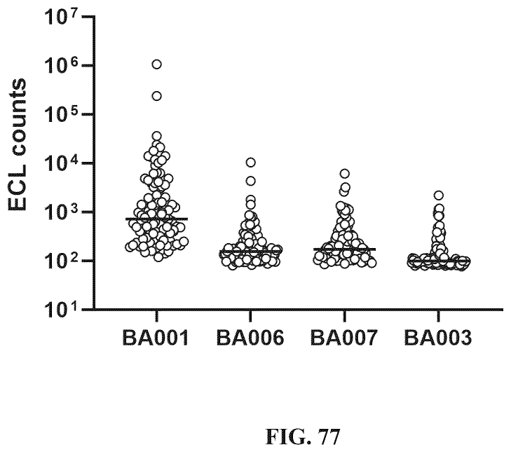

For the bispecific binding molecules BA006 and BA007, the response frequencies above the cut-off point in the bridging assay were 22% and 26%, respectively. This is similar to the 27% above the cut-off point shown by the 5 comparator antibody BA003 and lower than the 79% above cut-off point measured for the bispecific binding molecule BA001. The results was plotted and is shown in FIG. 77, wherein the median response for each tested molecule is shown with a black horizontal bar.

In summary, the data show that the reactivity from serum is reduced for both BA006 and BA007, as compared to BA001. In other words, the response detected against BA001 in serum could be decreased by the introduction of one or two point mutations in the hTfR1 binding module comprised in the bispecific binding molecule. The two mutated variants gave responses that were similar to the response observed against the comparator antibody BA003.

### Example 49

### SPR Analysis of Binding of Additional Bispecific Binding Molecules to hTfR1 and cTfR1

Binding of the purified bispecific binding molecules produced in Example 47 to human and cynomolgus TfR1 was evaluated using SPR on a Biacore 8K instrument (Cytiva). 30 μg/ml of DYKDDDDK Tag Monoclonal Antibody (FG4R) (ThermoFisher SCIENTIFIC, #14-6681-80) was immobilized on a CM5 sensor chip (Cytiva, #BR100399) using the amine coupling kit type 2 (Cytiva, #BR100633) according to the manufacturer's instruction. hTfR1 (SEQ ID NO:122) or cTfR1 (SEQ ID NO:123), each containing a FLAG tag, was captured on the chip via injection of a 5 μg/ml solution for 60 s at 10 μl/min flow rate. The tested bispecific binding molecules were injected over the chip using a 3-fold dilution series in five steps starting at 1500 nM (for hTfR1) or 2000 nM (for cTfR1). The interaction was measured using the single cycle kinetics method with a contact time of 120 s at a flow rate of 30 μl/min followed by a dissociation time of 600 s. Regeneration of the surface between cycles was done by injecting 10 mM glycine-HCl, pH 1.7. The binding data was fitted to a 1:1 interaction model. The tested molecules were diluted in HBS-EP+ (Cytiva, #BR100669). Experiments were performed at 25° C.

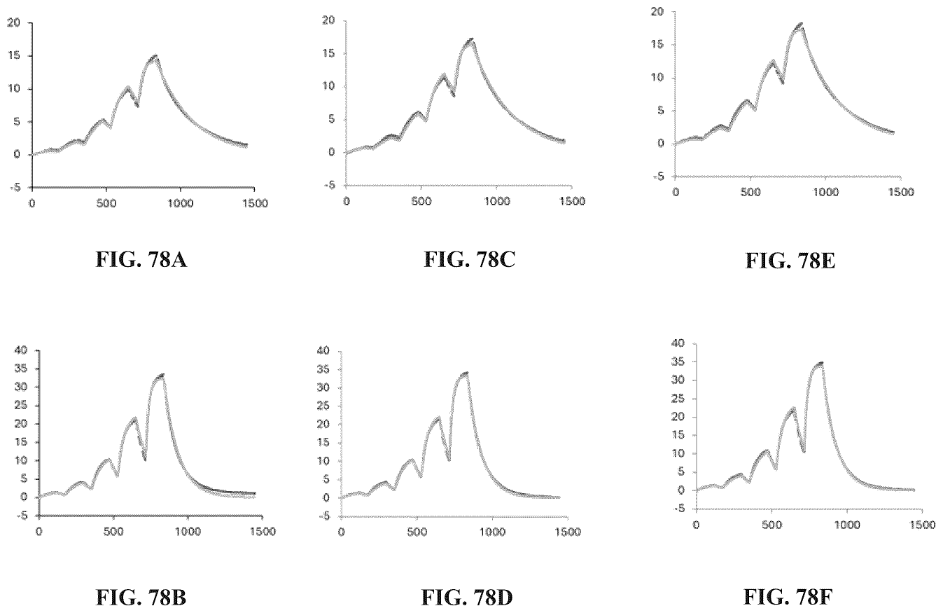

As expected, the binding to hTfR1 and cTfR1 were similar between the analyzed molecules. The results are shown in FIGS. 78A-78F, displaying representative sensorgrams for the binding of BA001 (A, B), BA007 (C, D) and BA006 (E, F) against hTfR1 (A, C, E) and cTfR1 (B, D, F). The calculated K_{D} values are given in Table 49 and Table 50 below.

TABLE 49
SPR analysis of binding to hTfR1

| Binding | ka (M⁻¹s⁻¹) | kd (s⁻¹) | KD (nM) |
|---|---|---|---|
| molecule | Mean ± SD | Mean ± SD | Mean ± SD |
| BA006 | 2.2 ± 0.08 E+04 | 4.1 ± 0.06 E−03 | 189 ± 7 |
| BA007 | 2.2 ± 0.09 E+04 | 4.0 ± 0.02 E−03 | 186 ± 8 |
| BA001 | 2.2 ± 0.3 E+04 | 4.2 ± 0.2 E−03 | 190 ± 26 |

TABLE 50
SPR analysis of binding to cTfR1

| Binding | ka (M⁻¹s⁻¹) | kd (s⁻¹) | KD (nM) |
|---|---|---|---|
| molecule | Mean ± SD | Mean ± SD | Mean ± SD |
| BA006 | 2.0 ± 0.07 E+04 | 1.1 ± 0.06 E−02 | 552 ± 35 |
| BA007 | 1.9 ± 0.03 E+04 | 1.1 ± 0.01 E−02 | 565 ± 8 |
| BA001 | 1.9 ± 0.02 E+04 | 1.0 ± 0.01 E−02 | 525 ± 7 |

### Example 50

### SPR Analysis of Binding of Additional Bispecific Binding Molecules to AβpE3

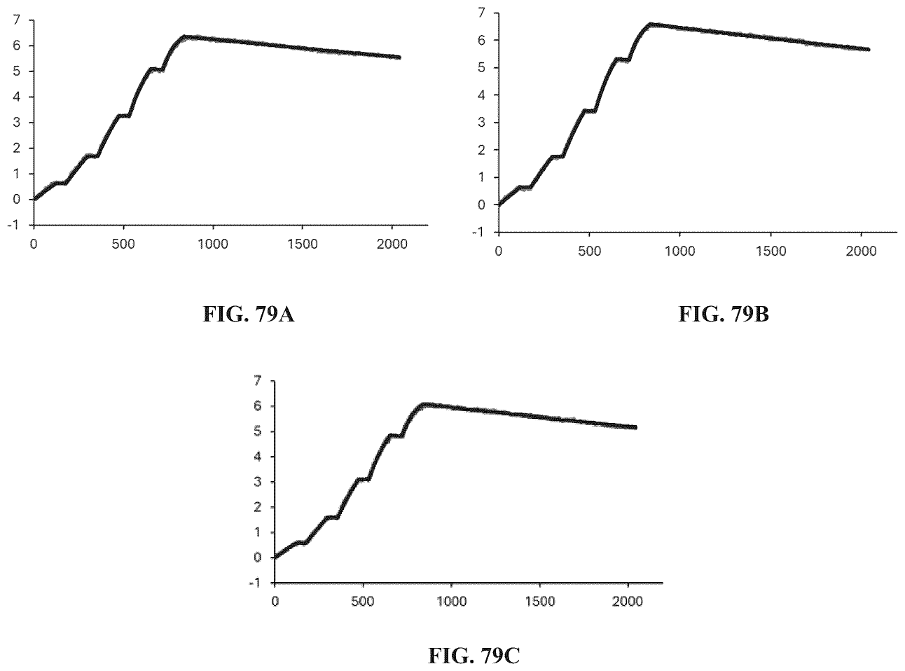

Binding of bispecific binding molecules BA006 and BA007 to AβpE3-40 monomers was analyzed by SPR as described in Example 32, and KD values were determined. Both binding molecules demonstrated binding to AβpE3-40 monomers at an affinity similar to that of BA001. Results are shown in FIGS. 79A-79C, displaying representative sensorgrams for the binding of BA006 (FIG. 79A), BA007 (FIG. 79B) and BA001 (FIG. 79C) against AβpE3-40 monomers. The calculated ka, kd and KD values are shown in Table 51.

TABLE 51
SPR analysis of binding to AβpE3-40 monomers

| Binding | ka (M⁻¹s⁻¹) | kd (s⁻¹) | KD (nM) |
|---|---|---|---|
| molecule | Mean ± SD | Mean ± SD | Mean ± SD |
| BA006 | 5.2 ± 0.1 e4 | 1.2 ± 0.05 e−4 | 2.3 ± 0.1 |
| BA007 | 5.4 ± 0.4 e4 | 1.2 ± 0.2 e−4 | 2.1 ± 0.2 |
| BA001 | 5.4 ± 0.2 e4 | 1.1 ± 0.2 e−4 | 2.1 ± 0.4 |

### Example 51

### Binding of Additional Bispecific Binding Molecules to Cell-Surface hTfR1

### Materials and Methods

To measure binding of bispecific binding molecules BA001, BA006 and BA007, and of the comparator antibody BA003, to endogenous hTfR1 receptors on the cell surface, K562 cells (Sigma/ECACC, #CB_89121407) were cultured and used in a cell binding experiment. Fc receptors were blocked (BD human Fc-Block #564220) for 15 min at 4° C. to exclude unspecific binding to cells via Fc receptors. Cells were then washed with staining buffer, seeded in a 96 well V-bottom plate (Costar, #3897) and incubated with serially diluted test compounds in staining buffer for 1 h at 4° C. Cells were washed twice with staining buffer to remove unbound molecules and then fixed for 15 min at 4° C. with 4% methanol-free PFA (ThermoScientific, #J61899.AP). After fixation, cells were washed once with staining buffer. To detect bound test compounds, cells were incubated with PE-conjugated anti-human IgG Fc-specific antibody (eBioscience, #12-4998-82) for 20 min at 4° C. Cells were then washed twice prior to acquisition on a Flow cytometer (BD Symphony A1 or BD Lyric). Samples were analyzed using FlowJo software (BD) and the mean fluorescence intensity of bound test compound was plotted against concentration.

### Results

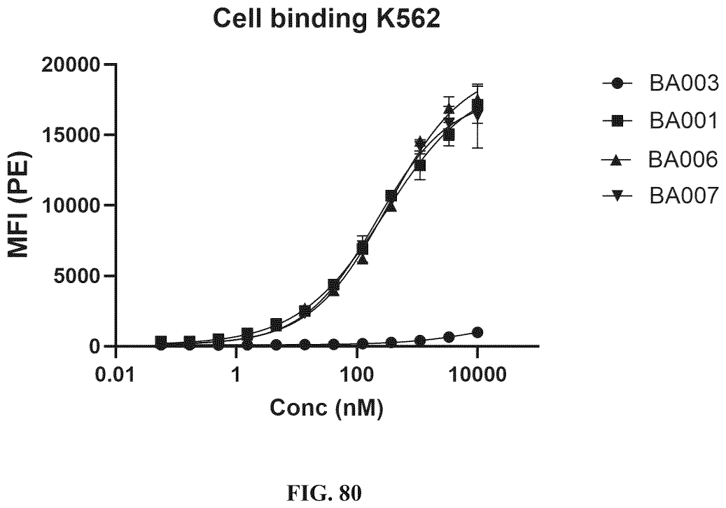

Results are shown in FIG. 80 and demonstrate that all tested TfR-binding molecules bind to hTfR1 on the surface of the cells in a highly similar and dose-dependent way. The comparator antibody, BA003, does not bind to hTfR1.

### ITEMIZED LISTING OF EMBODIMENTS

1. A bispecific binding molecule, comprising
   a first moiety M1, which is an AβpE3 binding moiety comprising a VH domain and a VL domain, said VH and VL domains forming a VH/VL pair comprising an antigen-binding surface, in which said antigen-binding surface is composed of three complementarity-determining regions (CDRs) from said VH domain and three CDRs from said VL domain, and in which said CDRs consist of the following amino acid sequences:

VHCDR1:
(SEQ ID NO: 1)
GX₁TX₂N

wherein
X₁ is selected from Y and F; and
X₂ is selected from L and M;

VHCDR2:
(SEQ ID NO: 2)
LINPYNGX₃TTYNX₄KFX₅G

wherein
X₃ is selected from I and V
X₄ is selected from P and Q; and
X₅ is selected from M and K;

VHCDR3:
(SEQ ID NO: 3)
EGNWEGVY
VLCDR1:
(SEQ ID NO: 4)
X₆SSQSLLDSNGKTYLH

wherein
X₆ is selected from K and R;

VLCDR2:
(SEQ ID NO: 5)
LVSX₇LDS

wherein
X₇ is selected from I and K; and

VLCDR3:
(SEQ ID NO: 6)
VQGTHFPFT;

a second moiety M2, which is a human transferrin receptor 1 (hTfR1) binding moiety comprising an immunoglobulin heavy chain variable domain (VH) and an immunoglobulin light chain variable domain (VL), said VH and VL domains forming a VH/VL pair comprising an antigen-binding surface, in which said antigen-binding surface provides the binding protein with the capacity to bind selectively to an epitope located in the protease-like domain of hTfR1 defined by amino acid residues 121-183 and 384-605 in SEQ ID NO:121.
2. Bispecific binding molecule according to item 1, wherein said VHCDR1, VHCDR2 and VLCDR2 regions in moiety M1 consist of the following amino acid sequences:

VHCDR1:
(SEQ ID NO: 7)
GFTMN
VHCDR2:
(SEQ ID NO: 8)
LINPYNGVTTYNQKFKG
VLCDR2:
(SEQ ID NO: 9)
LVSILDS.

3. Bispecific binding molecule according to any preceding item, wherein the VH domain in M1 comprises an amino acid sequence selected from
i) the group consisting of SEQ ID NO:15-22; and
ii) an amino acid sequence having at least 80% identity to any one of SEQ ID NO:15-22, provided that the three VHCDR regions consist of SEQ ID NO:7, SEQ ID NO:8 and SEQ ID NO:3.
4. Bispecific binding molecule according to item 3, wherein the M1 VH amino acid sequence in i) is selected from the group consisting of SEQ ID NO:15-21.
5. Bispecific binding molecule according to item 4, wherein the M1 VH amino acid sequence in i) is selected from the group consisting of SEQ ID NO:15-16 and 18-21.
6. Bispecific binding molecule according to item 4, wherein the M1 VH amino acid sequence in i) is selected from the group consisting of SEQ ID NO:15-20.
7. Bispecific binding molecule according to any one of items 5-6, wherein the M1 VH amino acid sequence in i) is selected from the group consisting of SEQ ID NO:15-16 and 18-20.
8. Bispecific binding molecule according to item 7, wherein the M1 VH amino acid sequence in i) is selected from the group consisting of SEQ ID NO:15 and SEQ ID NO:18.
9. Bispecific binding molecule according to item 8, wherein the M1 VH amino acid sequence in i) is SEQ ID NO:18.
10. Bispecific binding molecule according to any preceding item, wherein the amino acid sequence of VLCDR1 in moiety M1 is

(SEQ ID NO: 10)
RSSQSLLDSNGKTYLH.

11. Bispecific binding molecule according to item 10, wherein the VL domain in M1 comprises an amino acid sequence selected from
i) the group consisting of SEQ ID NO:23-24; and
ii) an amino acid sequence having at least 80% identity to any one of SEQ ID NO:23-24, provided that the three VLCDR regions consist of SEQ ID NO:10, SEQ ID NO:9 and SEQ ID NO:6.
12. Bispecific binding molecule according to item 11, wherein the M1 VL amino acid sequence in i) is SEQ ID NO:23.
13. Bispecific binding molecule according to any preceding item, wherein said M1 heavy chain variable domain is as defined in any one of items 3-9 and said M1 light chain variable domain is as defined in any one of items 11-12.
14. Bispecific binding molecule according to item 13, wherein the M1 heavy chain variable domain and the M1 light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24;
h) a heavy chain variable domain comprising SEQ ID NO:22 and a light chain variable domain comprising SEQ ID NO:23.
15. Bispecific binding molecule according to item 14, wherein the M1 heavy chain variable domain and the M1 light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24.
16. Bispecific binding molecule according to item 15, wherein the M1 heavy chain variable domain and the M1 light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
g) a heavy chain variable domain comprising SEQ ID NO:21 and a light chain variable domain comprising SEQ ID NO:24.
17. Bispecific binding molecule according to item 15, wherein the M1 heavy chain variable domain and the M1 light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23.

18. Bispecific binding molecule according to any one of items 16-17, wherein the M1 heavy chain variable domain and the M1 light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23.
19. Bispecific binding molecule according to item 18, wherein the M1 heavy chain variable domain and the M1 light chain variable domain are represented by the following VH/VL combinations:
a) a heavy chain variable domain comprising SEQ ID NO:18 and a light chain variable domain comprising SEQ ID NO:23;
b) a heavy chain variable domain comprising SEQ ID NO:15 and a light chain variable domain comprising SEQ ID NO:23.
20. Bispecific binding molecule according to item 19, wherein the M1 heavy chain variable domain and the M1 light chain variable domain are represented by the following VH/VL combination:
a) a heavy chain variable domain comprising SEQ ID NO:18 and a light chain variable domain comprising SEQ ID NO:23.
21. Bispecific binding molecule according to any one of items 1-9, in which the amino acid sequence of VLCDR1 in moiety M1 is

(SEQ ID NO: 11)
KSSQSLLDSNGKTYLH.

22. Bispecific binding molecule according to item 21, wherein the M1 heavy chain variable domain comprises an amino acid sequence selected from
i) SEQ ID NO:25; and
ii) an amino acid sequence having at least 80% identity to SEQ ID NO:25, provided that the three VHCDR regions consist of SEQ ID NO:7, SEQ ID NO:8 and SEQ ID NO:3.
23. Bispecific binding molecule according to any one of items 21-22, wherein the M1 light chain variable domain comprises an amino acid sequence selected from
i) SEQ ID NO:26; and
ii) an amino acid sequence having at least 80% identity to SEQ ID NO:26, provided that the three VLCDR regions consist of SEQ ID NO:11, SEQ ID NO:9 and SEQ ID NO:6.
24. Bispecific binding molecule according to any one of items 22-23, wherein said M1 heavy chain variable domain is as defined in item 22 and said M1 light chain variable domain is as defined in item 23.
25. Bispecific binding molecule according to item 1, wherein said VHCDR1, VHCDR2, VLCDR1 and VLCDR2 regions in moiety M1 consist of the following amino acid sequences:

VHCDR1:
(SEQ ID NO: 12)
GYTLN;
VHCDR2:
(SEQ ID NO: 13)
LINPYNGITTYNPKFMG;
VLCDR1:
(SEQ ID NO: 11)
KSSQSLLDSNGKTYLH
VLCDR2:
(SEQ ID NO: 14)
LVSKLDS.

26. Bispecific binding molecule according to item 25, wherein the M1 heavy chain variable domain comprises an amino acid sequence selected from
i) SEQ ID NO:27; and
ii) an amino acid sequence having at least 80% identity to SEQ ID NO:27, provided that the three VHCDR regions consist of SEQ ID NO:12, SEQ ID NO:13 and SEQ ID NO:3.
27. Bispecific binding molecule according to any one of items 25-26, wherein the M1 light chain variable domain comprises an amino acid sequence selected from
i) SEQ ID NO:28; and
ii) an amino acid sequence having at least 80% identity to SEQ ID NO:28, provided that the three VLCDR regions consist of SEQ ID NO:11, SEQ ID NO:14 and SEQ ID NO:6.
28. Bispecific binding molecule according to any one of items 26-27, wherein said M1 heavy chain variable domain is as defined in item 26 and said M1 light chain variable domain is as defined in item 27.
29. Bispecific binding molecule according to any preceding item, wherein said epitope of M2 located in the protease-like domain of hTfR1 comprises or consists of the amino acid residues 150, 151, 154, 158, 159, 161, 163 and 385 in SEQ ID NO:121.
30. Bispecific binding molecule according to item 29, in which said antigen-binding surface of M2 is composed of three complementarity-determining regions (CDRs) from said VH domain and three CDRs from said VL domain, and in which said CDRs comprise the following:

VHCDR1:
(SEQ ID NO: 37)
X1X2NMX3,

wherein
X1 is selected from D and A;
X2 is selected from Y and A; and
X3 is selected from D and A;

VHCDR2:
(SEQ ID NO: 38)
X4INPX5X6X7TTSX8X9X10KFKG,

wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;

VLCDR1:
(SEQ ID NO: 40)
KSSQSLLX11SX12NX13KNX14LA,

wherein
X11 is selected from Y and A;
X12 is selected from T and S;
X13 is selected from Q and R; and
X14 is selected from Y and A;

VLCDR2:
(SEQ ID NO: 41)
X15ASTRES

wherein
X15 is selected from W and A; and

VLCDR3:
(SEQ ID NO: 42)
QQX16X17X18X19PX20T

wherein
X16 is selected from Y and A;
X17 is selected from F and Y;
X18 is selected from I and N;
X19 is selected from Y and A; and
X20 is selected from R and Y.
31. Bispecific binding molecule according to item 30, said CDRs of M2 further comprising

VHCDR3:
(SEQ ID NO: 39)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.
32. Bispecific binding molecule according to any one of items 30-31, in which said VHCDR2 in moiety M2 is:

VHCDR2:
(SEQ ID NO: 43)
X4INPX5X6X7TTSX8NEKFKG,

wherein
X4 is selected from D and A;
X5 is selected from D and A;
X6 is selected from Y and A;
X7 is selected from D and A; and
X8 is selected from Y and A.
33. Bispecific binding molecule according to any one of items 30-32, in which said VLCDR1 in moiety M2 is:

VLCDR1:
(SEQ ID NO: 44)
KSSQSLLX11STNQKNX14LA,

wherein
X11 is selected from Y and A; and
X14 is selected from Y and A.
34. Bispecific binding molecule according to any one of items 30-33, in which said VLCDR3 in moiety M2 is:

VLCDR3:
(SEQ ID NO: 45)
QQX16FIX19PRT

wherein
X16 is selected from Y and A;
X19 is selected from Y and A.
35. Bispecific binding molecule according to any one of items 30-34, in which the amino acid sequence of said VHCDR1 in moiety M2 is selected from the group consisting of SEQ ID NO:46 and 52-54.
36. Bispecific binding molecule according to any one of items 30-35, in which the amino acid sequence of said VHCDR2 in moiety M2 is selected from the group consisting of SEQ ID NO:47, 55-59 and 70, for example selected from the group consisting of SEQ ID NO:47 and 55-59.
37. Bispecific binding molecule according to any one of items 30-36, in which the amino acid sequence of said VHCDR3 in moiety M2 is selected from the group consisting of SEQ ID NO:48, 60-64 and 71, for example selected from the group consisting of SEQ ID NO:48 and 60-64.
38. Bispecific binding molecule according to any one of items 30-37, in which the amino acid sequence of said VLCDR1 in moiety M2 is selected from the group consisting of SEQ ID NO:49, 65, 66 and 72, for example selected from the group consisting of SEQ ID NO:49, 65 and 66.
39. Bispecific binding molecule according to any one of items 30-38, in which the amino acid sequence of said VLCDR2 in moiety M2 is selected from the group consisting of SEQ ID NO:50 and 67.
40. Bispecific binding molecule according to any one of items 30-39, in which the amino acid sequence of said VLCDR3 in moiety M2 is selected from the group consisting of SEQ ID NO:51, 68, 69 and 73, for example selected from the group consisting of SEQ ID NO:51, 68 and 69.
41. Bispecific binding molecule according to any one of items 30-40, in which the amino acid sequences of the six CDRs in moiety M2 are the following:

VHCDR1:
(SEQ ID NO: 46)
DYNMD,
VHCDR2:
(SEQ ID NO: 47)
DINPDYDTTSYNEKFKG,
VHCDR3:
(SEQ ID NO: 48)
GGYSGSSYYHPMDY
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

42. Bispecific binding molecule according to any one of items 30-40, in which the amino acid sequences of the six CDRs in moiety M2 are the following:

VHCDR1:
(SEQ ID NO: 46)
DYNMD,
VHCDR2:
(SEQ ID NO: 57)
DINPDADTTSYNEKFKG,
VHCDR3:
(SEQ ID NO: 48)
GGYSGSSYYHPMDY,
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

43. Bispecific binding molecule according to item 30, in which the amino acid sequences of the six CDRs in moiety M2 are the following:

VHCDR1:
(SEQ ID NO: 46)
DYNMD,
VHCDR2:
(SEQ ID NO: 70)
DINPNYDTTSYSQKFKG,
VHCDR3:
(SEQ ID NO: 71)
SEAGNYYWYFDV,
VLCDR1:
(SEQ ID NO: 72)
KSSQSLLYSSNRKNYLA,
VLCDR2:
(SEQ ID NO: 50)
WASTRES,
and
VLCDR3:
(SEQ ID NO: 73)
QQYYNYPYT.

44. Bispecific binding molecule according to any preceding item, wherein said VH domain in moiety M2 comprises or consists of an amino acid sequence selected from
(i) the group consisting of SEQ ID NO:80-93, 101 and 103, for example the group consisting of SEQ ID NO:80-93, for example the group consisting of SEQ ID NO:80 and 86; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i).
45. Bispecific binding molecule according to any preceding item, wherein said VL domain in moiety M2 comprises or consists of an amino acid sequence selected from
(i) the group consisting of SEQ ID NO:94-100, 102 and 104, for example the group consisting of SEQ ID NO:94-100; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions a sequence defined in (i).
46. Bispecific binding molecule according to any one of items 44-45, wherein said VH domain in moiety M2 is as defined in item 44 and said VL domain in moiety M2 is as defined in item 45.
47. Bispecific binding molecule according to item 46, in which said VH domain in moiety M2 comprises SEQ ID NO:80 and said VL domain comprises a sequence selected from SEQ ID NO:94-100.
48. Bispecific binding molecule according to item 46, in which said VH domain in moiety M2 comprises a sequence selected from SEQ ID NO:80-93 and said VL domain in moiety M2 comprises SEQ ID NO:94.
49. Bispecific binding molecule according to any one of items 47-48, in which said VH domain in moiety M2 comprises SEQ ID NO:80 and said VL domain in moiety M2 comprises SEQ ID NO:94.
50. Bispecific binding molecule according to any one of items 47-48, in which said VH domain in moiety M2 comprises SEQ ID NO:86 and said VL domain in moiety M2 comprises SEQ ID NO:94.
51. Bispecific binding molecule according to any one of items 1-43, which comprises one first cysteine residue in said VH domain in moiety M2 and one second cysteine residue in said VL domain in moiety M2, said first and second cysteine residues being arranged such that they form a disulfide bridge connecting the VH and VL domains.
52. Bispecific binding molecule according to item 51, wherein said first cysteine residue is located at an amino acid position selected from M2 VH position 39-49, such as selected from M2 VH position 41-47, such as selected from M2 VH position 43-45, such as at M2 VH position 44, all as determined by reference to the Kabat numbering scheme.
53. Bispecific binding molecule according to item 51 or 52, wherein said second cysteine residue is located at an amino acid position selected from M2 VL position 95-105, such as selected from M2 VL position 97-103, such as selected from M2 VL position 99-101, such as at M2 VL position 100, all as determined by reference to the Kabat numbering scheme.
54. Bispecific binding molecule according to item 52 or 53, wherein said first cysteine residue is located at M2 VH position 44 and said second cysteine residue is located at M2 VL position 100, as determined by reference to the Kabat numbering scheme.
55. Bispecific binding molecule according to any one of items 51-54, wherein said VH domain in moiety M2 comprises or consists of an amino acid sequence selected from
   (i) the group consisting of SEQ ID NO:124-139, for example the group consisting of SEQ ID NO:124-137, for example the group consisting of SEQ ID NO:124 and 130; and
   (ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44.
56. Bispecific binding molecule according to any one of items 51-55, wherein said VL domain in moiety M2 comprises or consists of an amino acid sequence selected from
   (i) the group consisting of SEQ ID NO:141-149, for example the group consisting of SEQ ID NO:141-147; and
   (ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106.
57. Bispecific binding molecule according to any one of items 55-56, wherein said VH domain in moiety M2 is as defined in item 55 and said VL domain in moiety M2 is as defined in item 56.
58. Bispecific binding molecule according to item 57, in which said VH domain in moiety M2 comprises SEQ ID NO:124 and said VL domain in moiety M2 comprises a sequence selected from SEQ ID NO:141-147.
59. Bispecific binding molecule according to item 57, in which said VH domain in moiety M2 comprises a sequence selected from SEQ ID NO:124-137 and said VL domain in moiety M2 comprises SEQ ID NO:141.
60. Bispecific binding molecule according to any one of items 58-59, in which said VH domain in moiety M2 comprises SEQ ID NO:124 and said VL domain in moiety M2 comprises SEQ ID NO:141.
61. Bispecific binding molecule according to any one of items 58-59, in which said VH domain in moiety M2 comprises SEQ ID NO:130 and said VL domain in moiety M2 comprises SEQ ID NO:141.
62. Bispecific binding molecule according to any one of items 1-30, in which said antigen-binding surface in moiety M2 is composed of three complementarity-determining regions (CDRs) from said VH domain and three CDRs from said VL domain, and in which said CDRs comprise the following:

VHCDR1:
(SEQ ID NO: 74)
NYWLG,
VHCDR2:
(SEQ ID NO: 75)
DIFPGSDNTYYNEKFKG,
VHCDR3:
(SEQ ID NO: 76)
SGNFYAMDY,
VLCDR1:
(SEQ ID NO: 77)
SASSSVNYMN,
VLCDR2:
(SEQ ID NO: 78)
DTSKLAS,
VLCDR3:
(SEQ ID NO: 79)
FQGSGYPFT.

63. Bispecific binding molecule according to item 62, wherein said VH domain in moiety M2 comprises or consists of an amino acid sequence selected from SEQ ID NO:105 and a sequence having at least 80%, at least 90%, at least 92%, at least 94%, at least 95%, at least 96%, at least 97%, at least 98% or at least 99% identity to SEQ ID NO:105, provided that the sequences of the CDR regions are 100% identical to those of the CDR regions in SEQ ID NO:105.
64. Bispecific binding molecule according to any one of items 62-63, wherein said VL domain in moiety M2 comprises or consists of an amino acid sequence selected from SEQ ID NO:106 and a sequence having at least 80%, at least 90%, at least 92%, at least 94%, at least 95%, at least 96%, at least 97%, at least 98% or at least 99% identity to SEQ ID NO:106, provided that the sequences of the CDR regions are 100% identical to those of the CDR regions in SEQ ID NO:106.
65. Bispecific binding molecule according to any one of items 63-64, wherein said VH domain in moiety M2 is as defined in item 63 and said VL domain in moiety M2 is as defined in item 64.
66. Bispecific binding molecule according to item 62, wherein said VH domain in moiety M2 comprises or consists of an amino acid sequence selected from SEQ ID NO:140 and a sequence having at least 80%, at least 90%, at least 92%, at least 94%, at least 95%, at least 96%, at least 97%, at least 98% or at least 99% identity to SEQ ID NO:140, provided that the sequences of the CDR regions are 100% identical to those of the CDR regions in SEQ ID NO:140, and provided that the sequence comprises a cysteine residue at position 44.
67. Bispecific binding molecule according to any one of items 62 and 66, wherein said VL domain in moiety M2 comprises or consists of an amino acid sequence selected from SEQ ID NO:150 and a sequence having at least 80%, at least 90%, at least 92%, at least 94%, at least 95%, at least 96%, at least 97%, at least 98% or at least 99% identity to SEQ ID NO:150, provided that the sequences of the CDR regions are 100% identical to those of the CDR regions in SEQ ID NO:150, and provided that the sequence comprises a cysteine residue at position 99.
68. Bispecific binding molecule according to any one of items 66-67, wherein said VH domain in moiety M2 is as defined in item 66 and said VL domain in moiety M2 is as defined in item 67.
69. Bispecific binding molecule according to any preceding item, in which the VH/VL pair in moiety M2 forms part of an antibody construct.
70. Bispecific binding molecule according to item 69, wherein the VH/VL pair in moiety M2 is present in an antibody fragment selected from the group consisting of a Fab fragment, a single chain Fab (scFab) fragment, an Fv fragment and a single chain (scFv) fragment.
71. Bispecific binding molecule according to item 70, in which the VH/VL pair of the second moiety M2 forms part of an scFv, in which the VH and VL domains are coupled together by a peptide scFv linker.
72. Bispecific binding molecule according to item 71, in which said scFv linker is attached to the N-terminal amino acid residue of the VH domain and to the C-terminal amino acid residue of the VL domain.
73. Bispecific binding molecule according to item 71, in which said scFv linker is attached to the C-terminal amino acid residue of the VH domain and to the N-terminal amino acid residue of the VL domain.
74. Bispecific binding molecule according to any one of items 71-73, in which said scFv linker is a flexible peptide linker consisting of from 5 to 40 amino acid residues, for example from 10 to 30 amino acid residues, for example from 15 to 25 amino acid residues, for example about 15 amino acid residues, for example 15 amino acid residues, for example comprising or consisting of the sequence (G₄S)₃ (SEQ ID NO:166).
75. Bispecific binding molecule according to any preceding item, in which M1 and M2 are connected to each other by at least one peptide linker between M1 and M2.
76. Bispecific binding molecule according to item 75, in which said at least one peptide linker between M1 and M2 is attached, on the M2 side, to the C-terminal amino acid residue of the VH domain of M2 or to the N-terminal amino acid residue of the VL domain of M2.
77. Bispecific binding molecule according to any one of items 75-76, wherein said at least one peptide linker between M1 and M2 is a flexible linker.
78. Bispecific binding molecule according to item 77, wherein said flexible linker(s) comprise(s) glycine, serine, alanine and/or threonine residues.
79. Bispecific binding molecule according to item 78, wherein said linker(s) has a general formula selected from (GₙSₘ)ₚ and (SₙGₘ)ₚ, wherein, independently, n=1-7, m=0-7, n+m≤8 and p=1-10.
80. Bispecific binding molecule according to any one of items 75-79, wherein said at least one linker is between 10 and 50 amino acid residues long, such as between 10 and 30 amino acid residues long, such as between 15 and 25 amino acid residues long or between 10 and 20 amino acids long.
81. Bispecific binding molecule according to any preceding item, in which M1 is provided as a knob-into-hole antibody comprising two identical antibody light chains; one antibody hole heavy chain; and one antibody knob heavy chain; and M2 is provided as an scFv linked to the C-terminal amino acid residue of the knob heavy chain of M1.
82. Bispecific binding molecule according to item 81, in which the amino acid sequence of said M1 antibody light chain comprises or consists of SEQ ID NO:160, the amino acid sequence of said M1 antibody hole heavy chain comprises or consists of SEQ ID NO:161, and the amino acid sequence of said M1 antibody knob heavy chain with linked M2 scFv comprises or consists of a sequence selected from SEQ ID NO:162-164.
83. Bispecific binding molecule according to item 82, in which the amino acid sequence of said M1 antibody knob heavy chain with linked M2 scFv comprises or consists of SEQ ID NO:162.
84. Bispecific binding molecule according to item 82, in which the amino acid sequence of said M1 antibody knob heavy chain with linked M2 scFv comprises or consists of SEQ ID NO:163.
85. Bispecific binding molecule according to item 82, in which the amino acid sequence of said M1 antibody knob heavy chain with linked M2 scFv comprises or consists of SEQ ID NO:164.
86. Bispecific binding molecule according to any preceding item, wherein said AβpE3 bound by moiety M1 is in a form selected from the group consisting of monomers, protofibrils, fibrils and plaques.
87. Bispecific binding molecule according to any preceding item, which has a higher binding affinity for AβpE3 monomers than for Aβ1-X monomers.
88. Bispecific binding molecule according to item 87, which has at least 2× higher binding affinity for AβpE3 monomers than for Aβ1-X monomers, such as at least 10× higher, such as at least 100× higher, such as at least 1000× higher, such as at least 3000× higher binding affinity.
89. Bispecific binding molecule according to any preceding item, which has a higher binding affinity for protofibrils comprising AβpE3 than for AβpE3 monomers.
90. Bispecific binding molecule according to item 89, which has at least 2× higher binding affinity for protofibrils comprising AβpE3 than for AβpE3 monomers, such as at least 10× higher, such as at least 40× higher, such as at least 100× higher, such as at least 200× higher binding affinity.
91. Bispecific binding molecule according to any preceding item, which has a binding affinity for protofibrils comprising AβpE3 that corresponds to a K_{D} value of no more than 1 nM, such as between 1 and 200 μM, such as between 10 and 100 μM, as determined by surface plasmon resonance.
92. Bispecific binding molecule according to any preceding item, which has a binding affinity for AβpE3 monomers that corresponds to a K_{D} value of no more than 100 nM, such as between 0.1 and 50 nM, such as between 0.5 and 10 nM, as determined by surface plasmon resonance.
93. Pharmaceutical composition, comprising a bispecific binding molecule according to any preceding item and a pharmaceutically acceptable carrier or excipient.
94. Bispecific binding molecule according to any one of items 1-92 or a composition according to item 93 for use in treatment, such as for use in therapeutic treatment or for use in prophylactic treatment.
95. Bispecific binding molecule according to any one of items 1-92 or composition according to item 93 for use in diagnosis in vivo or prognosis in vivo.
96. Bispecific binding molecule or composition for use according to any one of items 94-95, wherein said therapy, prophylaxis, in vivo diagnosis or in vivo prognosis is with respect to a neurodegenerative disorder associated with amyloid beta peptide aggregation, for example a disorder selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.
97. Bispecific binding molecule for use according to item 96, wherein said neurodegenerative disorder is Alzheimer's disease.
98. A method of therapeutic or prophylactic treatment of a mammal having, or being at risk of developing, a neurodegenerative disorder, said method comprising administering to said mammal a therapeutically effective amount of a bispecific binding molecule according to any one of items 1-92 or a composition according to item 93.
99. A method according to item 98, wherein said neurodegenerative disorder is a disorder associated with amyloid beta peptide aggregation, for example a disorder selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.
100. A method according to item 99, wherein said neurodegenerative disorder is Alzheimer's disease.
101. A method of detecting AβpE3 peptides in vitro, comprising providing a sample suspected to contain Aβ peptides, contacting said sample with a bispecific binding molecule according to any one of items 1-92, and detecting the binding of said protein to indicate the presence of AβpE3 peptides in the sample.
102. A method of determining the amount of AβpE3 peptides present in a subject, comprising the steps of:
a) contacting the subject, or a sample isolated from the subject, with a bispecific binding molecule according to any one of items 1-92 or a composition according to item 93, and
b) obtaining a value corresponding to the amount of the antibody or antigen-binding fragment thereof or composition that has bound in said subject or to said sample.
103. Method according to item 102, further comprising a step of comparing said value to a reference.

1. A bispecific binding molecule, comprising
a first moiety M1, which is an AβpE3 binding moiety comprising a VH domain and a VL domain, said VH and VL domains forming a VH/VL pair comprising an antigen-binding surface, in which said antigen-binding surface is composed of three complementarity-determining regions (CDRs) from said VH domain and three CDRs from said VL domain, and in which said CDRs consist of the following amino acid sequences:
VHCDR1:
(SEQ ID NO: 1)
GX₁TX₂N
wherein
X₁ is selected from Y and F; and
X₂ is selected from L and M;
VHCDR2:
(SEQ ID NO: 2)
LINPYNGX₃TTYNX₄KFX₅G
wherein
X₃ is selected from I and V
X₄ is selected from P and Q; and
X₅ is selected from M and K;
VHCDR3:
(SEQ ID NO: 3)
EGNWEGVY
VLCDR1:
(SEQ ID NO: 4)
X₆SSQSLLDSNGKTYLH
wherein
X₆ is selected from K and R;
VLCDR2:
(SEQ ID NO: 5)
LVSX₇LDS
wherein
X₇ is selected from I and K; and
VLCDR3:
(SEQ ID NO: 6)
VQGTHFPFT;
and
a second moiety M2, which is a human transferrin receptor 1 (hTfR1) binding moiety comprising an immunoglobulin heavy chain variable domain (VH) and an immunoglobulin light chain variable domain (VL), said VH and VL domains forming a VH/VL pair comprising an antigen-binding surface, in which said antigen-binding surface provides the binding protein with the capacity to bind selectively to an epitope located in the protease-like domain of hTfR1 defined by amino acid residues 121-183 and 384-605 in SEQ ID NO:121.

2. The bispecific binding molecule according to claim 1, wherein the VH domain in M1 comprises an amino acid sequence selected from
i) the group consisting of SEQ ID NO:15-22; and
ii) an amino acid sequence having at least 80% identity to any one of SEQ ID NO:15-22, provided that the three VHCDR regions consist of SEQ ID NO:7, SEQ ID NO:8 and SEQ ID NO:3.

3. The bispecific binding molecule according to claim 2, wherein the M1 VH amino acid sequence in i) is SEQ ID NO:18.

4. The bispecific binding molecule according to claim 1, wherein the VL domain in M1 comprises an amino acid sequence selected from
i) the group consisting of SEQ ID NO:23-24; and
ii) an amino acid sequence having at least 80% identity to any one of SEQ ID NO:23-24, provided that the three VLCDR regions consist of SEQ ID NO:10, SEQ ID NO:9 and SEQ ID NO:6.

5. The bispecific binding molecule according to claim 4, wherein the M1 VL amino acid sequence in i) is SEQ ID NO:23.

6. The bispecific binding molecule according to claim 1, wherein the M1 heavy chain variable domain and the M1 light chain variable domain are represented by the following VH/VL combination:
a) a heavy chain variable domain comprising SEQ ID NO:18 and a light chain variable domain comprising SEQ ID NO:23.

7. The bispecific binding molecule according to claim 1, in which said antigen-binding surface of M2 is composed of three complementarity-determining regions (CDRs) from said VH domain and three CDRs from said VL domain, and in which said CDRs comprise the following:
VHCDR1:
(SEQ ID NO: 37)
X1X2NMX3,
wherein
X1 is selected from D and A;
X2 is selected from Y and A; and
X3 is selected from D and A;
VHCDR2:
(SEQ ID NO: 38)
X4INPX5X6X7TTSX8X9X10KFKG,
wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;
VLCDR1:
(SEQ ID NO: 40)
KSSQSLLX11SX12NX13KNX14LA,
wherein
X11 is selected from Y and A;
X12 is selected from T and S;
X13 is selected from Q and R; and
X14 is selected from Y and A;
VLCDR2:
(SEQ ID NO: 41)
X15ASTRES
wherein
X15 is selected from Wand A; and
VLCDR3:
(SEQ ID NO: 42)
QQX16X17X18X19PX20T
wherein
X16 is selected from Y and A;
X17 is selected from F and Y;
X18 is selected from I and N;
X19 is selected from Y and A; and
X20 is selected from R and Y,
optionally further comprising
VHCDR3:
(SEQ ID NO: 39)
GGX21SGSSX22X23HPMX24X25
wherein
X21 is selected from Y and A;
X22 is selected from Y and A;
X23 is selected from Y and A;
X24 is selected from D and A; and
X25 is selected from Y and A.

8. The bispecific binding molecule according to claim 7, in which the amino acid sequences of the six CDRs in moiety M2 are the following:
VHCDR1:
(SEQ ID NO: 46)
DYNMD,
VHCDR2:
(SEQ ID NO: 57)
DINPDADTTSYNEKFKG,
VHCDR3:
(SEQ ID NO: 48)
GGYSGSSYYHPMDY,
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

9. The bispecific binding molecule according to claim 1, which comprises one first cysteine residue in said VH domain in moiety M2 and one second cysteine residue in said VL domain in moiety M2, said first and second cysteine residues being arranged such that they form a disulfide bridge connecting the VH and VL domains, for example wherein said first cysteine residue is located at M2 VH position 44 and said second cysteine residue is located at M2 VL position 100, as determined by reference to the Kabat numbering scheme.

10. The bispecific binding molecule according to claim 9, wherein said VH domain in moiety M2 comprises or consists of an amino acid sequence selected from
(i) the group consisting of SEQ ID NO:124-139, for example the group consisting of SEQ ID NO:124-137, for example the group consisting of SEQ ID NO:124 and 130; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44.

11. The bispecific binding molecule according to claim 9, wherein said VL domain in moiety M2 comprises or consists of an amino acid sequence selected from
(i) the group consisting of SEQ ID NO:141-149, for example the group consisting of SEQ ID NO:141-147; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106.

12. The bispecific binding molecule according to claim 10, in which said VH domain in moiety M2 comprises SEQ ID NO:130 and said VL domain in moiety M2 comprises SEQ ID NO:141.

13. The bispecific binding molecule according to claim 1, in which the VH/VL pair of the second moiety M2 forms part of an scFv, in which the VH and VL domains are coupled together by a peptide scFv linker, optionally in which said scFv linker is a flexible peptide linker consisting of from 5 to 40 amino acid residues, for example from 10 to 30 amino acid residues, for example from 15 to 25 amino acid residues, for example about 15 amino acid residues, for example 15 amino acid residues, for example comprising or consisting of the sequence (G₄S)₃ (SEQ ID NO:166).

14. The bispecific binding molecule according to claim 1, in which M1 and M2 are connected to each other by at least one peptide linker between M1 and M2.

15. The bispecific binding molecule according to claim 1, in which M1 is provided as a knob-into-hole antibody comprising two identical antibody light chains; one antibody hole heavy chain; and one antibody knob heavy chain; and M2 is provided as an scFv linked to the C-terminal amino acid residue of the knob heavy chain of M1, optionally in which the amino acid sequence of said M1 antibody light chain comprises or consists of SEQ ID NO:160, the amino acid sequence of said M1 antibody hole heavy chain comprises or consists of SEQ ID NO:161, and the amino acid sequence of said M1 antibody knob heavy chain with linked M2 scFv comprises or consists of a sequence selected from SEQ ID NO:162-164.

16. The bispecific binding molecule according to claim 1, in which M1 is provided as a knob-into-hole antibody comprising two identical antibody light chains; one antibody hole heavy chain; and one antibody knob heavy chain; and M2 is provided as an scFv linked to the C-terminal amino acid residue of the knob heavy chain of M1, optionally in which the amino acid sequence of said M1 antibody light chain comprises or consists of SEQ ID NO:160, the amino acid sequence of said M1 antibody hole heavy chain comprises or consists of SEQ ID NO:161, and the amino acid sequence of said M1 antibody knob heavy chain with linked M2 scFv comprises or consists of a sequence selected from SEQ ID NO:189-190.

17. A bispecific binding molecule comprising
(a) an AβpE3 binding moiety comprising:
VHCDR1:
(SEQ ID NO: 7)
GFTMN,
VHCDR2:
(SEQ ID NO: 8)
LINPYNGVTTYNQKFKG,
VHCDR3:
(SEQ ID NO: 3)
EGNWEGVY,
VLCDR1:
(SEQ ID NO: 10)
RSSQSLLDSNGKTYLH,
VLCDR2:
(SEQ ID NO: 9)
LVSILDS,
and
VLCDR3:
(SEQ ID NO: 6)
VQGTHFPFT;
and
(b) a human transferrin receptor 1 (hTfR1) binding moiety comprising:
VHCDR1:
(SEQ ID NO: 46)
DYNMD,
VHCDR2:
(SEQ ID NO: 57)
DINPDADTTSYNEKFKG,
VHCDR3:
(SEQ ID NO: 48)
GGYSGSSYYHPMDY,
VLCDR1:
(SEQ ID NO: 49)
KSSQSLLYSTNQKNYLA,
VLCDR2:
(SEQ ID NO: 50)
WASTRES,
and
VLCDR3:
(SEQ ID NO: 51)
QQYFIYPRT.

18. The bispecific binding molecule of claim 17 comprising (a) an AβpE3 binding moiety comprising a variable heavy chain comprising an amino acid sequence at least 95% identical, or at least 98% identical, or 100% identical to the amino acid sequence of SEQ ID NO:18 and a variable light chain comprising an amino acid sequence at least 95% identical, or at least 98% identical, or 100% identical to the amino acid sequence of SEQ ID NO:23; and (b) a human transferrin receptor 1 (hTfR1) binding moiety comprising: a variable heavy chain comprising an amino acid sequence at least 95% identical, or at least 98% identical, or 100% identical to the amino acid sequence of SEQ ID NO:130 and a variable light chain comprising an amino acid sequence at least 95% identical, or at least 98% identical, or 100% identical to the amino acid sequence of SEQ ID NO:141.

19. The bispecific binding molecule of claim 17 comprising (a) an AβpE3 binding moiety comprising a variable heavy chain comprising an amino acid sequence at least 95% identical to the amino acid sequence of SEQ ID NO:18 and a variable light chain comprising an amino acid sequence at least 95% identical, or at least 98% identical, or 100% identical to the amino acid sequence of SEQ ID NO:23; and (b) a human transferrin receptor 1 (hTfR1) binding moiety comprising: a variable heavy chain comprising an amino acid sequence at least 95% identical, or at least 98% identical, or 100% identical to the amino acid sequence of SEQ ID NO:167 and a variable light chain comprising an amino acid sequence at least 95% identical, or at least 98% identical, or 100% identical to the amino acid sequence of SEQ ID NO:141.

20. The bispecific binding molecule of claim 17 comprising (a) an AβpE3 binding moiety comprising a variable heavy chain comprising an amino acid sequence at least 95% identical to the amino acid sequence of SEQ ID NO:18 and a variable light chain comprising an amino acid sequence at least 95% identical, or at least 98% identical, or 100% identical to the amino acid sequence of SEQ ID NO:23; and (b) a human transferrin receptor 1 (hTfR1) binding moiety comprising: a variable heavy chain comprising an amino acid sequence at least 95% identical, or at least 98% identical, or 100% identical to the amino acid sequence of SEQ ID NO:168 and a variable light chain comprising an amino acid sequence at least 95% identical, or at least 98% identical, or 100% identical to the amino acid sequence of SEQ ID NO:141.

21. The bispecific binding molecule of claim 17, wherein the AβpE3 binding moiety comprises a first and a second IgG heavy chain and two IgG light chains, and wherein the hTfR1 binding moiety comprises an scFv, wherein the scFv is fused, via an optional linker, to the first IgG heavy chain and wherein the first and the second IgG heavy chains are paired via knob-into-hole.

22. The bispecific binding molecule of claim 21, wherein
(i) the first IgG heavy chain comprises the amino acid sequence at least 95% identical, or at least 98% identical, or 100% identical to the amino acid sequence of positions 1 to 446 of the amino acid sequence of SEQ ID NO: 162; and
(ii) the second IgG heavy chain comprises the amino acid sequence at least 95% identical, or at least 98% identical, or 100% identical to the amino acid sequence of SEQ ID NO:161; and
(iii) the two IgG light chains each comprises the amino acid sequence at least 95% identical, or at least 98% identical, or 100% identical the amino acid sequence of SEQ ID NO:160.

23. The bispecific binding molecule of claim 21, wherein
(i) the first IgG heavy chain comprises the amino acid sequence at least 95% identical, or at least 98% identical, or 100% identical to the amino acid sequence of SEQ ID NO:161; and
(ii) the second IgG heavy chain comprises the amino acid sequence at least 95% identical, or at least 98% identical, or 100% identical to the amino acid sequence of positions 1 to 446 of the amino acid sequence of SEQ ID NO:162; and
(iii) the two IgG light chains each comprises the amino acid sequence at least 95% identical, or at least 98% identical, or 100% identical to the amino acid sequence of SEQ ID NO:160.

24. The bispecific binding molecule of claim 21, wherein the scFv comprises the amino acid sequence at least 95% identical, or at least 98% identical, or 100% identical to the amino acid sequence selected from the group consisting of SEQ ID NOs:151-157 and 178-188.

25. The bispecific binding molecule of claim 24, wherein the scFv comprises the amino acid sequence of the amino acid sequence of the SEQ ID NO: 154.

26. The bispecific binding molecule of claim 21, wherein
(i) the first IgG heavy chain fused to the scFv comprises the amino acid sequence of the amino acid sequence consisting of SEQ ID NO:162, and wherein
(ii) the second IgG heavy chain comprises the amino acid sequence of the amino acid sequence consisting of SEQ ID NO:161, and wherein
(iii) the two IgG light chains each comprise the amino acid sequence of the amino acid sequence consisting of SEQ ID NO:160.

27. The bispecific binding molecule of claim 21, wherein
(i) the first IgG heavy chain fused to the scFv comprises the amino acid sequence of the amino acid sequence of SEQ ID NO:162, and wherein
(ii) the second IgG heavy chain comprises the amino acid sequence of the amino acid sequence consisting of SEQ ID NO:161, and wherein
(iii) the two IgG light chains each comprise the amino acid sequence of the amino acid sequence of SEQ ID NO:160.

28. A method of treating or preventing a disease or disorder characterized by the presence of aggregates comprising N-terminally truncated, pyroglutamate-modified amyloid beta peptides in a patient in need thereof, wherein the method comprises to the patient the bispecific binding molecule of claim 1.

29. A pharmaceutical composition comprising the bispecific binding molecule of claim 1, optionally comprising a pharmaceutically acceptable carrier.

30. A set of nucleic acids comprising nucleic acid sequences encoding the bispecific binding molecule of claim 1.

31. A vector comprising the set of nucleic acids of claim 30.

32. A host cell comprising a set of nucleic acids or a vector comprising the set of nucleic acids, wherein the set of nucleic acids comprises nucleic acid sequences encoding the bispecific binding molecule of claim 1.

33. A method of making a bispecific binding molecule of claim 1 wherein the method comprises culturing a host cell under conditions suitable for expression of the bispecific binding molecule, and harvesting the bispecific binding molecule, and purifying the bispecific binding molecule, wherein the host cell comprises a set of nucleic acids or a vector comprising the set of nucleic acids, wherein the set of nucleic acids comprises nucleic acid sequences encoding the bispecific binding molecule of claim 1.
